(19) United States
(12) Patent Application Publication
(10) Pub. No.: US 2020/0069648 A1
(43) Pub. Date: Mar. 5, 2020

(54) HALOALLYLAMINE PYRAZOLE DERIVATIVE INHIBITORS OF LYSYL OXIDASES AND USES THEREOF

(71) Applicant: Pharmaxis Ltd., Frenchs Forest, New South Wales (AU)

(72) Inventors: Alison Dorothy FINDLAY, Frenchs Forest, New South Wales (AU); Craig Ivan TURNER, Frenchs Forest, New South Wales (AU); Mandar DEODHAR, Frenchs Forest, New South Wales (AU); Jonathan Stuart FOOT, Frenchs Forest, New South Wales (AU); Wenbin ZHOU, Frenchs Forest, New South Wales (AU); Wolfgang JAROLIMEK, Frenchs Forest, New South Wales (AU); Alan Duncan ROBERTSON, Warrawee, New South Wales (AU)

(21) Appl. No.: 16/490,220

(22) PCT Filed: Mar. 2, 2018
(86) PCT No.: PCT/AU2018/000010

§ 371 Date: Aug. 30, 2019

(30) Foreign Application Priority Data
Mar. 2, 2017 (AU) 2017900712

(51) Int. Cl. A61K 31/4155; C07D 231/12; A61K 31/415; C07D 231/14; C07D 231/16; A61K 45/06; A61P 1/16; A61K 31/5377; C07D 231/18; C07D 401/06; A61K 31/4439

(52) U.S. Cl. CPC A61K 31/4155; C07D 231/12; A61K 31/415; C07D 231/14; C07D 231/16; A61K 31/4439; A61P 1/16; A61K 31/5377; C07D 231/18; C07D 401/06; A61K 45/06

(57) ABSTRACT

The present invention relates to novel compounds which are capable of inhibiting certain amine oxidase enzymes. These compounds are useful for treatment of a variety of indications, e.g., fibrosis, cancer and/or angiogenesis in human subjects as well as in pets and livestock. In addition, the present invention relates to pharmaceutical compositions containing these compounds, as well as various uses thereof.

### TECHNICAL FIELD

The present invention relates to novel compounds which are capable of inhibiting certain amine oxidase enzymes. These compounds are useful for treatment of a variety of indications, e.g., fibrosis, cancer and/or angiogenesis in human subjects as well as in pets and livestock. In addition, the present invention relates to pharmaceutical compositions containing these compounds, as well as various uses thereof.

### BACKGROUND

A family of five closely relating enzymes have been linked to fibrotic disease and to metastatic cancer. The enzymes are related to lysyl oxidase (LOX), the first family member to be described and four closely related enzymes, LOX-like1 (LOXL1), LOXL2, LOXL3, and LOXL4 (Kagan H. M. and Li W., Lysyl oxidase: properties, specificity, and biological roles inside and outside of the cell. J Cell Biochem 2003; 88: 660-672). Lysyl oxidase isoenzymes are copper-dependent amine oxidases which initiate the covalent cross-linking of collagen and elastin. A major function of lysyl oxidase isoenzymes is to facilitate the cross-linking of collagen and elastin by the oxidative deamination of lysine and hydroxylysine amino acid side chains to aldehydes which spontaneously react with neighbouring residues. The resulting cross-linked strands contribute to extracellular matrix (ECM) stability. Lysyl oxidase activity is essential to maintain the tensile and elastic features of connective tissues of skeletal, pulmonary, and cardiovascular systems, among others. The biosynthesis of LOX is well understood; the protein is synthesized as a pre-proLOX that undergoes a series of post-translational modifications to yield a 50 kDa pro-enzyme which is secreted into the extracellular environment. For LOX and LOXL1 proteolysis by bone morphogenetic protein-1 (BMP-1) and other procollagen C-proteinases releases the mature and active form. LOXL2, LOXL3 and LOXL4 contain scavenger receptor cysteine-rich protein domains and are directly secreted as active forms.

Lysyl oxidase isoenzymes belong to a larger group of amine oxidases which include flavin-dependent and copper-dependent oxidases which are described by the nature of the catalytic co-factor. Flavin-dependent enzymes include monoamine oxidase-A (MAO-A), MAO-B, polyamine oxidase and lysine demethylase (LSD1), and the copper-dependent enzymes include semicarbazide sensitive amine oxidase (vascular adhesion protein-1, SSAO/VAP-1), retinal amine oxidase, diamine oxidase and the lysyl oxidase isoenzymes. The copper-dependent amine oxidases have a second co-factor which varies slightly from enzyme to enzyme. In SSAO/VAP-1 it is an oxidized tyrosine residue (TPQ, oxidized to a quinone), whereas in the lysyl oxidase isoenzymes the TPQ has been further processed by addition of a neighboring lysine residue (to form LTQ); see Kagan, H. M. and Li, W., Lysyl oxidase: Properties, specificity, and biological roles inside and outside of the cell. J Cell Biochem 2003; 88: 660-672.

Since lysyl oxidase isoenzymes exhibit different in vivo expression patterns it is likely that specific isoenzymes will have specific biological roles. Catalytically active forms of LOX have been identified in the cytosolic and nuclear compartments which suggest the existence of undefined roles of LOX in cellular homeostasis. Significant research is currently underway to define these roles. LOX itself, for example, plays a major role in epithelial-to-mesenchymal transition (EMT), cell migration, adhesion, transformation and gene regulation. Different patterns of LOX expression/activity have been associated with distinct pathological processes including fibrotic diseases, Alzheimer's disease and other neurodegenerative processes, as well as tumour progression and metastasis. See, for example, Woznick, A. R., et al. Lysyl oxidase expression in bronchogenic carcinoma. Am J Surg 2005; 189: 297-301. Catalytically active forms of LOXL2 can be also found in the nucleus (J Biol Chem. 2013; 288: 30000-30008) and can deaminate lysine 4 in histone H3 (Mol Cell 2012 46: 369-376).

Directed replacement of dead or damaged cells with connective tissue after injury represents a survival mechanism that is conserved throughout evolution and appears to be most pronounced in humans serving a valuable role following traumatic injury, infection or diseases. Progressive scarring can occur following more chronic and/or repeated injuries that causes impaired function to parts or all of the affected organ. A variety of causes, such as chronic infections, chronic exposure to alcohol and other toxins, autoimmune and allergic reactions or radio- and chemotherapy can all lead to fibrosis. This pathological process, therefore, can occur in almost any organ or tissue of the body and, typically, results from situations persisting for several weeks or months in which inflammation, tissue destruction and repair occur simultaneously. In this setting, fibrosis most frequently affects the lungs, liver, skin and kidneys.

Liver fibrosis occurs as a complication of haemochromatosis, Wilson's disease, alcoholism, schistosomiasis, viral hepatitis, bile duct obstruction, exposure to toxins and metabolic disorders. Liver fibrosis is characterized by the accumulation of extracellular matrix that can be distinguished qualitatively from that in normal liver. This fibrosis can progress to cirrhosis, liver failure, cancer and eventually death. This is reviewed in Kagan, H. M. Lysyl oxidase: Mechanism, regulation and relationship to liver fibrosis. Pathology —Research and Practice 1994; 190: 910-919.

Fibrotic tissues can accumulate in the heart and blood vessels as a result of hypertension, hypertensive heart disease, atherosclerosis and myocardial infarction where the accumulation of extracellular matrix or fibrotic deposition results in stiffening of the vasculature and stiffening of the cardiac tissue itself. See Lopez, B., et al. Role of lysyl oxidase in myocardial fibrosis: from basic science to clinical aspects. Am J Physiol Heart Circ Physiol 2010; 299: H1-H9.

A strong association between fibrosis and increased lysyl oxidase activity has been demonstrated. For example, in experimental hepatic fibrosis in rat (Siegel, R. C., Chen, K. H. and Acquiar, J. M, Biochemical and immunochemical study of lysyl oxidase in experimental hepatic fibrosis in the rat. Proc. Natl. Acad. Sci. USA 1978; 75: 2945-2949), in models of lung fibrosis (Counts, D. F., et al., Collagen lysyl oxidase activity in the lung decreases during bleomycin-induced lung fibrosis. J Pharmacol Exp Ther 1981; 219: 675-678) in arterial fibrosis (Kagan, H. M., Raghavan, J. and Hollander, W., Changes in aortic lysyl oxidase activity in diet-induced atherosclerosis in the rabbit. Arteriosclerosis 1981; 1: 287-291.), in dermal fibrosis (Chanoki, M., et al., Increased expression of lysyl oxidase in skin with scleroderma. Br J Dermatol 1995; 133: 710-715) and in adriamycin-induced kidney fibrosis in rat (Di Donato, A., et al., Lysyl oxidase expression and collagen cross-linking during chronic adriamycin nephropathy. Nephron 1997; 76: 192-200). Of these experimental models of human disease, the most striking increases in enzyme activity are seen in the rat model of CCl₄-induced liver fibrosis. In these studies, the low level of enzyme activity in the healthy liver increased 15- to 30-fold in fibrotic livers. The rationale for the consistent and strong inhibition of fibrosis by lysyl oxidase isoenzyme blockers is that the lack of cross-linking activity renders the collagen susceptible to matrix metalloproteinases and causes degradation. Hence, any type of fibrosis should be reversed by treatment with lysyl oxidase isoenzyme inhibitors. In humans, there is also a significant association between lysyl oxidase activity measured in the plasma and liver fibrosis progression. Lysyl oxidase activity level is normally negligible in the serum of healthy subjects, but significantly increased in chronic active hepatitis and even more in cirrhosis, therefore lysyl oxidase might serve as a marker of internal fibrosis.

BAPN (3-aminopropionitrile) is a widely used, nonselective lysyl oxidase inhibitor. Since the 1960s BAPN has been used in animal studies (mainly rat, mouse and hamster) and has been efficacious in reducing collagen content in various models (eg. CCl₄, bleomycin, quartz) and tissues (eg. liver, lung and dermis). See Kagan, H. M. and Li, W., Lysyl oxidase: Properties, specificity and biological roles inside and outside of the cell. J Cell Biochem 2003; 88: 660-672.

Lysyl oxidase isoenzymes are highly regulated by Hypoxia-Induced Factor 1α (HIF-1α) and TGF-β, the two most prominent growth factor that cause fibrosis (Halberg et al., Hypoxia-inducible factor 1α induces fibrosis and insulin resistance in white adipose tissue. Cell Biol 2009; 29: 4467-4483). Collagen cross linking occurs in every type of fibrosis, hence a lysyl oxidase isoenzyme inhibitor could be used in idiopathic pulmonary fibrosis, scleroderma, kidney or liver fibrosis. Lysyl oxidase isoenzymes are not only involved in the cross-linking of elastin and collagen during wound healing and fibrosis but also regulate cell movement and signal transduction. Its intracellular and intranuclear function is associated with gene regulation and can lead to tumorgenesis and tumor progression (Siddikiuzzaman, Grace, V. M and Guruvayoorappan, C., Lysyl oxidase: a potential target for cancer therapy. Inflammapharmacol 2011; 19: 117-129). Both down and upregulation of lysyl oxidase isoenzymes in tumour tissues and cancer cell lines have been described, suggesting a dual role for lysyl oxidase isoenzymes and LOX pro-peptide as a metastasis promoter gene as well as a tumour suppressor gene.

To date, an increase in lysyl oxidase isoenzymes mRNA and/or protein has been observed in breast, CNS cancer cell lines, head and neck squamous cell, prostatic, clear cell renal cell and lung carcinomas, and in melanoma and osteosarcoma cell lines. Statistically significant clinical correlations between lysyl oxidase isoenzymes expression and tumor progression have been observed in breast, head and neck squamous cell, prostatic and clear cell renal cell carcinomas. The role of lysyl oxidase isoenzymes in tumor progression has been most extensively studied in breast cancer using in vitro models of migration/invasion and in in vivo tumorgenesis and metastasis mouse models. Increased lysyl oxidase isoenzymes expression was found in hypoxic patients, and was associated with negative estrogen receptor status (ER−), decreased overall survival in ER− patients and node-negative patients who did not receive adjuvant systemic treatment, as well as shorter metastasis-free survival in ER− patients and node negative patients. Lysyl oxidase isoenzymes mRNA was demonstrated to be up-regulated in invasive and metastatic cell lines (MDA-MB-231 and Hs578T), as well as in more aggressive breast cancer cell lines and distant metastatic tissues compared with primary cancer tissues.

In head and neck squamous cell carcinomas, increased lysyl oxidase isoenzyme expression was found in association with CA-IX, a marker of hypoxia, and was associated with decreased cancer specific survival, decreased overall survival and lower metastasis-free survival. In oral squamous cell carcinoma, lysyl oxidase isoenzyme mRNA expression was upregulated compared to normal mucosa.

Gene expression profiling of gliomas identified over-expressed lysyl oxidase isoenzyme as part of a molecular signature indicative of invasion, and associated with higher-grade tumors that are strongly correlated with poor patient survival. Lysyl oxidase isoenzyme protein expression was increased in glioblastoma and astrocytoma tissues, and in invasive U343 and U251 cultured astrocytoma cells.

In tissues, lysyl oxidase isoenzyme mRNA was upregulated in prostate cancer compared to benign prostatic hypertrophy, correlated with Gleason score, and associated with both high grade and short time to recurrence (Stewart, G. D., et al., Analysis of hypoxia-associated gene expression in prostate cancer: lysyl oxidase and glucose transporter-I expression correlate with Gleason score. Oncol Rep 2008; 20: 1561-1567).

Up-regulation of lysyl oxidase isoenzyme mRNA expression was detected in renal cell carcinoma (RCC) cell lines and tissues. Clear cell RCC also demonstrated lysyl oxidase isoenzyme up-regulation. Indeed, LOX over expression appeared preferentially in clear cell RCC compared to mixed clear and granular, granular, oxyphil, tubulopapillary and chromophobe RCC/ontocytomas. In clear cell RCC, smoking was associated with allelic imbalances at chromosome 5q23.1, where the LOX gene is localized, and may involve duplication of the gene.

SiHa cervical cancer cells demonstrated increased invasion in vitro under hypoxic/anoxic conditions; this was repressed by inhibition of extracellular catalytically active lysyl oxidase activity by treatment with BAPN as well as LOX antisense oligos, LOX antibody, LOX shRNA or an extracellular copper chelator.

The scientific and patent literature describes small molecule inhibitors of lysyl oxidase isoenzymes and antibodies of LOX and LOXL2 with therapeutic effects in animal models of fibrosis and cancer metastasis. Some known MAO inhibitors also are reported to inhibit lysyl oxidase isoenzyme (e.g., the MAO-B inhibitor Mofegiline illustrated below). This inhibitor is a member of the haloallylamine family of MAO inhibitors; the halogen in Mofegiline is fluorine. Fluoroallylamine inhibitors are described in U.S. Pat. No. 4,454,158. There are issued patents claiming fluoroallylamines and chloroallylamines, for example MDL72274 (illustrated below) as inhibitors of lysyl oxidase (U.S. Pat. Nos. 4,943,593; 4,965,288; 5,021,456; 5,059,714; 5,182,297; 5,252,608). Many of the compounds claimed in these patents are also reported to be potent MAO-B and SSAO/VAP-1 inhibitors.

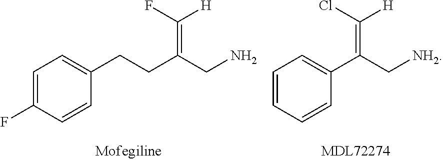

Additional fluoroallylamine inhibitors are described U.S. Pat. No. 4,699,928. Other examples structurally related to Mofegiline can be found in WO 2007/120528.

WO 2009/066152 discloses a family of 3-substituted 3-haloallylamines that are inhibitors of SSAO/VAP-I useful as treatment for a variety of indications, including inflammatory disease. None of these documents specifically disclose the fluoroallylamine compounds of formula (I) according to the present invention.

Antibodies to LOX and LOXL2 have been disclosed in US 2009/0053224 with methods to diagnostic and therapeutic applications. Anti-LOX and anti-LOXL2 antibodies can be used to identify and treat conditions such as a fibrotic condition, angiogenesis, or to prevent a transition from an epithelial cell state to a mesenchymal cell state: US 2011/0044907.

### SUMMARY

The present invention provides substituted fluoroallylamine compounds that inhibit lysyl oxidase (LOX), lysyl oxidase-like2 (LOXL2) and other lysyl oxidase isoenzymes. Surprisingly, modification of 3-substituted-3-fluoroallylamine structures described previously has led to the discovery of novel compounds that are potent inhibitors of the human LOX and LOXL isoenzymes. Furthermore, certain of these novel compounds also selectively inhibit certain LOX and LOXL isoenzymes with respect to the other enzymes in the amine oxidase family.

A first aspect of the invention provides for a compound of Formula I:

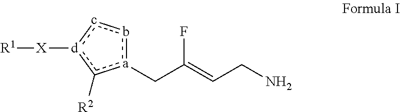

or a stereoisomer, pharmaceutically acceptable salt, polymorphic form, solvate, tautomeric form or prodrug thereof; wherein:

each [symbol] is independently a single or double bond arranged so as to provide a pyrazole ring;

a is C or N;

b is C(R³) or N;

c is C(R⁴) or N;

d is C or N;

and 2 of a, b, c and d are N, wherein the 2 N atoms are adjacent to each other;

R², R³ and R⁴ are independently selected from the group consisting of hydrogen, halogen, C₁₋₄alkyl, —C₃₋₅cycloalkyl, —O—C₁₋₄alkyl, —O—C₃₋₅cycloalkyl, —C(O)OR⁵, —C(O)NR⁶R⁷ and —NR⁶C(O)R⁸; wherein each C₁₋₄alkyl is a straight or branched chain alkyl; and wherein each C₁₋₄alkyl and C₃₋₅cycloalkyl is optionally substituted by one or more substituents selected from the group consisting of halogen, —OH, —SH, —C₁₋₃alkyl, —O—C₁₋₃alkyl, —CF₃, —CH₂CF₃ and —O—CF₃;

X is O or —(CH₂)ₘ—;

R¹ is selected from the group consisting of aryl and heteroaryl; wherein each R¹ is optionally substituted by one or more R⁹;

R⁵ is selected from the group consisting of hydrogen, —C₁₋₆alkyl, and —C₃₋₇cycloalkyl; wherein each C₁₋₆alkyl is a straight or branched chain alkyl, and wherein each C₁₋₆alkyl, and C₃₋₇cycloalkyl is optionally substituted by one or more substituents selected from the group consisting of halogen, —OH, —SH, —C₁₋₃alkyl, —O—C₁₋₃alkyl, —CF₃, —CH₂CF₃ and —O—CF₃;

R⁶ and R⁷ are independently selected from the group consisting of hydrogen, C₁₋₄alkyl and C₃₋₇cycloalkyl; wherein each C₁₋₆alkyl is a straight or branched chain alkyl; and wherein each C₁₋₆alkyl and C₃₋₇cycloalkyl is optionally substituted by one or more substituents selected from the group consisting of halogen, —OH, —SH, —C₁₋₃alkyl, —O—C₁₋₃alkyl, —CF₃, —CH₂CF₃, and —O—CF₃; or

R⁶ and R⁷ when attached to the same nitrogen atom are combined to form a 3- to 7-membered ring having from 0 to 2 additional heteroatoms as ring members;

R⁸ is selected from the group consisting of C₁₋₆alkyl and C₃₋₇cycloalkyl; wherein each C₁₋₆alkyl is a straight or branched chain alkyl; and wherein each C₁₋₆alkyl and C₃₋₇cycloalkyl is optionally substituted by one or more substituents selected from the group consisting of halogen, —OH, —SH, —C₁₋₃alkyl, —O—C₁₋₃alkyl, —CF₃, —CH₂CF₃, and —O—CF₃; and

each R⁹ is independently selected from the group consisting of halogen, C₁₋₆alkyl, —O—C₁₋₆alkyl, —S—C₁₋₆alkyl, C₃₋₇cycloalkyl, —O—C₃₋₇cycloalkyl, —C(O)OR⁵, —C(O)NR⁶R⁷, —NR⁶C(O)R⁸, —S(O₂)NR⁶R⁷, —NR⁶S(O₂)R⁸, —S(O)R⁸ and —S(O₂)R⁸; wherein each C₁₋₆alkyl is a straight or branched chain alkyl; and wherein each C₁₋₄alkyl and C₃₋₇cycloalkyl is optionally substituted by one or more substituents selected from the group consisting of halogen, —OH, —C₁₋₃alkyl, —O—C₁₋₃alkyl, —CF₃, —CH₂CF₃, and —O—CF₃; and

m is 0 or 1.

A second aspect of the invention provides for a pharmaceutical composition comprising a compound according to the first aspect of the invention, or a pharmaceutically acceptable salt, solvate or prodrug thereof, and at least one pharmaceutically acceptable excipient, carrier or diluent.

A third aspect of the invention provides for a method of inhibiting the amine oxidase activity of LOX, LOXL1, LOXL2, LOXL3 and LOXL4 in a subject in need thereof, comprising administering to the subject an effective amount of a compound according to the first aspect of the invention, or a pharmaceutically acceptable salt, solvate or prodrug thereof, or a pharmaceutical composition according to the second aspect of the invention.

A fourth aspect of the invention provides for a method of treating a condition associated with LOX, LOXL1, LOXL2, LOXL3 and LOXL4 protein, comprising administering to a subject in need thereof a therapeutically effective amount of compound according to the first aspect of the invention, or a pharmaceutically acceptable salt, solvate or prodrug thereof, or a pharmaceutical composition according to the second aspect of the invention.

A fifth aspect of the invention provides for use of a compound according to the first aspect of the invention, or a pharmaceutically acceptable salt, solvate or prodrug thereof, for the manufacture of a medicament for treating a condition associated with LOX, LOXL1, LOXL2, LOXL3 and LOXL4 protein.

A sixth aspect of the invention provides for a compound according to the first aspect of the invention, or a pharmaceutically acceptable salt, solvate or prodrug thereof, for use in treating a condition associated with LOX, LOXL1, LOXL2, LOXL3 and LOXL4 protein.

In one embodiment of the methods and uses of the present invention the condition is selected from a liver disorder, kidney disorder, cardiovascular disease, fibrosis, cancer and angiogenesis.

Contemplated herein is combination therapy in which the methods further comprise co-administering additional therapeutic agents that are used for the treatment of liver disorders, kidney disorders, cardiovascular diseases, cancer, fibrosis, angiogenesis and inflammation.

### Definitions

The following are some definitions that may be helpful in understanding the description of the present invention. These are intended as general definitions and should in no way limit the scope of the present invention to those terms alone, but are put forth for a better understanding of the following description.

Unless the context requires otherwise or specifically states to the contrary, integers, steps, or elements of the invention recited herein as singular integers, steps or elements clearly encompass both singular and plural forms of the recited integers, steps or elements.

Throughout this specification, unless the context requires otherwise, the word “comprise”, or variations such as “comprises” or “comprising”, will be understood to imply the inclusion of a stated step or element or integer or group of steps or elements or integers, but not the exclusion of any other step or element or integer or group of elements or integers. Thus, in the context of this specification, the term “comprising” means “including principally, but not necessarily solely”.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations or any two or more of said steps or features.

As used herein, the term “alkyl” includes within its meaning monovalent (“alkyl”) and divalent (“alkylene”) straight chain or branched chain saturated hydrocarbon radicals having from 1 to 6 carbon atoms, e.g., 1, 2, 3, 4, 5 or 6 carbon atoms. The straight chain or branched alkyl group is attached at any available point to produce a stable compound. For example, the term alkyl includes, but is not limited to, methyl, ethyl, 1-propyl, isopropyl, 1-butyl, 2-butyl, isobutyl, tert-butyl, amyl, 1,2-dimethylpropyl, 1,1-dimethylpropyl, pentyl, isopentyl, hexyl, 4-methylpentyl, 1-methylpentyl, 2-methylpentyl, 3-methylpentyl, 2,2-dimethylbutyl, 3,3-dimethylbutyl, 1,2-dimethylbutyl, 1,3-dimethylbutyl, 1,2,2-trimethylpropyl, 1,1,2-trimethylpropyl, and the like.

The term “alkoxy” or “alkyloxy” as used herein refers to straight chain or branched alkyloxy (i.e, O-alkyl) groups, wherein alkyl is as defined above. Examples of alkoxy groups include methoxy, ethoxy, n-propoxy, and isopropoxy.

The term “cycloalkyl” as used herein includes within its meaning monovalent (“cycloalkyl”) and divalent (“cycloalkylene”) saturated, monocyclic, bicyclic, polycyclic or fused analogs. In the context of the present disclosure the cycloalkyl group may have from 3 to 10 carbon atoms. In the context of the present disclosure the cycloalkyl group may also have from 3 to 7 carbon atoms. A fused analog of a cycloalkyl means a monocyclic ring fused to an aryl or heteroaryl group in which the point of attachment is on the non-aromatic portion. Examples of cycloalkyl and fused analogs thereof include cyclopropyl, cyclobutyl, cyclopentyl, cyclohexyl, cycloheptyl, tetrahydronaphthyl, decahydronaphthyl, indanyl, adamantyl and the like.

The term “aryl” or variants such as “arylene” as used herein refers to monovalent (“aryl”) and divalent (“arylene”) single, polynuclear, conjugated and fused analogs of aromatic hydrocarbons having from 6 to 10 carbon atoms. A fused analog of aryl means an aryl group fused to a monocyclic cycloalkyl or monocyclic heterocyclyl group in which the point of attachment is on the aromatic portion. Examples of aryl and fused analogs thereof include phenyl, naphthyl, indanyl, indenyl, tetrahydronaphthyl, 2,3-dihydrobenzofuranyl, dihydrobenzopyranyl, 1,3-benzodioxolyl, 1,4-benzodioxanyl, and the like. A “substituted aryl” is an aryl that is independently substituted, with one or more, preferably 1, 2 or 3 substituents, attached at any available atom to produce a stable compound.

The term “alkylaryl” as used herein, includes within its meaning monovalent (“aryl”) and divalent (“arylene”), single, polynuclear, conjugated and fused aromatic hydrocarbon radicals attached to divalent, saturated, straight or branched chain alkylene radicals. Examples of alkylaryl groups include benzyl.

The term “heteroaryl” and variants such as “heteroaromatic group” or “heteroarylene” as used herein, includes within its meaning monovalent (“heteroaryl”) and divalent (“heteroarylene”), single, polynuclear, conjugated and fused heteroaromatic radicals having from 5 to 10 atoms, wherein 1 to 4 ring atoms, or 1 to 2 ring atoms are heteroatoms independently selected from O, N, NH and S. Heteroaryl is also intended to include oxidized S or N, such as sulfinyl, sulfonyl and N-oxide of a tertiary ring nitrogen. A carbon or nitrogen atom is the point of attachment of the heteroaryl ring structure such that a stable compound is produced. The heteroaromatic group may be C₅₋₈ heteroaromatic. A fused analog of heteroaryl means a heteroaryl group fused to a monocyclic cycloalkyl or monocyclic heterocyclyl group in which the point of attachment is on the aromatic portion. Examples of heteroaryl groups and fused analogs thereof include pyrazolyl, pyridyl, oxazolyl, oxadiazolyl, thiadiazolyl, tetrazolyl, triazinyl, thienyl, benzoxazolyl, benzothiazolyl, benzimidazolyl, benzofuranyl, benzothiophenyl, furo(2,3-b)pyridyl, quinolyl, indolyl, isoquinolyl, pyrimidinyl, pyridazinyl, pyrazinyl, 2,2′-bipyridyl, phenanthrolinyl, quinolinyl, isoquinolinyl, imidazolinyl, thiazolinyl, pyrrolyl, furanyl, thiophenyl, oxazolyl, isoxazolyl, isothiazolyl, triazolyl, and the like. “Nitrogen containing heteroaryl” refers to heteroaryl wherein any heteroatoms are N. A “substituted heteroaryl” is a heteroaryl that is independently substituted, with one or more, preferably 1, 2 or 3 substituents, attached at any available atom to produce a stable compound.

The term “heterocyclyl” and variants such as “heterocycloalkyl” as used herein, includes within its meaning monovalent (“heterocyclyl”) and divalent (“heterocyclylene”), saturated, monocyclic, bicyclic, polycyclic or fused hydrocarbon radicals having from 3 to 10 ring atoms, wherein from 1 to 5, or from 1 to 3, ring atoms are heteroatoms independently selected from O, N, NH, or S, in which the point of attachment may be carbon or nitrogen. A fused analog of heterocyclyl means a monocyclic heterocycle fused to an aryl or heteroaryl group in which the point of attachment is on the non-aromatic portion. The heterocyclyl group may be C₃₋₈ heterocyclyl. The heterocycloalkyl group may be C₃₋₆ heterocyclyl. The heterocyclyl group may be C₃₋₅ heterocyclyl. Examples of heterocyclyl groups and fused analogs thereof include aziridinyl, pyrrolidinyl, thiazolidinyl, piperidinyl, piperazinyl, imidazolidinyl, 2,3-dihydrofuro(2,3-b)pyridyl, benzoxazinyl, tetrahydroquinolinyl, tetrahydroisoquinolinyl, dihydroindolyl, quinuclidinyl, azetidinyl, morpholinyl, tetrahydrothiophenyl, tetrahydrofuranyl, tetrahydropyranyl, and the like. The term also includes partially unsaturated monocyclic rings that are not aromatic, such as 2- or 4-pyridones attached through the nitrogen or N-substituted uracils.

The term “halogen” or variants such as “halide” or “halo” as used herein refers to fluorine, chlorine, bromine and iodine.

The term “heteroatom” or variants such as “hetero-” or “heterogroup” as used herein refers to O, N, NH and S.

In general, “substituted” refers to an organic group as defined herein (e.g., an alkyl group) in which one or more bonds to a hydrogen atom contained therein are replaced by a bond to non-hydrogen or non-carbon atoms. Substituted groups also include groups in which one or more bonds to a carbon(s) or hydrogen(s) atom are replaced by one or more bonds, including double or triple bonds, to a heteroatom. Thus, a substituted group will be substituted with one or more substituents, unless otherwise specified. In some embodiments, a substituted group is substituted with 1, 2, 3, 4, 5, or 6 substituents.

The term “optionally substituted” as used herein means the group to which this term refers may be unsubstituted, or may be substituted with one or more groups independently selected from alkyl, alkenyl, alkynyl, cycloalkyl, cycloalkenyl, heterocycloalkyl, halo, haloalkyl, haloalkynyl, hydroxyl, hydroxyalkyl, alkoxy, thioalkoxy, alkenyloxy, haloalkoxy, haloalkenyloxy, NO₂, NH(alkyl), N(alkyl)₂, nitroalkyl, nitroalkenyl, nitroalkynyl, nitroheterocyclyl, alkylamino, dialkylamino, alkenylamine, alkynylamino, acyl, alkenoyl, alkynoyl, acylamino, diacylamino, acyloxy, alkylsulfonyloxy, heterocycloxy, heterocycloamino, haloheterocycloalkyl, alkylsulfenyl, alkylcarbonyloxy, alkylthio, acylthio, phosphorus-containing groups such as phosphono and phosphinyl, aryl, heteroaryl, alkylaryl, aralkyl, alkylheteroaryl, cyano, cyanate, isocyanate, CO₂H, CO₂alkyl, C(O)NH₂, —C(O)NH(alkyl), and —C(O)N(alkyl)₂. Preferred substituents include halogen, C₁-C₆alkyl, C₂-C₆alkenyl, C₁-C₆haloalkyl, C₁-C₆alkoxy, hydroxy(C₁₋₆)alkyl, C₃-C₆cycloalkyl, C(O)H, C(O)OH, NHC(O)H, NHC(O)C₁-C₄alkyl, C(O)C₁-C₄alkyl, NH₂, NHC₁-C₄alkyl, N(C₁-C₄alkyl)₂, NO₂, OH and CN. Particularly preferred substituents include C₁₋₃alkyl, C₁₋₃alkoxy, halogen, OH, hydroxy(C₁₋₃)alkyl (e.g. CH₂OH), C(O)C₁-C₄alkyl (e.g. C(O)CH₃), and C₁₋₃haloalkyl (e.g. CF₃, CH₂CF₃). Further preferred optional substituents include halogen, —OH, —SH, —C₁₋₃alkyl, —O—C₁₋₃alkyl, —CF₃, —CH₂CF₃, and —O—CF₃.

The term “bioisostere” refers to a compound resulting from the exchange of an atom or of a group of atoms with another, broadly similar, atom or group of atoms. The objective of a bioiosteric replacement is to create a new compound with similar biological properties to the parent compound. The bioisosteric replacement may be physiochemically or topologically based.

The present invention includes within its scope all stereoisomeric and isomeric forms of the compounds disclosed herein, including all diastereomeric isomers, racemates, enantiomers and mixtures thereof. It is also understood that the compounds described by Formula I may be present as E and Z isomers, also known as cis and trans isomers. Thus, the present disclosure should be understood to include, for example, E, Z, cis, trans, (R), (S), (L), (D), (+), and/or (−) forms of the compounds, as appropriate in each case. Where a structure has no specific stereoisomerism indicated, it should be understood that any and all possible isomers are encompassed. Compounds of the present invention embrace all conformational isomers. Compounds of the present invention may also exist in one or more tautomeric forms, including both single tautomers and mixtures of tautomers. Also included in the scope of the present invention are all polymorphs and crystal forms of the compounds disclosed herein.

The present invention includes within its scope isotopes of different atoms. Any atom not specifically designated as a particular isotope is meant to represent any stable isotope of that atom. Thus, the present disclosure should be understood to include deuterium and tritium isotopes of hydrogen.

All references cited in this application are specifically incorporated by cross-reference in their entirety. Reference to any such documents should not be construed as an admission that the document forms part of the common general knowledge or is prior art.

In the context of this specification the term “administering” and variations of that term including “administer” and “administration”, includes contacting, applying, delivering or providing a compound or composition of the invention to an organism, or a surface by any appropriate means. In the context of this specification, the term “treatment”, refers to any and all uses which remedy a disease state or symptoms, prevent the establishment of disease, or otherwise prevent, hinder, retard, or reverse the progression of disease or other undesirable symptoms in any way whatsoever.

In the context of this specification the term “effective amount” includes within its meaning a sufficient but non-toxic amount of a compound or composition of the invention to provide a desired effect. Thus, the term “therapeutically effective amount” includes within its meaning a sufficient but non-toxic amount of a compound or composition of the invention to provide the desired therapeutic effect. The exact amount required will vary from subject to subject depending on factors such as the species being treated, the sex, age and general condition of the subject, the severity of the condition being treated, the particular agent being administered, the mode of administration, and so forth. Thus, it is not possible to specify an exact “effective amount”. However, for any given case, an appropriate “effective amount” may be determined by one of ordinary skill in the art using only routine experimentation.

### BRIEF DESCRIPTION OF THE FIGURES

FIG. 1 shows the ability of Compound 15 to reduce fibrosis in a mouse model of liver fibrosis.

FIG. 2 shows the ability of Compound 15 to reduce tongue cancer volume in a mouse model of oral metastatic cancer.

### DETAILED DESCRIPTION

The present invention relates to substituted fluoroallylamine derivatives which may inhibit lysyl oxidase (LOX), lysyl oxidase-like2 (LOXL2) and other lysyl oxidase isoenzymes. In particular the present invention relates to substituted fluoroallylamine derivatives with a pyrazole group.

In particular the present invention relates to compounds of Formula I:

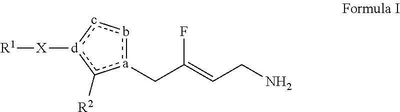

or a stereoisomer, pharmaceutically acceptable salt, polymorphic form, solvate, tautomeric form or prodrug thereof; wherein:

each [symbol] is independently a single or double bond arranged so as to provide a pyrazole ring;

a is C or N;

b is C(R³) or N;

c is C(R⁴) or N;

d is C or N;

and 2 of a, b, c and d are N, wherein the 2 N atoms are adjacent to each other;

R², R³ and R⁴ are independently selected from the group consisting of hydrogen, halogen, C₁₋₄alkyl, —C₃₋₅cycloalkyl, —O—C₁₋₄alkyl, —O—C₃₋₅cycloalkyl, —C(O)OR⁵, —C(O)NR⁶R⁷ and —NR⁶C(O)R⁸; wherein each C₁₋₄alkyl is a straight or branched chain alkyl; and wherein each C₁₋₄alkyl and C₃₋₅cycloalkyl is optionally substituted by one or more substituents selected from the group consisting of halogen, —OH, —SH, —C₁₋₃alkyl, —O—C₁₋₃alkyl, —CF₃, —CH₂CF₃ and —O—CF₃;

X is O or —(CH₂)ₘ—;

R¹ is selected from the group consisting of aryl and heteroaryl; wherein each R¹ is optionally substituted by one or more R⁹;

R⁵ is selected from the group consisting of hydrogen, —C₁₋₆alkyl, and —C₃₋₇cycloalkyl; wherein each C₁₋₆alkyl is a straight or branched chain alkyl, and wherein each C₁₋₆alkyl, and C₃₋₇cycloalkyl is optionally substituted by one or more substituents selected from the group consisting of halogen, —OH, —SH, —C₁₋₃alkyl, —O—C₁₋₃alkyl, —CF₃, —CH₂CF₃ and —O—CF₃;

R⁶ and R⁷ are independently selected from the group consisting of hydrogen, C₁₋₆alkyl and C₃₋₇cycloalkyl; wherein each C₁₋₆alkyl is a straight or branched chain alkyl; and wherein each C₁₋₆alkyl and C₃₋₇cycloalkyl is optionally substituted by one or more substituents selected from the group consisting of halogen, —OH, —SH, —C₁₋₃alkyl, —O—C₁₋₃alkyl, —CF₃, —CH₂CF₃, and —O—CF₃; or

R⁶ and R⁷ when attached to the same nitrogen atom are combined to form a 3- to 7-membered ring having from 0 to 2 additional heteroatoms as ring members;

R⁸ is selected from the group consisting of C₁₋₆alkyl and C₃₋₇cycloalkyl; wherein each C₁₋₆alkyl is a straight or branched chain alkyl; and wherein each C₁₋₆alkyl and C₃₋₇cycloalkyl is optionally substituted by one or more substituents selected from the group consisting of halogen, —OH, —SH, —C₁₋₃alkyl, —O—C₁₋₃alkyl, —CF₃, —CH₂CF₃, and —O—CF₃; and

each R⁹ is independently selected from the group consisting of halogen, C₁₋₄alkyl, —O—C₁₋₆alkyl, —S—C₁₋₆alkyl, C₃₋₇cycloalkyl, —O—C₃₋₇cycloalkyl, —C(O)OR⁵, —C(O)NR⁶R⁷, —NR⁶C(O)R⁸, —S(O₂)NR⁶R⁷, —NR⁶S(O₂)R⁸, —S(O)R⁸ and —S(O₂)R⁸; wherein each C₁₋₆alkyl is a straight or branched chain alkyl; and wherein each C₁₋₆alkyl and C₃₋₇cycloalkyl is optionally substituted by one or more substituents selected from the group consisting of halogen, —OH, —C₁₋₃alkyl, —O—C₁₋₃alkyl, —CF₃, —CH₂CF₃, and —O—CF₃; and

m is 0 or 1.

In one embodiment of compounds of the present invention a and b are N, c is C(R⁴) and d is C. In another embodiment of compounds of the present invention a is C, b is C(R³) and c and d are N.

In one embodiment of compounds of the present invention, the pyrazole ring in Formula I is represented by

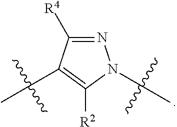

In another embodiment of compounds of the present invention, the pyrazole ring in Formula I is represented by

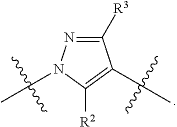

In one embodiment of compounds of the present invention R², R³ and R⁴ are independently selected from the group consisting of hydrogen, halogen, C₁₋₄alkyl, —C₃₋₅cycloalkyl, —O—C₁₋₄alkyl, —O—C₃₋₅cycloalkyl, —C(O)OR⁵, —C(O)NR⁶R⁷ and —NR⁶C(O)R⁸; wherein each C₁₋₄alkyl is a straight or branched chain alkyl; and wherein each C₁₋₄alkyl and C₃₋₅cycloalkyl is optionally substituted by one or more substituents selected from the group consisting of halogen, —OH, —SH, —C₁₋₃alkyl, —O—C₁₋₃alkyl, —CF₃, —CH₂CF₃ and —O—CF₃. In another embodiment of compounds of the present invention R², R³ and R⁴ are independently selected from the group consisting of hydrogen, halogen, C₁₋₄alkyl, —C(O)OR⁵, and —C(O)NR⁶R⁷; wherein each C₁₋₄alkyl is a straight or branched chain alkyl; and wherein each C₁₋₄alkyl is optionally substituted by one or more substituents selected from the group consisting of halogen, —OH and —O—C₁₋₃alkyl. In a further embodiment of compounds of the present invention R², R³ and R⁴ are independently selected from the group consisting of hydrogen, chlorine, methyl, ethyl, isopropyl, tert-butyl, —CF₃, —CH₂OH, CHOHCH₃, —C(CH₃)₂OH, —C(O)OEt, —C(O)OH, —C(O)N(CH₃)₂, —C(O)NHC(CH₃)₃, —CHCH₃OH and —CH₂OCH₃.

In one embodiment of compounds of the present invention X is O or —(CH₂)ₘ—; m is 0 or 1. In another embodiment of compounds of the present invention X is O. In a further embodiment of compounds of the present invention —(CH₂)ₘ— and m is 0 or 1. In one embodiment of compounds of present invention m is 1 so X is —CH₂—. In another embodiment of the present invention m is 0 so X is a bond between d and R¹.

In one embodiment of compounds of the present invention R¹ is aryl or heteroaryl where each R¹ is optionally substituted by one or more R⁹. In another embodiment of compounds of the present invention R¹ is aryl optionally substituted by one or more R⁹. In another embodiment of compounds of the present invention R¹ is phenyl substituted by one R⁹. In a further embodiment of compounds of the present invention R¹ is heteroaryl substituted by one or more R⁹. In a further embodiment of compounds of the present invention R¹ is selected from the group consisting of phenyl, naphthyl and pyridyl; substituted by one or more R⁹.

In one embodiment of compounds of the present invention R¹ is substituted by one R⁹. In another embodiment of compounds of the present invention R¹ is substituted by two R⁹. In another embodiment of compounds of the present invention R¹ is substituted by one or two R⁹. In a further embodiment of compounds of the present invention R¹ is substituted by three R⁹. In another embodiment of compounds of the present invention R¹ is substituted by four or five R⁹.

In one embodiment of compounds of the present invention R⁵ is selected from the group consisting of hydrogen, C₁₋₄alkyl and C₃₋₇cycloalkyl; wherein each C₁₋₆alkyl is a straight or branched chain alkyl; and wherein each C₁₋₆alkyl and C₃₋₇ cycloalkyl is optionally substituted by one or more substituents selected from the group consisting of halogen, —OH, —SH, —C₁₋₃alkyl, —O—C₁₋₃alkyl, —CF₃, —CH₂CF₃, and —O—CF₃. In another embodiment of compounds of the present invention R⁵ is hydrogen. In a further embodiment of compounds of the present invention R⁵ is C₁₋₆alkyl or C₃₋₇cycloalkyl. In a still further embodiment of compounds of the present invention R⁵ is hydrogen or C₁₋₆alkyl. In another embodiment of compounds of the present invention R⁵ is C₁₋₆alkyl. In another embodiment of compounds of the present invention R⁵ is C₁₋₃alkyl. In a further embodiment of compounds of the present invention R⁵ is methyl or ethyl. In another embodiment of compounds of the present invention R⁵ is selected from the group consisting of hydrogen, methyl and ethyl. In a further embodiment of compounds of the present invention R⁵ is hydrogen or ethyl.

In one embodiment of compounds of the present invention R⁶ and R⁷ are independently selected from the group consisting of hydrogen, C₁₋₆alkyl and C₃₋₇cycloalkyl; wherein each C₁₋₆alkyl is a straight or branched chain alkyl; and wherein each C₁₋₆alkyl and C₃₋₇cycloalkyl is optionally substituted by one or more substituents selected from the group consisting of halogen, —OH, —SH, —C₁₋₃alkyl, —O—C₁₋₃alkyl, —CF₃, —CH₂CF₃, and —O—CF₃. In another embodiment of compounds of the present invention R⁶ and R⁷ are independently selected from the group consisting of hydrogen, C₁₋₆alkyl and C₃₋₇cycloalkyl. In another embodiment of compounds of the present invention R⁶ and R⁷ are independently selected from the group consisting of hydrogen and C₁₋₆alkyl. In another embodiment of compounds of the present invention R⁶ and R⁷ are hydrogen. In a further embodiment of compounds of the present invention R⁶ and R⁷ are C₁₋₆alkyl. In another embodiment of compounds of the present invention R⁶ and R⁷ are both methyl. In a further embodiment of compounds of the present invention R⁶ and R¹ are independently selected from the group consisting of hydrogen and C₃₋₇cycloalkyl. In another embodiment of compounds of the present invention R⁶ is hydrogen and R⁷ is C₁₋₆alkyl. In one embodiment of compounds of the present invention R⁶ is hydrogen and R⁷ is methyl or isopropyl. In a further embodiment of compounds of the present invention R⁶ is methyl and R⁷ is isopropyl.

In one embodiment of compounds of the present invention R⁶ and R⁷ when attached to the same nitrogen atom are combined to form a 3- to 7-membered ring having from 0 to 2 additional heteroatoms as ring members. In another embodiment R⁶ and R⁷ when attached to the same nitrogen atom are combined to form a 3- to 7-membered ring having from 0 to 1 additional heteroatoms as ring members. In a further embodiment R⁶ and R⁷ when attached to the same nitrogen atom are combined to form a 3- to 7-membered ring having 1 additional heteroatom as ring members. In another embodiment R⁶ and R⁷ when attached to the same nitrogen atom are combined to form a 3- to 7-membered ring having 0 additional heteroatoms as ring members. In a further embodiment R⁶ and R⁷ when attached to the same nitrogen atom are combined to form a pyrrolidine ring. In another embodiment R⁶ and R⁷ when attached to the same nitrogen atom are combined to form a morpholine ring.

In one embodiment of compounds of the present invention R¹ is selected from the group consisting of C₁₋₆alkyl and C₃₋₇cycloalkyl; wherein each C₁₋₆alkyl is a straight or branched chain alkyl; and wherein each C₁₋₆alkyl and C₃₋₇cycloalkyl is optionally substituted by one or more substituents selected from the group consisting of halogen, —OH, —SH, —C₁₋₃alkyl, —O—C₁₋₃alkyl, —CF₃, —CH₂CF₃, and —O—CF₃. In another embodiment of compounds of the present invention R¹ is selected from the group consisting of C₁₋₄alkyl and C₃₋₇cycloalkyl. In another embodiment of compounds of the present invention R¹ is C₁₋₆alkyl. In a further embodiment of compounds of the present invention R¹ is selected from the group consisting of methyl, ethyl and isopropyl. In another embodiment of compounds of the present invention R¹ is methyl. In a further embodiment of compounds of the present invention R¹ is C₃₋₇cycloalkyl.

In one embodiment of compounds of the present invention each R⁹ is independently selected from the group consisting of halogen, C₁₋₆alkyl, —O—C₁₋₆alkyl, —S—C₁₋₆alkyl, C₃₋₇cycloalkyl, —O—C₃₋₇cycloalkyl, —C(O)OR⁵, —C(O)NR⁶R⁷, —NR⁶C(O)R⁸, —S(O₂)NR⁶R⁷, —NR⁶S(O₂)R⁸, —S(O)R⁸ and —S(O₂)R⁸; wherein each C₁₋₆alkyl is a straight or branched chain alkyl; and wherein each C₁₋₆alkyl and C₃₋₇cycloalkyl is optionally substituted by one or more substituents selected from the group consisting of halogen, —OH, —C₁₋₃alkyl, —O—C₁₋₃alkyl, —CF₃, —CH₂CF₃, and —O—CF₃. In a further embodiment of compounds of the present invention each R⁹ is independently selected from the group consisting of halogen, C₁₋₆alkyl, —O—C₁₋₆alkyl, —C(O)NR⁶R⁷, —S(O₂)NR⁶R⁷, and —S(O₂)R⁸; wherein each C₁₋₆alkyl is a straight or branched chain alkyl; and wherein each C₁₋₆alkyl is optionally substituted by one or more halogen. In another embodiment of compounds of the present invention each R⁹ is independently selected from the group consisting of fluorine, chlorine, CF₃, —OCF₃, —C(O)N(CH₃)₂, —S(O₂)NR⁶R⁷, —S(O₂)CF₃, —S(O₂)CH(CH₃)₂ and —S(O₂)CH₃. In a further embodiment of compounds of the present invention one R¹ is selected from the group consisting of —S(O₂)NR⁶R⁷ and —S(O₂)R⁸. In another embodiment of compounds of the present invention one R⁹ is —S(O₂)NR⁶R⁷. In a further embodiment of compounds of the present invention one R⁹ is —S(O₂)N(CH₃)₂.

In one embodiment the present invention also relates to compounds of Formula Ia

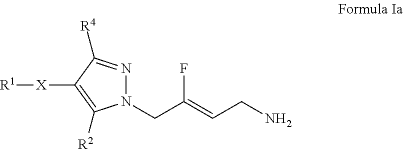

or a pharmaceutically acceptable salt or solvate thereof, wherein:

R² and R⁴ are independently selected from the group consisting of hydrogen, halogen, C₁₋₄alkyl, —C₃₋₅cycloalkyl, —O—C₁₋₄alkyl, —O—C₃₋₅cycloalkyl, —C(O)OR⁵, —C(O)NR⁶R⁷ and —NR⁶C(O)R⁸; wherein each C₁₋₄alkyl is a straight or branched chain alkyl; and wherein each C₁₋₄alkyl and C₃₋₅cycloalkyl is optionally substituted by one or more substituents selected from the group consisting of halogen, —OH, —SH, —C₁₋₃alkyl, —O—C₁₋₃alkyl, —CF₃, —CH₂CF₃ and —O—CF₃;

X is 0 or —(CH₂)ₘ—;

R¹ is selected from the group consisting of aryl and heteroaryl; wherein each R¹ is optionally substituted by one or more R⁹;

R⁵ is selected from the group consisting of hydrogen, —C₁₋₄alkyl, and —C₃₋₇cycloalkyl;

wherein each C₁₋₆alkyl is a straight or branched chain alkyl, and wherein each C₁₋₄alkyl, and C₃₋₇cycloalkyl is optionally substituted by one or more substituents selected from the group consisting of halogen, —OH, —SH, —C₁₋₃alkyl, —O—C₁₋₃alkyl, —CF₃, —CH₂CF₃ and —O—CF₃;

R⁶ and R⁷ are independently selected from the group consisting of hydrogen, C₁₋₆alkyl and C₃₋₇cycloalkyl; wherein each C₁₋₆alkyl is a straight or branched chain alkyl; and wherein each C₁₋₆alkyl and C₃₋₇cycloalkyl is optionally substituted by one or more substituents selected from the group consisting of halogen, —OH, —SH, —C₁₋₃alkyl, —O—C₁₋₃alkyl, —CF₃, —CH₂CF₃, and —O—CF₃; or

R⁶ and R⁷ when attached to the same nitrogen atom are combined to form a 3- to 7-membered ring having from 0 to 2 additional heteroatoms as ring members;

R⁸ is selected from the group consisting of C₁₋₆alkyl and C₃₋₇cycloalkyl; wherein each C₁₋₆alkyl is a straight or branched chain alkyl; and wherein each C₁₋₆alkyl and C₃₋₇cycloalkyl is optionally substituted by one or more substituents selected from the group consisting of halogen, —OH, —SH, —C₁₋₃alkyl, —O—C₁₋₃alkyl, —CF₃, —CH₂CF₃, and —O—CF₃; and

each R⁹ is independently selected from the group consisting of halogen, C₁₋₆alkyl, —O—C₁₋₄alkyl, —S—C₁₋₆alkyl, C₃₋₇cycloalkyl, —O—C₃₋₇cycloalkyl, —C(O)OR⁵, —C(O)NR⁶R⁷, —NR⁶C(O)R⁸, —S(O₂)NR⁶R⁷, —NR⁶S(O₂)R⁸, —S(O)R⁵ and —S(O₂)R⁸; wherein each C₁₋₆alkyl is a straight or branched chain alkyl; and wherein each C₁₋₆alkyl and C₃₋₇cycloalkyl is optionally substituted by one or more substituents selected from the group consisting of halogen, —OH, —C₁₋₃alkyl, —O—C₁₋₃alkyl, —CF₃, —CH₂CF₃, and —O—CF₃; and

m is 0 or 1.

In one embodiment of compounds of Formula Ia of the invention m is 0 so X is a direct bond; R² and R⁴ are independently selected from hydrogen, methyl and chlorine; and R¹ is phenyl substituted by —S(O₂)N(CH₃)₂.

In another embodiment of compounds of Formula Ia of the invention X is CH₂; R² and R⁴ are independently selected from are independently selected from the group consisting of hydrogen, halogen, C₁₋₄alkyl, —C(O)OR⁵, and —C(O)NR⁶R⁷; wherein each C₁₋₄alkyl is a straight or branched chain alkyl; and wherein each C₁₋₄alkyl is optionally substituted by one or more substituents selected from the group consisting of halogen, —OH and —O—C₁₋₃alkyl; R¹ is phenyl, naphthyl or pyridyl substituted by one or more R⁹; R⁵ is hydrogen or C₁₋₆alkyl; R⁶ and R⁷ are independently selected from the group consisting of hydrogen and C₁₋₆alkyl; wherein each C₁₋₆alkyl is a straight or branched chain alkyl; or R⁶ and R⁷ when attached to the same nitrogen atom are combined to form a 3- to 7-membered ring having from 0 to 2 additional heteroatoms as ring members; R⁸ is C₁₋₆alkyl; and each R⁹ is independently selected from the group consisting of halogen, C₁₋₆alkyl, —O—C₁₋₆alkyl, —C(O)NR⁶R⁷, —S(O₂)NR⁶R⁷, and —S(O₂)R⁸; wherein each C_{1-b}alkyl is a straight or branched chain alkyl; and wherein each C₁₋₆alkyl is optionally substituted by one or more halogen. In a further embodiment of compounds of Formula Ia of the invention X is CH₂; R² and R⁴ are independently selected from are independently selected from the group consisting of chlorine, methyl, ethyl, isopropyl, tert-butyl, —C(O)OEt, —C(O)OH, —C(O)N(CH₃)₂, —CHCH₃OH and —CH₂OCH₃; R¹ is phenyl, naphthyl or pyridyl substituted by one or more R⁹; R⁵ is hydrogen or ethyl; R⁶ and R⁷ are independently selected from the group consisting of hydrogen, methyl, and isopropyl; or R⁶ and R⁷ when attached to the same nitrogen atom are combined to form a pyrrolidine or morpholine ring; and each R⁹ is independently selected from the group consisting of fluorine, chlorine, —CF₃, —OCF₃, —C(O)N(CH₃)₂, —S(O₂)NR⁶R⁷, and —S(O₂)CH₃.

In another embodiment the present invention also relates to compounds of Formula Ib

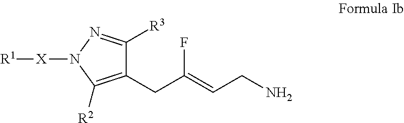

or a pharmaceutically acceptable salt or solvate thereof, wherein:

R² and R³ are independently selected from the group consisting of hydrogen, halogen, C₁₋₄alkyl, —C₃₋₅cycloalkyl, —O—C₁₋₄alkyl, —O—C₃₋₅cycloalkyl, —C(O)OR⁵, —C(O)NR⁶R⁷ and —NR⁶C(O)R⁸; wherein each C₁₋₄alkyl is a straight or branched chain alkyl; and wherein each C₁₋₄alkyl and C₃₋₅cycloalkyl is optionally substituted by one or more substituents selected from the group consisting of halogen, —OH, —SH, —C₁₋₃alkyl, —O—C₁₋₃alkyl, —CF₃, —CH₂CF₃ and —O—CF₃;

X is O or —(CH₂)ₘ—;

R¹ is selected from the group consisting of aryl and heteroaryl; wherein each R¹ is optionally substituted by one or more R⁹;

R⁵ is selected from the group consisting of hydrogen, —C₁₋₆alkyl, and —C₃₋₇cycloalkyl; wherein each C₁₋₆alkyl is a straight or branched chain alkyl, and wherein each C₁₋₆alkyl, and C₃₋₇cycloalkyl is optionally substituted by one or more substituents selected from the group consisting of halogen, —OH, —SH, —C₁₋₃alkyl, —O—C₁₋₃alkyl, —CF₃, —CH₂CF₃ and —O—CF₃;

R⁶ and R⁷ are independently selected from the group consisting of hydrogen, C₁₋₆alkyl and C₃₋₇cycloalkyl; wherein each C₁₋₆alkyl is a straight or branched chain alkyl; and wherein each C₁₋₆alkyl and C₃₋₇cycloalkyl is optionally substituted by one or more substituents selected from the group consisting of halogen, —OH, —SH, —C₁₋₃alkyl, —O—C₁₋₃alkyl, —CF₃, —CH₂CF₃, and —O—CF₃; or

R⁶ and R⁷ when attached to the same nitrogen atom are combined to form a 3- to 7-membered ring having from 0 to 2 additional heteroatoms as ring members;

R⁸ is selected from the group consisting of C₁₋₆alkyl and C₃₋₇cycloalkyl; wherein each C₁₋₆alkyl is a straight or branched chain alkyl; and wherein each C₁₋₆alkyl and C₃₋₇cycloalkyl is optionally substituted by one or more substituents selected from the group consisting of halogen, —OH, —SH, —C₁₋₃alkyl, —O—C₁₋₃alkyl, —CF₃, —CH₂CF₃, and —O—CF₃; and

each R⁹ is independently selected from the group consisting of halogen, C₁₋₆alkyl, —O—C₁₋₆alkyl, —S—C₁₋₆alkyl, C₃₋₇cycloalkyl, —O—C₃₋₇cycloalkyl, —C(O)OR⁵, —C(O)NR⁶R⁷, —NR⁶C(O)R, —S(O₂)NR⁶R⁷, —NR⁶S(O₂)R⁸, —S(O)R⁸ and —S(O₂)R⁵; wherein each C₁₋₆alkyl is a straight or branched chain alkyl; and wherein each C₁₋₆alkyl and C₃₋₇cycloalkyl is optionally substituted by one or more substituents selected from the group consisting of halogen, —OH, —C₁₋₃alkyl, —O—C₁₋₃alkyl, —CF₃, —CH₂CF₃, and —O—CF₃; and

m is 0 or 1.

In one embodiment of compounds of Formula Ib of the invention X is CH₂; R² and R³ are methyl; R¹ is phenyl substituted by one or more R⁹; R⁶ and R⁷ are independently selected from the group consisting of hydrogen and methyl; and each R⁹ is independently selected from the group consisting of fluorine, chlorine, —CF₃, —S(O₂)NR⁶R⁷, and —S(O₂)CH₃.

In the context of the present disclosure, any one or more aspect(s) or embodiment(s) may be combined with any other aspect(s) or embodiment(s).

Exemplary compounds according to the present invention include the compounds set forth in Table 1:

TABLE 1

| | | | m/z |
|---|---|---|---|
| No. | Structure | Name | [M + H]+ |
| 1 | 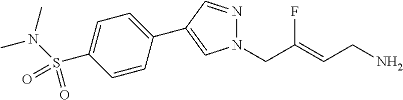 [SMILES: CN(C)S(=O)(=O)c1ccc(-c2cnn(C/C(F)=C/CN)c2)cc1] | (Z)-4-(1-(4-amino-2-fluorobut-2- en-1-yl)-1H-pyrazol-4-yl)-N,N- dimethylbenzenesulfonamide | 339.0 |
| 2 | 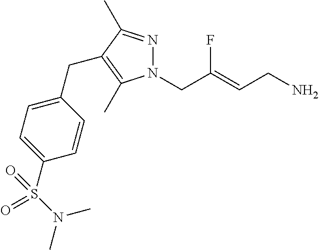 [SMILES: Cc1nn(C/C(F)=C/CN)c(C)c1Cc1ccc(S(=O)(=O)N(C)C)cc1] | (Z)-4-((1-(4-amino-2-fluorobut-2- en-1-yl)-3,5-dimethyl-1H-pyrazol- 4-yl)methyl)-N,N- dimethylbenzenesulfonamide | 381.0 |
| 3 | 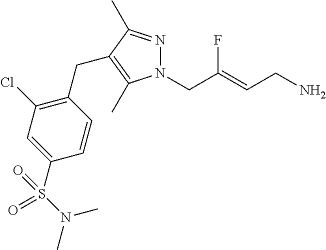 [SMILES: Cc1nn(C/C(F)=C/CN)c(C)c1Cc1ccc(S(=O)(=O)N(C)C)cc1Cl] | (Z)-4-((1-(4-amino-2-fluorobut-2- en-1-yl)-3,5-dimethyl-1H-pyrazol- 4-yl)methyl)-3-chloro-N,N- dimethylbenzenesulfonamide | 415.0 |
| 4 | 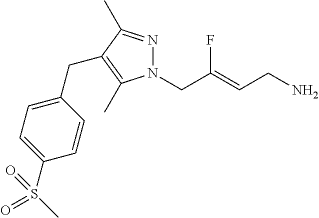 [SMILES: Cc1nn(C/C(F)=C/CN)c(C)c1Cc1ccc(S(C)(=O)=O)cc1] | (Z)-4-(3,5-dimethyl-4-(4- (methylsulfonyl)benzyl)-1H- pyrazol-1-yl)-3-fluorobut-2-en-1- amine | 352.0 |
| 5 | 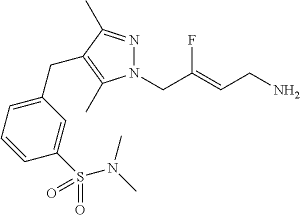 [SMILES: Cc1nn(C/C(F)=C/CN)c(C)c1Cc1cccc(S(=O)(=O)N(C)C)c1] | (Z)-3-((1-(4-amino-2-fluorobut-2- en-1-yl)-3,5-dimethyl-1H-pyrazol- 4-yl)methyl)-N,N- dimethylbenzenesulfonamide | 381.0 |
| 6 | 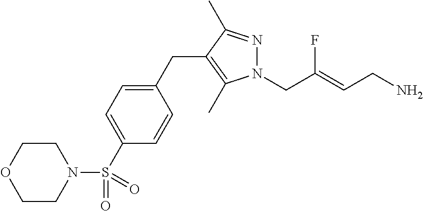 [SMILES: Cc1nn(C/C(F)=C/CN)c(C)c1Cc1ccc(S(=O)(=O)N2CCOCC2)cc1] | (Z)-4-(3,5-dimethyl-4-(4- (morpholinosulfonyl)benzyl)-1H- pyrazol-1-yl)-3-fluorobut-2-en-1- amine | 423.2 |
| 7 | 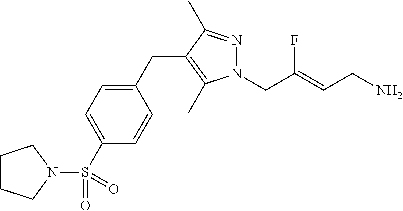 [SMILES: Cc1nn(C/C(F)=C/CN)c(C)c1Cc1ccc(S(=O)(=O)N2CCCC2)cc1] | (Z)-4-(3,5-dimethyl-4-(4- (pyrrolidin-1-ylsulfonyl)benzyl)- 1H-pyrazol-1-yl)-3-fluorobut-2-en- 1-amine | 407.2 |
| 8 | 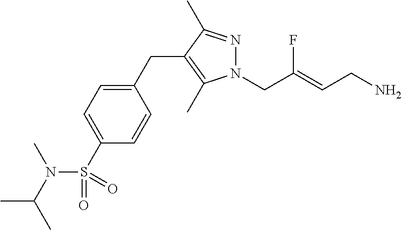 [SMILES: Cc1nn(C/C(F)=C/CN)c(C)c1Cc1ccc(S(=O)(=O)N(C)C(C)C)cc1] | (Z)-4-((1-(4-amino-2-fluorobut-2- en-1-yl)-3,5-dimethyl-1H-pyrazol- 4-yl)methyl)-N-isopropyl-N- methylbenzenesulfonamide | 409.2 |
| 9 | 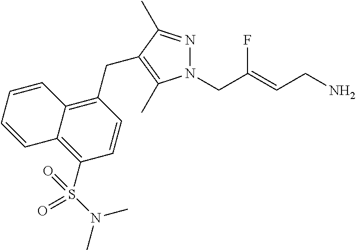 [SMILES: Cc1nn(C/C(F)=C/CN)c(C)c1Cc1ccc(S(=O)(=O)N(C)C)c2ccccc12] | (Z)-4-((1-(4-amino-2-fluorobut-2- en-1-yl)-3,5-dimethyl-1H-pyrazol- 4-yl)methyl)-N,N- dimethylnaphthalene-1- sulfonamide | 431.2 |
| 10 | 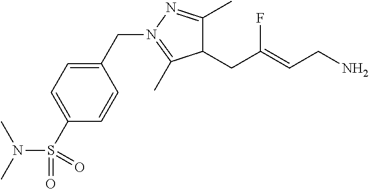 | (Z)-4-((4-(4-amino-2-fluorobut-2- en-1-yl)-3,5-dimethyl-1H-pyrazol- 1-yl)methyl)-N,N- dimethylbenzenesulfonamide | 381.0 |
| 11 | 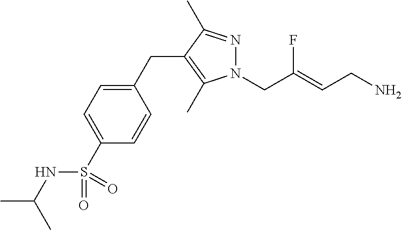 [SMILES: Cc1nn(C/C(F)=C/CN)c(C)c1Cc1ccc(S(=O)(=O)NC(C)C)cc1] | (Z)-4-((1-(4-amino-2-fluorobut-2- en-1-yl)-3,5-dimethyl-1H-pyrazol- 4-yl)methyl)-N- isopropylbenzenesulfonamide | 395.0 |
| 12 | 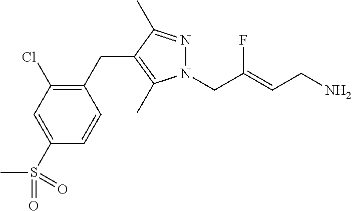 [SMILES: Cc1nn(C/C(F)=C/CN)c(C)c1Cc1ccc(S(C)(=O)=O)cc1Cl] | (Z)-4-(4-(2-chloro-4- (methylsulfonyl)benzyl)-3,5- dimethyl-1H-pyrazol-1-yl)-3- fluorobut-2-en-1-amine | 387.0 |
| 13 | 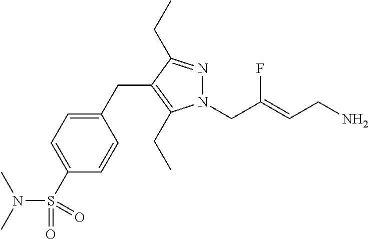 [SMILES: CCc1nn(C/C(F)=C/CN)c(CC)c1Cc1ccc(S(=O)(=O)N(C)C)cc1] | (Z)-4-((1-(4-amino-2-fluorobut-2- en-1-yl)-3,5-diethyl-1H-pyrazol-4- yl)methyl)-N,N- dimethylbenzenesulfonamide | 409.2 |
| 14 | 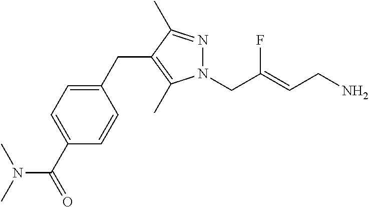 [SMILES: Cc1nn(C/C(F)=C/CN)c(C)c1Cc1ccc(C(=O)N(C)C)cc1] | (Z)-4-((1-(4-amino-2-fluorobut-2- en-1-yl)-3,5-dimethyl-1H-pyrazol- 4-yl)methyl)-N,N- dimethylbenzamide | 345.2 |
| 15 | 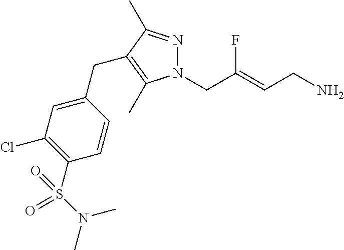 [SMILES: Cc1nn(C/C(F)=C/CN)c(C)c1Cc1ccc(S(=O)(=O)N(C)C)c(Cl)c1] | (Z)-4-((1-(4-amino-2-fluorobut-2- en-1-yl)-3,5-dimethyl-1H-pyrazol- 4-yl)methyl)-2-chloro-N,N- dimethylbenzenesulfonamide | 415.2 |
| 16 | 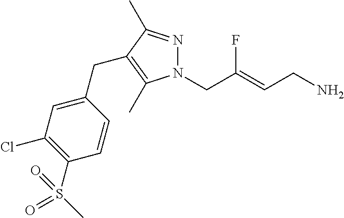 [SMILES: Cc1nn(C/C(F)=C/CN)c(C)c1Cc1ccc(S(C)(=O)=O)c(Cl)c1] | (Z)-4-(4-(3-chloro-4- (methylsulfonyl)benzyl)-3,5- dimethyl-1H-pyrazol-1-yl)-3- fluorobut-2-en-1-amine | 386.0 |
| 17 | 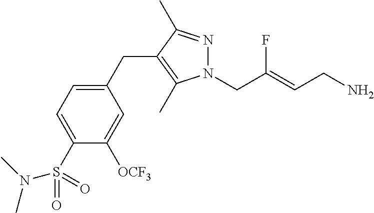 [SMILES: Cc1nn(C/C(F)=C/CN)c(C)c1Cc1ccc(S(=O)(=O)N(C)C)c(OC(F)(F)F)c1] | (Z)-4-((1-(4-amino-2-fluorobut-2- en-1-yl)-3,5-dimethyl-1H-pyrazol- 4-yl)methyl)-N,N-dimethyl-2- (trifluoromethoxy)benzene- sulfonamide | 465.0 |
| 18 | 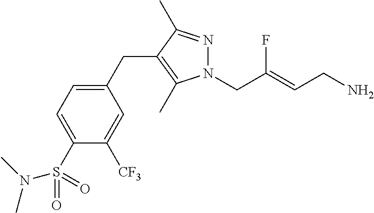 [SMILES: Cc1nn(C/C(F)=C/CN)c(C)c1Cc1ccc(S(=O)(=O)N(C)C)c(C(F)(F)F)c1] | (Z)-4-((1-(4-amino-2-fluorobut-2- en-1-yl)-3,5-dimethyl-1H-pyrazol- 4-yl)methyl)-N,N-dimethyl-2- (trifluoromethyl)benzene- sulfonamide | 449.0 |
| 19 | 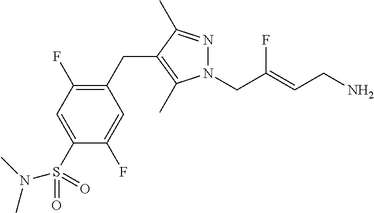 [SMILES: Cc1nn(C/C(F)=C/CN)c(C)c1Cc1cc(F)c(S(=O)(=O)N(C)C)cc1F] | (Z)-4-((1-(4-amino-2-fluorobut-2- en-1-yl)-3,5-dimethyl-1H-pyrazol- 4-yl)methyl)-2,5-difluoro-N,N- dimethylbenzenesulfonamide | 417.0 |
| 20 | 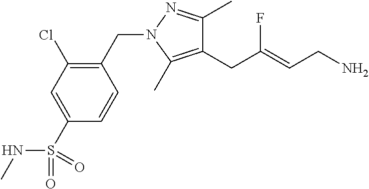 [SMILES: CNS(=O)(=O)c1ccc(Cn2nc(C)c(C/C(F)=C/CN)c2C)c(Cl)c1] | (Z)-4-((4-(4-amino-2-fluorobut-2- en-1-yl)-3,5-dimethyl-1H-pyrazol- 1-yl)methyl)-3-chloro-N- methylbenzenesulfonamide | 401.0 |
| 21 | 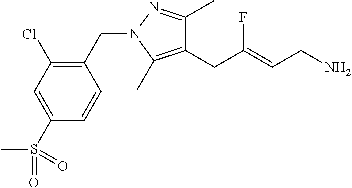 [SMILES: Cc1nn(Cc2ccc(S(C)(=O)=O)cc2Cl)c(C)c1C/C(F)=C/CN] | (Z)-4-(1-(2-chloro-4- (methylsulfonyl)benzyl)-3,5- dimethyl-1H-pyrazol-4-yl)-3- fluorobut-2-en-1-amine | 386.0 |
| 22 | 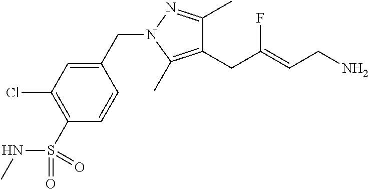 [SMILES: CNS(=O)(=O)c1ccc(Cn2nc(C)c(C/C(F)=C/CN)c2C)cc1Cl] | (Z)-4-((4-(4-amino-2-fluorobut-2- en-1-yl)-3,5-dimethyl-1H-pyrazol- 1-yl)methyl)-2-chloro-N- methylbenzenesulfonamide | 401.0 |
| 23 | 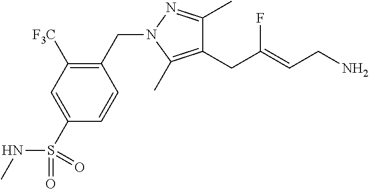 [SMILES: CNS(=O)(=O)c1ccc(Cn2nc(C)c(C/C(F)=C/CN)c2C)c(C(F)(F)F)c1] | (Z)-4-((4-(4-amino-2-fluorobut-2- en-1-yl)-3,5-dimethyl-1H-pyrazol- 1-yl)methyl)-N-methyl-3- (trifluoromethyl)benzene sulfonamide | 435.0 |
| 24 | 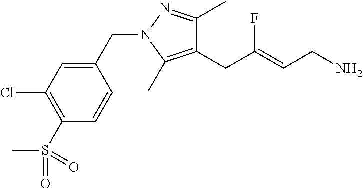 [SMILES: Cc1nn(Cc2ccc(S(C)(=O)=O)c(Cl)c2)c(C)c1C/C(F)=C/CN] | (Z)-4-(1-(3-chloro-4- (methylsulfonyl)benzyl)-3,5- dimethyl-1H-pyrazol-4-yl)-3- fluorobut-2-en-1-amine | 386.0 |
| 25 | 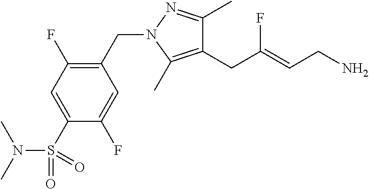 [SMILES: Cc1nn(Cc2cc(F)c(S(=O)(=O)N(C)C)cc2F)c(C)c1C/C(F)=C/CN] | (Z)-4-((4-(4-amino-2-fluorobut-2- en-1-yl)-3,5-dimethyl-1H-pyrazol- 1-yl)methyl)-2,5-difluoro-N,N- dimethylbenzenesulfonamide | 417.0 |
| 26 | 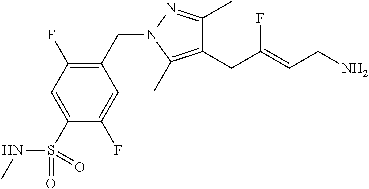 [SMILES: CNS(=O)(=O)c1cc(F)c(Cn2nc(C)c(C/C(F)=C/CN)c2C)cc1F] | (Z)-4-((4-(4-amino-2-fluorobut-2- en-1-yl)-3,5-dimethyl-1H-pyrazol- 1-yl)methyl)-2,5-difluoro-N- methylbenzenesulfonamide | 403.0 |
| 27 | 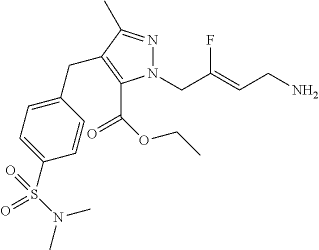 [SMILES: CCOC(=O)c1c(Cc2ccc(S(=O)(=O)N(C)C)cc2)c(C)nn1C/C(F)=C/CN] | ethyl (Z)-1-(4-amino-2-fluorobut-2- en-1-yl)-4-(4-(N,N- dimethylsulfamoyl)benzyl)-3- methyl-1H-pyrazole-5-carboxylate | 439.0 |
| 28 | 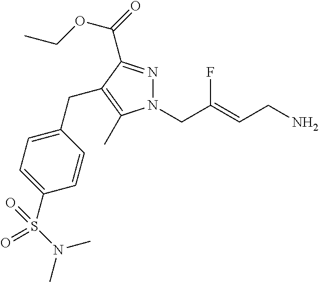 [SMILES: CCOC(=O)c1nn(C/C(F)=C/CN)c(C)c1Cc1ccc(S(=O)(=O)N(C)C)cc1] | ethyl (Z)-1-(4-amino-2-fluorobut-2- en-1-yl)-4-(4-(N,N- dimethylsulfamoyl)benzyl)-5- methyl-1H-pyrazole-3-carboxylate | 439.0 |
| 29 | 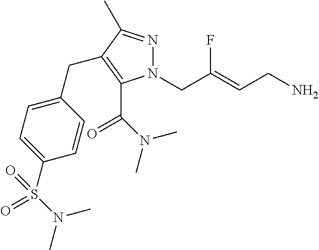 [SMILES: Cc1nn(C/C(F)=C/CN)c(C(=O)N(C)C)c1Cc1ccc(S(=O)(=O)N(C)C)cc1] | (Z)-1-(4-amino-2-fluorobut-2-en-1- yl)-4-(4-(N,N- dimethylsulfamoyl)benzyl)-N,N,3- trimethyl-1H-pyrazole-5- carboxamide | 438.2 |
| 30 | 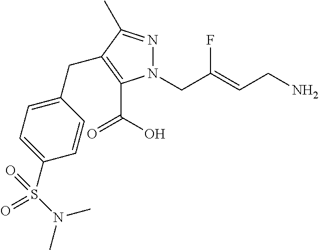 [SMILES: Cc1nn(C/C(F)=C/CN)c(C(=O)O)c1Cc1ccc(S(=O)(=O)N(C)C)cc1] | (Z)-1-(4-amino-2-fluorobut-2-en-1- yl)-4-(4-(N,N- dimethylsulfamoyl)benzyl)-3- methyl-1H-pyrazole-5-carboxylic acid | 411.3 |
| 31 | 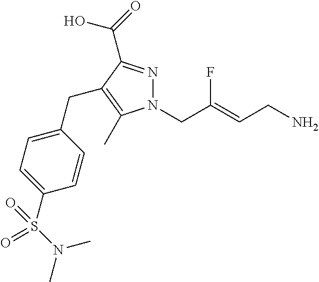 [SMILES: Cc1c(Cc2ccc(S(=O)(=O)N(C)C)cc2)c(C(=O)O)nn1C/C(F)=C/CN] | (Z)-1-(4-amino-2-fluorobut-2-en-1- yl)-4-(4-(N,N- dimethylsulfamoyl)benzyl)-5- methyl-1H-pyrazole-3-carboxylic acid | 411.4 |
| 32 | 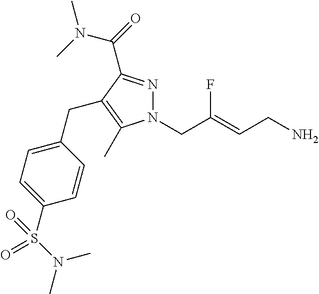 [SMILES: Cc1c(Cc2ccc(S(=O)(=O)N(C)C)cc2)c(C(=O)N(C)C)nn1C/C(F)=C/CN] | (Z)-1-(4-amino-2-fluorobut-2-en-1- yl)-4-(4-(N,N- dimethylsulfamoyl)benzyl)-N,N,5- trimethyl-1H-pyrazole-3- carboxamide | 438.4 |
| 33 | 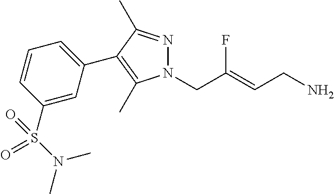 [SMILES: Cc1nn(C/C(F)=C/CN)c(C)c1-c1cccc(S(=O)(=O)N(C)C)c1] | (Z)-3-(1-(4-amino-2-fluorobut-2- en-1-yl)-3,5-dimethyl-1H-pyrazol- 4-yl)-N,N- dimethylbenzenesulfonamide | 367.2 |
| 34 | 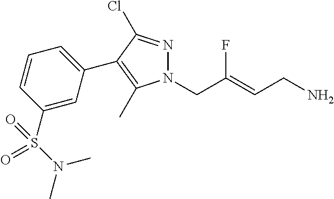 [SMILES: Cc1c(-c2cccc(S(=O)(=O)N(C)C)c2)c(Cl)nn1C/C(F)=C/CN] | (Z)-3-(1-(4-amino-2-fluorobut-2- en-1-yl)-3-chloro-5-methyl-1H- pyrazol-4-yl)-N,N- dimethylbenzenesulfonamide | 387.0 |
| 35 | 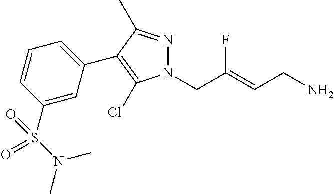 [SMILES: Cc1nn(C/C(F)=C/CN)c(Cl)c1-c1cccc(S(=O)(=O)N(C)C)c1] | (Z)-3-(1-(4-amino-2-fluorobut-2- en-1-yl)-5-chloro-3-methyl-1H- pyrazol-4-yl)-N,N- dimethylbenzenesulfonamide | 387.0 |
| 36 | 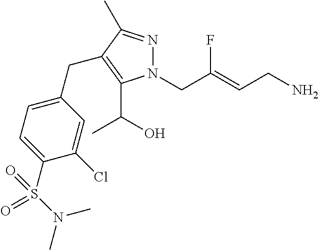 [SMILES: Cc1nn(C/C(F)=C/CN)c(C(C)O)c1Cc1ccc(S(=O)(=O)N(C)C)c(Cl)c1] | (Z)-4-((1-(4-amino-2-fluorobut-2- en-1-yl)-5-(1-hydroxyethyl)-3- methyl-1H-pyrazol-4-yl)methyl)-2- chloro-N,N- dimethylbenzenesulfonamide | 445.2 |
| 37 | 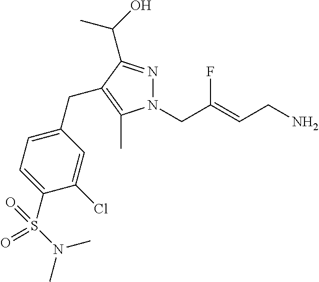 [SMILES: Cc1c(Cc2ccc(S(=O)(=O)N(C)C)c(Cl)c2)c(C(C)O)nn1C/C(F)=C/CN] | (Z)-4-((1-(4-amino-2-fluorobut-2- en-1-yl)-3-(1-hydroxyethyl)-5- methyl-1H-pyrazol-4-yl)methyl)-2- chloro-N,N- dimethylbenzenesulfonamide | 445.2 |
| 38 | 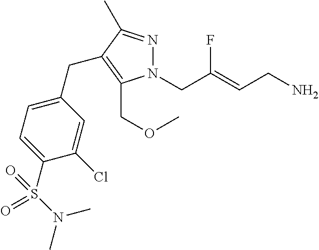 [SMILES: COCc1c(Cc2ccc(S(=O)(=O)N(C)C)c(Cl)c2)c(C)nn1C/C(F)=C/CN] | (Z)-4-((1-(4-amino-2-fluorobut-2- en-1-yl)-5-(methoxymethyl)-3- methyl-1H-pyrazol-4-yl)methyl)-2- chloro-N,N- dimethylbenzenesulfonamide | 445.3 |
| 39 | 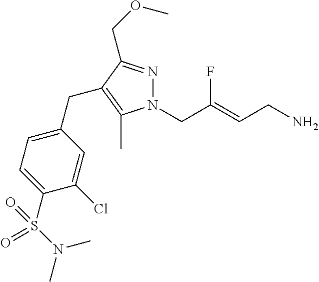 [SMILES: COCc1nn(C/C(F)=C/CN)c(C)c1Cc1ccc(S(=O)(=O)N(C)C)c(Cl)c1] | (Z)-4-((1-(4-amino-2-fluorobut-2- en-1-yl)-3-(methoxymethyl)-5- methyl-1H-pyrazol-4-yl)methyl)-2- chloro-N,N- dimethylbenzenesulfonamide | 445.3 |
| 40 | 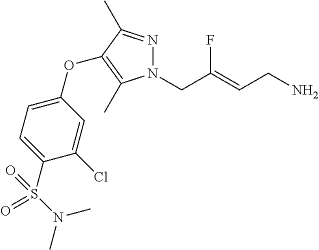 [SMILES: Cc1nn(C/C(F)=C/CN)c(C)c1Oc1ccc(S(=O)(=O)N(C)C)c(Cl)c1] | (Z)-4-((1-(4-amino-2-fluorobut-2- en-1-yl)-3,5-dimethyl-1H-pyrazol- 4-yl)oxy)-2-chloro-N,N- dimethylbenzenesulfonamide | 417.0 |
| 41 | 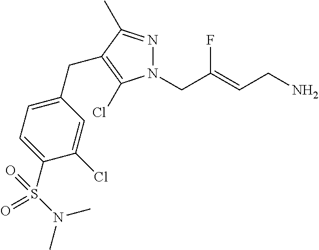 [SMILES: Cc1nn(C/C(F)=C/CN)c(Cl)c1Cc1ccc(S(=O)(=O)N(C)C)c(Cl)c1] | (Z)-4-((1-(4-amino-2-fluorobut-2- en-1-yl)-5-chloro-3-methyl-1H- pyrazol-4-yl)methyl)-2-chloro-N,N- dimethylbenzenesulfonamide | 435.0 |
| 42 | 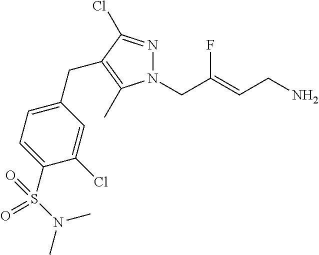 [SMILES: Cc1c(Cc2ccc(S(=O)(=O)N(C)C)c(Cl)c2)c(Cl)nn1C/C(F)=C/CN] | (Z)-4-((1-(4-amino-2-fluorobut-2- en-1-yl)-3-chloro-5-methyl-1H- pyrazol-4-yl)methyl)-2-chloro-N,N- dimethylbenzenesulfonamide | 435.0 |
| 43 | 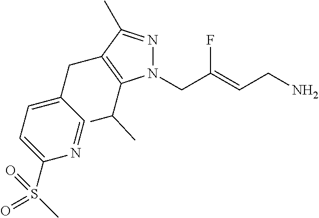 [SMILES: Cc1nn(C/C(F)=C/CN)c(C(C)C)c1Cc1ccc(S(C)(=O)=O)nc1] | (Z)-3-fluoro-4-(5-isopropyl-3- methyl-4-((6- (methylsulfonyl)pyridin-3- yl)methyl)-1H-pyrazol-1-yl)but-2- en-1-amine | 381.3 |
| 44 | 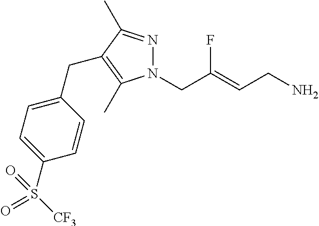 [SMILES: Cc1nn(C/C(F)=C/CN)c(C)c1Cc1ccc(S(=O)(=O)C(F)(F)F)cc1] | (Z)-4-(3,5-dimethyl-4-(4- ((trifluoromethyl)sulfonyl)-benzyl)- 1H-pyrazol-1-yl)-3-fluorobut-2-en- 1-amine | 406.0 |
| 45 | 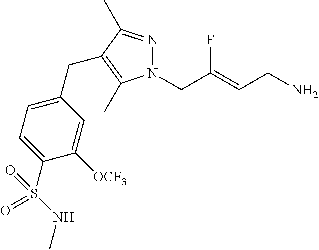 [SMILES: CNS(=O)(=O)c1ccc(Cc2c(C)nn(C/C(F)=C/CN)c2C)cc1OC(F)(F)F] | (Z)-4-((1-(4-amino-2-fluorobut-2- en-1-yl)-3,5-dimethyl-1H-pyrazol- 4-yl)methyl)-N-methyl-2- (trifluoromethoxy)- benzenesulfonamide | 451.0 |
| 46 | 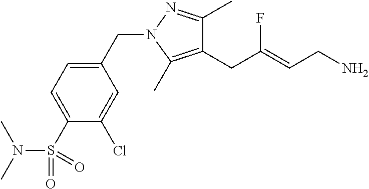 [SMILES: Cc1nn(Cc2ccc(S(=O)(=O)N(C)C)c(Cl)c2)c(C)c1C/C(F)=C/CN] | (Z)-4-((4-(4-amino-2-fluorobut-2- en-1-yl)-3,5-dimethyl-1H-pyrazol- 1-yl)methyl)-2-chloro-N,N- dimethylbenzenesulfonamide | 415.0 |
| 47 | 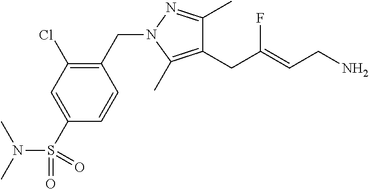 [SMILES: Cc1nn(Cc2ccc(S(=O)(=O)N(C)C)cc2Cl)c(C)c1C/C(F)=C/CN] | (Z)-4-((4-(4-amino-2-fluorobut-2- en-1-yl)-3,5-dimethyl-1H-pyrazol- 1-yl)methyl)-3-chloro-N,N- dimethylbenzenesulfonamide | 415.0 |
| 48 | 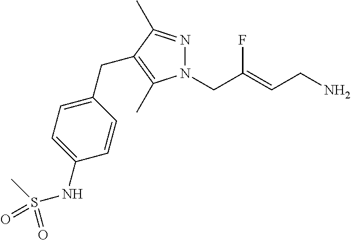 [SMILES: Cc1nn(C/C(F)=C/CN)c(C)c1Cc1ccc(NS(C)(=O)=O)cc1] | (Z)-N-(4-((1-(4-amino-2-fluorobut- 2-en-1-yl)-3,5-dimethyl-1H- pyrazol-4- yl)methyl)phenyl)methane- sulfonamide | 367.2 |
| 49 | 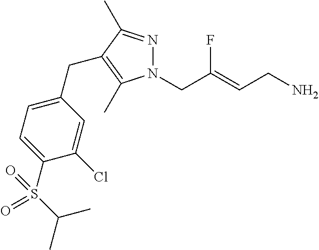 [SMILES: Cc1nn(C/C(F)=C/CN)c(C)c1Cc1ccc(S(=O)(=O)C(C)C)c(Cl)c1] | (Z)-4-(4-(3-chloro-4- (isopropylsulfonyl)benzyl)-3,5- dimethyl-1H-pyrazol-1-yl)-3- fluorobut-2-en-1-amine | 414.0 |
| 50 | 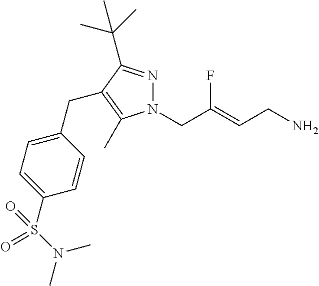 [SMILES: Cc1c(Cc2ccc(S(=O)(=O)N(C)C)cc2)c(C(C)(C)C)nn1C/C(F)=C/CN] | (Z)-4-((1-(4-amino-2-fluorobut-2- en-1-yl)-3-(tert-butyl)-5-methyl- 1H-pyrazol-4-yl)methyl)-N,N- dimethylbenzenesulfonamide | 423.2 |
| 51 | 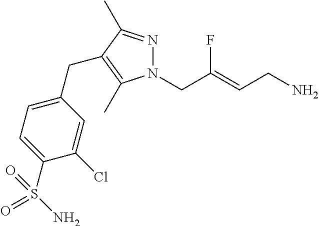 [SMILES: Cc1nn(C/C(F)=C/CN)c(C)c1Cc1ccc(S(N)(=O)=O)c(Cl)c1] | (Z)-4-((1-(4-amino-2-fluorobut-2- en-1-yl)-3,5-dimethyl-1H-pyrazol- 4-yl)methyl)-2- chlorobenzenesulfonamide | 387.0 |
| 52 | 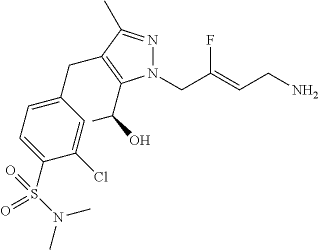 [SMILES: Cc1nn(C/C(F)=C/CN)c([C@H](C)O)c1Cc1ccc(S(=O)(=O)N(C)C)c(Cl)c1] | (Z)-4-((1-(4-amino-2-fluorobut-2- en-1-yl)-5-(1-hydroxyethyl)-3- methyl-1H-pyrazol-4-yl)methyl)-2- chloro-N,N- dimethylbenzenesulfonamide (Enantiomer 1) | 445.3 |
| 53 | 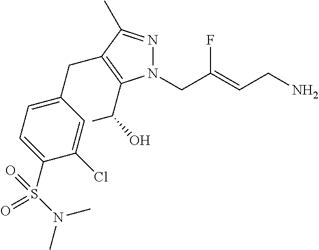 [SMILES: Cc1nn(C/C(F)=C/CN)c([C@@H](C)O)c1Cc1ccc(S(=O)(=O)N(C)C)c(Cl)c1] | (Z)-4-((1-(4-amino-2-fluorobut-2- en-1-yl)-5-(1-hydroxyethyl)-3- methyl-1H-pyrazol-4-yl)methyl)-2- chloro-N,N- dimethylbenzenesulfonamide (Enantiomer 2) | 445.3 |
| 54 | 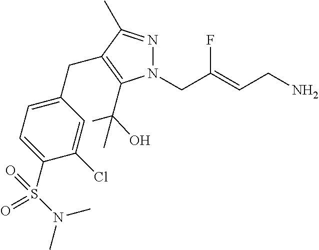 [SMILES: Cc1nn(C/C(F)=C/CN)c(C(C)(C)O)c1Cc1ccc(S(=O)(=O)N(C)C)c(Cl)c1] | (Z)-4-((1-(4-amino-2-fluorobut-2- en-1-yl)-5-(2-hydroxypropan-2-yl)- 3-methyl-1H-pyrazol-4-yl)methyl)- 2-chloro-N,N- dimethylbenzenesulfonamide | 459.2 |
| 55 | 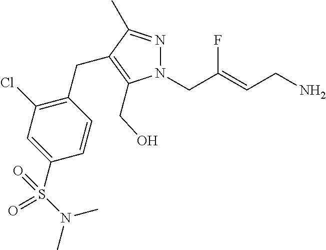 [SMILES: Cc1nn(C/C(F)=C/CN)c(CO)c1Cc1ccc(S(=O)(=O)N(C)C)cc1Cl] | (Z)-4-((1-(4-amino-2-fluorobut-2- en-1-yl)-5-(hydroxymethyl)-3- methyl-1H-pyrazol-4-yl)methyl)-3- chloro-N,N- dimethylbenzenesulfonamide | 431.1 |
| 56 | 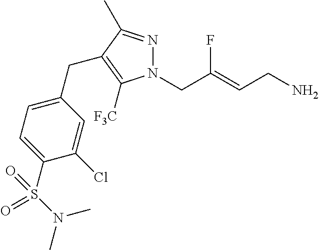 [SMILES: Cc1nn(C/C(F)=C/CN)c(C(F)(F)F)c1Cc1ccc(S(=O)(=O)N(C)C)c(Cl)c1] | (Z)-4-((1-(4-amino-2-fluorobut-2- en-1-yl)-3-methyl-5- (trifluoromethyl)-1H-pyrazol-4- yl)methyl)-2-chloro-N,N- dimethylbenzenesulfonamide | 469.1 |
| 57 | 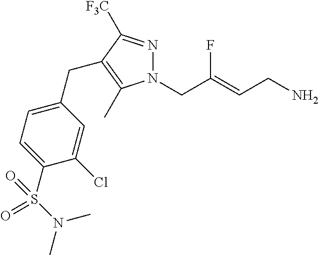 [SMILES: Cc1c(Cc2ccc(S(=O)(=O)N(C)C)c(Cl)c2)c(C(F)(F)F)nn1C/C(F)=C/CN] | (Z)-4-((1-(4-amino-2-fluorobut-2- en-1-yl)-5-methyl-3- (trifluoromethyl)-1H-pyrazol-4- yl)methyl)-2-chloro-N,N- dimethylbenzenesulfonamide | 469.1 |
| 58 | 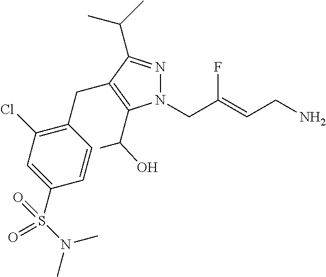 [SMILES: CC(C)c1nn(C/C(F)=C/CN)c(C(C)O)c1Cc1ccc(S(=O)(=O)N(C)C)cc1Cl] | (Z)-4-((1-(4-amino-2-fluorobut-2- en-1-yl)-5-(l-hydroxyethyl)-3- isopropyl-1H-pyrazol-4-yl)methyl)- 3-chloro-N,N- dimethylbenzenesulfonamide | 473.2 |
| 59 | 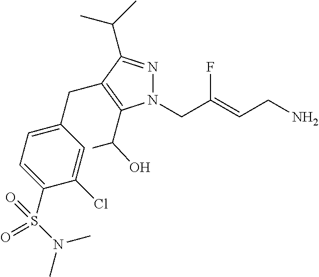 [SMILES: CC(C)c1nn(C/C(F)=C/CN)c(C(C)O)c1Cc1ccc(S(=O)(=O)N(C)C)c(Cl)c1] | (Z)-4-((1-(4-amino-2-fluorobut-2- en-1-yl)-5-(1-hydroxyethyl)-3- isopropyl-1H-pyrazol-4-yl)methyl)- 2-chloro-N,N- dimethylbenzenesulfonamide | 473.2 |
| 60 | 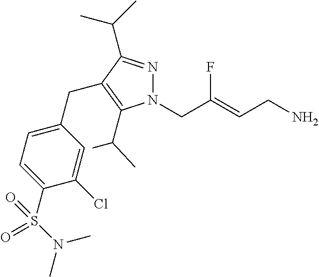 [SMILES: CC(C)c1nn(C/C(F)=C/CN)c(C(C)C)c1Cc1ccc(S(=O)(=O)N(C)C)c(Cl)c1] | (Z)-4-((1-(4-amino-2-fluorobut-2- en-1-yl)-3,5-diisopropyl-1H- pyrazol-4-yl)methyl)-2-chloro-N,N- dimethylbenzenesulfonamide | 471.2 |
| 61 | 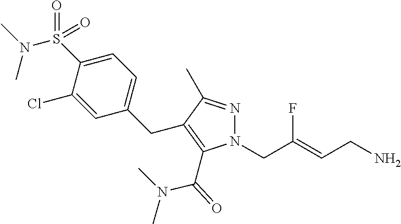 [SMILES: Cc1nn(C/C(F)=C/CN)c(C(=O)N(C)C)c1Cc1ccc(S(=O)(=O)N(C)C)c(Cl)c1] | (Z)-1-(4-amino-2-fluorobut-2-en-1- yl)-4-(3-chloro-4-(N,N- dimethylsulfamoyl)benzyl)-N,N,3- trimethyl-1H-pyrazole-5- carboxamide | 472.1 |
| 62 | 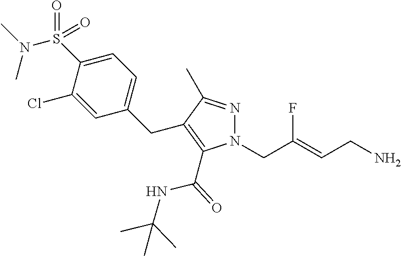 [SMILES: Cc1nn(C/C(F)=C/CN)c(C(=O)NC(C)(C)C)c1Cc1ccc(S(=O)(=O)N(C)C)c(Cl)c1] | (Z)-1-(4-amino-2-fluorobut-2-en-1- yl)-N-(tert-butyl)-4-(3-chloro-4- (N,N-dimethylsulfamoyl)benzyl)-3- methyl-1H-pyrazole-5-carboxamide | 500.2 |
| 63 | 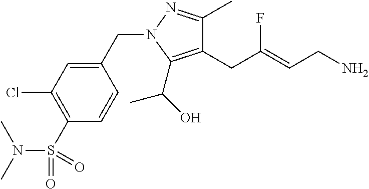 [SMILES: Cc1nn(Cc2ccc(S(=O)(=O)N(C)C)c(Cl)c2)c(C(C)O)c1C/C(F)=C/CN] | (Z)-4-((4-(4-amino-2-fluorobut-2- en-1-yl)-5-(1-hydroxyethyl)-3- methyl-1H-pyrazol-1-yl)methyl)-2- chloro-N,N- dimethylbenzenesulfonamide | 445.2 |
| 64 | 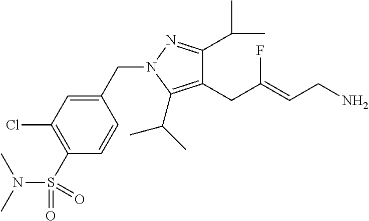 [SMILES: CC(C)c1nn(Cc2ccc(S(=O)(=O)N(C)C)c(Cl)c2)c(C(C)C)c1C/C(F)=C/CN] | (Z)-4-((4-(4-amino-2-fluorobut-2- en-1-yl)-3,5-diisopropyl-1H- pyrazol-1-yl)methyl)-2-chloro-N,N- dimethylbenzenesulfonamide | 471.3 |
| 65 | 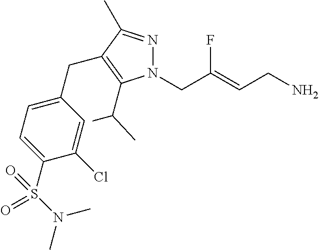 [SMILES: Cc1nn(C/C(F)=C/CN)c(C(C)C)c1Cc1ccc(S(=O)(=O)N(C)C)c(Cl)c1] | (Z)-4-((1-(4-amino-2-fluorobut-2- en-1-yl)-5-isopropyl-3-methyl-1H- pyrazol-4-yl)methyl)-2-chloro-N,N- dimethylbenzenesulfonamide | 443.2 |
| 66 | 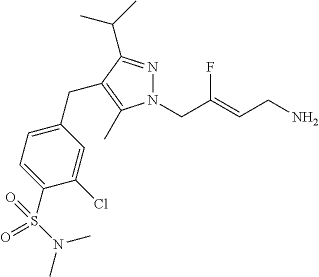 [SMILES: Cc1c(Cc2ccc(S(=O)(=O)N(C)C)c(Cl)c2)c(C(C)C)nn1C/C(F)=C/CN] | (Z)-4-((1-(4-amino-2-fluorobut-2- en-1-yl)-3-isopropyl-5-methyl-1H- pyrazol-4-yl)methyl)-2-chloro-N,N- dimethylbenzenesulfonamide | 443.2 |
| 67 | 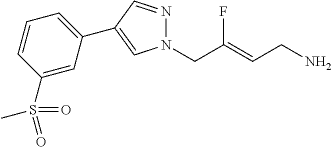 [SMILES: CS(=O)(=O)c1cccc(-c2cnn(C/C(F)=C/CN)c2)c1] | (Z)-3-fluoro-4-(4-(3- (methylsulfonyl)phenyl)-1H- pyrazol-1-yl)but-2-en-1-amine | 310.1 |

### Preparation of Compounds of Formula I

Compounds of Formula I can be readily prepared by those skilled in the art using methods and materials known in the art and with reference to standard textbooks, such as “Advanced Organic Chemistry” by Jerry March (third edition, 1985, John Wiley and Sons) or “Comprehensive Organic Transformations” by Richard C. Larock (1989, VCH Publishers).

Compounds of Formula I may be synthesised as described below. The following schemes provide an overview of representative non-limiting embodiments of the invention. Those skilled in the art will recognize that analogues of Formula I, including different isomeric forms, may also be prepared from the analogous starting materials.

### Scheme 1:

The preparation of compounds described by Formula Ia wherein X is —CH₂— is described in Scheme 1 below.

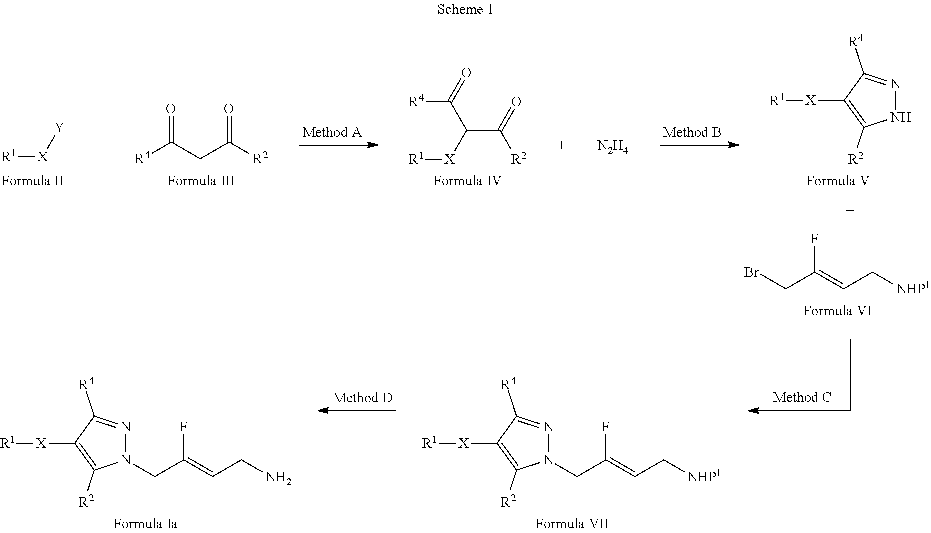

P¹ is a functional group used to protect a nitrogen functionality. Examples of P¹ are carbonates such as the tert-butyloxycarbonyl (BOC), the 9-fluorenylmethyloxycarbonyl (FMOC), and the benzyloxycarbonyl (CBZ) groups.

In this general scheme the R¹ starting material described by Formula II (in which Y is an appropriate leaving group such as Br, I, OTs or OMs) can be prepared by many methods well known in the art. It is reacted with the anion derived from an appropriately substituted 1,3-dicarbonyl compound, as is described by Formula III. For example, a solution of a compounds described by Formula III in a solvent such as ethanol or tetrahydrofuran (THF) can be treated with a base, such as sodium ethoxide or lithium bis(trimethylsilyl)amide, before the addition of a solution of a compound described by Formula II in a solvent such as ethanol or tetrahydrofuran (THF), at temperatures between 0° C. and 80° C. for between 0.5 and 3 hours. The product described by Formula IV can be recovered by standard work-up procedures.

One convenient protocol for the conversion of compounds described by Formula IV to compounds described by Formula V is Method B which involves reaction with hydrazine in ethanol under reflux for several hours. The product described by Formula V can be recovered by standard work-up procedures.

Whilst there are many ways to achieve the reaction described by Method C, one convenient protocol involves reaction of compounds described by Formula V with a base such as sodium hydride in a solvent such as tetrahydrofuran (THF) or dimethylformamide (DMF), before the addition of the compound described by Formula VI at ambient temperature for approximately 1 hour. Following standard extraction and purification methods the product described by Formula VII can be obtained in good yield and purity.

There are many well established chemical procedures for the deprotection of the compounds described by Formula VII to the compounds described by Formula Ia (Method D). For example if P¹ is a BOC protecting group, compounds described by Formula VII can be treated with an acidic substance such as dry hydrogen chloride in a solvent such as diethyl ether to furnish the compounds described by Formula Ia as the hydrochloride salts. In general, the free amino compounds are converted to acid addition salts for ease of handling and for improved chemical stability. Examples of acid addition salts include but are not limited to hydrochloride, hydrobromide, 2,2,2-trifluroacetic acid and methanesulfonate salts.

### Scheme 2:

The preparation of compounds described by Formula Ia wherein X is a bond between R¹ and the pyrazole ring is described in Scheme 2 below.

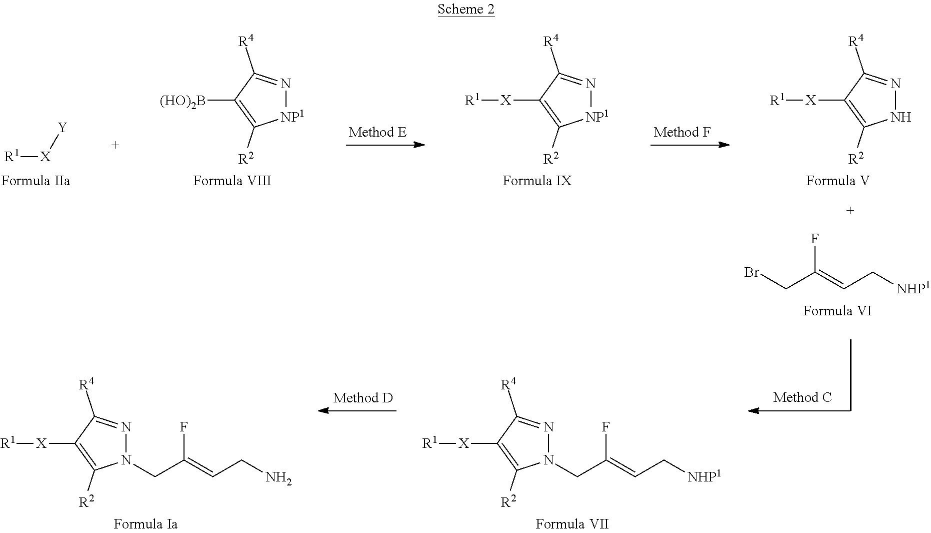

In general Scheme 2 a Suzuki coupling reaction is employed to combine the compounds described by Formulae IIa (in which Y is Br or I) and VIII. There are numerous variants of the Suzuki reaction described in the literature. For example, a solution of the compounds described by Formulae IIa and VIII, in the presence of K₂CO₃, can be dissolved in a solvent such as aqueous dioxane under an atmosphere of nitrogen, then treated with a catalytic amount of palladium tetrakis triphenylphosphine under reflux for several hours. Following standard extraction and purification methods, the coupled product described by Formula IX can be obtained in good yield and purity. Conversion of the protected compound described by Formula IX to compounds described by Formula V is readily achieved by the method best suited to removal of the particular protective group.

### Scheme 3

The preparation of compounds described by Formula Ib wherein X is —CH₂— is described in Scheme 3 below.

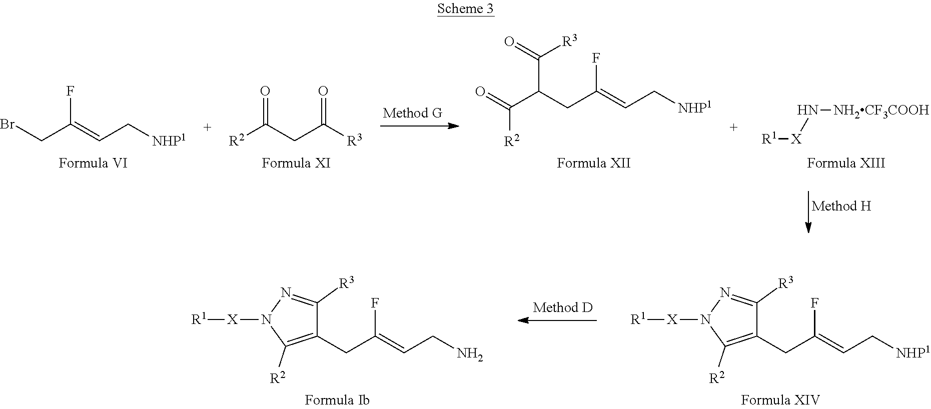

In general Scheme 3, compounds of the general formula XII can be prepared by reaction of the starting material described by Formula VI with an appropriately substituted 1,3-dicarbonyl compound, as described by Formula XI. For example, a solution of a compound described by Formula XI in a solvent such as ethanol or tetrahydrofuran (THF) can be treated with a base, such as sodium ethoxide or lithium bis(trimethylsilyl)amide, before the addition of a solution of a compound described by Formula VI in a solvent such as ethanol or tetrahydrofuran (THF) at temperatures between 0° C. and 80° C. for between 0.5 and 3 hours. The product described by Formula XII can be recovered by standard work-up procedures.

One convenient protocol for the reaction of compounds described by Formula XII with compounds described by Formula XIII is Method H which involves treatment of a solution of a compound described by Formula XIII in a solvent such as ethanol with a base such as diisopropylamine, followed by the addition of a compound described by Formula XII, at temperatures between ambient and 80° C. for between 0.5 and 3 hours. The product described by Formula XIV can be recovered by standard work-up procedures.

Cis/trans (E/Z) mixtures may be separated into constituent isomers by conventional techniques well known to those skilled in the art. For example, by employment of chromatography and/or fractional crystallisation.

Racemic mixtures may be separated into constituent R and S enantiomers by conventional techniques well known to those skilled in the art. For example, by employment of chiral chromatography.

Diastereoisomeric mixtures may be separated into constituent isomers by conventional techniques well known to those skilled in the art. For example, by employment of chromatography and/or fractional crystallisation.

### Therapeutic Uses and Formulations

Another aspect of the present invention relates to a pharmaceutical composition comprising a compound of Formula I, or a pharmaceutically acceptable salt or stereoisomer thereof, together with a pharmaceutically acceptable diluent, excipient or adjuvant.

The present invention also relates to use of the compounds of Formula I in therapy, in particular to inhibit members of the lysyl oxidase family members, LOX, LOXL1, LOXL2, LOXL3 and LOXL4. In one embodiment the invention provides for the selective inhibition of specific lysyl oxidase isoenzymes. In another embodiment the invention provides for the simultaneous inhibition of 2, 3 or 4 LOX isoenzymes. The relative inhibitory potencies of the compounds can be determined by the amount needed to inhibit the amine oxidase activity of LOX, LOXL1, LOXL2, LOXL3 and LOXL4 in a variety of ways, e.g., in an in vitro assay with recombinant or purified human protein or with recombinant or purified non-human enzyme, in cellular assays expressing normal rodent enzyme, in cellular assays which have been transfected with human protein, in in vivo tests in rodent and other mammalian species, and the like.

Accordingly, a further aspect of the invention is directed to a method of inhibiting the amine oxidase activity of LOX, LOXL1, LOXL2, LOXL3 and LOXL4 in a subject in need thereof, comprising administering to the subject an effective amount of a compound of Formula I, or a pharmaceutically acceptable salt or solvate thereof, or a pharmaceutical composition thereof.

In one embodiment the present invention is directed to a method of inhibiting the amine oxidase activity of LOXL2. In another embodiment the present invention is directed towards inhibiting the amine oxidase activity of LOX and LOXL2.

As discussed previously, LOX and LOXL1-4 enzymes are members of a large family of flavin-dependent and copper-dependent amine oxidases, which includes SSAO/VAP-I, monoamine oxidase-B (MAO-B) and diamine oxidase (DAO). In one embodiment compounds of the present invention selectively inhibit members of the lysyl oxidase isoenzyme family with respect to SSAO/VAP-1, MAO-B, DAO and other members of the amine oxidase family.

The present invention also discloses methods to use the compounds described by Formula I to inhibit one or more lysyl oxidase isoenzymes (LOX, LOXL1, LOXL2, LOXL3 and LOXL4) in patients suffering from a fibrotic disease, and methods to treat fibrotic diseases. Furthermore, the present invention discloses methods to use the compounds described by Formula I to inhibit one or more lysyl oxidase isoenzymes (LOX, LOXL1, LOXL2, LOXL3 and LOXL4) in patients suffering from cancer, including metastatic cancer, and methods to treat cancer and metastatic cancer.

In a further aspect of the invention there is provided a method of treating a condition associated with LOX, LOXL1, LOXL2, LOXL3 and LOXL4 protein, comprising administering to a subject in need thereof a therapeutically effective amount of compound of Formula I, or a pharmaceutically acceptable salt or solvate thereof, or a pharmaceutical composition thereof.

In another aspect there is a provided a method of treating a condition modulated by LOX, LOXL1, LOXL2, LOXL3 and LOXL4, comprising administering to a subject in need thereof a therapeutically effective amount of compound of Formula I, or a pharmaceutically acceptable salt or solvate thereof, or a pharmaceutical composition thereof.

In one embodiment of the methods of the present invention the condition is selected from the group consisting of fibrosis, cancer and angiogenesis.

In another aspect, the present invention provides a method for decreasing extracellular matrix formation by treating human subjects, pets and livestock with fluoroallylamine inhibitors of lysyl oxidase isoenzyme family of Formula I as described herein.

The above-described methods are applicable wherein the condition is a liver disorder. As described herein the term “liver disorder” includes any disorder affecting the liver, and in particular any acute or chronic liver disease that involves the pathological disruption, inflammation, degeneration, and/or proliferation of liver cells. In particular, the liver disorder is liver fibrosis, liver cirrhosis, or any other liver disease in which the level in the plasma of some markers of hepatocellular injury, alteration or necrosis, is elevated when compared to normal plasma levels. These biochemical markers associated to liver activity and status can be selected among those disclosed in the literature and in particular Alanine aminotransferase (ALAT), Aspartate aminotransfersase (ASAT), Alkaline Phosphatase (AP), Gamma Glutamyl transpeptidase (GGT), Cytokeratin-18 (CK-18) or Resistin. In a particular embodiment, the liver disorder is a fatty liver disease in which the elevation of one or more of these markers is associated to a more or less significant steatosis in the liver, as it can be confirmed by a liver biopsy. A non-exhaustive list of fatty liver diseases includes non-alcoholic fatty liver disease (NAFLD), nonalcoholic steatohepatitis (NASH), and fatty liver disease associated to disorders such as hepatitis or metabolic syndrome (obesity, insulin resistance, hypertriglyceridemia, and the like). In one embodiment the liver disorder is selected from the group consisting of biliary atresia, cholestatic liver disease, chronic liver disease, nonalcoholic steatohepatitis (NASH), non-alcoholic fatty liver disease (NAFLD), hepatitis C infection, alcoholic liver disease, primary biliary cirrhosis (PBC), primary schlerosing cholangitis (PSC), liver damage due to progressive fibrosis, liver fibrosis and liver cirrhosis.

The above-described methods are applicable wherein the condition is a kidney disorder. In one embodiment the kidney disorder is selected from the group consisting of kidney fibrosis, renal fibrosis, acute kidney injury, chronic kidney disease, diabetic nephropathy, glomerulosclerosis, vesicoureteral reflux, tubulointerstitial renal fibrosis and glomerulonephritis.

The above-described methods are applicable wherein the condition is a cardiovascular disease. In one embodiment the cardiovascular disease is selected from the group consisting of atherosclerosis, arteriosclerosis, hypercholesteremia, and hyperlipidemia.

The above-described methods are applicable wherein the condition is fibrosis. As employed here “fibrosis” includes such diseases as cystic fibrosis, idiopathic pulmonary fibrosis, liver fibrosis, kidney fibrosis, scleroderma, radiation-induced fibrosis, ocular fibrosis, Peyronie's disease, scarring and other diseases where excessive fibrosis contributes to disease pathology including Crohn's disease and inflammatory bowel disease.

In one embodiment the fibrosis is selected from the group consisting of liver fibrosis, lung fibrosis, kidney fibrosis, cardiac fibrosis, cystic fibrosis, idiopathic pulmonary fibrosis, radiation-induced fibrosis and scleroderma or is associated with respiratory disease, abnormal wound healing and repair, post-surgical operations, cardiac arrest and all conditions where excess or aberrant deposition of fibrous material is associated with disease. In another embodiment the fibrosis is selected from the group consisting of liver fibrosis, lung fibrosis, kidney fibrosis, cardiac fibrosis, and scleroderma.

In one embodiment, kidney fibrosis includes, but is not limited to, diabetic nephropathy, vesicoureteral reflux, tubulointerstitial renal fibrosis; glomerulonephritis or glomerular nephritis, including focal segmental glomerulosclerosis and membranous glomerulonephritis, and mesangiocapillary glomerular nephritis. In one embodiment, liver fibrosis results in cirrhosis, and includes associated conditions such as chronic viral hepatitis, non-alcoholic fatty liver disease (NAFLD), alcoholic steatohepantis (ASH), non-alcoholic steatohepatiris (NASH), primary biliary cirrhosis (PBC), biliary cirrhosis, and autoimmune hepatitis.

The above-described methods are also applicable wherein the condition is cancer. In one embodiment the cancer is selected from the group consisting of lung cancer; breast cancer; colorectal cancer, anal cancer; pancreatic cancer; prostate cancer; ovarian carcinoma; liver and bile duct carcinoma; esophageal carcinoma; non-Hodgkin's lymphoma; bladder carcinoma; carcinoma of the uterus; glioma, glioblastoma, medullablastoma, and other tumors of the brain; kidney cancer; myelofibrosis, cancer of the head and neck; cancer of the stomach; multiple myeloma; testicular cancer; germ cell tumor; neuroendocrine tumor, cervical cancer; oral cancer; carcinoids of the gastrointestinal tract, breast, and other organs; signet ring cell carcinoma; mesenchymal tumors including sarcomas, fibrosarcomas, haemangioma, angiomatosis, haemangiopericytoma, pseudoangiomatous stromal hyperplasia, myofibroblastoma, fibromatosis, inflammatory myofibroblastic tumour, lipoma, angiolipoma, granular cell tumour, neurofibroma, schwannoma, angiosarcoma, liposarcoma, rhabdomyosarcoma, osteosarcoma, leiomyoma or a leiomysarcoma.

In one embodiment the cancer is selected from the group consisting of breast cancer, head and neck squamous cell carcinoma, brain cancer, prostate cancer, renal cell carcinoma, liver cancer, lung cancer, oral cancer, cervical cancer and tumour metastasis.

In one embodiment lung cancer includes lung adenocarcinoma, squamous cell carcinoma, large cell carcinoma, bronchoalveolar carcinoma, non-small-cell carcinoma, small cell carcinoma and mesothelioma. In one embodiment breast cancer includes ductal carcinoma, lobular carcinoma, inflammatory breast cancer, clear cell carcinoma, and mucinous carcinoma. In one embodiment colorectal cancer includes colon cancer and rectal cancer. In one embodiment pancreatic cancer includes pancreatic adenocarcinoma, islet cell carcinoma and neuroendocrine tumors.

In one embodiment ovarian carcinoma includes ovarian epithelial carcinoma or surface epithelial-stromal tumour including serous tumour, endometrioid tumor and mucinous cystadenocarcinoma, and sex-cord-stromal tumor. In one embodiment liver and bile duct carcinoma includes hepatocelluar carcinoma, cholangiocarcinoma and hemangioma. In one embodiment esophageal carcinoma includes esophageal adenocarcinoma and squamous cell carcinoma. In one embodiment carcinoma of the uterus includes endometrial adenocarcinoma, uterine papillary serous carcinoma, uterine clear-cell carcinoma, uterine sarcomas and leiomyosarcomas and mixed mullerian tumors. In one embodiment kidney cancer includes renal cell carcinoma, clear cell carcinoma and Wilm's tumor. In one embodiment cancer of the head and neck includes squamous cell carcinomas. In one embodiment cancer of the stomach includes stomach adenocarcinoma and gastrointestinal stromal tumor.

In one embodiment, the cancer is selected from the group consisting of colon cancer, ovarian cancer, lung cancer, esophageal carcinoma, breast cancer and prostate cancer.

The above-described methods are applicable wherein the condition is angiogenesis.

In one embodiment of the methods of the present invention the subject is selected from the group consisting of humans, pets and livestock. In another embodiment of the methods of the present invention the subject is a human.

A further aspect of the invention provides for use of a compound of Formula I, or a pharmaceutically acceptable salt or solvate thereof, for the manufacture of a medicament for treating a condition associated with LOX, LOXL1, LOXL2, LOXL3 and LOXL4 protein.

Another aspect of the invention provides for use of a compound of Formula I, or a pharmaceutically acceptable salt or solvate thereof, for the manufacture of a medicament for treating a condition modulated by LOX, LOXL1, LOXL2, LOXL3 and LOXL4.

Pharmaceutical and/or Therapeutic Formulations

In another embodiment of the present invention, there are provided compositions comprising a compound having Formula I and at least one pharmaceutically acceptable excipient, carrier or diluent thereof. The compound(s) of Formula I may also be present as suitable salts, including pharmaceutically acceptable salts.

The phrase “pharmaceutically acceptable carrier” refers to any carrier known to those skilled in the art to be suitable for the particular mode of administration. In addition, the compounds may be formulated as the sole pharmaceutically active ingredient in the composition or may be combined with other active ingredients.

The phrase “pharmaceutically acceptable salt” refers to any salt preparation that is appropriate for use in a pharmaceutical application. By pharmaceutically acceptable salt it is meant those salts which, within the scope of sound medical judgement, are suitable for use in contact with the tissues of humans and lower animals without undue toxicity, irritation, allergic response and the like, and are commensurate with a reasonable benefit/risk ratio. Pharmaceutically acceptable salts are well known in the art and include acid addition and base salts. Hemisalts of acids and bases may also be formed. Pharmaceutically acceptable salts include amine salts of mineral acids (e.g., hydrochlorides, hydrobromides, sulfates, and the like); and

amine salts of organic acids (e.g., formates, acetates, lactates, malates, tartrates, citrates, ascorbates, succinates, maleates, butyrates, valerates, fumarates, and the like).

For compounds of formula (I) having a basic site, suitable pharmaceutically acceptable salts may be acid addition salts. For example, suitable pharmaceutically acceptable salts of such compounds may be prepared by mixing a pharmaceutically acceptable acid such as hydrochloric acid, sulfuric acid, methanesulfonic acid, succinic acid, fumaric acid, maleic acid, benzoic acid, phosphoric acid, acetic acid, oxalic acid, carbonic acid, tartaric acid, or citric acid with the compounds of the invention.

S. M. Berge et al. describe pharmaceutically acceptable salts in detail in J. Pharmaceutical Sciences, 1977, 66:1-19. The salts can be prepared in situ during the final isolation and purification of the compounds of the invention, or separately by reacting the free base function with a suitable organic acid. Representative acid addition salts include acetate, adipate, alginate, ascorbate, asparate, benzenesulfonate, benzoate, bisulfate, borate, butyrate, camphorate, camphorsulfonate, citrate, digluconate, cyclopentanepropionate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Suitable base salts are formed from bases that form non-toxic salts. Examples include the aluminium, arginine, benzathine, calcium, choline, diethylamine, diolamine, glycine, lysine, magnesium, meglumine, olamine, potassium, sodium, tromethamine and zinc salts. Representative alkali or alkaline earth metal salts include sodium, lithium potassium, calcium, magnesium, and the like, as well as non-toxic ammonium, quaternary ammonium, and amine cations, including, but not limited to ammonium, tetramethylammonium, tetraethylammonium, methylamine, dimethylamine, trimethylamine, triethylamine, ethylamine, triethanolamine and the like.

Pharmaceutically acceptable salts of compounds of formula I may be prepared by methods known to those skilled in the art, including for example:

(i) by reacting the compound of formula I with the desired acid or base;
(ii) by removing an acid- or base-labile protecting group from a suitable precursor of the compound of formula I or by ring-opening a suitable cyclic precursor, for example, a lactone or lactam, using the desired acid or base; or
(iii) by converting one salt of the compound of formula I to another by reaction with an appropriate acid or base or by means of a suitable ion exchange column.

The above reactions (i)-(iii) are typically carried out in solution. The resulting salt may precipitate out and be collected by filtration or may be recovered by evaporation of the solvent. The degree of ionisation in the resulting salt may vary from completely ionised to almost non-ionised.

Thus, for instance, suitable pharmaceutically acceptable salts of compounds according to the present invention may be prepared by mixing a pharmaceutically acceptable acid such as hydrochloric acid, sulfuric acid, methanesulfonic acid, succinic acid, fumaric acid, maleic acid, benzoic acid, phosphoric acid, acetic acid, oxalic acid, carbonic acid, tartaric acid, or citric acid with the compounds of the invention. Suitable pharmaceutically acceptable salts of the compounds of the present invention therefore include acid addition salts.

The compounds of the invention may exist in both unsolvated and solvated forms. The term ‘solvate’ is used herein to describe a molecular complex comprising the compound of the invention and a stoichiometric amount of one or more pharmaceutically acceptable solvent molecules, for example, ethanol. The term ‘hydrate’ is employed when the solvent is water.

In one embodiment the compounds of Formula I may be administered in the form of a “prodrug”. The phrase “prodrug” refers to a compound that, upon in vivo administration, is metabolized by one or more steps or processes or otherwise converted to the biologically, pharmaceutically or therapeutically active form of the compound. Prodrugs can be prepared by modifying functional groups present in the compound in such a way that the modifications are cleaved, either in routine manipulation or in vivo, to a compound described herein. For example, prodrugs include compounds of the present invention wherein a hydroxy, amino, or sulfhydryl group is bonded to any group that, when administered to a mammalian subject, can be cleaved to form a free hydroxyl, free amino, or free sulfhydryl group, respectively. Representative prodrugs include, for example, amides, esters, enol ethers, enol esters, acetates, formates, benzoate derivatives, and the like of alcohol and amine functional groups in the compounds of the present invention. The prodrug form can be selected from such functional groups as —C(O)alkyl, —C(O)cycloalkyl, —C(O)aryl, —C(O)-arylalkyl, C(O)heteroaryl, —C(O)-heteroarylalkyl, or the like. By virtue of knowledge of pharmacodynamic processes and drug metabolism in vivo, those of skill in this art, once a pharmaceutically active compound is known, can design prodrugs of the compound (see, e.g., Nogrady (1985) Medicinal Chemistry A Biochemical Approach, Oxford University Press, New York, pages 388-392).

Compositions herein comprise one or more compounds provided herein. The compounds are, in one embodiment, formulated into suitable pharmaceutical preparations such as solutions, suspensions, tablets, creams, gels, dispersible tablets, pills, capsules, powders, sustained release formulations or elixirs, for oral administration or in sterile solutions or suspensions for parenteral administration, as well as transdermal patch preparation and dry powder inhalers. In one embodiment, the compounds described above are formulated into pharmaceutical compositions using techniques and procedures well known in the art (see, e.g., Ansel Introduction to Pharmaceutical Dosage Forms, Fourth Edition 1985, 126).

In the compositions, effective concentrations of one or more compounds or pharmaceutically acceptable derivatives thereof is (are) mixed with a suitable pharmaceutical carrier. The compounds may be derivatized as the corresponding salts, esters, enol ethers or esters, acetals, ketals, orthoesters, hemiacetals, hemiketals, acids, bases, solvates, hydrates or prodrugs prior to formulation, as described above. The concentrations of the compounds in the compositions are effective for delivery of an amount, upon administration, that treats, prevents, or ameliorates one or more of the symptoms of diseases or disorders to be treated.

In one embodiment, the compositions are formulated for single dosage administration. To formulate a composition, the weight fraction of compound is dissolved, suspended, dispersed or otherwise mixed in a selected carrier at an effective concentration such that the treated condition is relieved, prevented, or one or more symptoms are ameliorated.

The active compound is included in the pharmaceutically acceptable carrier in an amount sufficient to exert a therapeutically useful effect in the absence of undesirable side effects on the patient treated. The therapeutically effective concentration may be determined empirically by testing the compounds in in vitro and in vivo systems described herein and in PCT publication WO 04/018997, and then extrapolated from there for dosages for humans.

The concentration of active compound in the pharmaceutical composition will depend on absorption, distribution, inactivation and excretion rates of the active compound, the physicochemical characteristics of the compound, the dosage schedule, and amount administered as well as other factors known to those of skill in the art.

In one embodiment, a therapeutically effective dosage should produce a serum concentration of active ingredient of from about 0.1 ng/mL to about 50-100 μg/mL. The pharmaceutical compositions, in another embodiment, should provide a dosage of from about 0.001 mg to about 2000 mg of compound per kilogram of body weight per day. Pharmaceutical dosage unit forms are prepared to provide from about 0.01 mg, 0.1 mg or 1 mg to about 500 mg, 1000 mg or 2000 mg, and in one embodiment from about 10 mg to about 500 mg of the active ingredient or a combination of essential ingredients per dosage unit form.

Dosing may occur at intervals of minutes, hours, days, weeks, months or years or continuously over any one of these periods. Suitable dosages lie within the range of about 0.1 ng per kg of body weight to 1 g per kg of body weight per dosage. The dosage is preferably in the range of 1 μg to 1 g per kg of body weight per dosage, such as is in the range of 1 mg to 1 g per kg of body weight per dosage. Suitably, the dosage is in the range of 1 μg to 500 mg per kg of body weight per dosage, such as 1 g to 200 mg per kg of body weight per dosage, or 1 μg to 100 mg per kg of body weight per dosage. Other suitable dosages may be in the range of 1 mg to 250 mg per kg of body weight, including 1 mg to 10, 20, 50 or 100 mg per kg of body weight per dosage or 10 μg to 100 mg per kg of body weight per dosage.

Suitable dosage amounts and dosing regimens can be determined by the attending physician and may depend on the particular condition being treated, the severity of the condition, as well as the general health, age and weight of the subject.

In instances in which the compounds exhibit insufficient solubility, methods for solubilizing compounds may be used. Such methods are known to those of skill in this art, and include, but are not limited to, using cosolvents, such as dimethylsulfoxide (DMSO), using surfactants, such as TWEEN®, dissolution in aqueous sodium bicarbonate, formulating the compounds of interest as nanoparticles, and the like. Derivatives of the compounds, such as prodrugs of the compounds may also be used in formulating effective pharmaceutical compositions.

Upon mixing or addition of the compound(s), the resulting mixture may be a solution, suspension, emulsion or the like. The form of the resulting mixture depends upon a number of factors, including the intended mode of administration and the solubility of the compound in the selected carrier or vehicle. The effective concentration is sufficient for ameliorating the symptoms of the disease, disorder or condition treated and may be empirically determined.

The pharmaceutical compositions are provided for administration to humans and animals in unit dosage forms, such as tablets, capsules, pills, powders, granules, sterile parenteral solutions or suspensions, and oral solutions or suspensions, and oil-water emulsions containing suitable quantities of the compounds or pharmaceutically acceptable derivatives thereof. The pharmaceutically therapeutically active compounds and derivatives thereof are, in one embodiment, formulated and administered in unit-dosage forms or multiple-dosage forms. The active ingredient may be administered at once, or may be divided into a number of smaller doses to be administered at intervals of time. Unit-dose forms as used herein refers to physically discrete units suitable for human and animal subjects and packaged individually as is known in the art. Each unit-dose contains a predetermined quantity of the therapeutically active compound sufficient to produce the desired therapeutic effect, in association with the required pharmaceutical carrier, vehicle or diluent. Examples of unit-dose forms include ampoles and syringes and individually packaged tablets or capsules. Unit-dose forms may be administered in fractions or multiples thereof. A multiple-dose form is a plurality of identical unit-dosage forms packaged in a single container to be administered in segregated unit-dose form. Examples of multiple-dose forms include vials, bottles of tablets or capsules or bottles of pints or gallons. Hence, multiple dose form is a multiple of unit-doses which are not segregated in packaging.

Actual methods of preparing such dosage forms are known, or will be apparent, to those skilled in this art; for example, see Remington's Pharmaceutical Sciences, Mack Publishing Company, Easton, Pa., 15th Edition, 1975.

Dosage forms or compositions containing active ingredient in the range of 0.005% to 100% (wt %) with the balance made up from non-toxic carrier may be prepared. Methods for preparation of these compositions are known to those skilled in the art. The contemplated compositions may contain 0.001%-100% (wt %) active ingredient, in one embodiment 0.1-95% (wt %), in another embodiment 75-85% (wt %).

### Modes of Administration

Convenient modes of administration include injection (subcutaneous, intravenous, etc.), oral administration, inhalation, transdermal application, topical creams or gels or powders, vaginal or rectal administration. Depending on the route of administration, the formulation and/or compound may be coated with a material to protect the compound from the action of enzymes, acids and other natural conditions which may inactivate the therapeutic activity of the compound. The compound may also be administered parenterally or intraperitoneally.

### Compositions for Oral Administration

Oral pharmaceutical dosage forms are either solid, gel or liquid. The solid dosage forms are tablets, capsules, granules, and bulk powders. Types of oral tablets include compressed, chewable lozenges and tablets which may be enteric-coated, sugar-coated or film-coated. Capsules may be hard or soft gelatin capsules, while granules and powders may be provided in non-effervescent or effervescent form with the combination of other ingredients known to those skilled in the art.

### Solid Compositions for Oral Administration

In certain embodiments, the formulations are solid dosage forms, in one embodiment, capsules or tablets. The tablets, pills, capsules, troches and the like can contain one or more of the following ingredients, or compounds of a similar nature: a binder; a lubricant; a diluent; a glidant;

a disintegrating agent; a coloring agent; a sweetening agent; a flavoring agent; a wetting agent; an emetic coating; and a film coating. Examples of binders include microcrystalline cellulose, gum tragacanth, glucose solution, acacia mucilage, gelatin solution, molasses, polyvinylpyrrolidine, povidone, crospovidones, sucrose and starch paste. Lubricants include talc, starch, magnesium or calcium stearate, lycopodium and stearic acid. Diluents include, for example, lactose, sucrose, starch, kaolin, salt, mannitol and dicalcium phosphate. Glidants include, but are not limited to, colloidal silicon dioxide. Disintegrating agents include croscarmellose sodium, sodium starch glycolate, alginic acid, corn starch, potato starch, bentonite, methylcellulose, agar and carboxymethylcellulose. Coloring agents include, for example, any of the approved certified water soluble FD and C dyes, mixtures thereof; and water insoluble FD and C dyes suspended on alumina hydrate. Sweetening agents include sucrose, lactose, mannitol and artificial sweetening agents such as saccharin, and any number of spray dried flavors. Flavoring agents include natural flavors extracted from plants such as fruits and synthetic blends of compounds which produce a pleasant sensation, such as, but not limited to peppermint and methyl salicylate. Wetting agents include propylene glycol monostearate, sorbitan monooleate, diethylene glycol monolaurate and polyoxyethylene laural ether. Emetic-coatings include fatty acids, fats, waxes, shellac, ammoniated shellac and cellulose acetate phthalates. Film coatings include hydroxyethylcellulose, sodium carboxymethylcellulose, polyethylene glycol 4000 and cellulose acetate phthalate.

The compound, or pharmaceutically acceptable derivative thereof, could be provided in a composition that protects it from the acidic environment of the stomach. For example, the composition can be formulated in an enteric coating that maintains its integrity in the stomach and releases the active compound in the intestine. The composition may also be formulated in combination with an antacid or other such ingredient.

When the dosage unit form is a capsule, it can contain, in addition to material of the above type, a liquid carrier such as a fatty oil. In addition, dosage unit forms can contain various other materials which modify the physical form of the dosage unit, for example, coatings of sugar and other enteric agents. The compounds can also be administered as a component of an elixir, suspension, syrup, wafer, sprinkle, chewing gum or the like. A syrup may contain, in addition to the active compounds, sucrose as a sweetening agent and certain preservatives, dyes and colorings and flavors.

The active materials can also be mixed with other active materials which do not impair the desired action, or with materials that supplement the desired action, such as antacids, H2 blockers, and diuretics. The active ingredient is a compound or pharmaceutically acceptable derivative thereof as described herein. Higher concentrations, up to about 98% by weight of the active ingredient may be included.

In all embodiments, tablets and capsules formulations may be coated as known by those of skill in the art in order to modify or sustain dissolution of the active ingredient. Thus, for example, they may be coated with a conventional enterically digestible coating, such as phenylsalicylate, waxes and cellulose acetate phthalate.

### Liquid Compositions for Oral Administration

Liquid oral dosage forms include aqueous solutions, emulsions, suspensions, solutions and/or suspensions reconstituted from non-effervescent granules and effervescent preparations reconstituted from effervescent granules. Aqueous solutions include, for example, elixirs and syrups. Emulsions are either oil-in-water or water-in-oil.

Liquid pharmaceutically administrable compositions can, for example, be prepared by dissolving, dispersing, or otherwise mixing an active compound as defined above and optional pharmaceutical adjuvants in a carrier, such as, for example, water, saline, aqueous dextrose, glycerol, glycols, ethanol, and the like, to thereby form a solution or suspension. If desired, the pharmaceutical composition to be administered may also contain minor amounts of nontoxic auxiliary substances such as wetting agents, emulsifying agents, solubilizing agents, pH buffering agents and the like, for example, acetate, sodium citrate, cyclodextrine derivatives, sorbitan monolaurate, triethanolamine sodium acetate, triethanolamine oleate, and other such agents.

Elixirs are clear, sweetened, hydroalcoholic preparations. Pharmaceutically acceptable carriers used in elixirs include solvents. Syrups are concentrated aqueous solutions of a sugar, for example, sucrose, and may contain a preservative. An emulsion is a two-phase system in which one liquid is dispersed in the form of small globules throughout another liquid. Pharmaceutically acceptable carriers used in emulsions are non-aqueous liquids, emulsifying agents and preservatives. Suspensions use pharmaceutically acceptable suspending agents and preservatives. Pharmaceutically acceptable substances used in non-effervescent granules, to be reconstituted into a liquid oral dosage form, include diluents, sweeteners and wetting agents. Pharmaceutically acceptable substances used in effervescent granules, to-be reconstituted into a liquid oral dosage form, include organic acids and a source of carbon dioxide. Coloring and flavoring agents are used in all of the above dosage forms.

Solvents include glycerin, sorbitol, ethyl alcohol and syrup. Examples of preservatives include glycerin, methyl and propylparaben, benzoic acid, sodium benzoate and ethanol. Examples of non-aqueous liquids utilized in emulsions include mineral oil and cottonseed oil. Examples of emulsifying agents include gelatin, acacia, tragacanth, bentonite, and surfactants such as polyoxyethylene sorbitan monooleate. Suspending agents include sodium carboxymethylcellulose, pectin, tragacanth, Veegum and acacia. Sweetening agents include sucrose, syrups, glycerin and artificial sweetening agents such as saccharin. Wetting agents include propylene glycol monostearate, sorbitan monooleate, diethylene glycol monolaurate and polyoxyethylene lauryl ether. Organic acids include citric and tartaric acid. Sources of carbon dioxide include sodium bicarbonate and sodium carbonate. Coloring agents include any of the approved certified water soluble FD and C dyes, and mixtures thereof. Flavoring agents include natural flavors extracted from plants such fruits, and synthetic blends of compounds which produce a pleasant taste sensation.

For a solid dosage form, the solution or suspension, in for example propylene carbonate, vegetable oils or triglycerides, is in one embodiment encapsulated in a gelatin capsule. For a liquid dosage form, the solution, e.g., for example, in a polyethylene glycol, may be diluted with a sufficient quantity of a pharmaceutically acceptable liquid carrier, e.g., water, to be easily measured for administration.

Alternatively, liquid or semi-solid oral formulations may be prepared by dissolving or dispersing the active compound or salt in vegetable oils, glycols, triglycerides, propylene glycol esters (e.g., propylene carbonate) and other such carriers, and encapsulating these solutions or suspensions in hard or soft gelatin capsule shells. Other useful formulations include those set forth in U.S. Pat. Nos. RE28,819 and 4,358,603. Briefly, such formulations include, but are not limited to, those containing a compound provided herein, a dialkylated mono- or poly-alkylene glycol, including, but not limited to, 1,2-dimethoxymethane, diglyme, triglyme, tetraglyme, polyethylene glycol-350-dimethyl ether, polyethylene glycol-550-dimethyl ether, polyethylene glycol-750-dimethyl ether wherein 350, 550 and 750 refer to the approximate average molecular weight of the polyethylene glycol, and one or more antioxidants, such as butylated hydroxytoluene (BHT), butylated hydroxyanisole (BHA), propyl gallate, vitamin E, hydroquinone, hydroxycoumarins, ethanolamine, lecithin, cephalin, ascorbic acid, malic acid, sorbitol, phosphoric acid, thiodipropionic acid and its esters, and dithiocarbamates.

Other formulations include, but are not limited to, aqueous alcoholic solutions including a pharmaceutically acceptable acetal. Alcohols used in these formulations are any pharmaceutically acceptable water-miscible solvents having one or more hydroxyl groups, including, but not limited to, propylene glycol and ethanol. Acetals include, but are not limited to, di(lower alkyl) acetals of lower alkyl aldehydes such as acetaldehyde diethyl acetal.

### Injectables, Solutions and Emulsions

Parenteral administration, in one embodiment characterized by injection, either subcutaneously, intramuscularly or intravenously is also contemplated herein. Injectables can be prepared in conventional forms, either as liquid solutions or suspensions, solid forms suitable for solution or suspension in liquid prior to injection, or as emulsions. The injectables, solutions and emulsions also contain one or more excipients. Suitable excipients are, for example, water, saline, dextrose, glycerol or ethanol. In addition, if desired, the pharmaceutical compositions to be administered may also contain minor amounts of non-toxic auxiliary substances such as wetting or emulsifying agents, pH buffering agents, stabilizers, solubility enhancers, and other such agents, such as for example, sodium acetate, sorbitan monolaurate, triethanolamine oleate and cyclodextrins.

Implantation of a slow-release or sustained-release system, such that a constant level of dosage is maintained is also contemplated herein. Briefly, a compound provided herein is dispersed in a solid inner matrix, e.g., polymethylmethacrylate, polybutylmethacrylate, plasticized or unplasticized polyvinylchloride, plasticized nylon, plasticized polyethyleneterephthalate, natural rubber, polyisoprene, polyisobutylene, polybutadiene, polyethylene, ethylene-vinylacetate copolymers, silicone rubbers, polydimethylsiloxanes, silicone carbonate copolymers, hydrophilic polymers such as hydrogels of esters of acrylic and methacrylic acid, collagen, cross-linked polyvinylalcohol and cross-linked partially hydrolyzed polyvinyl acetate, that is surrounded by an outer polymeric membrane, e.g., polyethylene, polypropylene, ethylene/propylene copolymers, ethylene/ethyl acrylate copolymers, ethylene/vinylacetate copolymers, silicone rubbers, polydimethyl siloxanes, neoprene rubber, chlorinated polyethylene, polyvinylchloride, vinylchloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer, that is insoluble in body fluids. The compound diffuses through the outer polymeric membrane in a release rate controlling step. The percentage of active compound contained in such parenteral compositions is highly dependent on the specific nature thereof, as well as the activity of the compound and the needs of the subject.

Parenteral administration of the compositions includes intravenous, subcutaneous and intramuscular administrations. Preparations for parenteral administration include sterile solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use and sterile emulsions. The solutions may be either aqueous or nonaqueous.

If administered intravenously, suitable carriers include physiological saline or phosphate buffered saline (PBS), and solutions containing thickening and solubilizing agents, such as glucose, polyethylene glycol, and polypropylene glycol and mixtures thereof.

Pharmaceutically acceptable carriers used in parenteral preparations include aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances.

Examples of aqueous vehicles include Sodium Chloride Injection, Ringers Injection, Isotonic Dextrose Injection, Sterile Water Injection, Dextrose and Lactated Ringers Injection. Nonaqueous parenteral vehicles include fixed oils of vegetable origin, olive oil, cottonseed oil, corn oil, sesame oil and peanut oil. Antimicrobial agents in bacteriostatic or fungistatic concentrations must be added to parenteral preparations packaged in multiple-dose containers which include phenols or cresols, mercurials, benzyl alcohol, chlorobutanol, methyl and propyl p-hydroxybenzoic acid esters, thimerosal, benzalkonium chloride and benzethonium chloride. Isotonic agents include sodium chloride and dextrose. Buffers include phosphate and citrate. Antioxidants include sodium bisulfate. Local anesthetics include procaine hydrochloride. Suspending and dispersing agents include sodium carboxymethylcelluose, hydroxypropyl methylcellulose and polyvinylpyrrolidone. Emulsifying agents include Polysorbate 80 (TWEEN® 80). A sequestering or chelating agent of metal ions include EDTA. Pharmaceutical carriers also include ethyl alcohol, polyethylene glycol and propylene glycol for water miscible vehicles; and sodium hydroxide, hydrochloric acid, citric acid or lactic acid for pH adjustment.

The concentration of the pharmaceutically active compound is adjusted so that an injection provides an effective amount to produce the desired pharmacological effect. The exact dose depends on the age, weight and condition of the patient or animal as is known in the art.

The unit-dose parenteral preparations are packaged in an ampule, a vial or a syringe with a needle. All preparations for parenteral administration must be sterile, as is known and practiced in the art.

Illustratively, intravenous or intraarterial infusion of a sterile aqueous solution containing an active compound is an effective mode of administration. Another embodiment is a sterile aqueous or oily solution or suspension containing an active material injected as necessary to produce the desired pharmacological effect.

Injectables are designed for local and systemic administration. In one embodiment, a therapeutically effective dosage is formulated to contain a concentration of at least about 0.1% w/w up to about 90% w/w or more, in certain embodiments more than 1% w/w of the active compound to the treated tissue(s).

The compound may be suspended in micronized or other suitable form or may be derivatized to produce a more soluble active product or to produce a prodrug. The form of the resulting mixture depends upon a number of factors, including the intended mode of administration and the solubility of the compound in the selected carrier or vehicle. The effective concentration is sufficient for ameliorating the symptoms of the condition and may be empirically determined.

### Lyophilized Powders

Of interest herein are also lyophilized powders, which can be reconstituted for administration as solutions, emulsions and other mixtures. They may also be reconstituted and formulated as solids or gels.

The sterile, lyophilized powder is prepared by dissolving a compound provided herein, or a pharmaceutically acceptable derivative thereof, in a suitable solvent. The solvent may contain an excipient which improves the stability or other pharmacological component of the powder or reconstituted solution, prepared from the powder. Excipients that may be used include, but are not limited to, dextrose, sorbital, fructose, corn syrup, xylitol, glycerin, glucose, sucrose or other suitable agent. The solvent may also contain a buffer, such as citrate, sodium or potassium phosphate or other such buffer known to those of skill in the art at, in one embodiment, about neutral pH. Subsequent sterile filtration of the solution followed by lyophilization under standard conditions known to those of skill in the art provides the desired formulation. In one embodiment, the resulting solution will be apportioned into vials for lyophilization. Each vial will contain a single dosage or multiple dosages of the compound. The lyophilized powder can be stored under appropriate conditions, such as at about 4° C. to room temperature.

Reconstitution of this lyophilized powder with water for injection provides a formulation for use in parenteral administration. For reconstitution, the lyophilized powder is added to sterile water or other suitable carrier. The precise amount depends upon the selected compound. Such amount can be empirically determined.

### Topical Administration

Topical mixtures are prepared as described for the local and systemic administration. The resulting mixture may be a solution, suspension, emulsions or the like and are formulated as creams, gels, ointments, emulsions, solutions, elixirs, lotions, suspensions, tinctures, pastes, foams, aerosols, irrigations, sprays, suppositories, bandages, dermal patches or any other formulations suitable for topical administration.

The compounds or pharmaceutically acceptable derivatives thereof may be formulated as aerosols for topical application, such as by inhalation. These formulations for administration to the respiratory tract can be in the form of an aerosol or solution for a nebulizer, or as a microfine powder for insufflation, alone or in combination with an inert carrier such as lactose. In such a case, the particles of the formulation will, in one embodiment, have diameters of less than 50 microns, in one embodiment less than 10 microns.

The compounds may be formulated for local or topical application, such as for topical application to the skin and mucous membranes, such as in the eye, in the form of gels, creams, and lotions and for application to the eye or for intracisternal or intraspinal application. Topical administration is contemplated for transdermal delivery and also for administration to the eyes or mucosa, or for inhalation therapies. Nasal solutions of the active compound alone or in combination with other pharmaceutically acceptable excipients can also be administered.

These solutions, particularly those intended for ophthalmic use, may be formulated as 0.01%-10% (vol %) isotonic solutions, pH about 5-7, with appropriate salts.

### Compositions for Other Routes of Administration

Other routes of administration, such as transdermal patches, including iontophoretic and electrophoretic devices, vaginal and rectal administration, are also contemplated herein.

Transdermal patches, including iontophoretic and electrophoretic devices, are well known to those of skill in the art. For example, pharmaceutical dosage forms for rectal administration are rectal suppositories, capsules and tablets for systemic effect. Rectal suppositories are used herein mean solid bodies for insertion into the rectum which melt or soften at body temperature releasing one or more pharmacologically or therapeutically active ingredients. Pharmaceutically acceptable substances utilized in rectal suppositories are bases or vehicles and agents to raise the melting point. Examples of bases include cocoa butter (theobroma oil), glycerin-gelatin, carbowax (polyoxyethylene glycol) and appropriate mixtures of mono-, di- and triglycerides of fatty acids. Combinations of the various bases may be used. Agents to raise the melting point of suppositories include spermaceti and wax. Rectal suppositories may be prepared either by the compressed method or by molding. The weight of a rectal suppository, in one embodiment, is about 2 to 3 gm.

Tablets and capsules for rectal administration are manufactured using the same pharmaceutically acceptable substance and by the same methods as for formulations for oral administration.

### Targeted Formulations

The compounds provided herein, or pharmaceutically acceptable derivatives thereof, may also be formulated to be targeted to a particular tissue, receptor, or other area of the body of the subject to be treated. Many such targeting methods are well known to those of skill in the art. All such targeting methods are contemplated herein for use in the instant compositions.

In one embodiment, liposomal suspensions, including tissue-targeted liposomes, such as tumor-targeted liposomes, may also be suitable as pharmaceutically acceptable carriers. These may be prepared according to methods known to those skilled in the art. For example, liposome formulations may be prepared as described in U.S. Pat. No. 4,522,811. Briefly, liposomes such as multilamellar vesicles (MLV's) may be formed by drying down egg phosphatidyl choline and brain phosphatidyl serine (7:3 molar ratio) on the inside of a flask. A solution of a compound provided herein in phosphate buffered saline lacking divalent cations (PBS) is added and the flask shaken until the lipid film is dispersed. The resulting vesicles are washed to remove unencapsulated compound, pelleted by centrifugation, and then resuspended in PBS.

Co-Administration with Other Drugs

In accordance with another aspect of the present invention, it is contemplated that compounds of Formula I as described herein may be administered to a subject in need thereof in combination with medication considered by those of skill in the art to be current standard of care for the condition of interest. Such combinations provide one or more advantages to the subject, e.g., requiring reduced dosages to achieve similar benefit, obtaining the desired palliative effect in less time, and the like.

Compounds in accordance with the present invention may be administered as part of a therapeutic regimen with other drugs. It may desirable to administer a combination of active compounds, for example, for the purpose of treating a particular disease or condition. Accordingly, it is within the scope of the present invention that two or more pharmaceutical compositions, at least one of which contains a compound of Formula (I) according to the present invention, may be combined in the form of a kit suitable for co-administration of the compositions.

In one embodiment of the methods of the present inventions a compound of Formula I may be administered with a second therapeutic agent. In one embodiment the second therapeutic agent is selected from the group consisting of an anti-cancer agent, an anti-inflammatory agent, an anti-hypertensive agent, an anti-fibrotic agent, an anti-angiogenic agent and an immunosuppressive agent.

When two or more active ingredients are co-administered, the active ingredients may be administered simultaneously, sequentially or separately. In one embodiment the compound of Formula I is co-administered simultaneously with a second therapeutic agent. In another embodiment the compound of Formula I and the second therapeutic agent are administered sequentially. In a further embodiment the compound of Formula I and the second therapeutic agent are administered separately.

The invention will now be described in greater detail, by way of illustration only, with reference to the following non-limiting examples. The examples are intended to serve to illustrate the invention and should not be construed as limiting the generality of the disclosure of the description throughout this specification.

### Example 1

### Preparation of (Z)-tert-butyl (4-bromo-3-fluorobut-2-en-1-yl)carbamate

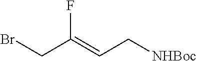 [SMILES: CC(C)(C)OC(=O)NC/C=C(\F)CBr]

### Procedure A: Preparation of tert-butyl 2-oxoethylcarbamate

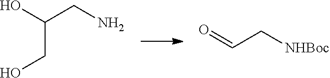 [SMILES: NCC(O)CO ; CC(C)(C)OC(=O)NCC=O]

To a stirring solution of 3-amino-1,2-propanediol (20.0 g, 0.22 mol) in water (200 mL) at 0-5° C. was added di-tert-butyl dicarbonate (55.5 mL, 0.24 mol). After adjusting the alkalinity of the solution to pH-9 by addition of aq. NaOH (6 N), the mixture was left to stir at rt for 18 h. The reaction mixture was cooled to 0-5° C. and then acidified to pH-6 before the addition of sodium metaperiodate (56.3 g, 0.26 mol). The resulting suspension was stirred at rt for 2 h. The mixture was filtered to remove all solids and the filtrate was transferred to a separatory funnel and extracted with ethyl acetate (200 mL). Sodium chloride was added to the aqueous layer until a saturated solution was obtained. The aqueous layer was then extracted further with ethyl acetate (100 mL). The combined organics were dried-over Na₂SO₄ and then concentrated in vacuo to give crude tert-butyl 2-oxoethylcarbamate (45.7 g) as a yellow gum. The crude material was used in the subsequent step without purification.

### Procedure B: Preparation of (E)-ethyl 4-(tert-butoxycarbonylamino)-2-fluorobut-2-enoate and (Z)-ethyl 4-(tert-butoxycarbonylamino)-2-fluorobut-2-enoate

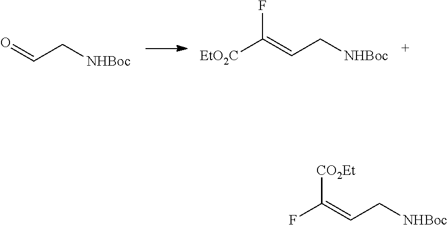 [SMILES: CC(C)(C)OC(=O)NCC=O ; CCOC(=O)/C(F)=C/CNC(=O)OC(C)(C)C ; CCOC(=O)/C(F)=C\CNC(=O)OC(C)(C)C]

To a stirring suspension of crude tert-butyl 2-oxoethylcarbamate (43.7 g, 0.22 mol) and magnesium sulfate (32.0 g) in acetonitrile (200 mL) at 0° C. under N₂ was added sequentially ethyl 2-fluorophosphonoacetate (55.7 mL, 0.27 mol) and 1,8-diazabicyclo[5.4.0]undec-7-ene (32.8 mL, 0.22 mol). The reaction mixture was allowed to warm to rt and stirring was continued for 3 h. After removing the solvent under reduced pressure the residue was taken up in ethyl acetate (200 mL) and then transferred to a separatory funnel. The organics were washed successively with aq. HCl (2 M; 100 mL×2), aq. NaOH (2 M; 100 mL×2) and brine (100 mL). After drying over MgSO₄, the organics were concentrated in vacuo to give the crude, desired product as a mixture of E/Z isomers (2:3; 57.0 g). This crude material was progressed to the next step without purification.

### Procedure C: Preparation of (E)-tert-butyl 3-fluoro-4-hydroxybut-2-enylcarbamate and (Z)-tert-butyl 3-fluoro-4-hydroxybut-2-enylcarbamate

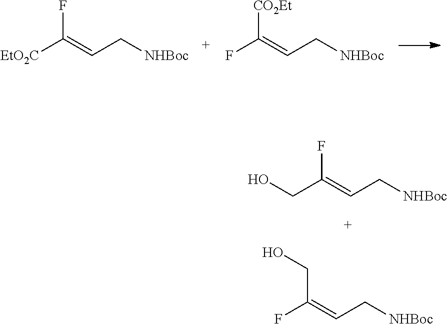 [SMILES: CCOC(=O)/C(F)=C/CNC(=O)OC(C)(C)C ; CCOC(=O)/C(F)=C\CNC(=O)OC(C)(C)C ; CC(C)(C)OC(=O)NC/C=C(\F)CO ; CC(C)(C)OC(=O)NC/C=C(/F)CO]

To a stirring solution of crude E/Z-ethyl 4-(tert-butoxycarbonylamino)-2-fluorobut-2-enoate (18.0 g, 72.8 mmol) in THF (150 mL) at 0° C. under N₂ was added diisobutylaluminum hydride (1 M in toluene, 182 mL, 182 mmol) dropwise over 45 min. After complete addition, the mixture was left to stir at 0° C. for 3 h. The reaction mixture was transferred to a separatory funnel and added dropwise to a stirring mixture of ice (100 g) and aq. NaOH (2 M; 200 mL). Following addition the mixture was stirred for 2 h. The quenched reaction mixture was extracted with diethyl ether (100 mL×2) and the combined organics were washed with brine (100 mL). After drying over MgSO₄ the organics were concentrated in vacuo to give the crude alcohol as a mixture of E/Z isomers. This mixture was purified over silica gel (135 g), eluting with 25% ethyl acetate in n-hexane to give (Z)-tert-butyl 3-fluoro-4-hydroxybut-2-enylcarbamate (6.20 g, 30% over three steps) and (E)-tert-butyl 3-fluoro-4-hydroxybut-2-enylcarbamate (1.85 g, 8.9% over three steps). (E)-tert-butyl 3-fluoro-4-hydroxybut-2-enylcarbamate: ¹H-NMR (200 MHz; CDCl₃) δ ppm: 1.43 (9H, s), 3.72 (2H, dd, J 7.5, 5.4 Hz), 4.25 (2H, d, J 21.5 Hz), 4.85 (1H, br. s), 5.18 (1H, dt, J 19.2, 8.5 Hz). (Z)-tert-butyl 3-fluoro-4-hydroxybut-2-enylcarbamate: ¹H-NMR (300 MHz; CDCl₃) δ ppm: 1.46 (9H, s), 3.84 (2H, dd, J 6.2, 6.2 Hz), 4.13 (2H, d, J 13.9 Hz), 4.68 (1H, br. s), 5.03 (1H, dt, J 36.0, 7.1 Hz).

### Procedure D: Preparation of (Z)-tert-butyl 4-bromo-3-fluorobut-2-enylcarbamate

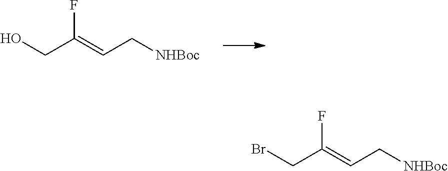 [SMILES: CC(C)(C)OC(=O)NC/C=C(\F)CO ; CC(C)(C)OC(=O)NC/C=C(\F)CBr]

To a stirring solution of (Z)-tert-butyl 3-fluoro-4-hydroxybut-2-enylcarbamate (6.20 g, 30.2 mmol) and triethylamine (6.32 mL, 45.3 mmol) in acetone (100 mL) at 0° C. was added methanesulfonyl chloride (2.81 mL, 36.3 mmol) dropwise. After complete addition the mixture was left to stir at 0° C. for 30 min. After this time, lithium bromide (13.1 g, 0.15 mol) was added portionwise and the resulting suspension was stirred for a further 2 h. The reaction mixture was filtered to remove all solids and the filtrate was concentrated under reduced pressure. The residue was partitioned between water (50 mL) and CH₂Cl₂ (50 mL) and the aqueous layer was extracted with further CH₂Cl₂ (50 mL×2). The combined organics were dried over Na₂SO₄ and concentrated in vacuo. The crude residue was purified over silica gel (100 g) eluting with n-hexane followed by 25% ethyl acetate in n-hexane to afford (Z)-tert-butyl 4-bromo-3-fluorobut-2-enylcarbamate (7.00 g, 86%) as a colourless solid. ¹H-NMR (300 MHz; CDCl₃) δ ppm: 1.46 (9H, s), 3.85 (2H, dd, J 6.2, 6.2 Hz), 3.93 (2H, d, J 19.5 Hz), 4.66 (1H, br. s), 5.16 (1H, dt, J 34.0, 6.5 Hz)

### Example 2

### Preparation of (Z)-4-((1-(4-amino-2-fluorobut-2-en-1-yl)-3,5-dimethyl-1H-pyrazol-4-yl)methyl)-N,N-dimethylbenzenesulfonamide hydrochloride (Compound 2)

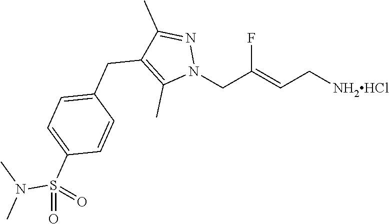

Procedure E: Preparation of 4-(bromomethyl)-N,N-dimethylbenzenesulfonamide

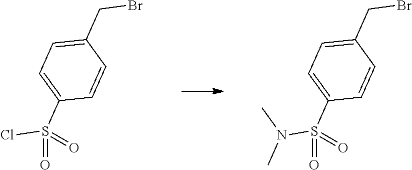

To a stirring solution of 4-(bromomethyl)benzenesulfonyl chloride (5.00 g, 18.6 mmol) in CH₂Cl₂ (40 mL) at 0° C. was added N,N-dimethylamine (5.80 mL, 46.4 mmol) dropwise. Following addition the resulting mixture was left to stir at this temperature for 45 min before partitioning between aq. HCl (1 M, 100 mL) and CH₂Cl₂ (50 mL). The organic layer was washed with further aq. HCl (1 M, 100 mL), water (50 mL), dried over Na₂SO₄ and concentrated in vacuo to give 4-(bromomethyl)-N,N-dimethylbenzenesulfonamide (2.20 g, 43%) as an off-white solid. ¹H-NMR (300 MHz; CD₃OD) δ ppm: 2.74 (6H, s), 4.52 (2H, s), 7.58 (2H, d, J 8.4 Hz), 7.77 (2H, d, J 8.3 Hz).

### Procedure F: Preparation of 4-(2-acetyl-3-oxobutyl)-N,N-dimethylbenzenesulfonamide

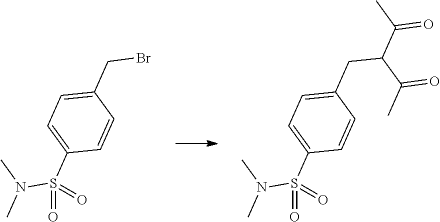 [SMILES: CN(C)S(=O)(=O)c1ccc(CBr)cc1 ; CC(=O)C(Cc1ccc(S(=O)(=O)N(C)C)cc1)C(C)=O]

To a stirring solution of sodium ethoxide (48.9 mg, 0.72 mmol) in ethanol (2 mL) was added pentane-2,4-dione (0.22 mL, 2.16 mmol) and the resulting solution was warmed to 50° C. A solution of 4-(bromomethyl)-N,N-dimethylbenzenesulfonamide (200 mg, 0.72 mmol) in ethanol (2 mL) was then added slowly (over 10 min) and the resulting reaction mixture was heated at reflux for 2 h. The reaction mixture was concentrated in vacuo to remove ethanol and the resulting residue was then partitioned between ethyl acetate (10 mL) and water (10 mL). The phases were separated and the aqueous phase extracted with ethyl acetate (10 mL). The organics were combined, washed with brine, dried over Na₂SO₄ and concentrated in vacuo. Purification by flash chromatography, eluting with 30-50% ethyl acetate/hexane, afforded 4-(2-acetyl-3-oxobutyl)-N,N-dimethylbenzenesulfonamide (120 mg, 56%) as a colourless oil. ¹H-NMR (300 MHz; CD₃OD) (1:0.7 ratio enol:keto tautomers; asterix denotes discrete signal corresponding to minor keto tautomer) δ ppm: 2.09 (3H, s), 2.18 (3H, s), 2.72 (3H, s), 2.74 (3H, s), 3.24* (0.8H, d, J 7.5 Hz), 3.77 (1.2H, s), 4.03* (0.4H, t, J 7.5 Hz), 7.32-7.39 (2H, m), 7.69-7.77 (2H, m).

### Procedure G: Preparation of 4-((3,5-dimethyl-1H-pyrazol-4-yl)methyl)-N,N-dimethylbenzene sulfonamide

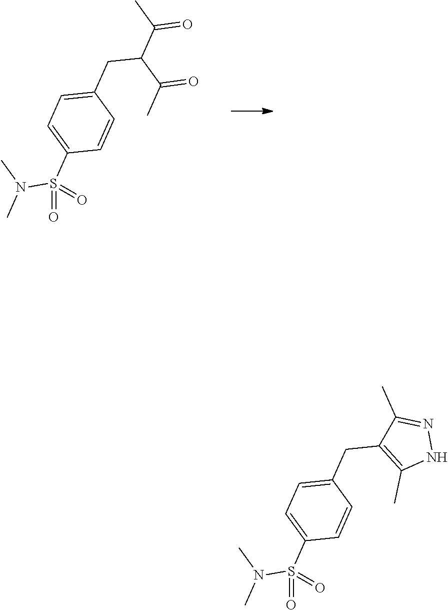 [SMILES: CC(=O)C(Cc1ccc(S(=O)(=O)N(C)C)cc1)C(C)=O ; Cc1n[nH]c(C)c1Cc1ccc(S(=O)(=O)N(C)C)cc1]

To a stirring solution of 4-(2-acetyl-3-oxobutyl)-N,N-dimethylbenzenesulfonamide (120 mg, 0.40 mmol) in ethanol (1.5 mL) was added hydrazine hydrate (21.5 μL, 0.44 mmol) and the resulting solution was heated at reflux for 2 h. The reaction mixture was then concentrated in vacuo and the resulting residue partitioned between ethyl acetate (10 mL) and water (10 mL). The phases were separated and the aqueous phase extracted with ethyl acetate (10 mL). The organics were then combined, washed (water ×3, brine), dried over Na₂SO₄ and concentrated in vacuo to afford 4-((3,5-dimethyl-1H-pyrazol-4-yl)methyl)-N,N-dimethylbenzenesulfonamide (101 mg, 85%) as an off-white solid. ¹H-NMR (300 MHz; CDCl₃) δ ppm: 2.17 (6H, s), 2.71 (6H, s), 3.84 (2H, s), 7.29 (2H, d, J 8.5 Hz), 7.69 (2H, d, J 8.5 Hz).

### Procedure H: Preparation of (Z)-tert-butyl (4-(4-(4-(N,N-dimethylsulfamoyl)benzyl)-3,5-dimethyl-1H-pyrazol-1-yl)-3-fluorobut-2-en-1-yl)carbamate

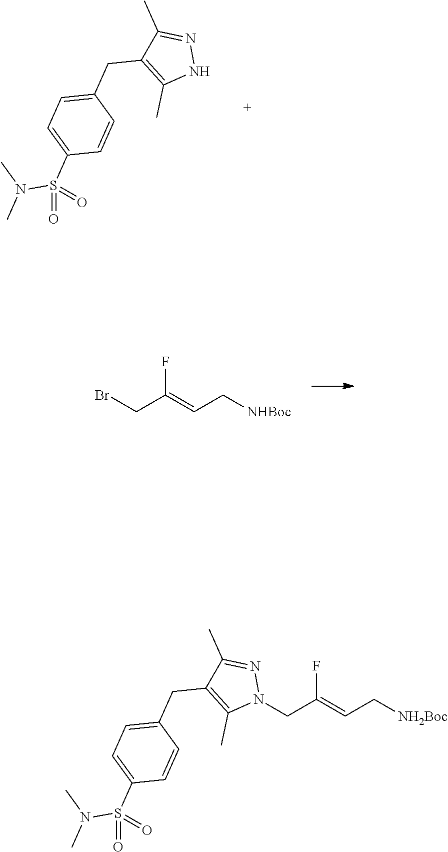

To a stirring solution of 4-((3,5-dimethyl-1H-pyrazol-4-yl)methyl)-N,N-dimethylbenzenesulfonamide (50.0 mg, 0.17 mmol) in DMF (1 mL) at 0° C. was added sodium hydride (60% in mineral oil; 7.50 mg, 0.19 mmol). The resulting solution was stirred at 0° C. for 10 min before the addition of (Z)-tert-butyl (4-bromo-3-fluorobut-2-en-1-yl)carbamate (54.8 mg, 0.20 mmol) in one lot. The resulting mixture was stirred at 0° C. for 30 min, warmed to rt and stirred for a further 5 min and then quenched by the addition of water (10 mL). Ethyl acetate (10 mL) was added and the phases were separated. The aqueous phase was extracted again with ethyl acetate (10 mL) and the organics were combined, washed (water ×4, brine), dried over Na₂SO₄ and concentrated in vacuo. The crude material was purified by flash column, eluting with 100% ethyl acetate to 1% methanol/ethyl acetate to afford (Z)-tert-butyl (4-(4-(4-(N,N-dimethylsulfamoyl)benzyl)-3,5-dimethyl-1H-pyrazol-1-yl)-3-fluorobut-2-en-1-yl)carbamate (63.0 mg, 77%) as a pale yellow oil. ¹H-NMR (300 MHz; CDCl₃) δ ppm: 1.45 (9H, s), 2.11 (3H, s), 2.17 (3H, s), 2.71 (6H, s), 3.78-3.89 (4H, m), 4.70 (2H, d, J 11.5 Hz), 4.83 (1H, dt, J 35.6, 7.0 Hz), 7.42 (2H, d, J 8.4 Hz), 7.69 (2H, d, J 8.4 Hz).

### Procedure I: Preparation of (Z)-4-((1-(4-amino-2-fluorobut-2-en-1-yl)-3,5-dimethyl-1H-pyrazol-4-yl)methyl)-N,N-dimethylbenzenesulfonamide hydrochloride (Compound 2)

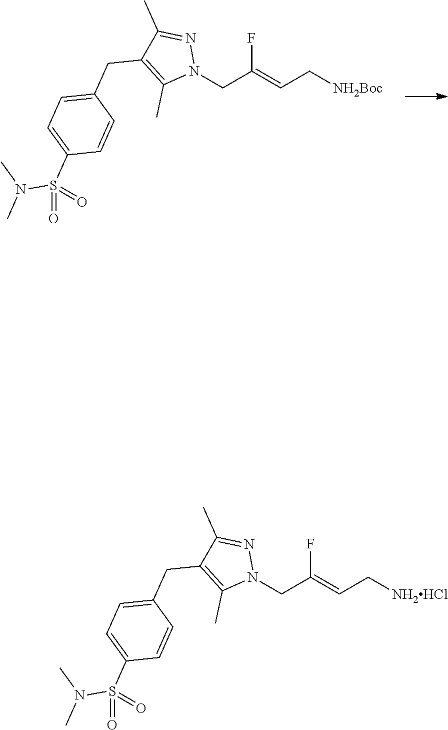

To a stirring solution of (Z)-tert-butyl (4-(4-(4-(N,N-dimethylsulfamoyl)benzyl)-3,5-dimethyl-1H-pyrazol-1-yl)-3-fluorobut-2-en-1-yl)carbamate (63.0 mg, 0.13 mmol) in CH₂Cl₂ (2 mL) at rt was added trifluoroacetic acid (0.5 mL). The resulting solution was stirred at rt for 2 h. All volatiles were removed under reduced pressure and the resulting residue was taken up in ethyl acetate (2 mL). Ethereal HCl (2 M; 0.5 mL) was added at which time a solid white precipitate formed. The solid was collected and dried to afford (Z)-4-((1-(4-amino-2-fluorobut-2-en-1-yl)-3,5-dimethyl-1H-pyrazol-4-yl)methyl)-N,N-dimethylbenzenesulfonamide hydrochloride (38.0 mg, 76%) as a white solid. White solid; m.p. 209-211° C.; ¹H-NMR (300 MHz; CD₃OD) δ ppm: 2.28 (3H, s), 2.37 (3H, s), 2.68 (6H, s), 3.69 (2H, d, J 7.3 Hz), 4.00 (2H, s), 5.13 (2H, d, J 14.4 Hz), 5.32 (1H, dt, J 34.2, 7.4 Hz), 7.42 (2H, d, J 8.5 Hz), 7.73 (2H, d, J 8.4 Hz).

### Example 3

The following compound was prepared according to procedures E, J, G, H and I using appropriate starting materials.

### Preparation of (Z)-4-((1-(4-amino-2-fluorobut-2-en-1-yl)-3,5-diethyl-1H-pyrazol-4-yl)methyl)-N,N-dimethyl benzenesulfonamide hydrochloride (Compound 13)

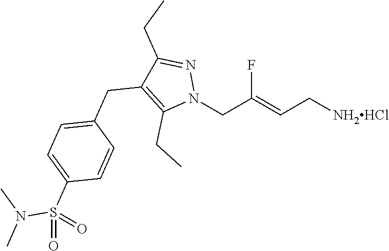

### Procedure J: Preparation of N,N-dimethyl-4-(3-oxo-2-propionylpentyl)benzenesulfonamide

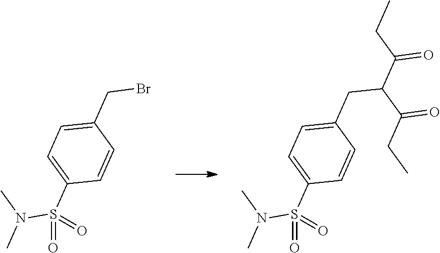 [SMILES: CN(C)S(=O)(=O)c1ccc(CBr)cc1 ; CCC(=O)C(Cc1ccc(S(=O)(=O)N(C)C)cc1)C(=O)CC]

To a stirring solution of sodium ethoxide (122 mg, 1.80 mmol) in ethanol (8 mL) at rt was added heptane-3,5-dione (691 mg, 5.39 mmol) dropwise. The resulting mixture was left to stir for 15 min. To this was added a solution of 4-(bromomethyl)-N,N-dimethylbenzenesulfonamide (500 mg, 1.80 mmol) in THF/ethanol (2 mL; 1:1) dropwise over 5 min. The reaction mixture was warmed to 60° C. and stirring continued at this temperature for 2 h. The reaction mixture was partitioned between water (20 mL) and ethyl acetate (20 mL) and the aqueous layer was extracted with further ethyl acetate (20 mL). The combined organics were dried over Na₂SO₄, concentrated in vacuo and purified by flash column chromatography, eluting with 30-50% ethyl acetate/hexane to afford N,N-dimethyl-4-(3-oxo-2-propionylpentyl)benzenesulfonamide (506 mg, 87%) as a white solid (note that the product was obtained as a complex mixture of keto and enol tautomers, with both the Z and E forms of the enol tautomer present).

### (Z)-4-((1-(4-amino-2-fluorobut-2-en-1-yl)-3,5-diethyl-1H-pyrazol-4-yl)methyl)-N,N-dimethyl benzenesulfonamide hydrochloride (Compound 13)

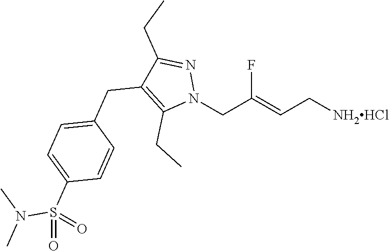

White solid; ¹H-NMR (300 MHz; CD₃OD) δ ppm: 1.11 (3H, t, J 7.5 Hz), 1.20 (3H, t, J 7.5 Hz), 2.67 (6H, s), 2.73 (2H, q, J 7.5 Hz), 2.84 (2H, q, J 7.5 Hz), 3.72 (2H, d, J 6.6 Hz), 4.07 (2H, s), 5.26 (2H, d, J 14.2 Hz), 5.46 (1H, dt, J 34.0, 7.0 Hz), 7.45 (2H, d, J 7.7 Hz), 7.74 (2H, d, J 8.0 Hz).

### Example 4

The following compounds were prepared according to procedures E, F, G, H and I using appropriate starting materials.

### (Z)-3-((1-(4-amino-2-fluorobut-2-en-1-yl)-3,5-dimethyl-1H-pyrazol-4-yl)methyl)-N,N-dimethyl benzenesulfonamide hydrochloride (Compound 5)

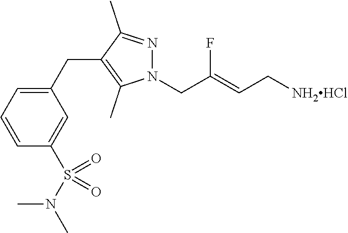

White powder; ¹H-NMR (300 MHz; CD₃OD) δ ppm: 2.23 (3H, s), 2.34 (3H, s), 2.67 (6H, s), 3.67 (2H, d, J 7.2 Hz), 3.97 (2H, s), 5.08 (2H, d, J 13.2 Hz), 5.20 (1H, dt, J 33.9, 7.1 Hz), 7.48-7.53 (2H, m), 7.56 (1H, t, J 7.5 Hz), 7.63 (1H, d, J 7.5 Hz).

### (Z)-4-(3,5-dimethyl-4-(4-(morpholinosulfonyl)benzyl)-1H-pyrazol-1-yl)-3-fluorobut-2-en-1-amine hydrochloride (Compound 6)

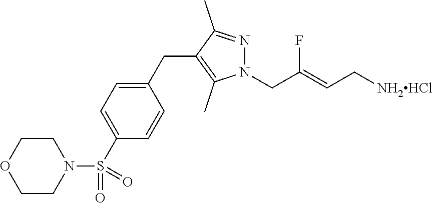

White solid; m.p. 190-197° C.; ¹H-NMR (300 MHz; d₆-DMSO) δ ppm: 1.99 (3H, s), 2.19 (3H, s), 2.80-2.86 (4H, in), 3.44-3.55 (2H, m), 3.62 (4H, dd, J 4.8, 4.8 Hz), 3.82 (2H, s), 5.87 (2H, d, J 13.2 Hz), 5.97 (1H, dt, J 35.8, 7.1 Hz), 7.39 (2H, d, J 8.3 Hz), 7.65 (2H, d, J 8.4 Hz).

### (Z)-4-(3,5-dimethyl-4-(4-(pyrrolidin-1-ylsulfonyl)benzyl)-1H-pyrazol-1-yl)-3-fluorobut-2-en-1-amine hydrochloride (Compound 7)

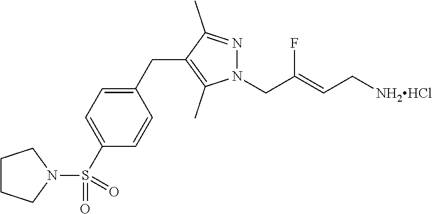

White powder; m.p. 181-184° C.; ¹H-NMR (300 MHz; CD₃OD) δ ppm: 1.72-1.77 (4H, m), 2.24 (3H, s), 2.35 (3H, s), 3.21-3.25 (4H, m), 3.68 (2H, d, J 6.9 Hz), 3.97 (2H, s), 5.07 (2H, d, J 14.1 Hz), 5.25 (1H, dt, J 33.9, 7.5 Hz), 7.40 (2H, d, J 8.7 Hz), 7.78 (2H, d, J 8.4 Hz).

### (Z)-4-((1-(4-amino-2-fluorobut-2-en-1-yl)-3,5-dimethyl-1H-pyrazol-4-yl)methyl)-N-isopropyl-N-methylbenzenesulfonamide hydrochloride (Compound 8)

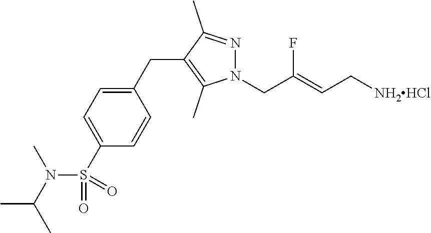

Off-white solid; ¹H-NMR (300 MHz; d₆-DMSO) δ ppm: 0.87 (6H, d, J 6.6 Hz), 1.96 (3H, s), 2.18 (3H, s), 2.62 (3H, s), 3.45-3.53 (1H, m), 3.79 (2H, s), 3.99-4.08 (1H, m), 4.86 (2H, d, J 13.5 Hz), 4.95 (1H, dt, J 35.7, 7.2 Hz), 7.32 (2H, d, J 8.4 Hz), 7.68 (2H, d, J 8.4 Hz), 7.93 (3H, br. s).

### (Z)-4-((1-(4-amino-2-fluorobut-2-en-1-yl)-3,5-dimethyl-1H-pyrazol-4-yl)methyl)-N,N-dimethyl naphthalene-1-sulfonamide hydrochloride (Compound 9)

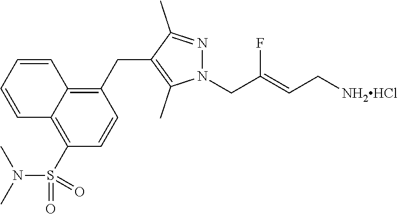

Pale yellow powder; m.p. 70-80° C.; ¹H-NMR (300 MHz; d₆-DMSO) δ ppm: 1.95 (3H, s), 2.14 (3H, s), 2.75 (6H, s), 3.47-3.55 (2H, m), 4.26 (2H, s), 4.92 (2H, d, J 12.8 Hz), 4.94 (1H, dt, J 35.7, 7.3 Hz), 7.10 (1H, d, J 7.7 Hz), 7.71-7.79 (2H, m), 8.01 (1H, d, J 7.6 Hz), 8.08 (3H, br. s), 8.35-8.40 (1H, m), 8.69-8.74 (1H, m).

### Example 5

The following compound was prepared according to procedure K, L, F, G, H and I using appropriate starting materials.

### Preparation of (Z)-4-((1-(4-amino-2-fluorobut-2-en-1-yl)-3,5-dimethyl-1H-pyrazol-4-yl)methyl)-N-isopropyl benzenesulfonamide hydrochloride (Compound 11)

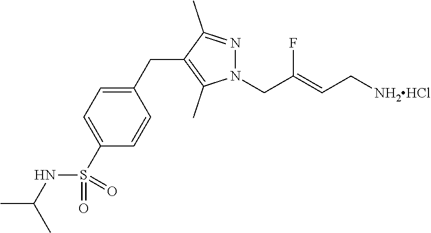

### Procedure K: Preparation of N-(4-methoxybenzyl)propan-2-amine

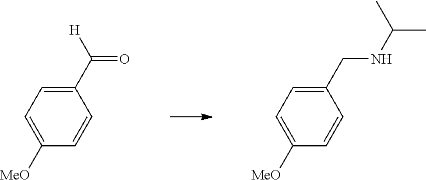

To a stirring solution of 4-methoxybenzaldehyde (2.72 g, 20.0 mmol) in methanol (25 mL) at rt was added isopropylamine (1.75 g, 29.6 mmol). The resulting solution was stirred for 30 min before the addition of sodium cyanoborohydride (2.00 g, 31.8 mmol) in 3 portions. The reaction mixture was stirred at rt for a further 24 h before the removal of methanol under vacuum. Water (20 mL) was added and the product was extracted with ethyl acetate (20 mL×3). The combined organics were then washed with aq. HCl (1 M; 20 mL×3). The combined aqueous extract was basified to pH 12 using aq. NaOH (20%) and the product was extracted with CH₂Cl₂ (20 mL×3). The combined CH₂Cl₂ extracts were dried over Na₂SO₄ and concentrated in vacuo to afford N-(4-methoxybenzyl)propan-2-amine (2.40 g, 67%) as a colourless oil. ¹H-NMR (300 MHz; CD₃OD) δ ppm: 1.10 (6H, d, J 6.5 Hz), 2.86 (1H, sept, J 6.3 Hz), 3.73 (2H, s), 3.81 (3H, s), 6.85 (2H, d, J 8.6 Hz), 7.25 (2H, d, J 8.4 Hz).

### Procedure L: Preparation of 4-(bromomethyl)-N-isopropyl-N-(4-methoxybenzyl) benzene sulfonamide

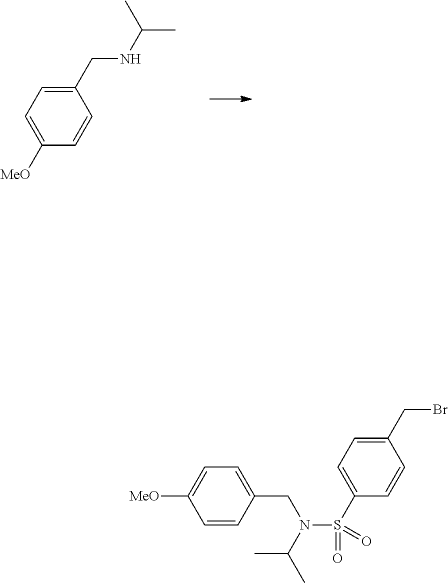 [SMILES: COc1ccc(CNC(C)C)cc1 ; COc1ccc(CN(C(C)C)S(=O)(=O)c2ccc(CBr)cc2)cc1]

To a stirring mixture of N-(4-methoxybenzyl)propan-2-amine (1.79 g, 9.99 mmol), triethylamine (1.40 mL, 10.0 mmol) and 1,2 dichloroethane (20 mL) at 0° C. was added 4-(bromomethyl)benzenesulfonyl chloride (2.95 g, 10.9 mmol) portionwise over 10 min. The resulting suspension was stirred at rt for 3 h before transferring to a separatory funnel and washing with aq. HCl (1 M; 20 mL×2). The organic phase was then dried over Na₂SO₄ and concentrated in vacuo. Purification by flash column, eluting with 20-30% ethyl acetate/hexane afforded 4-(bromomethyl)-N-isopropyl-N-(4-methoxybenzyl)benzenesulfonamide (1.20 g, 29%) as a white foam. ¹H-NMR (300 MHz; CDCl₃) δ ppm: 1.00 (6H, d, J 6.8 Hz), 3.82 (3H, s), 4.17 (1H, sept, J 6.8 Hz), 4.35 (2H, s), 4.50 (2H, s), 6.85 (2H, d, J 8.9 Hz), 7.30 (2H, d, J 8.9 Hz), 7.50 (2H, d, J 8.5 Hz), 7.70 (2H, d, J 8.4 Hz).

### (Z)-4-((1-(4-amino-2-fluorobut-2-en-1-yl)-3,5-dimethyl-1H-pyrazol-4-yl)methyl)-N-isopropyl benzenesulfonamide hydrochloride (Compound 11)

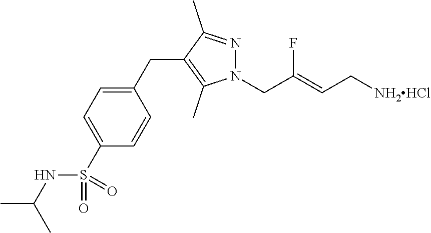

White powder; ¹H-NMR (300 MHz; d₆-DMSO) δ ppm: 0.92 (6H, d, J 6.6 Hz), 1.97 (3H, s), 2.18 (3H, s), 3.15-3.26 (1H, m), 3.46-3.50 (2H, m), 3.77 (2H, s), 4.86 (2H, d, J 13.5 Hz), 4.97 (1H, dt, J 35.7, 7.3 Hz), 7.30 (2H, d, J 8.4 Hz), 7.50 (1H, d, J 7.3 Hz), 7.70 (2H, d, J 8.4 Hz), 8.02 (3H, br. s).

### Example 6

The following compound was prepared according to procedures E, M, N, O, F, G, H and AF using appropriate starting materials.

### Preparation of (Z)-4-((1-(4-amino-2-fluorobut-2-en-1-yl)-3,5-dimethyl-1H-pyrazol-4-yl)methyl)-2-chloro-N,N-dimethylbenzenesulfonamide hydrochloride (Compound 15)

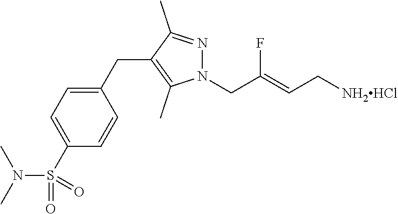

### Procedure M: Preparation of methyl 3-chloro-4-(N,N-dimethylsulfamoyl)benzoate

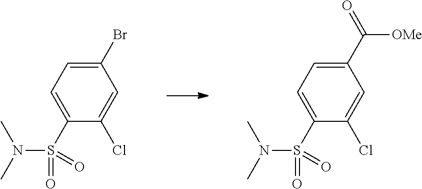 [SMILES: CN(C)S(=O)(=O)c1ccc(Br)cc1Cl ; COC(=O)c1ccc(S(=O)(=O)N(C)C)c(Cl)c1]

A stirred solution of 4-bromo-2-chloro-N,N-dimethylbenzenesulfonamide (250 mg, 0.84 mmol), diacetoxypalladium (18.8 mg, 0.08 mmol) and 1,3-bis(diphenylphosphino)propane (34.5 mg, 0.08 mmol) in methanol (1 mL) and DMF (2 mL) was evacuated and purged with CO gas (×3). Triethylamine (233 μL, 1.67 mmol) was added and the resulting solution was heated at 70° C. under a CO atmosphere for 2 hours. After this time the reaction mixture was diluted with ethyl acetate (40 mL), cooled and washed (sat. aq. NH₄Cl, brine), dried over Na₂SO₄ and concentrated in vacuo. Purification by flash column, eluting with 20% ethyl acetate/hexane, yielded methyl 3-chloro-4-(N,N-dimethylsulfamoyl)benzoate (127 mg, 52%) as a colourless oil. ¹H-NMR (300 MHz; CDCl₃) δ ppm: 2.92 (6H, s), 3.98 (3H, s), 8.03 (1H, dd, J 8.2, 1.6 Hz), 8.14 (1H, d, J 8.2 Hz), 8.18 (1H, d, J 1.6 Hz).

### Procedure N: Preparation of 2-chloro-4-(hydroxymethyl)-N,N-dimethylbenzenesulfonamide

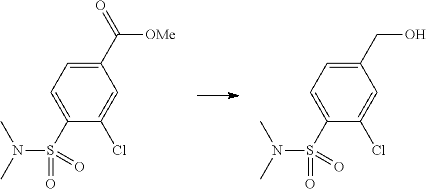 [SMILES: COC(=O)c1ccc(S(=O)(=O)N(C)C)c(Cl)c1 ; CN(C)S(=O)(=O)c1ccc(CO)cc1Cl]

To a stirred solution of methyl 3-chloro-4-(dimethylsulfamoyl)benzoate (250 mg, 0.90 mmol) in CH₂Cl₂ (6 mL) at 0° C. was added DIBAL-H (1 M in CH₂Cl₂; 2.70 mL, 2.70 mmol) slowly. The resulting solution was allowed to warm to rt and stirred for 1 h. The reaction was then quenched by the addition of sat. aq. potassium sodium tartrate (10 mL) and the resulting mixture stirred vigorously until the organic and aqueous phases clearly separated (approx. 30 min). The phases were separated and the aqueous phase extracted with CH₂Cl₂ (10 mL). The combined organic layers were washed (sat. aq. NaHCO₃, brine), dried over Na₂SO₄ and concentrated in vacuo to afford 2-chloro-4-(hydroxymethyl)-N,N-dimethylbenzenesulfonamide (220 mg, 98%) as a colourless oil. ¹H-NMR (300 MHz; CDCl₃) δ ppm: 2.88 (6H, s), 4.77 (2H, s), 7.33-7.34 (1H, m), 7.53-7.55 (1H, m), 7.97 (1H, d, J 8.1 Hz).

### Procedure O: Preparation of 4-(bromomethyl)-2-chloro-N,N-dimethylbenzenesulfonamide

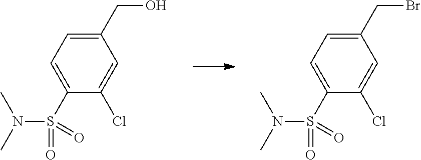

To a stirred solution of 2-chloro-4-(hydroxymethyl)-N,N-dimethylbenzenesulfonamide (220 mg, 0.88 mmol) and triethylamine (0.18 mL, 1.32 mmol) in acetone (4 mL) at 0° C. was added methanesulfonyl chloride (0.08 mL, 1.06 mmol) dropwise. The resulting solution was stirred at 0° C. for 30 min and then filtered and washed (acetone, 2 mL). The filtrate obtained was then cooled to 0° C. whereupon lithium bromide (383 mg, 4.41 mmol) was added portionwise (3 portions, over 10 min). The resulting mixture was allowed to warm to rt and stirred for a further 30 min. The reaction mixture was then partitioned between ethyl acetate (30 mL) and water (30 mL) and the phases separated. The aqueous phase was extracted again with ethyl acetate (20 mL) and the organics were combined, dried over Na₂SO₄ and concentrated in vacuo to give 4-(bromomethyl)-2-chloro-N,N-dimethylbenzenesulfonamide (260 mg, 94%) as a tan oil. ¹H-NMR (300 MHz; CDCl₃) δ ppm: 2.92 (6H, s), 4.45 (2H, s), 7.42 (1H, dd, J 8.2, 1.8 Hz), 7.57 (1H, d, J 1.8 Hz), 8.04 (1H, d, J 8.2 Hz).

### (Z)-4-((1-(4-amino-2-fluorobut-2-en-1-yl)-3,5-dimethyl-1H-pyrazol-4-yl)methyl)-2-chloro-N,N-dimethylbenzenesulfonamide hydrochloride (Compound 15)

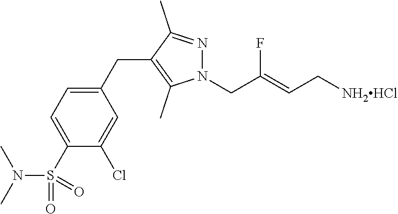

Yellow solid; ¹H-NMR (300 MHz; d₆-DMSO) δ ppm: 2.00 (3H, s), 2.19 (3H, s), 2.79 (6H, s), 3.43-3.54 (2H, m), 4.30 (2H, s), 4.87 (2H, d, J 13.4 Hz), 4.99 (1H, dt, J 36.1, 7.7 Hz), 7.25 (1H, dd, J 8.1, 1.8 Hz), 7.41 (1H, d, J 1.8 Hz), 7.85 (1H, d, J 8.1 Hz).

### Example 7

The following compounds were prepared according to procedures set forth in Example 6 using appropriate starting materials.

### (Z)-4-((1-(4-amino-2-fluorobut-2-en-1-yl)-3,5-dimethyl-1H-pyrazol-4-yl)methyl)-3-chloro-N,N-dimethylbenzenesulfonamide hydrochloride (Compound 3)

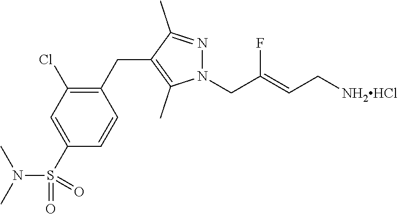

Pale yellow solid; m.p 185-187° C.; ¹H-NMR (300 MHz; CD₃OD) δ ppm: 2.13 (3H, s), 2.27 (3H, s), 2.72 (6H, s), 3.67 (2H, d, J 7.0 Hz), 3.99 (2H, s), 4.99 (2H, d, J 13.3 Hz), 5.12 (1H, dt, J 34.1, 6.9 Hz), 7.28 (1H, d, J 8.1 Hz), 7.64 (1H, dd, J 8.1, 1.7 Hz), 7.82 (1H, d, J 1.7 Hz).

### (Z)-4-((1-(4-amino-2-fluorobut-2-en-1-yl)-3,5-dimethyl-1H-pyrazol-4-yl)methyl)-N,N-dimethyl-2-(trifluoromethoxy)benzenesulfonamide hydrochloride (Compound 17)

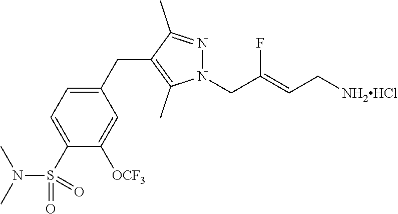

Tan coloured gum; ¹H-NMR (300 MHz; CD₃OD) δ ppm: 2.26 (3H, s), 2.37 (3H, s), 2.81 (6H, s), 3.69 (2H, br. d, J 6.6 Hz), 4.02 (2H, s), 5.11 (2H, d, J 14.5 Hz), 5.21-5.41 (1H, m), 7.27-7.39 (2H, m), 7.91 (1H, br.d, J 6.1 Hz).

### (Z)-4-((1-(4-amino-2-fluorobut-2-en-1-yl)-3,5-dimethyl-1H-pyrazol-4-yl)methyl)-N,N-dimethyl-2-(trifluoromethyl)benzenesulfonamide hydrochloride (Compound 18)

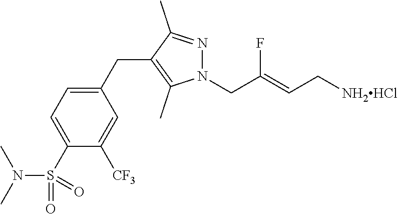

Colourless solid; ¹H-NMR (300 MHz; CD₃OD) δ ppm: 2.22 (3H, s), 2.33 (3H, s), 2.87 (6H, s), 3.67 (2H, d, J 7.2 Hz), 4.01 (2H, s), 5.04 (2H, d, J 14.1 Hz), 5.21 (1H, dt, J 33.6, 7.2 Hz), 7.58 (1H, d, J 8.4 Hz), 7.48 (1H, s), 7.98 (1H, d, J 8.1 Hz).

### (Z)-4-((1-(4-amino-2-fluorobut-2-en-1-yl)-3,5-dimethyl-1H-pyrazol-4-yl)methyl)-2,5-difluoro-N,N-dimethylbenzenesulfonamide hydrochloride (Compound 19)

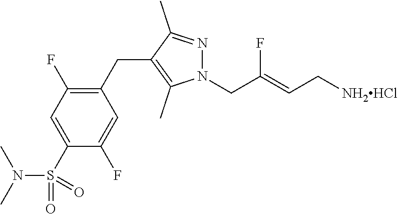

Tan solid; ¹H-NMR (300 MHz; CD₃OD) δ ppm: 2.32 (3H, s), 2.41 (3H, s), 2.82 (6H, s), 3.69 (2H, d, J 5.5 Hz), 3.98 (2H, s), 5.15 (2H, d, J 14.4 Hz), 5.37 (1H, dt, J 33.7, 6.2 Hz), 7.19 (1H, dd, J 9.0, 5.0 Hz), 7.48 (1H, dd, J 8.8, 5.2 Hz).

### Example 8

The following compound was prepared according to procedures P, Q, O, F, G, H and I using appropriate starting materials.

### Preparation of (Z)-4-(4-(3-chloro-4-(methylsulfonyl)benzyl)-3,5-dimethyl-1H-pyrazol-1-yl)-3-fluoro but-2-en-1-amine hydrochloride (Compound 16)

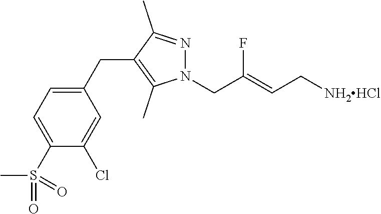

### Procedure P: Preparation of 3-chloro-4-(methylsulfonyl)benzaldehyde

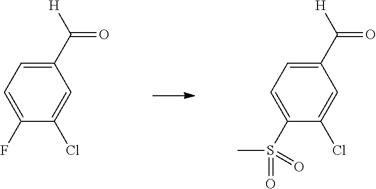

To a stirred solution of 3-chloro-4-fluorobenzaldehyde (634 mg, 4.00 mmol) in DMSO (4 mL) at rt was added sodium methanesulfinate (480 mg, 4.00 mmol) and the resulting solution was heated at 80° C. overnight. The reaction was allowed to cool, quenched by the addition of water (50 mL) and extracted with ethyl acetate (45 mL). The organic layer was then washed (water, brine ×3), dried over MgSO₄, and concentrated in vacuo to afford 3-chloro-4-(methylsulfonyl)benzaldehyde (749 mg, 86%) as a yellow oil. ¹H-NMR (300 MHz; CDCl₃) δ ppm: 3.33 (3H, s), 7.98 (1H, dd, J 8.0, 1.5 Hz), 8.07 (1H, d, J 1.5 Hz), 8.37 (1H, d, J 8.0 Hz), 10.10 (1H, s).

### Procedure Q: Preparation of (3-chloro-4-(methylsulfonyl)phenyl)methanol

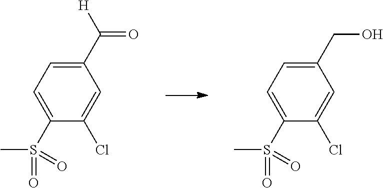

To a stirred solution of 3-chloro-4-(methylsulfonyl)benzaldehyde (736 mg, 3.37 mmol) in ethanol (16 mL) at 0° C. was added sodium borohydride (191 mg, 5.05 mmol) and the resulting solution was stirred at rt for 25 min. The solvent was removed in vacuo and the resulting residue taken up in ethyl acetate (50 mL). The organic phase was washed (water, brine), dried over MgSO₄ and concentrated in vacuo to afford the title compound (3-chloro-4-(methylsulfonyl)phenyl)methanol (536 mg, 72%) as a clear oil. ¹H-NMR (300 MHz; CD₃OD) δ ppm: 2.22 (1H, t, J 5.9 Hz), 3.28 (3H, s), 4.81 (2H, d, J 5.7 Hz), 7.42-7.47 (1H, m), 7.59-7.47 (1H, m), 8.11 (1H, d, J 8.1 Hz).

### (Z)-4-(4-(3-chloro-4-(methylsulfonyl)benzyl)-3,5-dimethyl-1H-pyrazol-1-yl)-3-fluorobut-2-en-1-amine hydrochloride (Compound 16)

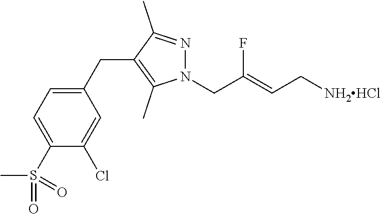

White solid; m.p. 105-115° C.; ¹H-NMR (300 MHz; CD₃OD) δ ppm: 2.23 (3H, s), 2.33 (3H, s), 3.29 (3H, s), 3.67 (2H, d, J 6.6 Hz), 3.97 (2H, s), 5.05 (2H, d, J 14.1 Hz), 5.23 (1H, dt, J 33.9, 7.5 Hz), 7.34 (1H, dd, J 8.1, 1.8 Hz), 7.45 (1H, d, J 1.8 Hz), 8.05 (1H, d, J 8.1 Hz).

### Example 9

The following compounds were prepared according to procedures set forth in Example 8 using appropriate starting materials.

### (Z)-4-(3,5-dimethyl-4-(4-(methylsulfonyl)benzyl)-1H-pyrazol-1-yl)-3-fluorobut-2-en-1-amine hydrochloride (Compound 4)

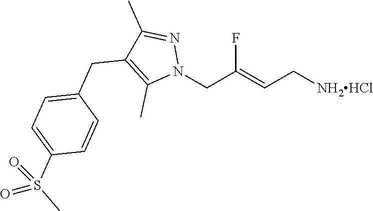

White powder, m.p. 127-137° C.; ¹H-NMR (300 MHz; d₆-DMSO) δ ppm: 1.99 (3H, s), 2.18 (3H, s), 3.16 (3H, s), 3.42-3.50 (2H, m), 3.80 (2H, s), 4.86 (2H, d, J 16.2 Hz), 4.97 (1H, dt, J 35.7, 7.5 Hz), 7.36 (2H, d, J 8.7 Hz), 7.81 (2H, d, J 6.6 Hz), 8.11 (3H, br. s).

### (Z)-4-(4-(2-chloro-4-(methylsulfonyl)benzyl)-3,5-dimethyl-1H-pyrazol-1-yl)-3-fluorobut-2-en-1-amine hydrochloride (Compound 12)

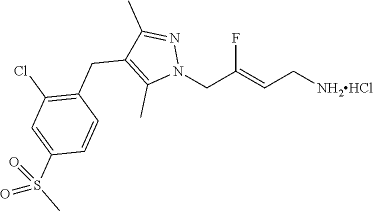

White powder; ¹H-NMR (300 MHz; CD₃OD) δ ppm: 2.22 (3H, s), 2.33 (3H, s), 3.16 (3H, s), 3.69 (2H, d, J 7.2 Hz), 4.06 (2H, s), 5.12 (2H, d, J 14.1 Hz), 5.30 (1H, dt, J 33.9, 6.9 Hz), 7.35 (1H, d, J 8.1 Hz), 7.84 (1H, dd, J 8.1, 1.5 Hz), 8.02 (1H, d, J 1.5 Hz).

### Example 10

The following compound was prepared according to procedures R, S, F, G, H and I using appropriate starting materials.

### Preparation of (Z)-4-((1-(4-amino-2-fluorobut-2-en-1-yl)-3,5-dimethyl-1H-pyrazol-4-yl)methyl)-N,N-dimethylbenzamide hydrochloride (Compound 14)

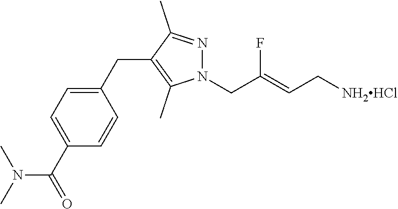

### Procedure R: Preparation of N,N-4-trimethylbenzamide

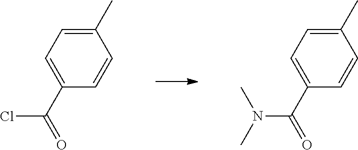

To a stirred solution of N,N-dimethylamine hydrochloride (1.14 g, 14.0 mmol) in DMF (5 mL) at 0° C. was added triethylamine (2.59 mL, 18.6 mmol) and the resulting solution stirred for 5 min. A solution of 4-methylbenzoyl chloride (719 mg, 4.65 mmol) in DMF (5 mL) was then added dropwise over 5 min. The reaction was allowed to stir at 0° C. for a further 30 minutes before warming to rt and partitioning between ethyl acetate (50 mL) and water (50 mL). The phases were separated and the aqueous phase was extracted with ethyl acetate (25 mL×3). The organic layers were then combined, washed (water ×3, brine), dried over Na₂SO₄ and concentrated in vacuo to afford N,N-4-trimethylbenzamide (760 mg, 100%) as a white solid. ¹H-NMR (300 MHz; CDCl₃) δ ppm: 2.39 (3H, s), 3.01 (3H, br. s), 3.11 (3H, br. s), 7.19-7.23 (2H, m), 7.31-7.36 (2H, m).

### Procedure S: Preparation of 4-(bromomethyl)-N,N-dimethylbenzamide

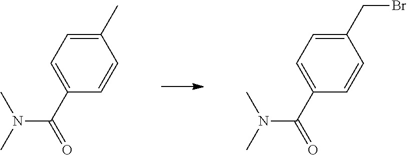

To a stirred solution of N,N-4-trimethylbenzamide (380 mg, 2.33 mmol) in 1,2-dichloroethane (15 mL) at rt was added NBS (415 mg, 2.33 mmol) followed by (E)-2,2′-(diazene-1,2-diyl)bis(2-methylpropanenitrile) (38.0 mg, 0.23 mmol) and the resulting reaction mixture was heated at 80° C. overnight. The reaction was then cooled to rt and the solvent removed in vacuo. The resulting residue was taken up in CH₂Cl₂, adsorbed onto silica and purified by flash column chromatography eluting with 40-70% ethyl acetate/hexane to afford the title compound contaminated with succinimide. This material was then taken up in ethyl acetate (25 mL) and washed with aq. NaOH (2 M, 20 mL) followed by brine (20 mL) and concentrated in vacuo to afford 4-(bromomethyl)-N,N-dimethylbenzamide (163 mg, 29%). ¹H-NMR (300 MHz; CDCl₃) δ ppm: 3.00 (3H, br. s), 3.12 (3H, s), 4.51 (2H, s), 7.38-7.46 (4H, m).

### (Z)-4-((1-(4-amino-2-fluorobut-2-en-1-yl)-3,5-dimethyl-1H-pyrazol-4-yl)methyl)-N,N-dimethyl benzamide hydrochloride (Compound 14)

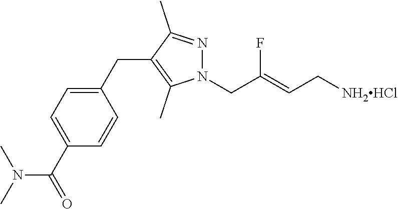

White powder; m.p. 75-82° C.; ¹H-NMR (300 MHz; CD₃OD) δ ppm: 2.31 (3H, s), 2.39 (3H, s), 3.01 (3H, br. s), 3.11 (3H, br. s), 3.69 (2H, d, J 6.6 Hz), 3.95 (2H, s), 5.18 (2H, d, J 14.7 Hz), 5.39 (1H, dt, J 33.9, 6.6 Hz), 7.27 (2H, d, J 7.8 Hz), 7.39 (2H, d, J 7.8 Hz).

### Example 11

The following compound was prepared according to procedures E, T, U, V, W and I using appropriate starting materials.

### Preparation of (Z)-4-((4-(4-amino-2-fluorobut-2-en-1-yl)-3,5-dimethyl-1H-pyrazol-1-yl)methyl)-N,N-dimethylbenzenesulfonamide hydrochloride (Compound 10)

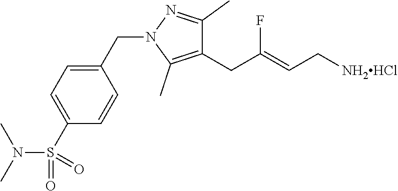

### Procedure T: Preparation of (Z)-tert-butyl (5-acetyl-3-fluoro-6-oxohept-2-en-1-yl)carbamate

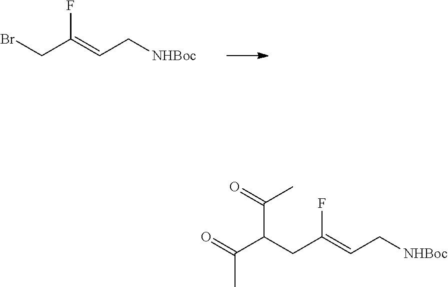 [SMILES: CC(C)(C)OC(=O)NC/C=C(\F)CBr ; CC(=O)C(C/C(F)=C/CNC(=O)OC(C)(C)C)C(C)=O]

To a stirred solution of pentane-2,4-dione (3.08 mL, 30 mmol) in THF (30 mL) at 0° C. was added sodium bis(trimethylsilyl)amide (1 M in THF; 20.0 mL, 20.0 mmol). The resulting suspension was stirred at 0° C. for 5 min before the addition of a solution of (Z)-tert-butyl (4-bromo-3-fluorobut-2-en-1-yl)carbamate (2.68 g, 10.0 mmol) in THF (5 mL) slowly (over 10 min). The resulting reaction mixture was stirred at 5° C. for 5 min before warming to 50° C. and heating for a further 4 h. After this time the reaction was quenched by pouring into water (100 mL). The aqueous phase was extracted with ethyl acetate (25 mL×2) and the organics were then combined, dried over Na₂SO₄ and concentrated in vacuo. The resulting material was purified by flash column chromatography, eluting with 20-40% ethyl acetate/hexane to afford (Z)-tert-butyl (4-bromo-3-fluorobut-2-en-1-yl)carbamate (1.70 g, 59%) as a white solid. ¹H-NMR (300 MHz; CDCl₃) (1:0.1 ratio enol:keto tautomers; asterix denotes discrete signal corresponding to minor keto tautomer) δ ppm: 1.46 (9H, s), 2.16 (6H, s), 2.73* (0.2H, d, J 19.0, 7.3 Hz), 3.13 (1.9H, d, J 9.0, 1.2 Hz), 3.81 (2H, dd, J 6.2, 6.2 Hz), 3.94* (0.1H, t, J 7.1 Hz), 4.72 (1H, dt, J 36.2, 7.5 Hz), 4.79 (1H, dt, J 36.4, 7.1 Hz).

### Procedure U: Preparation of tert-butyl 4-(N,N-dimethylsulfamoyl)benzylcarbamate

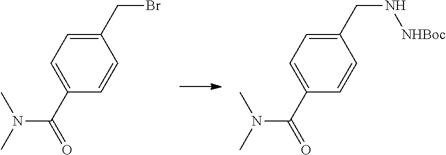 [SMILES: CN(C)C(=O)c1ccc(CBr)cc1 ; CN(C)C(=O)c1ccc(CNNC(=O)OC(C)(C)C)cc1]

To a stirred solution of 4-(bromomethyl)-N,N-dimethylbenzenesulfonamide (200 mg, 0.72 mmol) in DMF (2 mL) at rt was added potassium carbonate (119 mg, 0.86 mmol) and tert-butyl carbazate (475 mg, 3.59 mmol) and the resulting reaction mixture was heated at 80° C. for 30 min. After cooling, the reaction mixture was diluted with ethyl acetate (50 mL) and washed (sat. aq. NH₄Cl, brine), dried over Na₂SO₄ and concentrated in vacuo. Purification by flash column chromatography, eluting with 20% ethyl acetate/CH₂Cl₂ afforded tert-butyl 4-(N,N-dimethylsulfamoyl)benzylcarbamate (240 mg, 100%) as a white solid. ¹H-NMR (300 MHz; CDCl₃) δ ppm: 1.45 (9H, s), 2.71 (6H, s), 4.09 (2H, s), 6.12 (1H, s), 7.54 (2H, d, J 8.3 Hz), 7.74 (2H, d, J 8.4 Hz).

### Procedure V: Preparation of 4-(aminomethyl)-N,N-dimethylbenzenesulfonamide 2,2,2-trifluoroacetic Acid

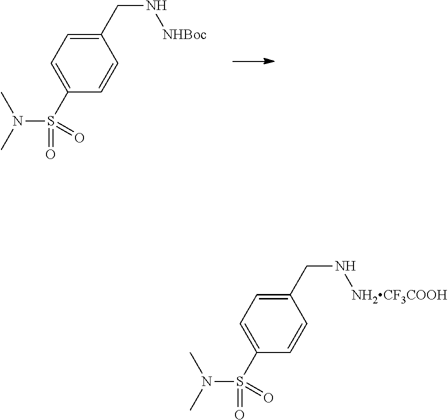

To a stirred solution of tert-butyl 4-(N,N-dimethylsulfamoyl)benzylcarbamate (240 mg, 0.73 mmol) in CH₂Cl₂ (4 mL) at rt was added trifluoroacetic acid (1.00 mL, 13.0 mmol). The solution was stirred at rt for 30 min before further trifluoroacetic acid (1.00 mL, 13.0 mmol) was added. The resulting solution was stirred at rt for 20 min before concentration in vacuo and co-evaporation with ethyl acetate (10 mL×2) to afford 4-(aminomethyl)-N,N-dimethylbenzenesulfonamide trifluoroacetate (340 mg, 100%) as a yellow oil. ¹H-NMR (300 MHz; CD₃OD) δ ppm: 2.70 (6H, s), 4.24 (2H, s), 7.89 (2H, d, J 8.6 Hz), 7.84 (2H, d, J 8.5 Hz).

### Procedure W: Preparation of (Z)-tert-butyl (4-(1-(4-(N,N-dimethylsulfamoyl)benzyl)-3,5-dimethyl-1H-pyrazol-4-yl)-3-fluorobut-2-en-1-yl)carbamate

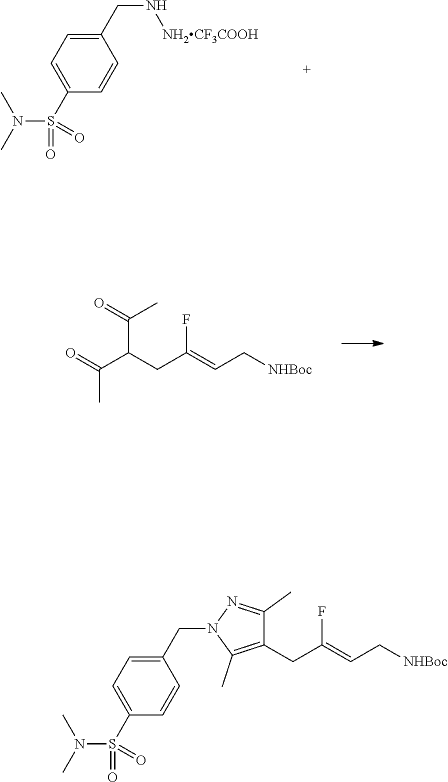

To a stirred solution of 4-(aminomethyl)-N,N-dimethylbenzenesulfonamide trifluoroacetate (170 mg, 0.37 mmol) in ethanol (2 mL) at rt was added N,N-diisopropylethylamine (128 μL, 0.73 mmol). The resulting solution was stirred for 10 min before the addition of (Z)-tert-butyl (5-acetyl-3-fluoro-6-oxohept-2-en-1-yl)carbamate (105 mg, 0.36 mmol) and heated at reflux for 2 hours. After this time the reaction mixture was cooled to rt and concentrated in vacuo. Purification by flash column chromatography, eluting with 20% ethyl acetate/CH₂Cl₂ followed by 2% MeOH in 20% ethyl acetate/hexane, yielded impure material (140 mg) which required subsequent purification via Boc protection/flash column chromatography. As such the combined crude material (140 mg) was taken up in CH₂Cl₂ (4 mL) and triethylamine (0.15 mL, 1.10 mmol) and di-tert-butyl dicarbonate (200 mg, 0.92 mmol) were added at rt. The resulting solution was stirred at rt overnight before concentration in vacuo and purification by flash column chromatography, eluting with 50% ethyl acetate/hexane followed by 2% MeOH in 50% ethyl acetate/hexane, to afford (Z)-tert-butyl (4-(I-(4-(N,N-dimethylsulfamoyl)benzyl)-3,5-dimethyl-1H-pyrazol-4-yl)-3-fluorobut-2-en-1-yl)carbamate (90.0 mg, 51%) as a yellow oil. ¹H-NMR (300 MHz; CD₃OD) δ ppm: 1.44 (9H, s), 2.11 (3H, s), 2.21 (3H, s), 2.70 (6H, s), 3.24 (2H, d, J 12.3 Hz), 3.77 (1H, t, J 6.2 Hz), 4.49-4.72 (3H, m), 7.18 (2H, d, J 8.6 Hz), 7.73 (2H, d, J 8.4 Hz).

### (Z)-4-((4-(4-amino-2-fluorobut-2-en-1-yl)-3,5-dimethyl-1H-pyrazol-1-yl)methyl)-N,N-dimethylbenzenesulfonamide hydrochloride (Compound 10)

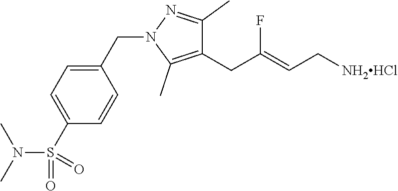

White powder; m.p. 180-185° C.; ¹H-NMR (300 MHz; CD₃OD) δ ppm: 2.11 (3H, s), 2.14 (3H, s), 2.60 (6H, s), 3.35 (2H, d, J 13.6 Hz), 3.40-3.44 (2H, m), 4.76 (1H, dt, J 36.2, 7.3 Hz), 5.34 (2H, s), 7.32 (2H, d, J 8.4 Hz), 7.73 (2H, d, J 8.3 Hz), 8.04 (3H, br. s).

### Example 12

The following compounds were prepared according to procedures set forth in Example 11 using appropriate starting materials.

### (Z)-4-((4-(4-amino-2-fluorobut-2-en-1-yl)-3,5-dimethyl-1H-pyrazol-1-yl)methyl)-3-chloro-N-methylbenzenesulfonamide hydrochloride (Compound 20)

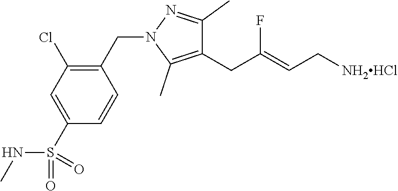

White powder, m.p. 80-86° C.; ¹H-NMR (300 MHz; d₆-DMSO) δ ppm: 2.10 (3H, s), 2.16 (3H, s), 2.43 (3H, d, J 4.4 Hz), 3.37 (2H, d, J 13.4 Hz), 3.41-3.50 (2H, m), 4.76 (1H, dt, J 35.7, 6.8 Hz), 5.35 (2H, s), 6.82 (1H, d, J 8.1 Hz), 7.66 (1H, q, J 4.5 Hz), 7.72 (1H, d, J 8.1 Hz), 7.84 (1H, d, J 1.7 Hz), 8.03 (2H, br. s).

### (Z)-4-((4-(4-amino-2-fluorobut-2-en-1-yl)-3,5-dimethyl-1H-pyrazol-1-yl)methyl)-2-chloro-N-methylbenzenesulfonamide hydrochloride (Compound 22)

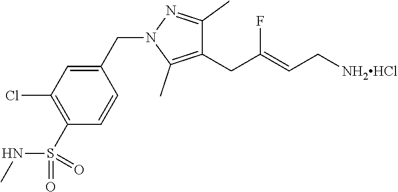

Light yellow powder, ¹H-NMR (300 MHz; CD₃OD) δ ppm: 2.33 (3H, s), 2.34 (3H, s), 2.55 (3H, s), 3.54 (2H, d, J 12.0 Hz), 3.61 (2H, d, J 7.2 Hz), 4.92 (1H, dt, J 30.9, 7.5 Hz), 5.50 (2H, s), 7.21 (1H, dd, J 8.1, 1.5 Hz), 7.43 (1H, d, J 1.2 Hz), 8.05 (1H, d, J 8.1 Hz).

### (Z)-4-((4-(4-amino-2-fluorobut-2-en-1-yl)-3,5-dimethyl-1H-pyrazol-1-yl)methyl)-N-methyl-3-(trifluoromethyl)benzenesulfonamide hydrochloride (Compound 23)

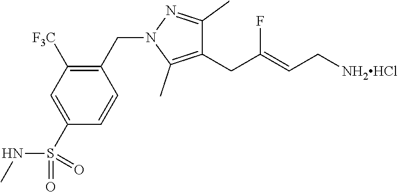

White powder; m.p. 171-172° C.; ¹H-NMR (300 MHz; d₆-DMSO) δ ppm: 2.12 (3H, s), 2.13 (3H, s), 2.43 (3H, d, J 4.7 Hz), 3.39 (2H, d, J 13.2 Hz), 3.42-3.50 (2H, m), 4.77 (1H, dt, J 36.0, 6.8 Hz), 5.46 (2H, s), 6.78 (1H, d, J 8.2 Hz), 7.76 (1H, br. s), 7.72 (1H, d, J 8.1 Hz), 8.09 (1H, s).

### (Z)-4-((4-(4-amino-2-fluorobut-2-en-1-yl)-3,5-dimethyl-1H-pyrazol-1-yl)methyl)-2,5-difluoro-N,N-dimethylbenzenesulfonamide hydrochloride (Compound 25)

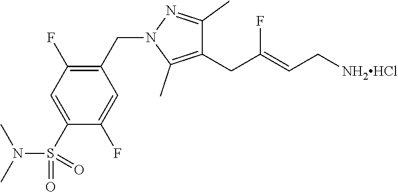

Pale yellow solid; ¹H-NMR (300 MHz; CD₃OD) δ ppm: 2.26 (3H, s), 2.31 (3H, s), 2.83 (6H, d, J 1.9 Hz), 3.49 (2H, d, J 13.7 Hz), 3.60 (2H, d, J 7.6 Hz), 4.85 (1H, dt, J 34.0, 7.5 Hz), 5.44 (2H, s), 6.92 (1H, dd, J 9.8, 5.6 Hz), 7.65 (1H, dd, J 9.0, 5.3 Hz).

### (Z)-4-((4-(4-amino-2-fluorobut-2-en-1-yl)-3,5-dimethyl-1H-pyrazol-1-yl)methyl)-2,5-difluoro-N-methylbenzenesulfonamide hydrochloride (Compound 26)

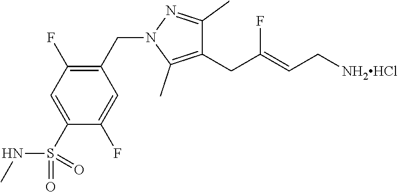

White solid; m.p. 100-103° C. ¹H-NMR (300 MHz; d₆-DMSO) δ ppm: 2.08 (3H, s), 2.20 (3H, s), 2.53 (3H, d, J 5.1 Hz), 3.38 (2H, d, J 14.8 Hz), 3.39-3.47 (2H, m), 4.78 (1H, dt, J 36.1, 6.8 Hz), 5.31 (2H, s), 6.91-7.00 (1H, m), 7.60 (1H, dd, J 8.8, 5.7 Hz), 7.88-8.17 (3H, m).

### Example 13

The following compounds were prepared according to procedures P, Q, O, T, U, V, W and I using appropriate starting materials.

### (Z)-4-(1-(2-chloro-4-(methylsulfonyl)benzyl)-3,5-dimethyl-1H-pyrazol-4-yl)-3-fluorobut-2-en-1-amine hydrochloride (Compound 21)

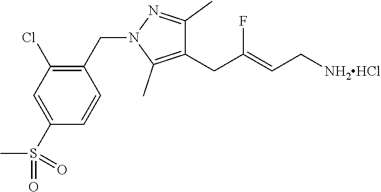

Yellow glass; ¹H-NMR (300 MHz; d₆-DMSO) δ ppm: 2.09 (3H, s), 2.16 (3H, s), 3.27 (3H, s), 3.37 (2H, d, J 13.5 Hz), 3.42-3.45 (2H, m), 4.75 (1H, dt, J 36.0, 7.2 Hz), 5.37 (2H, s), 6.84 (1H, d, J 8.1 Hz), 7.86 (1H, d, J 8.1, 1.5 Hz), 8.11 (3H, br. s), 8.03 (1H, d, J 1.8 Hz).

### (Z)-4-(1-(3-chloro-4-(methylsulfonyl)benzyl)-3,5-dimethyl-1H-pyrazol-4-yl)-3-fluorobut-2-en-1-amine hydrochloride (Compound 24)

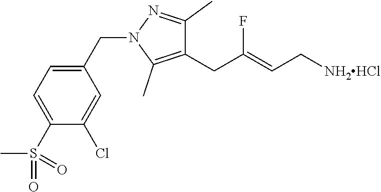

Light yellow powder, m.p. 92-94° C.; ¹H-NMR (300 MHz; CD₃OD) δ ppm: 2.35 (6H, s), 3.31 (3H, s), 3.55 (2H, d, J 13.8 Hz), 3.61 (2H, br. s), 4.95 (1H, dt, partially obscured by H₂O peak), 5.56 (2H, s), 7.31 (1H, d, J 6.6 Hz), 7.51 (1H, s), 8.13 (1H, d, J 6.6 Hz).

### Example 14

The following compounds were prepared according to procedures X, Y, H and I using appropriate starting materials.

### Preparation of (Z)-4-(1-(4-amino-2-fluorobut-2-en-1-yl)-1H-pyrazol-4-yl)-N,N-dimethyl benzenesulfonamide hydrochloride (Compound 1)

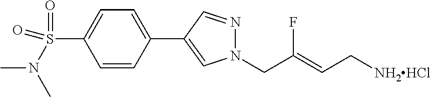

### Procedure X: Preparation of 4-bromo-N,N-dimethylbenzenesulfonamide

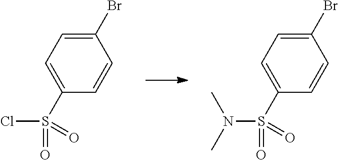

To a stirred solution of N,N-dimethylamine hydrochloride (3.83 g, 47.0 mmol) and pyridine (8.00 mL, 98.9 mmol) in CH₂Cl₂ (16 mL) at 0° C. was added a solution of 4-bromobenzenesulfonyl chloride (4.00 g, 15.7 mmol) in CH₂Cl₂/THF (8 mL, 1:1). The resulting solution was stirred at 0° C. for 30 min and then allowed to warm to rt and stirred overnight. The reaction was quenched by the addition of aq. HCl (2 M; 25 mL) and the organics removed in vacuo. The aqueous phase was then extracted with ethyl acetate (25 mL×2) and the combined organics then washed (water, brine), dried over Na₂SO₄ and concentrated in vacuo to afford 4-bromo-N,N-dimethylbenzenesulfonamide (3.57 g, 86%) as an orange solid. ¹H-NMR (300 MHz; CDCl₃) δ ppm: 2.74 (6H, s), 7.65-7.73 (4H, m).

### Procedure Y: Preparation of N,N-dimethyl-4-(1H-pyrazol-4-yl)benzenesulfonamide

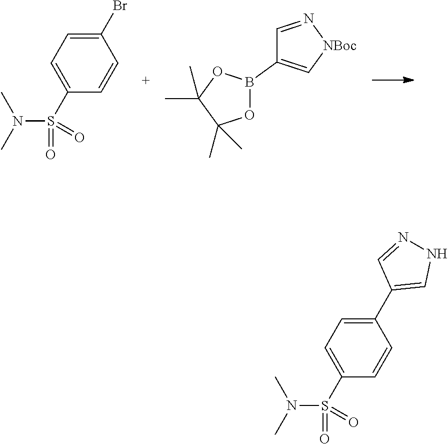 [SMILES: CN(C)S(=O)(=O)c1ccc(Br)cc1 ; CC(C)(C)OC(=O)n1cc(B2OC(C)(C)C(C)(C)O2)cn1 ; CN(C)S(=O)(=O)c1ccc(-c2cn[nH]c2)cc1]

A mixture of 4-bromo-N,N-dimethyl-benzenesulfonamide (200 mg, 0.76 mmol), tert-butyl 4-(4,4,5,5-tetramethyl-1,3,2-dioxaborolan-2-yl)pyrazole-1-carboxylate (267 mg, 0.91 mmol) and cesium carbonate (863 mg, 2.65 mmol) in dioxane/H₂O (5 mL, 4:1) was flushed with nitrogen for 5 min before the addition of tetrakis(triphenylphosphine)palladium(0) (87.5 mg, 0.08 mmol). The resulting mixture was heated at 90° C. overnight before quenching with the addition of water (20 mL) and extraction with ethyl acetate (20 mL×3). The organic phases were combined, dried over Na₂SO₄ and purified by flash column chromatography, eluting with 2% methanol/CH₂Cl₂, to afford N,N-dimethyl-4-(1H-pyrazol-4-yl)benzenesulfonamide (140 mg, 53%) as a white solid. ¹H-NMR (300 MHz; CDCl₃) δ ppm: 2.76 (6H, s), 7.69 (2H, d, J 8.5 Hz), 7.83 (2H, d, J 8.4 Hz), 8.04 (2H, br. s).

### (Z)-4-(1-(2-chloro-4-(methylsulfonyl)benzyl)-3,5-dimethyl-1H-pyrazol-4-yl)-3-fluorobut-2-en-1-amine (Compound 1)

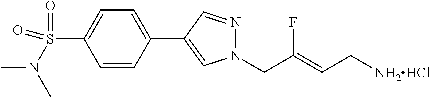

White solid; m.p. 231-234° C.; ¹H-NMR (300 MHz; d₆-DMSO) δ ppm: 2.62 (6H, s), 3.49-3.56 (2H, m), 5.09 (2H, d, J 16.0 Hz), 5.27 (1H, dt, J 35.3, 7.0 Hz), 7.72 (2H, d, J 8.5 Hz), 7.87 (2H, d, J 8.2 Hz), 8.12 (1H, s), 8.45 (1H, s).

### (Z)-3-(1-(4-amino-2-fluorobut-2-en-1-yl)-3,5-dimethyl-1H-pyrazol-4-yl)-N,N-dimethylbenzenesulfonamide (Compound 33)

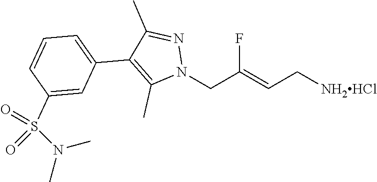

¹H-NMR (300 MHz, DMSO-d₆) δ 8.19 (s, 3H), 7.76-7.62 (m, 3H), 7.55 (t, J=1.7 Hz, 1H), 5.12 (dt, J=35.7, 7.2 Hz, 1H), 4.98 (d, J=14.0 Hz, 2H), 3.50 (t, J=6.6 Hz, 2H), 2.66 (s, 6H), 2.30 (s, 3H), 2.18 (s, 3H).

### Example 15

The following compounds were prepared according to procedures AA-AG using appropriate starting materials.

### (Z)-ethyl 1-(4-amino-2-fluorobut-2-enyl)-4-(4-(N,N-dimethylsulfamoyl)benzyl)-3-methyl-1H-pyrazole-5-carboxylate hydrochloride (Compound 27)

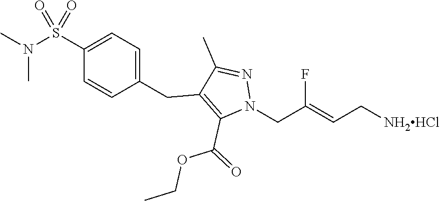

### (Z)-ethyl 1-(4-amino-2-fluorobut-2-enyl)-4-(4-(N,N-dimethylsulfamoyl)benzyl)-5-methyl-1H-pyrazole-3-carboxylate hydrochloride (Compound 28)

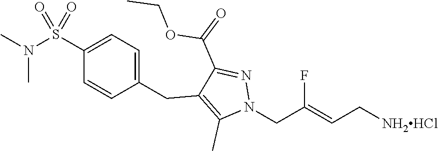

### Procedure AA: Preparation of (Z)-ethyl 2-(methoxyimino)-4-oxopentanoate

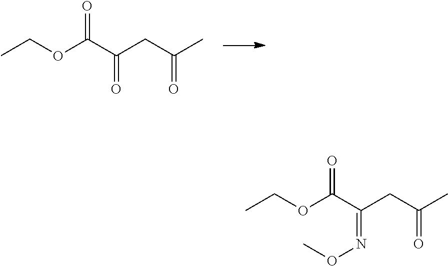

To a solution of ethyl 2,4-dioxopentanoate (1.00 g, 6.32 mmol) in DMF (6 mL) at rt under N₂ was added O-methylhydroxylamine hydrochloride (528 mg, 6.32 mmol) followed by powdered 4 Å molecular sieves (2.00 g). The resulting mixture was left to stir at rt for 48 h. The reaction mixture was partitioned between water (40 mL) and ethyl acetate (50 mL) and the aqueous layer was extracted with further ethyl acetate (40 mL). The combined organics were washed with water (3×40 mL), dried over Na₂SO₄ and concentrated in vacuo to give an orange non-viscous oil. The crude material was purified over silica gel (50 g) eluting with 30% ethyl acetate in hexanes to afford (Z)-ethyl 2-(methoxyimino)-4-oxopentanoate (0.56 g, 48%) as a straw coloured oil. ¹H-NMR (300 MHz; CDCl₃) δ ppm: 1.35 (3H, t, J 7.1 Hz), 2.21 (3H, s), 3.71 (2H, s), 4.07 (3H, s), 4.34 (2H, q, J 7.1 Hz).

### Procedure AB: Preparation of 4-(bromomethyl)-N,N-dimethylbenzenesulfonamide

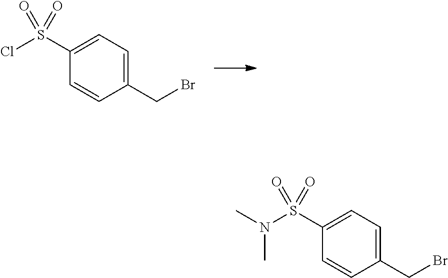

To a stirring solution of 4-(bromomethyl)benzenesulfonyl chloride (5.00 g, 18.6 mmol) in CH₂Cl₂ (40 mL) at 0° C. was added dimethylamine (5.80 mL, 46.4 mmol) drop-wise. Following addition the resulting mixture was left to stir at this temperature for 45 min before partitioning between aq. HCl (1 M, 100 mL) and CH₂Cl₂ (50 mL). The organic layer was washed further with aq. HCl (1 M, 100 mL) and water (50 mL), dried over Na₂SO₄ and concentrated in vacuo to give 4-(bromomethyl)-N,N-dimethylbenzenesulfonamide (2.20 g, 43%) as an off-white solid. ¹H-NMR (300 MHz; CD₃OD) δ ppm: 2.74 (6H, s), 4.52 (2H, s), 7.58 (2H, d, J 8.4 Hz), 7.77 (2H, d, J 8.3 Hz).

### Procedure AC: Preparation of (Z)-ethyl 3-(4-(N,N-dimethylsulfamoyl)benzyl)-2-(methoxyimino)-4-oxopentanoate

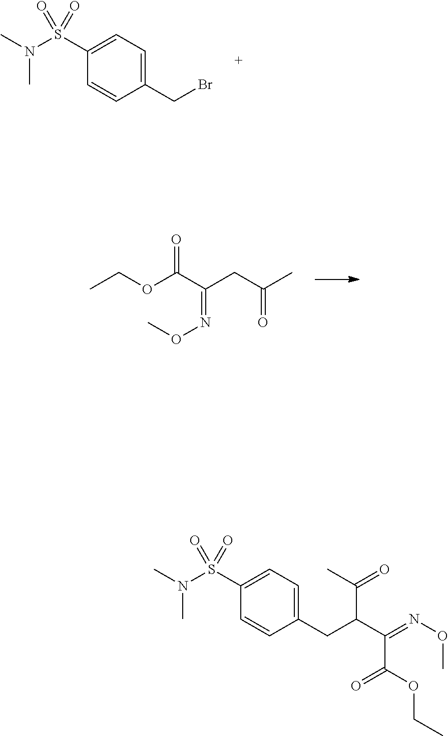 [SMILES: CN(C)S(=O)(=O)c1ccc(CBr)cc1 ; CCOC(=O)/C(CC(C)=O)=N\OC ; CCOC(=O)/C(=N\OC)C(Cc1ccc(S(=O)(=O)N(C)C)cc1)C(C)=O]

To a stirring suspension of ethyl (2Z)-2-methoxyimino-4-oxo-pentanoate (0.56 g, 3.01 mmol) and potassium carbonate (1.04 g, 7.53 mmol) in DMF (10 mL) at rt was added 4-(bromomethyl)-N,N-dimethylbenzenesulfonamide (0.84 g, 3.01 mmol) in one lot. The mixture was left to stir at rt for 3 h. The reaction mixture was partitioned between water (40 mL) and ethyl acetate (40 mL) and the organic layer was washed with further water (3×40 mL), dried over Na₂SO₄ and concentrated in vacuo. The crude material was purified over silica gel (50 g) eluting with 50% ethyl acetate in hexane to afford (Z)-ethyl 3-(4-(N,N-dimethylsulfamoyl)benzyl)-2-(methoxyimino)-4-oxopentanoate (517 mg, 45%) as an off-white solid. ¹H-NMR (300 MHz; CDCl₃) δ ppm: 1.24 (3H, t, J 7.1 Hz), 2.06 (3H, s), 2.65 (6H, s), 2.98 (1H, dd, J 14.0, 9.1 Hz), 3.43 (1H, dd, J 14.0, 5.8 Hz), 3.98 (3H, s), 4.19 (2H, q, J 6.9 Hz), 4.23 (1H, m), 7.28 (2H, d, J 8.1 Hz), 7.62 (2H, d, J 8.1 Hz).

### Procedure AD: Preparation of ethyl 4-(4-(N,N-dimethylsulfamoyl benzyl)-3-methyl-1H-pyrazole-5-carboxylate

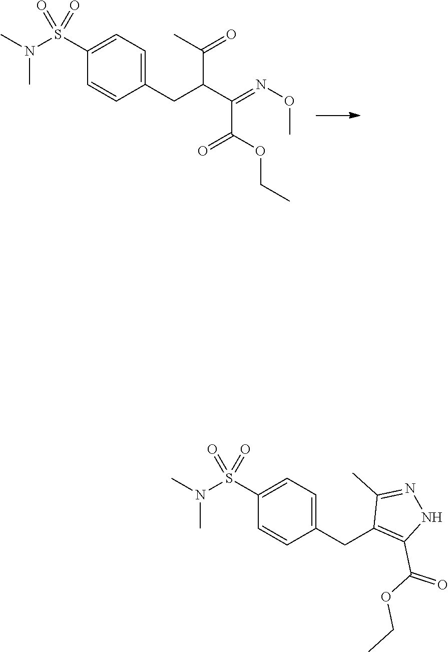

To a stirring solution of (Z)-ethyl 3-(4-(N,N-dimethylsulfamoyl)benzyl)-2-(methoxyimino)-4-oxopentanoate (0.25 g, 0.65 mmol) and 4 Å molecular sieves (250 mg) in ethanol (5 mL) and water (2 mL) was added hydrazine sulfate (0.10 g, 0.78 mmol). The resulting mixture was heated at reflux for 2 h and then stirring was continued at rt overnight. The reaction mixture was partitioned between aq. HCl (2 M; 20 mL) and ethyl acetate (20 mL) and the aqueous layer was extracted with further ethyl acetate (20 mL). The combined organics were washed with sat. aq. NaHCO₃ (20 mL), dried over Na₂SO₄ and then concentrated in vacuo to give a pale yellow oil that solidified on standing. The crude material was purified over silica gel (15 g) eluting with 50% ethyl acetate in hexane followed by 100% ethyl acetate to afford ethyl 4-(4-(N,N-dimethylsulfamoyl)benzyl)-3-methyl-1H-pyrazole-5-carboxylate (0.22 g, 96%) as a white solid. ¹H-NMR (300 MHz; CDCl₃) δ ppm: 1.28 (3H, t, J 7.2 Hz), 2.22 (3H, s), 2.68 (6H, s), 4.18 (2H, s), 4.33 (2H, q, J 7.2 Hz), 7.31 (2H, d, J 8.5 Hz), 7.65 (2H, d, J 8.4 Hz).

### Procedure AE: Preparation of (Z)-ethyl 1-(4-(tert-butoxycarbonylamino)-2-fluorobut-2-enyl)-4-(4-(N,N-dimethylsulfamoyl)benzyl)-3-methyl-1H-pyrazole-5-carboxylate and (Z)-ethyl 1-(4-(tert-butoxycarbonylamino)-2-fluorobut-2-enyl)-4-(4-(N,N-dimethylsulfamoyl)benzyl)-5-methyl-1H-pyrazole-3-carboxylate

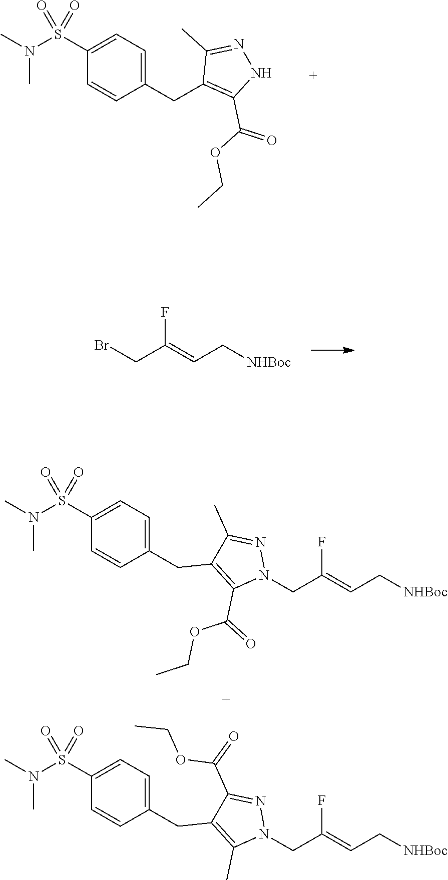

To a stirring suspension of ethyl 4-(4-(N,N-dimethylsulfamoyl)benzyl)-3-methyl-1H-pyrazole-5-carboxylate (50.0 mg, 0.14 mmol) and cesium carbonate (116 mg, 0.36 mmol) in DMF (1.5 mL) at rt under N₂ was added (Z)-tert-butyl 4-bromo-3-fluorobut-2-enylcarbamate (38.2 mg, 0.14 mmol) in one lot. The resulting mixture was heated at 60° C. for 2 h. The reaction mixture was partitioned between water (20 mL) and ethyl acetate (20 mL) and the organic layer was washed with further water (20 mL×3); dried over Na₂SO₄ and concentrated in vacuo to give a colourless oil. The crude material was purified over silica gel (10 g) eluting with 60% ethyl acetate in hexanes to give (Z)-ethyl 1-(4-(tert-butoxycarbonylamino)-2-fluorobut-2-enyl)-4-(4-(N,N-dimethylsulfamoyl)benzyl)-3-methyl-1H-pyrazole-5-carboxylate (50 mg, 65%) followed by (Z)-ethyl 1-(4-(tert-butoxycarbonylamino) but-2-enyl)-4-(4-(N,N-dimethylsulfamoyl)-benzyl)-5-methyl-1H-pyrazole-3-carboxylate (22 mg, 29%).

### Procedure AF: Preparation of (Z)-ethyl 1-(4-amino-2-fluorobut-2-enyl)-4-(4-(N,N-dimethylsulfamoyl)benzyl)-3-methyl-1H-pyrazole-5-carboxylate hydrochloride (Compound 27)

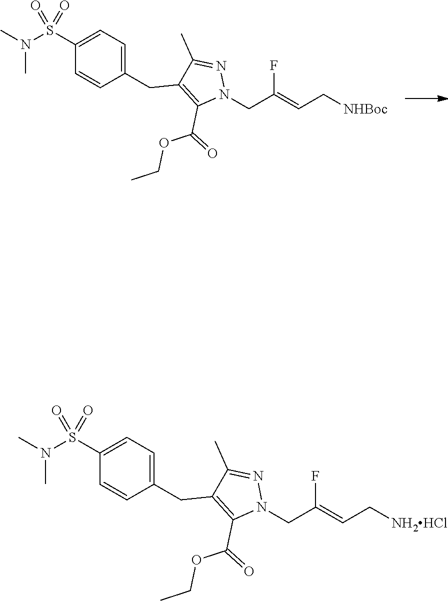

To a stirring solution of (Z)-ethyl 1-(4-(tert-butoxycarbonylamino)-2-fluorobut-2-enyl)-4-(4-(N,N-dimethylsulfamoyl)benzyl)-3-methyl-1H-pyrazole-5-carboxylate (50.0 mg, 0.09 mmol) in ethanol (1.0 mL) at rt was added ethereal HCl (2 M; 1.16 mL, 2.32 mmol) and the resulting mixture was left to stir for 6 h. The reaction mixture was concentrated in vacuo and then diethyl ether was added to the residue at which time a solid precipitate formed. The contents were transferred to a vial and the solid was spun down in a centrifuge. The supernatant was decanted and the solid “cake” was washed by addition of ethyl acetate and brief sonication. The solid was once again spun down on the centrifuge. After decanting the supernatant the solid was dried under high vacuum to give (Z)-ethyl 1-(4-amino-2-fluorobut-2-enyl)-4-(4-(N,N-dimethylsulfamoyl)benzyl)-3-methyl-1H-pyrazole-5-carboxylate hydrochloride (32.0 mg, 73%). White solid; m.p. 157-159° C.; ¹H-NMR (300 MHz; CD₃OD) δ ppm: 1.22 (3H, t, J 7.1 Hz), 2.19 (3H, s), 2.67 (6H, s), 3.64 (2H, br. d, J 7.3 Hz), 4.23 (2H, s), 4.31 (2H, q, J 7.1 Hz), 5.04 (1H, dt, J 33.7, 7.5 Hz), 5.37 (2H, d, J 12.5 Hz), 7.38 (2H, d, J 8.6 Hz), 7.70 (2H, d, J 8.4 Hz).

### Procedure AG: Preparation of (Z)-ethyl 1-(4-amino-2-fluorobut-2-enyl)-4-(4-(N,N-dimethylsulfamoyl)benzyl)-5-methyl-1H-pyrazole-3-carboxylate hydrochloride (Compound 28)

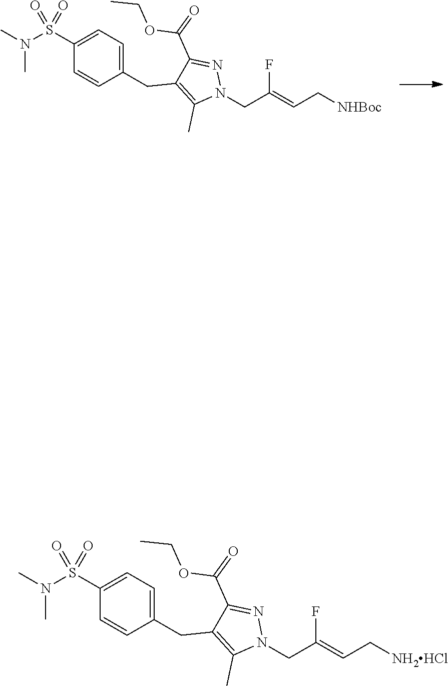

To a stirring solution of (Z)-ethyl 1-(4-(tert-butoxycarbonylamino)-2-fluorobut-2-enyl)-4-(4-(N,N-dimethylsulfamoyl)benzyl)-5-methyl-1H-pyrazole-3-carboxylate (22.0 mg, 0.04 mmol) in ethanol (1.0 mL) at rt was added ethereal HCl (2 M; 0.50 mL, 1.02 mmol) and the resulting mixture was left to stir for 4 h. The reaction mixture was concentrated in vacuo to give (Z)-ethyl 1-(4-amino-2-fluorobut-2-enyl)-4-(4-(N,N-dimethylsulfamoyl)benzyl)-5-methyl-1H-pyrazole-3-carboxylate hydrochloride (12.0 mg, 62%). White foamy solid; ¹H-NMR (300 MHz; CD₃OD) δ ppm: 1.26 (3H, t, J 6.8 Hz), 2.33 (3H, s), 2.67 (6H, s) 3.67 (2H, br. d, J 6.3 Hz), 4.22 (2H, s), 4.30 (2H, q, J 7.0 Hz), 5.06 (2H, d, J 13.3 Hz), 5.15 (1H, dt, J 33.6, 6.9 Hz), 7.40 (2H, d, J 8.3 Hz), 7.68 (2H, d, J 8.4 Hz).

### Example 16

The following compounds were prepared according to procedures AB-AE, AH and AF using appropriate starting materials.

### (Z)-1-(4-amino-2-fluorobut-2-enyl)-4-(4-(N,N-dimethylsulfamoyl)benzyl)-3-methyl-1H-pyrazole-5-carboxylic acid hydrochloride (Compound 30)

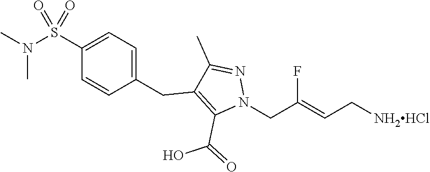

### (Z)-1-(4-amino-2-fluorobut-2-enyl)-4-(4-(N,N-dimethylsulfamoyl)benzyl)-5-methyl-1H-pyrazole-3-carboxylic acid hydrochloride (Compound 31)

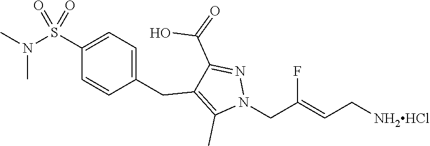

### Procedure AH: Preparation of (Z)-1-(4-(tert-butoxycarbonylamino)-2-fluorobut-2-enyl)-4-(4-(N,N-dimethylsulfamoyl)benzyl)-3-methyl-1H-pyrazole-5-carboxylic Acid

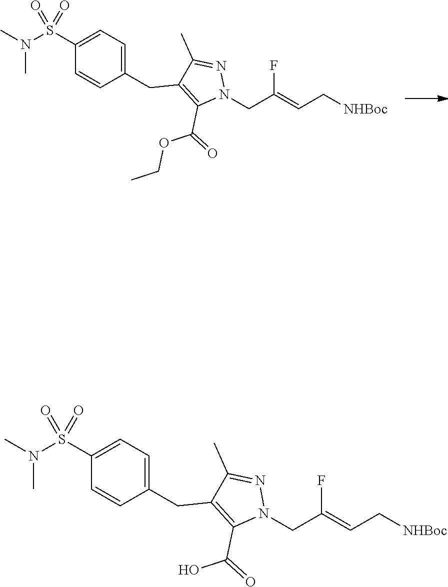 [SMILES: CCOC(=O)c1c(Cc2ccc(S(=O)(=O)N(C)C)cc2)c(C)nn1C/C(F)=C/CNC(=O)OC(C)(C)C ; Cc1nn(C/C(F)=C/CNC(=O)OC(C)(C)C)c(C(=O)O)c1Cc1ccc(S(=O)(=O)N(C)C)cc1]

To a stirred solution of (Z)-ethyl 1-(4-(tert-butoxycarbonylamino)-2-fluorobut-2-enyl)-4-(4-(N,N-dimethylsulfamoyl)benzyl)-3-methyl-1H-pyrazole-5-carboxylate (200 mg, 0.37 mmol) in methanol (1 mL) at rt was added aqueous potassium hydroxide (10% w/w; 1.00 mL, 15.5 mmol). The resulting solution was stirred at this temperature for 1 h. After concentrating the reaction mixture in vacuo, water (5 mL) was added and the pH was adjusted to 5 by addition of aqueous HCl (2 M). The resulting off-white solid was collected by filtration and the filter-cake was washed with water. The solid was then redissolved in CH₂Cl₂, dried over Na₂SO₄ and concentrated in vacuo to afford (Z)-1-(4-(tert-butoxycarbonylamino)-2-fluorobut-2-enyl)-4-(4-(N,N-dimethylsulfamoyl)benzyl)-3-methyl-1H-pyrazole-5-carboxylic acid (170 mg, 90%) as a yellow gum. ¹H-NMR (300 MHz; CDCl₃) δ ppm: 1.43 (9H, s), 2.16 (3H, s), 2.70 (6H, s), 3.74-3.84 (2H, m), 4.19 (2H, s), 4.72-5.00 (2H, m), 5.25 (2H, d, J 11.6 Hz), 7.31 (2H, d, J 8.2 Hz), 7.66 (2H, d, J 8.3 Hz).

### (Z)-1-(4-amino-2-fluorobut-2-enyl)-4-(4-(N,N-dimethylsulfamoyl)benzyl)-3-methyl-1H-pyrazole-5-carboxylic acid hydrochloride (Compound 30)

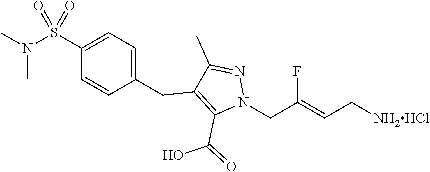

White solid; m.p. 164-166° C.; ¹H-NMR (300 MHz; d₆-DMSO) δ ppm: 2.08 (3H, s), 2.59 (6H, s), 3.45-3.53 (2H, m), 4.16 (2H, s), 4.99 (1H, dt, J 35.6, 7.2 Hz), 5.32 (2H, d, J 13.2 Hz), 7.37 (2H, d, J 8.5 Hz), 7.66 (2H, d, J 8.4 Hz), 8.01 (3H, br. s).

### (Z)-1-(4-amino-2-fluorobut-2-enyl)-4-(4-(N,N-dimethylsulfamoyl)benzyl)-5-methyl-1H-pyrazole-3-carboxylic acid hydrochloride (Compound 31)

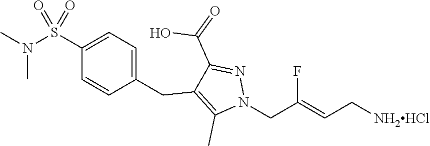

Off-white solid; m.p. 262-264° C.; ¹H-NMR (300 MHz; d₆-DMSO) δ ppm: 2.25 (3H, s), 2.58 (6H, s), 3.46-3.55 (2H, m), 4.15 (2H, s), 5.09 (2H, d, J 14.0 Hz), 5.10 (1H, dt, J 35.5, 7.1 Hz), 7.39 (2H, d, J 8.1 Hz), 7.64 (2H, d, J 8.4 Hz), 8.01 (3H, br. s).

### Example 17

The following compounds were prepared according to procedures AB-AE, AH, AI and AF using appropriate starting materials.

### (Z)-1-(4-amino-2-fluorobut-2-enyl)-4-(4-(N,N-dimethylsulfamoyl)benzyl)-N,N,3-trimethyl-1H-pyrazole-5-carboxamide hydrochloride (Compound 29)

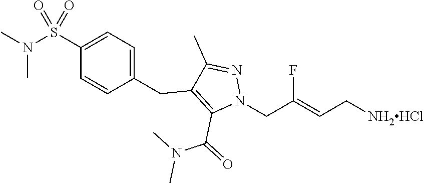

### (Z)-1-(4-amino-2-fluorobut-2-enyl)-4-(4-(N,N-dimethylsulfamoyl)benzyl)-N,N,5-trimethyl-1H-pyrazole-3-carboxamide hydrochloride (Compound 32)

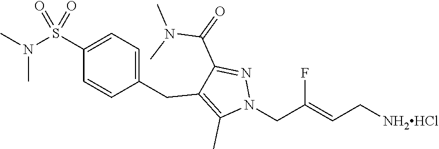

### Procedure AI: Preparation of (Z)-tert-butyl 4-(3-(dimethylcarbamoyl)-4-(4-(N,N-dimethylsulfamoyl)benzyl)-5-methyl-1H-pyrazol-1-yl)-3-fluorobut-2-enylcarbamate

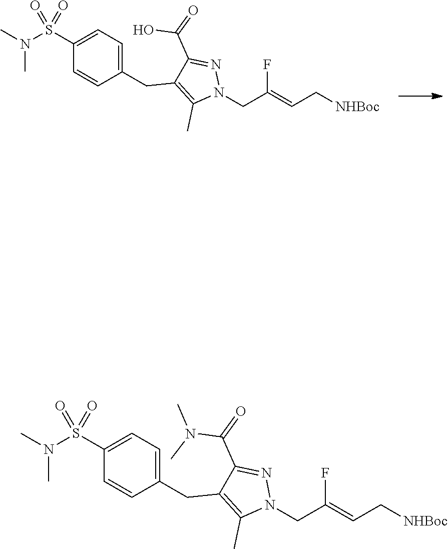 [SMILES: Cc1c(Cc2ccc(S(=O)(=O)N(C)C)cc2)c(C(=O)O)nn1C/C(F)=C/CNC(=O)OC(C)(C)C ; Cc1c(Cc2ccc(S(=O)(=O)N(C)C)cc2)c(C(=O)N(C)C)nn1C/C(F)=C/CNC(=O)OC(C)(C)C]

To a stirring solution of dimethylamine hydrochloride (12 mg, 0.15 mmol) in DMF (1 mL) was added triethylamine (68 μL, 050 mmol). The resulting mixture was stirred for 15 min. To this was added (Z)-1-(4-(tert-butoxycarbonylamino)-2-fluorobut-2-enyl)-4-(4-(N,N-dimethylsulfamoyl)-benzyl)-5-methyl-1H-pyrazole-3-carboxylic acid (50 mg, 0.10 mmol) and HATU (45 mg, 0.12 mmol) and stirring was continued for 2 h. The reaction mixture was partitioned between water (15 mL) and ethyl acetate (20 mL) and the aqueous layer was extracted with further ethyl acetate (2×20 mL). The combined organics were washed with aqueous HCl (1 M; 15 mL), sat. aq. NH₄Cl (15 mL), sat aq. NaCl (15 mL), dried over Na₂SO₄ and concentrated in vacuo. The crude material was purified over silica gel eluting with 80% ethyl acetate in hexane followed by 10% methanol in CH₂Cl₂ to afford (Z)-tert-butyl 4-(3-(dimethylcarbamoyl)-4-(4-(N,N-dimethylsulfamoyl)benzyl)-5-methyl-1H-pyrazol-1-yl)-3-fluorobut-2-enylcarbamate (50 mg, 95%). ¹H-NMR (300 MHz; CDCl₃) δ ppm: 1.44 (9H, s), 2.17 (3H, s), 2.69 (6H, s), 3.03 (3H, s), 3.07 (3H, s), 3.82 (2H, app. t, J 5.8 Hz), 4.00 (2H, s), 4.64 (1H, br. s), 4.74 (2H, d, J 12.3 Hz), 4.87 (1H, dt, J 35.3, 7.1 Hz), 7.36 (2H, d, J 8.4 Hz), 7.66 (2H, d, J 8.3 Hz).

### (Z)-1-(4-amino-2-fluorobut-2-enyl)-4-(4-(N,N-dimethylsulfamoyl)benzyl)-N,N,3-trimethyl-1H-pyrazole-5-carboxamide hydrochloride (Compound 29)

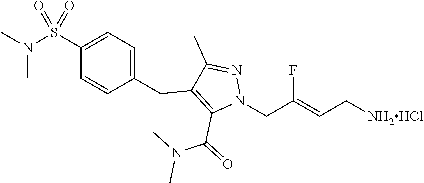

Pale yellow solid; m.p. 77-80° C.; ¹H-NMR (300 MHz; CD₃OD) δ ppm: 2.17 (3H, s), 2.67 (6H, s), 2.87 (3H, s), 2.94 (3H, s), 3.63 (2H, br. d, J 6.8 Hz), 3.90 (2H, br. s), 4.81 (2H, d, J 12.6 Hz), 5.10 (1H, dt, J 33.6, 7.2 Hz), 7.40 (2H, d, J 8.1 Hz), 7.70 (2H, d, J 8.1 Hz).

### (Z)-1-(4-amino-2-fluorobut-2-enyl)-4-(4-(N,N-dimethylsulfamoyl)benzyl)-N,N,5-trimethyl-1H-pyrazole-3-carboxamide hydrochloride (Compound 32)

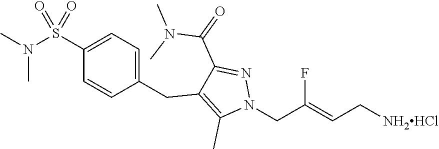

Off-white solid; ¹H-NMR (300 MHz; d₆-DMSO) δ ppm: 2.23 (3H, s), 2.58 (6H, s), 2.89 (3H, s), 2.92 (3H, s), 3.50 (2H, br. s), 3.91 (2H, s), 5.01 (2H, d, J 13.8 Hz), 5.06 (1H, dt, J 36.3, 7.1 Hz), 7.40 (2H, d, J 7.9 Hz), 7.64 (2H, d, J 7.9 Hz), 8.01 (3H, br. s).

### Example 18

The following compounds were prepared according to procedures AA-AE, AJ, AK and I using appropriate starting materials.

### (Z)-4-((1-(4-amino-2-fluorobut-2-en-1-yl)-5-(methoxymethyl)-3-methyl-1H-pyrazol-4-yl)methyl)-2-chloro-N,N-dimethylbenzenesulfonamide (compound 38)

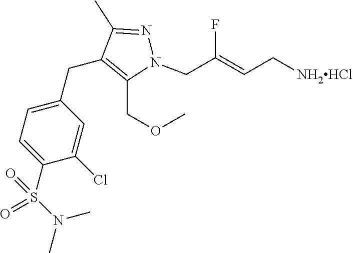

### (Z)-4-((1-(4-amino-2-fluorobut-2-en-1-yl)-3-(methoxymethyl)-5-methyl-1H-pyrazol-4-yl)methyl)-2-chloro-N,N-dimethylbenzenesulfonamide (Compound 39)

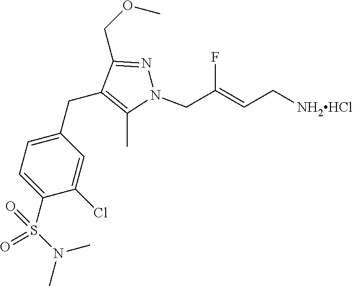

### Procedure AJ: Preparation of tert-butyl (Z)-(4-(4-(3-chloro-4-(N,N-dimethylsulfamoyl)benzyl)-5-(hydroxymethyl)-3-methyl-1H-pyrazol-1-yl)-3-fluorobut-2-en-1-yl)carbamate

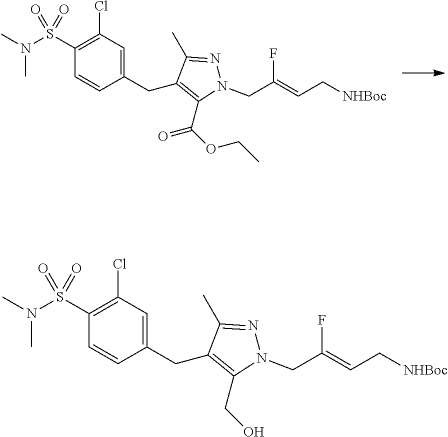 [SMILES: CCOC(=O)c1c(Cc2ccc(S(=O)(=O)N(C)C)c(Cl)c2)c(C)nn1C/C(F)=C/CNC(=O)OC(C)(C)C ; Cc1nn(C/C(F)=C/CNC(=O)OC(C)(C)C)c(CO)c1Cc1ccc(S(=O)(=O)N(C)C)c(Cl)c1]

To a stirring solution of ethyl (Z)-1-(4-((tert-butoxycarbonyl)amino)-2-fluorobut-2-en-1-yl)-4-(3-chloro-4-(N,N-dimethylsulfamoyl)benzyl)-3-methyl-1H-pyrazole-5-carboxylate (620 mg, 1.08 mmol) in CH₂Cl₂ (15.0 mL) at 0° C. was added diisobutylaluminum hydride (1.0 M in CH₂Cl₂; 3.25 mL, 3.25 mmol) was added slowly. The resulting solution was stirred at this temperature for 30 mins. The reaction mixture was poured into saturated aqueous potassium sodium tartrate (100 mL), and the mixture was stirred vigorously for one hour. After transferring to a separatory funnel, the aqueous phase extracted with CH₂Cl₂ (30 mL×3). The combined organic layers were washed with NaHCO₃ and brine, dried over Na₂SO₄, and concentrated in vacuo to afford crude tert-butyl (Z)-(4-(4-(3-chloro-4-(N,N-dimethylsulfamoyl)benzyl)-5-(hydroxymethyl)-3-methyl-1H-pyrazol-1-yl)-3-fluorobut-2-en-1-yl)carbamate (600 mg, 100%) as a yellow oil. This material was progressed to the next step without purification.

### Procedure AK: Preparation of tert-butyl (Z)-(4-(4-(3-chloro-4-(N,N-dimethylsulfamoyl)benzyl)-5-(methoxymethyl)-3-methyl-1H-pyrazol-1-yl)-3-fluorobut-2-en-1-yl)carbamate

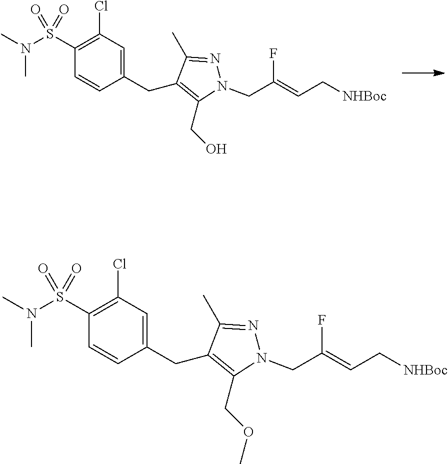 [SMILES: Cc1nn(C/C(F)=C/CNC(=O)OC(C)(C)C)c(CO)c1Cc1ccc(S(=O)(=O)N(C)C)c(Cl)c1 ; COCc1c(Cc2ccc(S(=O)(=O)N(C)C)c(Cl)c2)c(C)nn1C/C(F)=C/CNC(=O)OC(C)(C)C]

To a stirring solution of tert-butyl (Z)-(4-(4-(3-chloro-4-(N,N-dimethylsulfamoyl)benzyl)-5-(hydroxymethyl)-3-methyl-1H-pyrazol-1-yl)-3-fluorobut-2-en-1-yl)carbamate (175 mg, 0.33 mmol) in THF (3.0 mL) at 0° C. under Ar was added sodium hydride (60% dispersion in mineral oil; 19.8 mg, 0.49 mmol). The resulting mixture was stirred at 0° C. for 15 min. To this was then added iodomethane (0.03 mL, 0.49 mmol), and after warming to rt, stirring was continued for a further 30 min. The reaction mixture was partitioned between sat. aq. NaCl (30 mL) and ethyl acetate (30 mL), and the aqueous layer was extracted with further ethyl acetate (20 mL). The combined organics were dried over Na₂SO₄, and then concentrated in vacuo. The crude material was first purified over silica gel, eluting with 65% ethyl acetate in hexane followed by ethyl acetate to afford the crude desired product. This material was then purified further using reverse-phase chromatography to give tert-butyl (Z)-(4-(4-(3-chloro-4-(N,N-dimethylsulfamoyl)benzyl)-5-(methoxymethyl)-3-methyl-1H-pyrazol-1-yl)-3-fluorobut-2-en-1-yl)carbamate (85.0 mg, 47%). ¹H-NMR (300 MHz, CDCl₃) δ ppm: 7.95 (d, J=8.2 Hz, 1H), 7.28 (s, 1H), 7.13 (dd, J=8.1, 1.8 Hz, 1H), 4.91 (dt, J=35.1, 7.0 Hz, 1H), 4.82 (d, J=13.3 Hz, 2H), 4.68 (s, 1H), 4.36 (s, 2H), 3.89-3.77 (m, 4H), 3.29 (s, 3H), 2.88 (s, 6H), 2.12 (s, 3H), 1.44 (s, 9H).

### (Z)-4-((1-(4-amino-2-fluorobut-2-en-1-yl)-5-(methoxymethyl)-3-methyl-1H-pyrazol-4-yl)methyl)-2-chloro-N,N-dimethylbenzenesulfonamide (compound 38)

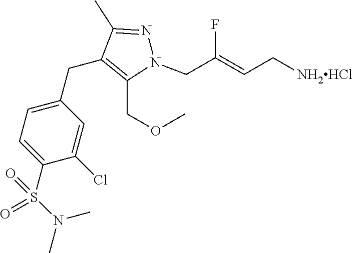

¹H-NMR (300 MHz, CD₃OD) δ ppm: 7.94 (d, J=8.2 Hz, 1H), 7.43 (d, J=1.7 Hz, 1H), 7.30 (dd, J=8.2, 1.7 Hz, 1H), 5.15 (dt, J=34.1, 7.7 Hz, 1H), 5.04 (d, J=13.6 Hz, 2H), 4.54 (s, 2H), 3.97 (s, 2H), 3.67 (d, J=7.5 Hz, 2H), 3.34 (s, 3H), 2.85 (s, 6H), 2.17 (s, 3H).

### (Z)-4-((1-(4-amino-2-fluorobut-2-en-1-yl)-3-(methoxymethyl)-5-methyl-1H-pyrazol-4-yl)methyl)-2-chloro-N,N-dimethylbenzenesulfonamide (Compound 39)

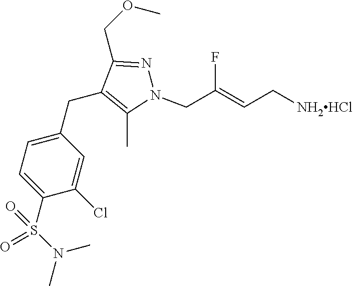

¹H-NMR (300 MHz, CD₃OD) δ ppm: 7.93 (d, J=8.2 Hz, 1H), 7.45 (d, J=1.7 Hz, 1H), 7.31 (dd, J=8.2, 1.7 Hz, 1H), 5.15 (dt, J=33.8, 7.5 Hz, 1H), 5.03 (d, J=13.4 Hz, 2H), 4.40 (s, 2H), 3.96 (s, 2H), 3.71-3.62 (m, 2H), 3.33 (s, 3H), 2.86 (s, 6H), 2.31 (s, 3H).

### Example 19

The following compounds were prepared according to procedures AA-AE, AJ, AL, AM and I using appropriate starting materials.

### (Z)-4-((1-(4-amino-2-fluorobut-2-en-1-yl)-5-(1-hydroxyethyl)-3-methyl-1H-pyrazol-4-yl)methyl)-2-chloro-N,N-dimethylbenzenesulfonamide (Compound 36)

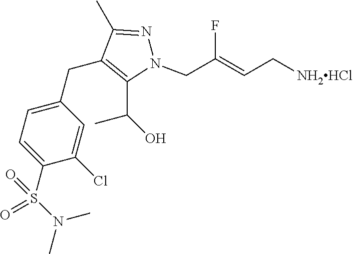

### (Z)-4-((1-(4-amino-2-fluorobut-2-en-1-yl)-3-(1-hydroxyethyl)-5-methyl-1H-pyrazol-4-yl)methyl)-2-chloro-N,N-dimethylbenzenesulfonamide (Compound 37)

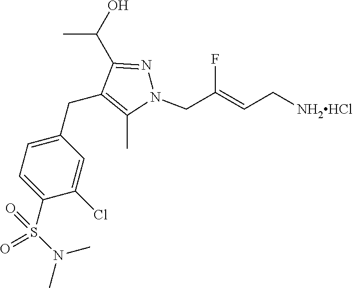

### Procedure AL: Preparation of tert-butyl (Z)-(4-(4-(3-chloro-4-(N,N-dimethylsulfamoyl)benzyl)-5-formyl-3-methyl-1H-pyrazol-1-yl)-3-fluorobut-2-en-1-yl)carbamate

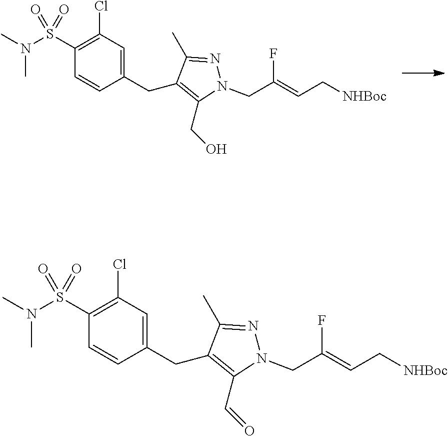 [SMILES: Cc1nn(C/C(F)=C/CNC(=O)OC(C)(C)C)c(CO)c1Cc1ccc(S(=O)(=O)N(C)C)c(Cl)c1 ; Cc1nn(C/C(F)=C/CNC(=O)OC(C)(C)C)c(C=O)c1Cc1ccc(S(=O)(=O)N(C)C)c(Cl)c1]

To a stirring solution of tert-butyl (Z)-(4-(4-(3-chloro-4-(N,N-dimethylsulfamoyl)benzyl)-5-(hydroxymethyl)-3-methyl-1H-pyrazol-1-yl)-3-fluorobut-2-en-1-yl)carbamate (150 mg, 0.28 mmol) in CH₂Cl₂ (2.5 mL) at 0° C. was added Dess-Martin periodinane (144 mg, 0.34 mmol) in one lot. The resulting solution was stirred at this temperature for 1 h. The reaction mixture was poured into a mixture of sat. aq. Na₂S₂O₅ and sat. aq. NaHCO₃ (1:1, 100 mL). After stirring for 5 mins, the mixture was extracted with CH₂Cl₂ (30 mL×3). The combined organic layer was dried over Na₂SO₄, and then concentrated in vacuo to afford crude tert-butyl (Z)-(4-(4-(3-chloro-4-(N,N-dimethylsulfamoyl)benzyl)-5-formyl-3-methyl-1H-pyrazol-1-yl)-3-fluorobut-2-en-1-yl)carbamate (150 mg, 100%) as a yellow oil. This material was progressed to the next step without further purification.

### Procedure AM: Preparation of tert-butyl (Z)-(4-(4-(3-chloro-4-(N,N-dimethylsulfamoyl)benzyl)-5-(l-hydroxyethyl)-3-methyl-1H-pyrazol-1-yl)-3-fluorobut-2-en-1-yl)carbamate

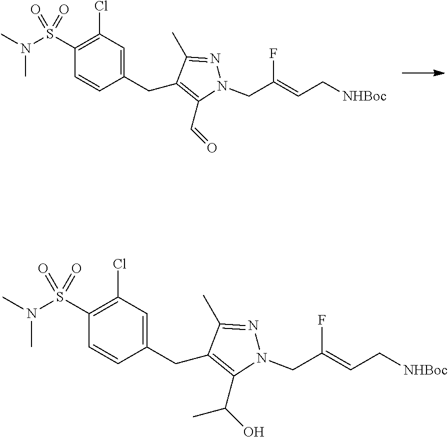 [SMILES: Cc1nn(C/C(F)=C/CNC(=O)OC(C)(C)C)c(C=O)c1Cc1ccc(S(=O)(=O)N(C)C)c(Cl)c1 ; Cc1nn(C/C(F)=C/CNC(=O)OC(C)(C)C)c(C(C)O)c1Cc1ccc(S(=O)(=O)N(C)C)c(Cl)c1]

To a stirring solution of tert-butyl (Z)-(4-(4-(3-chloro-4-(N,N-dimethylsulfamoyl)benzyl)-5-formyl-3-methyl-1H-pyrazol-1-yl)-3-fluorobut-2-en-1-yl)carbamate (150 mg, 0.28 mmol) in THF (4.0 mL) at 0° C. was added bromo(methyl)magnesium (284 uL, 0.85 mmol) slowly. The resulting solution was stirred at 0° C. for 30 mins. The reaction mixture was poured into sat. aq. NH₄Cl (50 mL), and the mixture stirred for 5 min and then extracted with ethyl acetate (20 mL×3). The combined organic layer was washed with brine (30 mL), dried over Na₂SO₄ and then concentrated in vacuo. The crude material was purified by reverse-phase chromatography to afford tert-butyl (Z)-(4-(4-(3-chloro-4-(N,N-dimethylsulfamoyl)benzyl)-5-(1-hydroxyethyl)-3-methyl-1H-pyrazol-1-yl)-3-fluorobut-2-en-1-yl)carbamate (150 mg, 97%) as a yellow oil. ¹H-NMR (300 MHz, CDCl₃) δ ppm: 7.88 (d, J=8.2 Hz, 1H), 7.25 (d, J=1.7 Hz, 1H), 7.09 (dd, J=8.2, 1.7 Hz, 1H), 5.02 (q, J=6.7 Hz, 1H), 4.83-4.89 (m, J=8.3, 3H), 4.68 (dt, J=34.9, 6.9 Hz, 1H), 3.89 (s, 2H), 3.77 (t, J=6.0 Hz, 2H), 2.86 (s, 6H), 2.03 (s, 3H), 1.45-1.36 (m, 12H).

### (Z)-4-((-(4-amino-2-fluorobut-2-en-1-yl)-5-(1-hydroxyethyl)-3-methyl-1H-pyrazol-4-yl)methyl)-2-chloro-N,N-dimethylbenzenesulfonamide (Compound 36)

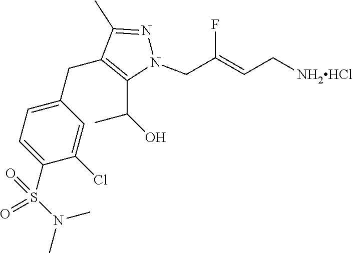

¹H-NMR (300 MHz, CD₃OD) δ ppm: 7.95 (d, J=8.2 Hz, 1H), 7.42 (d, J=1.7 Hz, 1H), 7.29 (dd, J=8.2, 1.7 Hz, 1H), 5.33-5.06 (m, 4H), 4.02 (s, 2H), 3.68 (d, J=7.4 Hz, 2H), 2.86 (s, 6H), 2.17 (s, 3H), 1.41 (d, J=6.7 Hz, 3H).

### (Z)-4-((1-(4-amino-2-fluorobut-2-en-1-yl)-3-(1-hydroxyethyl)-5-methyl-1H-pyrazol-4-yl)methyl)-2-chloro-N,N-dimethylbenzenesulfonamide (Compound 37)

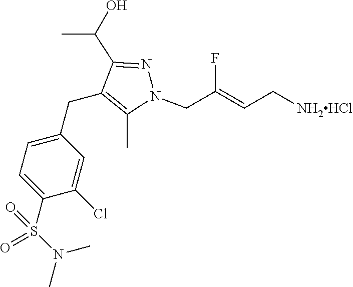

¹H-NMR (300 MHz, d₆-DMSO) δ ppm: 8.00 (s, 3H), 7.83 (d, J=8.1 Hz, 1H), 7.48 (d, J=1.7 Hz, 1H), 7.31 (dd, J=8.2, 1.7 Hz, 1H), 5.03 (dt, J=35.1, 7.2 Hz, 1H), 4.91 (d, J=14.1 Hz, 2H), 4.69 (q, J=6.5 Hz, 1H), 3.94 (s, 2H), 3.49 (t, J=6.4 Hz, 2H), 2.78 (s, 6H), 2.13 (s, 3H), 1.30 (d, J=6.5 Hz, 3H).

### Example 20

The following compounds were prepared according to procedures AN-AQ, H and I using appropriate starting materials.

### (Z)-4-((1-(4-amino-2-fluorobut-2-en-1-yl)-3,5-dimethyl-1H-pyrazol-4-yl)oxy)-2-chloro-N,N-dimethylbenzenesulfonamide (Compound 40)

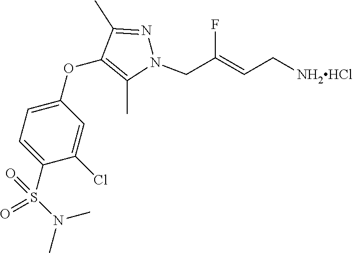

### Procedure AN: Preparation of 2-chloro-4-methoxy-N,N-dimethylbenzenesulfonamide

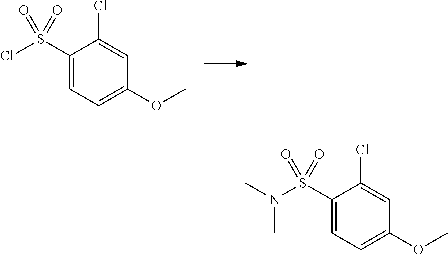

To a stirring solution of aqueous dimethylamine (2.50 mL, 19.7 mmol) in THF (7.0 mL) at 0° C. was added a solution of 2-chloro-4-methoxybenzenesulfonyl chloride (1.00 g, 4.15 mmol) in THF (2 mL) drop-wise over 15 min. The mixture was stirred at 0° C. for 15 min, and then warmed to rt. Stirring at rt was continued overnight. The reaction mixture was concentrated in vacuo, and the resulting residue was partitioned between water and ethyl acetate. The aqueous layer was extracted with further ethyl acetate, and the combined organics were washed with sat. aq. NaCl. After drying over Na₂SO₄, the solvent was removed in vacuo to afford 2-chloro-4-methoxy-N,N-dimethylbenzenesulfonamide (1.10 g, 100%) as a white solid. ¹H-NMR (300 MHz, CDCl₃) δ ppm: 7.99 (d, J=8.9 Hz, 1H), 7.04 (d, J=2.5 Hz, 1H), 6.88 (dd, J=8.9, 2.6 Hz, 1H), 3.88 (s, 3H), 2.87 (s, 6H).

### Procedure AO: Preparation of 2-chloro-4-hydroxy-N,N-dimethylbenzenesulfonamide

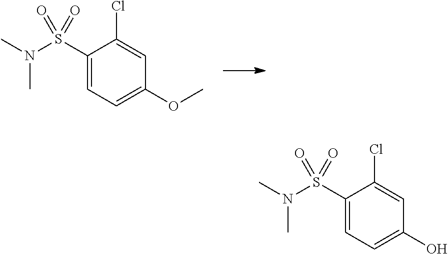

To a stirring solution of 2-chloro-4-methoxy-N,N-dimethylbenzenesulfonamide (1.18 g, 4.73 mmol) in CH₂Cl₂ (10.0 mL) was added boron tribromide (1.37 mL, 14.2 mmol) at 0° C. The reaction mixture was stirred at this temperature for 1.5 h. The reaction was then poured into ice cold water. After transferring to a separatory funnel, sodium hydroxide (1.0 M, 40 mL) was added, and the mixture was washed with CH₂Cl₂ (20 mL×2). The aqeuous phase was acidified to pH=1 with HCl (5.0M), and then extracted with ethyl acetate (20 mL×3). The combined organics were washed with water and brine, then dried over Na₂SO₄, and concentrated in vacuo to afford. 2-chloro-4-hydroxy-N,N-dimethylbenzenesulfonamide (1.00 g, 90%) as a white solid. ¹H NMR (300 MHz, CDCl₃) δ ppm: 7.91 (d, J=8.8 Hz, 1H), 7.03 (d, J=2.5 Hz, 1H), 6.85 (dd, J=8.7, 2.5 Hz, 1H), 2.88 (s, 6H).

### Procedure AP: Preparation of 2-chloro-4-((2,4-dioxopentan-3-yl)oxy)-N,N-dimethylbenzenesulfonamide

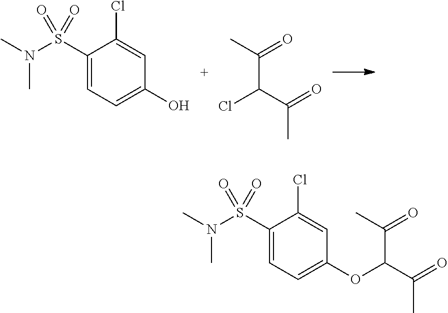

To a stirring mixture of 2-chloro-4-hydroxy-N,N-dimethylbenzenesulfonamide (200 mg, 0.85 mmol) and cesium carbonate (553 mg, 1.70 mmol) in acetone (10.0 mL), was added 3-chloropentane-2,4-dione (115 uL, 1.02 mmol). The resulting mixture was stirred under reflux for 8 h. After cooling to rt, the reaction mixture was filtered to remove inorganics. The filtrate was then concentrated in vacuo to afford crude 2-chloro-4-((2,4-dioxopentan-3-yl)oxy)-N,N-dimethylbenzenesulfonamide (300 mg, 100%) as a yellow gum. This material was progressed to the next step without further purification.

### Procedure AO: Preparation of 2-chloro-4-((3,5-dimethyl-1H-pyrazol-4-yl)oxy)-N,N-dimethylbenzenesulfonamide

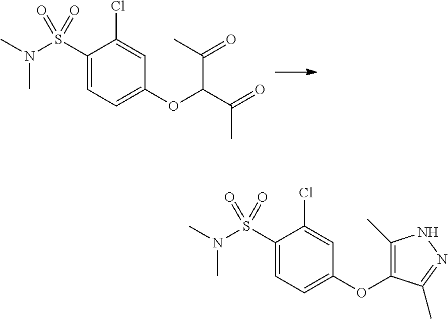 [SMILES: CC(=O)C(Oc1ccc(S(=O)(=O)N(C)C)c(Cl)c1)C(C)=O ; Cc1n[nH]c(C)c1Oc1ccc(S(=O)(=O)N(C)C)c(Cl)c1]

A mixture of 2-chloro-4-((2,4-dioxopentan-3-yl)oxy)-N,N-dimethylbenzenesulfonamide (300 mg, 0.90 mmol) and hydrazine hydrate (0.07 mL, 2.16 mmol) in EtOH (10.0 mL) was stirred under reflux for 2 h. After cooling to rt, the reaction mixture was concentrated in vacuo. The resulting residue was then dissolved in ethyl acetate (50 mL), and washed with water (20 mL×2). The organic layer was dried over Na₂SO₄, and then concentrated in vacuo. The crude material was purified over silica gel, eluting with 50% ethyl acetate in hexane, followed by 50% ethyl acetate, 2% MeOH in hexane to 2-chloro-4-((3,5-dimethyl-1H-pyrazol-4-yl)oxy)-N,N-dimethylbenzenesulfonamide (33.0 mg, 11%) as a brown oil. ¹H-NMR (300 MHz, CDCl₃) δ ppm: 8.00 (d, J=8.9 Hz, 1H), 7.04 (d, J=2.5 Hz, 1H), 6.88 (dd, J=8.9, 2.5 Hz, 1H), 2.90 (s, 6H), 2.14 (s, 6H).

### (Z)-4-((1-(4-amino-2-fluorobut-2-en-1-yl)-3,5-dimethyl-1H-pyrazol-4-yl)oxy)-2-chloro-N,N-dimethylbenzenesulfonamide (Compound 40)

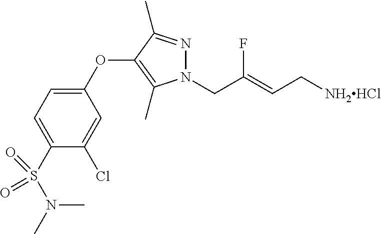

¹H-NMR (300 MHz, CD₃OD) δ ppm: 8.00 (d, J=8.9 Hz, 1H), 7.13 (d, J=2.5 Hz, 1H), 6.98 (dd, J=8.9, 2.5 Hz, 1H), 5.11 (dt, J=33.7, 7.5 Hz, 1H), 4.93 (d, J=13.2 Hz, 2H), 3.68 (d, J=7.5 Hz, 2H), 2.86 (s, 6H), 2.19 (s, 3H), 2.06 (s, 3H).

### Example 21

The following compounds were prepared according to procedures X, AR-AV using appropriate starting materials.

### (Z)-3-(1-(4-amino-2-fluorobut-2-en-1-yl)-3-chloro-5-methyl-1H-pyrazol-4-yl)-N,N-dimethylbenzenesulfonamide (Compound 34)

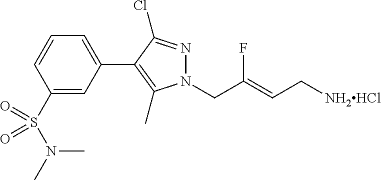

### (Z)-3-(1-(4-amino-2-fluorobut-2-en-1-yl)-5-chloro-3-methyl-1H-pyrazol-4-yl)-N,N-dimethylbenzenesulfonamide (Compound 35)

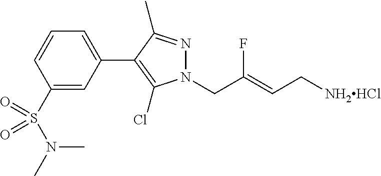

### Procedure AR: Preparation of 5-chloro-3-methyl-1-((2-(trimethylsilyl)ethoxy)methyl)-1H-pyrazole

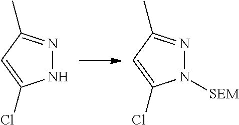

To a stirring solution of 5-chloro-3-methyl-1H-pyrazole (5.00 g, 43.1 mmol) in THF (50.0 mL), SEM-Cl (7.90 g, 47.4 mmol) and NaH (2.27 g, 47.4 mmol) were added at 0° C. The resulting reaction mixture was stirred at rt for 2 h. The reaction mixture was quenched with ice-cold water, and extracted with ethyl acetate. The organics were washed with water, dried over Na₂SO₄ and concentrated in vacuo to afford 5-chloro-3-methyl-1-((2-(trimethylsilyl)ethoxy)methyl)-1H-pyrazole (6.00 g, 60%) as a colorless liquid.

### Procedure AS: Preparation of 3-(5-chloro-3-methyl-1-((2-(trimethylsilyl)ethoxy)methyl)-1H-pyrazol-4-yl-N,N-dimethylbezenesulfonamide

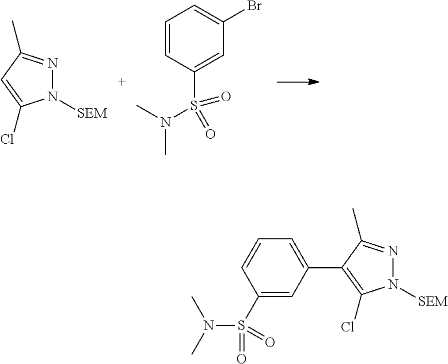 [SMILES: Cc1cc(Cl)n(COCC[Si](C)(C)C)n1 ; CN(C)S(=O)(=O)c1cccc(Br)c1 ; Cc1nn(COCC[Si](C)(C)C)c(Cl)c1-c1cccc(S(=O)(=O)N(C)C)c1]

To a stirring solution of 5-chloro-3-methyl-1-((2-(trimethylsilyl)ethoxy)methyl)-1H-pyrazole (6.00 g, 24.4 mmol) in N,N-dimethylacetamide (60.0 mL) at rt was added 3-bromo-N,N-dimethylbenzenesulfonamide (9.62 g, 36.6 mmol), KOAc (7.18 g, 73.2 mmol) and Pd(OAc)₂ (1.64 g, 2.43 mmol). The resulting reaction mixture was heated to 120° C., and stirring was continued for 16 h. The reaction mixture was diluted with ethyl acetate and filtered through a Celite™ pad and the filtrate was concentrated in vacuo. The crude compound was purified over silica gel, eluting with 20% ethyl acetate in hexane to afford 3-(5-chloro-3-methyl-1-((2-(trimethylsilyl)ethoxy)methyl)-1H-pyrazol-4-yl)-N,N-dimethylbenzenesulfonamide (6.00 g, 60%) as a colorless liquid.

### Procedure AT: Preparation of 3-(5-chloro-3-methyl-1H-pyrazol-4-yl)-N,N-dimethylbenzenesulfonamide

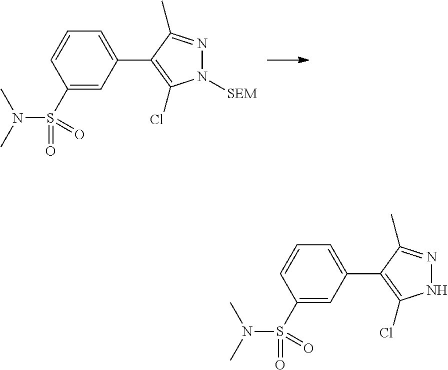 [SMILES: Cc1nn(COCC[Si](C)(C)C)c(Cl)c1-c1cccc(S(=O)(=O)N(C)C)c1 ; Cc1n[nH]c(Cl)c1-c1cccc(S(=O)(=O)N(C)C)c1]

To a stirring solution of 3-(5-chloro-3-methyl-1-((2-(trimethylsilyl)ethoxy)methyl)-1H-pyrazol-4-yl)-N,N-dimethylbenzenesulfonamide (5.00 g, 11.6 mmol) in CH₂Cl₂ (50 mL) at 0° C. was added TFA (20.0 mL). The resulting mixture was heated to 80° C., and stirring was continued for 7 h. The reaction mixture was partitioned between CH₂Cl₂ and sat. aq. NaHCO₃. The organic layer was washed with water and brine, dried over Na₂SO₄ and then concentrated in vacuo to afford 3-(5-chloro-3-methyl-1H-pyrazol-4-yl)-N,N-dimethylbenzenesulfonamide (2.80 g, 82%) as a white solid.

### Procedure AU: Preparation of tert-butyl (Z)-(4-(3-chloro-4-(3-(N,N-dimethylsulfamoyl)phenyl)-5-methyl-H-pyrazol-1-yl)-3-fluorobut-2-en-1-yl)carbamate and tert-butyl (Z)-(4-(5-chloro-4-(3-(N,N-dimethylsulfamoyl)phenyl)-3-methyl-1H-pyrazol-1-yl)-3-fluorobut-2-en-1-yl)carbamate

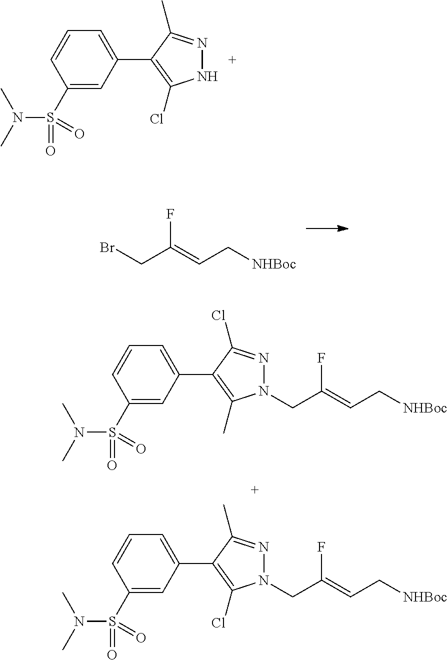 [SMILES: Cc1n[nH]c(Cl)c1-c1cccc(S(=O)(=O)N(C)C)c1 ; CC(C)(C)OC(=O)NC/C=C(\F)CBr ; Cc1c(-c2cccc(S(=O)(=O)N(C)C)c2)c(Cl)nn1C/C(F)=C/CNC(=O)OC(C)(C)C ; Cc1nn(C/C(F)=C/CNC(=O)OC(C)(C)C)c(Cl)c1-c1cccc(S(=O)(=O)N(C)C)c1]

To a stirring solution of 3-(5-chloro-3-methyl-1H-pyrazol-4-yl)-N,N-dimethylbenzenesulfonamide (0.35 g, 12.0 mmol) in DMF (3.0 mL) at rt was added Cs₂CO₃ (0.57 g, 17.5 mmol) followed by tert-butyl (Z)-(4-bromo-3-fluorobut-2-en-1-yl)carbamate (0.27 g, 12.86 mol). The resulting suspension was stirred at 60° C. for 2 h. The reaction mixture was cooled to room temperature, and then partitioned between ethyl acetate and water. The two layers were separated and the organic phase washed with water, dried over Na₂SO₄ and concentrated in vacuo to get crude mixture of tert-butyl (Z)-(4-(5-chloro-4-(3-(N,N-dimethylsulfamoyl)phenyl)-3-methyl-1H-pyrazol-1-yl)-3-fluorobut-2-en-1-yl)carbamate and tert-butyl (Z)-(4-(3-chloro-4-(3-(N,N-dimethylsulfamoyl)phenyl)-5-methyl-1H-pyrazol-1-yl)-3-fluorobut-2-en-1-yl)carbamate (combined 300 mg, 53%). This mixture was progressed to the next step without purification.

### Procedure AV: Preparation of (Z)-3-(1-(4-amino-2-fluorobut-2-en-1-yl)-3-chloro-5-methyl-1H-pyrazol-4-yl)-N,N-dimethylbenzenesulfonamide hydrochloride (Compound 34) and (Z)-3-(1-(4-amino-2-fluorobut-2-en-1-yl)-5-chloro-3-methyl-1H-pyrazol-4-yl)-N,N-dimethylbenzenesulfonamide hydrochloride (Compound 35)

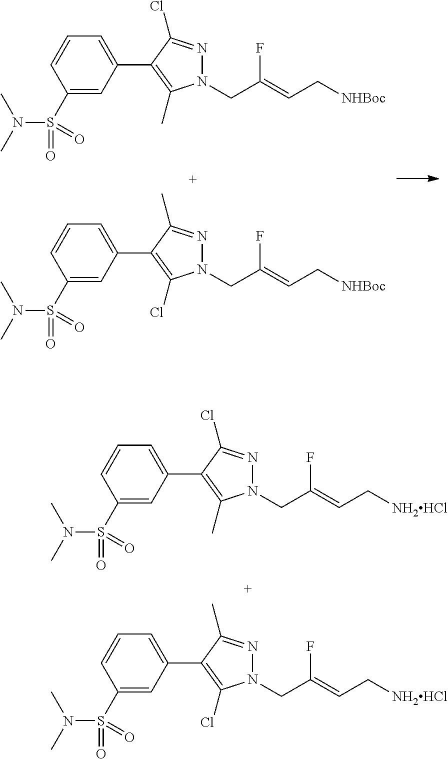

To a stirring suspension of a crude mixture of tert-butyl (Z)-(4-(5-chloro-4-(3-(N,N-dimethylsulfamoyl)phenyl)-3-methyl-H-pyrazol-1-yl)-3-fluorobut-2-en-1-yl)carbamate and tert-butyl (Z)-(4-(3-chloro-4-(3-(N,N-dimethylsulfamoyl)phenyl)-5-methyl-1H-pyrazol-1-yl)-3-fluorobut-2-en-1-yl)carbamate (1.00 g, 2.05 mmol) in 1,4-dioxane (2.0 mL) at rt was added HCl in (4.0 M in 1,4-dioxane, 2.0 mL). The resulting reaction mixture was stirred at rt for 2 h. The solvent was evaporated, and the solid was washed with diethyl ether, and then dried under vacuum. The crude mixture (0.9 g) was purified by SFC (Chiralpak AS-H (250×21) mm; 75.0% CO₂, 25.0% co-solvent: 0.5% diethylamine in methanol) to afford (Z)-3-(1-(4-amino-2-fluorobut-2-en-1-yl)-3-chloro-5-methyl-1H-pyrazol-4-yl)-N,N-dimethyl-benzenesulfonamide hydrochloride (106 mg, 12% over two steps) and (Z)-3-(1-(4-amino-2-fluorobut-2-en-1-yl)-5-chloro-3-methyl-1H-pyrazol-4-yl)-N,N-dimethylbenzenesulfonamide hydrochloride (60 mg, 7% over two steps).

### (Z)-3-(1-(4-amino-2-fluorobut-2-en-1-yl)-3-chloro-5-methyl-1H-pyrazol-4-yl)-N,N-dimethylbenzenesulfonamide hydrochloride (Compound 34)

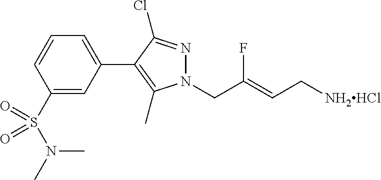

¹H-NMR (300 MHz, d₆-DMSO) δ ppm: 8.02 (s, 3H), 7.79-7.70 (m, 3H), 7.68 (dt, J=2.3, 1.1 Hz, 1H), 5.19 (dt, J=35.2, 7.2 Hz, 1H), 5.09 (d, J=14.6 Hz, 2H), 3.53 (dt, J=7.2 Hz, 2H), 3.32 (s, 6H), 2.36 (s, 3H).

### (Z)-3-(1-(4-amino-2-fluorobut-2-en-1-yl)-5-chloro-3-methyl-1H-pyrazol-4-yl)-N,N-dimethylbenzenesulfonamide hydrochloride (Compound 35)

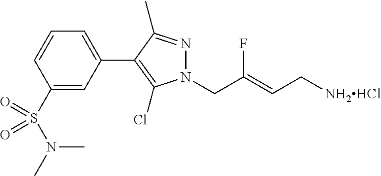

¹H-NMR (300 MHz, d₆-DMSO) δ ppm: 8.07 (s, 3H), 7.80-7.67 (m, 4H), 5.24 (dt, J 35.5, 7.2 Hz, 1H), 5.08 (d, J=15.0 Hz, 2H), 3.53 (t, J=6.3 Hz, 2H), 3.36 (s, 6H), 2.27 (s, 3H).

### Example 22

The following compounds were prepared according to procedures AW, AX, AR, AY-AAD using appropriate starting materials.

### (Z)-4-((1-(4-amino-2-fluorobut-2-en-1-yl)-5-chloro-3-methyl-1H-pyrazol-4-yl)methyl)-2-chloro-N,N-dimethylbenzenesulfonamide (Compound 41)

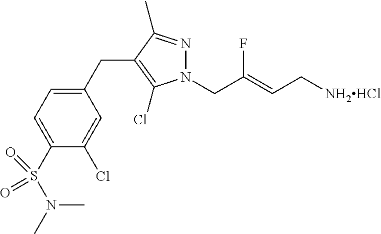

### (Z)-4-((1-(4-amino-2-fluorobut-2-en-1-yl)-3-chloro-5-methyl-1H-pyrazol-4-yl)methyl)-2-chloro-N,N-dimethylbenzenesulfonamide (Compound 42)

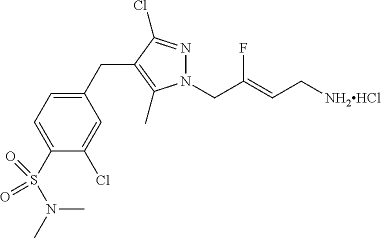

### Procedure AW: Preparation of 2-chloro-4-cyano-N,N-dimethylbenzenesulfonamide

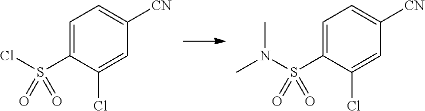

To a stirred solution of 2-chloro-4-cyanobenzenesulfonyl chloride (15.0 g, 63.5 mmol) in THF (100 mL) was added a solution of N,N-dimethylamine (2.0 M in THF, 47.7 mL, 95.4 mmol) drop wise at 0° C. The resulting reaction mixture was stirred at rt for 18 h. After completion of the reaction, the precipitated solid was removed by filtration, and the filtrate was concentrated under vacuum to afford 2-chloro-4-cyano-N,N-dimethylbenzenesulfonamide (15.0 g, 96%) as a pale yellow solid.

### Procedure AX: Preparation of 2-chloro-4-formyl-N,N-dimethylbenzenesulfonamide

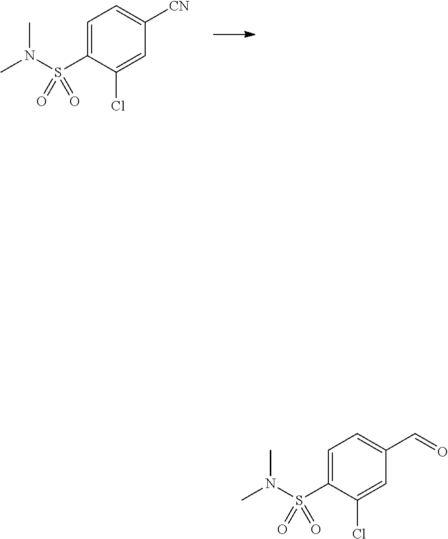

To an ice-cold solution of 2-chloro-4-cyano-N,N-dimethylbenzenesulfonamide (9.00 g, 36.8 mmol) in toluene (60.0 mL), was added diisobutylaluminium hydride (1.5 M in toluene, 55.0 mL, 55.2 mmol) at 0° C. The resulting reaction mixture was stirred at 0° C. for 2 h. After completion of the reaction, the reaction mixture was quenched with conc. HCl. The reaction mixture was extracted with diethyl ether, dried over Na₂SO₄, and concentrated in vacuo. The crude residue was purified over silica gel, eluting with 10% ethyl acetate in hexane to afford 2-chloro-4-formyl-N,N-dimethylbenzenesulfonamide (6.00 g, 66%) as a yellow solid.

### Procedure AY: Preparation of 5-chloro-4-iodo-3-methyl-1-((2-(trimethylsilyl)ethoxy)methyl)-1H-pyrazole

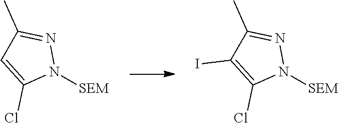 [SMILES: Cc1cc(Cl)n(COCC[Si](C)(C)C)n1 ; Cc1nn(COCC[Si](C)(C)C)c(Cl)c1I]

To a stirred solution of 2-chloro-4-formyl-N,N-dimethylbenzenesulfonamide (7.00 g, 28.36 mmol) in acetonitrile (70 mL) at rt was added NIS (7.0 g, 31.19 mmol). The resulting reaction mixture was stirred at 80° C. for 18 h. After completion of the reaction, the reaction mixture was cooled to rt, and then concentrated under reduced pressure. The residue was diluted with ethyl acetate, and then washed with saturated hypo solution, water and brine solution. The organic layer was dried over Na₂SO₄ and concentrated in vacuo. The crude residue was purified over silica gel, eluting with 5% ethyl acetate in hexane to afford 5-chloro-4-iodo-3-methyl-1-((2-(trimethylsilyl)ethoxy)methyl)-1H-pyrazole (7.00 g, 66%) as a pale yellow solid.

### Procedure AZ: Preparation of 2-chloro-4-((5-chloro-3-methyl-1-((2-(trimethylsilyl)ethoxy)methyl)-1H-pyrazol-4-yl)(hydroxy)methyl)-N,N-dimethylbenzenesulfonamide

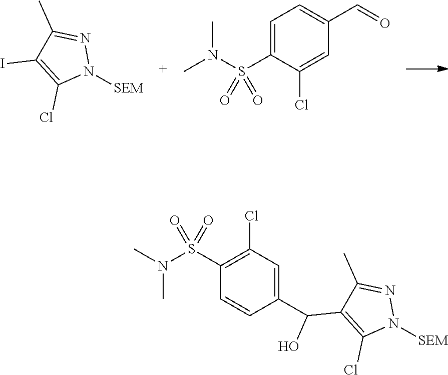 [SMILES: Cc1nn(COCC[Si](C)(C)C)c(Cl)c1I ; CN(C)S(=O)(=O)c1ccc(C=O)cc1Cl ; Cc1nn(COCC[Si](C)(C)C)c(Cl)c1C(O)c1ccc(S(=O)(=O)N(C)C)c(Cl)c1]

To a stirring solution of 5-chloro-4-iodo-3-methyl-1-((2-(trimethylsilyl)ethoxy)methyl)-1H-pyrazole (6.00 g, 16.1 mmol) in THF (60 mL) at rt was added 2-chloro-4-formyl-N,N-dimethylbenzenesulfonamide (4.18 g, 16.1 mmol), followed by isopropylmagnesium chloride lithium chloride complex (1.3 M in THF; 28.0 mL, 37.0 mmol). The resulting reaction mixture was stirred at rt for 4 h. After completion of the reaction, the reaction was quenched with sat. aq. NH₄Cl solution and then extracted with ethyl acetate. The combined organic layer was dried over Na₂SO₄ and concentrated under reduced pressure. The crude residue was purified over silica gel column chromatography eluting with 10% ethyl acetate in hexane to afford 2-chloro-4-((5-chloro-3-methyl-1-((2-(trimethylsilyl)ethoxy)methyl)-1H-pyrazol-4-yl)(hydroxy)methyl)-N,N-dimethylbenzenesulfonamide (2.50 g, 31%) as a pale yellow oil.

### Procedure AAA: Preparation of 2-chloro-4-((5-chloro-3-methyl-1H-pyrazol-4-yl)methyl)-N,N-dimethylbenzenesulfonamide hydrochloride

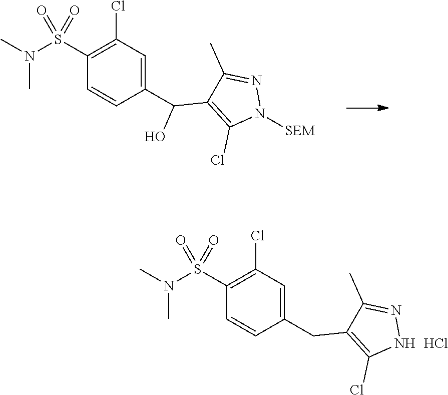 [SMILES: Cc1nn(COCC[Si](C)(C)C)c(Cl)c1C(O)c1ccc(S(=O)(=O)N(C)C)c(Cl)c1 ; Cc1n[nH]c(Cl)c1Cc1ccc(S(=O)(=O)N(C)C)c(Cl)c1 ; Cl]

To a stirred solution of 2-chloro-4-((5-chloro-3-methyl-1-((2-(trimethylsilyl)ethoxy)methyl)-1H-pyrazol-4-yl)(hydroxy)methyl)-N,N-dimethylbenzenesulfonamide (2.50 g, 5.05 mmol) in TFA (20 mL) at rt was added triethylsilane (6.5 mL, 40.4 mmol). The resulting reaction mixture was stirred at 100° C. for 18 h. After completion of reaction, the solvent was concentrated under reduced pressure. The residue was co-distilled with 4 N HCl in 1,4-dioxane, and the resulting solid was washed with diethyl ether, dried under vacuum to give 2-chloro-4-((5-chloro-3-methyl-1H-pyrazol-4-yl)methyl)-N,N-dimethylbenzenesulfon-amide hydrochloride (1.30 g, 73%) as a white solid.

### Procedure AAB: Preparation of tert-butyl (Z)-(4-(5-chloro-4-(3-chloro-4-(N,N-dimethylsulfamoyl)benzyl)-3-methyl-1H-pyrazol-1-yl)-3-fluorobut-2-en-1-yl)carbamate and tert-butyl (Z)-(4-(3-chloro-4-(3-chloro-4-(N,N-dimethylsulfamoyl)benzyl)-5-methyl-1H-pyrazol-1-yl)-3-fluorobut-2-en-1-yl)carbamate

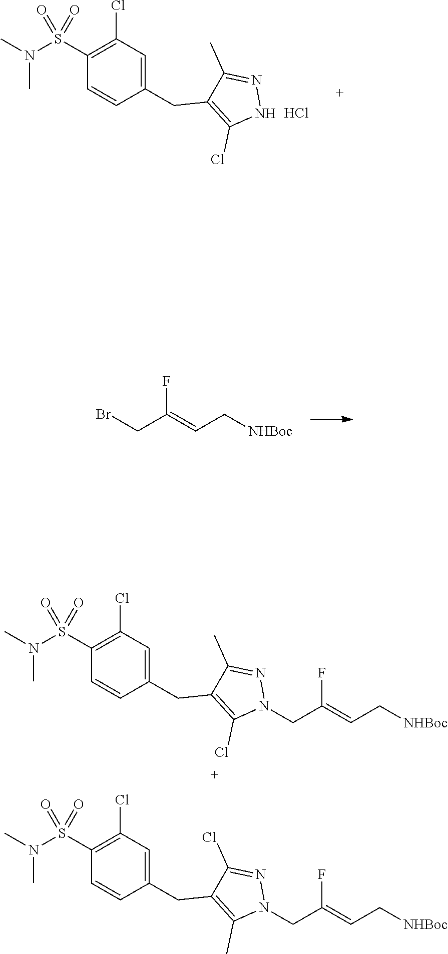 [SMILES: Cc1n[nH]c(Cl)c1Cc1ccc(S(=O)(=O)N(C)C)c(Cl)c1 ; Cl ; CC(C)(C)OC(=O)NC/C=C(\F)CBr ; Cc1nn(C/C(F)=C/CNC(=O)OC(C)(C)C)c(Cl)c1Cc1ccc(S(=O)(=O)N(C)C)c(Cl)c1 ; Cc1c(Cc2ccc(S(=O)(=O)N(C)C)c(Cl)c2)c(Cl)nn1C/C(F)=C/CNC(=O)OC(C)(C)C]

To a solution of 2-chloro-4-((5-chloro-3-methyl-1H-pyrazol-4-yl)methyl)-N,N-dimethylbenzenesulfon-amide hydrochloride (1.30 g, 3.37 mmol) in DMF (6.0 mL) at rt was added Cs₂CO₃ (1.64 g, 5.05 mmol) and tert-butyl (Z)-(4-bromo-3-fluorobut-2-en-1-yl)carbamate (0.90 g, 3.37 mmol). The resulting suspension was stirred at 60° C. for 2 h. After completion of reaction, the reaction mixture was cooled to rt and then partitioned between ethyl acetate and water. The two layers were separated and the organic phase was washed with water, dried over anhydrous Na₂SO₄ and concentrated under reduced pressure to give mixture of regioisomers. Purification was performed by SFC to afford tert-butyl (Z)-(4-(5-chloro-4-(3-chloro-4-(N,N-dimethylsulfamoyl)benzyl)-3-methyl-1H-pyrazol-1-yl)-3-fluorobut-2-en-1-yl)carbamate and tert-butyl (Z)-(4-(3-chloro-4-(3-chloro-4-(N,N-dimethylsulfamoyl)benzyl)-5-methyl-1H-pyrazol-1-yl)-3-fluorobut-2-en-1-yl)carbamate (1.30 g, 72%).

### Procedure AAC: Preparation of (Z)-4-((1-(4-amino-2-fluorobut-2-en-1-yl)-5-chloro-3-methyl-1H-pyrazol-4-yl)methyl)-2-chloro-N,N-dimethylbenzenesulfonamide hydrochloride (Compound 41)

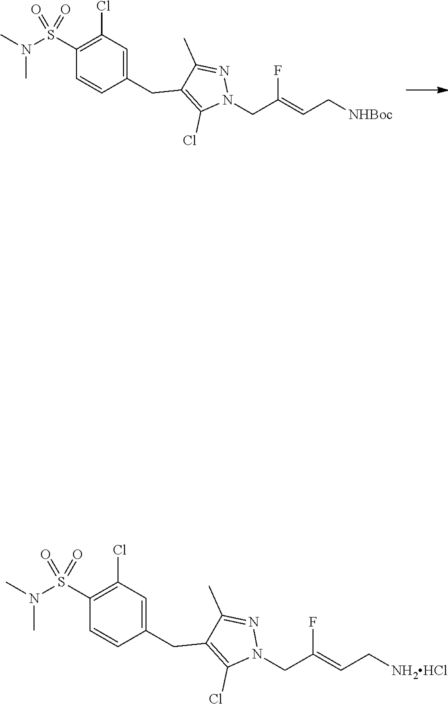

To a stirred suspension of tert-butyl (Z)-(4-(5-chloro-4-(3-chloro-4-(N,N-dimethylsulfamoyl)benzyl)-3-methyl-1H-pyrazol-1-yl)-3-fluorobut-2-en-1-yl)carbamate (0.35 g, 0.65 mmol) in methanol (2.0 mL) at rt was added HCl in (4.0 M in diethyl ether, 2.00 mL, 8.00 mmol). The resulting reaction mixture was stirred at rt for 2 h. After completion of reaction, the solvent was concentrated under reduced pressure, and the residue was washed with diethyl ether, dried under vacuum to give (Z)-4-((1-(4-amino-2-fluorobut-2-en-1-yl)-5-chloro-3-methyl-1H-pyrazol-4-yl)methyl)-2-chloro-N,N-dimethylbenzenesulfon-amide hydrochloride (0.22 g, 73%). White solid. ¹H-NMR (300 MHz, CD₃OD) δ ppm: 7.95 (d, J=8.2 Hz, 1H), 7.42-7.39 (m, 1H), 7.32-7.27 (m, 1H), 5.12 (dt, J=33.4, 7.5 Hz, 1H), 4.97 (dd, J=13.5, 0.9 Hz, 2H), 3.90 (s, 2H), 3.69-3.62 (m, 2H), 2.86 (s, 6H), 2.15 (s, 3H).

### Procedure AAD: Preparation of (Z)-4-((1-(4-amino-2-fluorobut-2-en-1-yl)-3-chloro-5-methyl-1H-pyrazol-4-yl)methyl)-2-chloro-N,N-dimethylbenzenesulfonamide hydrochloride (Compound 42)

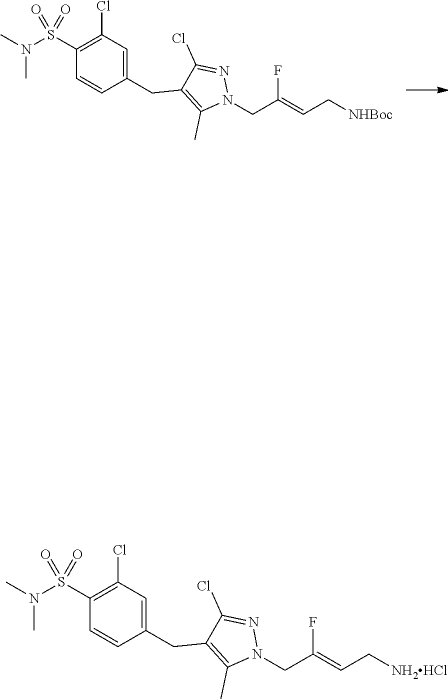

To a stirred suspension of tert-butyl (Z)-(4-(3-chloro-4-(3-chloro-4-(N,N-dimethylsulfamoyl)benzyl)-5-methyl-1H-pyrazol-1-yl)-3-fluorobut-2-en-1-yl)carbamate (0.22 g, 0.41 mmol) in methanol (0.5 mL) at rt was added HCl (4.0 M in diethyl ether, 2.0 mL, 8.0 mmol). The resulting reaction mixture was stirred at rt for 2 h. After completion of reaction, the solvent was concentrated under reduced pressure, and the residue was washed with diethyl ether, dried under vacuum to give (Z)-4-((1-(4-amino-2-fluorobut-2-en-1-yl)-3-chloro-5-methyl-1H-pyrazol-4-yl)methyl)-2-chloro-N,N-dimethylbenzenesulfonamide hydrochloride (0.12 g, 62%) as a white solid. ¹H-NMR (300 MHz, CD₃OD) δ ppm: 7.94 (d, J=8.2 Hz, 1H), 7.42-7.39 (m, 1H), 7.30 (ddd, J=8.1, 1.7, 0.8 Hz, 1H), 5.20-5.02 (m, 1H), 4.93 (d, J=13.5 Hz, 2H), 3.89 (s, 2H), 3.65 (ddt, J=7.5, 1.7, 0.8 Hz, 2H), 2.86 (s, 6H), 2.30 (s, 3H).

### Example 23

The following compound was prepared according to procedures AAE, AAF, O, F, G, H and I using appropriate starting materials.

### (Z)-3-fluoro-4-(5-isopropyl-3-methyl-4-((6-(methylsulfonyl)pyridin-3-yl)methyl)-1H-pyrazol-1-yl)but-2-en-1-amine dihydrochloride (Compound 43)

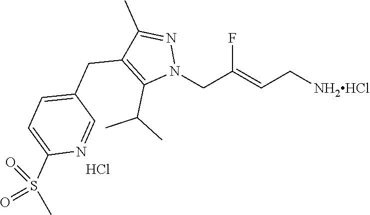

### Procedure AAE: Preparation of methyl 6-(methylsulfonyl)nicotinate

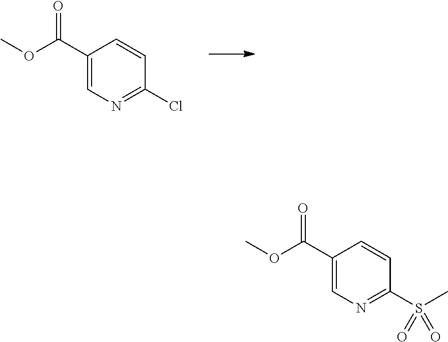

To a stirring solution of methyl 6-chloronicotinate (2.00 g, 11.7 mmol) in DMSO (10 mL), was added sodium methanesulfinate (1.78 g, 17.5 mmol) at rt in one lot. The resulting mixture was heated to 100° C. for 1 h. The reaction mixture was cooled to rt, and then dilute with water (100 mL). The product was then extracted with ethyl acetate (30 mL×3) and the combined organics were washed with water (20 mL) and brine (20 mL). After drying over Na₂SO₄, the solvent was removed in vacuo to afford methyl 6-(methylsulfonyl)nicotinate (2.20 g, 88%) as an off-white solid. ¹H-NMR (300 MHz, CDCl₃) δ ppm: 9.31 (dd, J=2.1, 0.9 Hz, 1H), 8.59 (dd, J=8.1, 2.0 Hz, 1H), 8.20 (dd, J=8.1, 0.9 Hz, 1H), 4.04 (s, 3H), 3.30 (s, 3H).

### Procedure AAF: Preparation of (6-methylsulfonyl)pyridin-3-yl)methanol

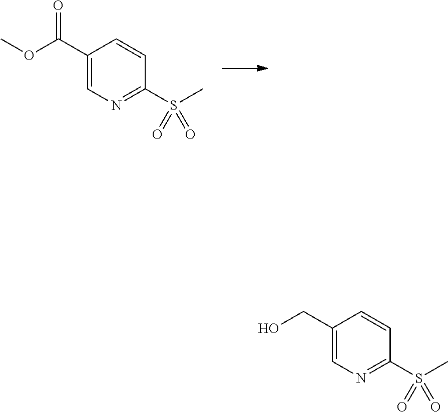

A stirring mixture of methyl 6-(methylsulfonyl)nicotinate (9.23 g, 42.9 mmol), calcium chloride (4.76 g, 42.9 mmol) in ethanol/THF (2:1; 60 mL) was cooled to −5° C. Sodium borohydride (3.24 g, 85.8 mmol) was then added portion-wise, keeping the temp below 0° C. Following complete addition, the ice bath was removed and then stirring was continued for 1 hour at rt. The reaction was quenched by addition of aqueous NaOH (2 M, 15.0 mL). The mixture was stirred at rt for 10 min and then the solvents were removed under vacuum. The resulting residue was dissolved in ethyl acetate (60 mL), and then anhydrous magnesium sulfate (10 g) was added in portions. The thick suspension was stirred at rt for 10 min and then filtered. The filter cake was washed with ethyl acetate (20 mL×5) and the combined organics were then concentrated in vacuo to afford (6-(methylsulfonyl)pyridin-3-yl)methanol (8.68 g, 100%) as a pale yellow oil. ¹H NMR (300 MHz, CDCl₃) δ ppm: 8.69 (dq, J=2.2, 0.7 Hz, 1H), 8.04 (dd, J=8.0, 0.8 Hz, 1H), 7.98 (ddt, J=8.1, 2.1, 0.8 Hz, 1H), 4.86 (d, J=0.8 Hz, 2H), 3.23 (s, 3H).

### (Z)-3-fluoro-4-(5-isopropyl-3methyl-4-((6-(methylsulfonyl)pyridin-3-yl)methyl)-1H-pyrazol-1-yl)but-2-en-1-amine dihydrochloride (Compound 43)

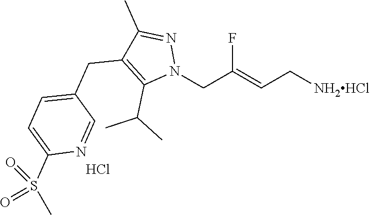

¹H-NMR (300 MHz, CD₃OD) δ ppm: 8.55 (d, J=1.5 Hz, 1H), 8.04 (dd, J=8.1, 0.8 Hz, 1H), 7.82 (dd, J=8.1, 2.2 Hz, 1H), 5.20 (dt, J=34.9, 7.4 Hz, 1H), 5.12 (d, J=13.0 Hz, 2H), 4.16 (s, 2H), 3.69 (d, J=7.4 Hz, 2H), 3.28 (hept, J=7.1 Hz, 1H), 2.16 (d, J=0.8 Hz, 3H), 1.29 (d, J=7.1 Hz, 6H).

### Example 24

The following compound was prepared according to procedures AAG, AAR, O, F, G, H and I using appropriate starting materials.

### (Z)-4-(3,5-dimethyl-4-(4-((trifluoromethyl)sulfonyl)benzyl)-1H-pyrazol-1-yl)-3-fluorobut-2-en-1-amine hydrochloride (Compound 44)

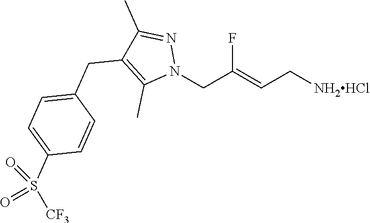

### Procedure AAG: Preparation of 4-((trifluoromethyl)sulfonyl)benzaldehyde

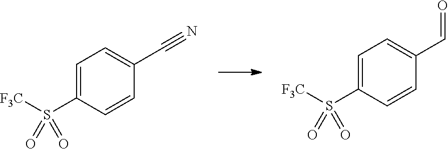 [SMILES: N#Cc1ccc(S(=O)(=O)C(F)(F)F)cc1 ; O=Cc1ccc(S(=O)(=O)C(F)(F)F)cc1]

To a stirring solution of 4-(trifluoromethylsulfonyl)benzonitrile (1.50 g, 6.38 mmol) in formic acid (7.50 mL) was added Raney nickel (1.00 g, 6.38 mmol) as a suspension in water (2.5 mL). The resulting suspension was then heated at reflux for 2 h. After cooling to rt, the reaction mixture was diluted with ethyl acetate (40 mL) and filtered through Celite®. The filtrate was washed with sat. aq. NaHCO₃, sat. aq. NaCl, dried over Na₂SO₄, and then concentrated in vacuo. The crude material was purified over silica gel (40 g), eluting with 10%-50% ethyl acetate in hexanes to afford the title compound 4-(trifluoromethylsulfonyl)benzaldehyde (1.07 g, 70%) as a colorless, non-viscous oil. ¹H NMR (300 MHz, CDCl₃) δ ppm: 10.21 (s, 1H), 8.30-8.22 (m, 2H), 8.22-8.17 (m, 2H).

### (Z)-4-(3,5-dimethyl-4-(4-((trifluoromethyl)sulfonyl)benzyl)-1H-pyrazol-1-yl)-3-fluorobut-2-en-1-amine hydrochloride (Compound 44)

¹H-NMR (300 MHz, CD₃OD) δ ppm: 8.02 (d, J=7.9 Hz, 2H), 7.58 (d, J=8.1 Hz, 2H), 5.20 (dt, J=33.7, 7.3 Hz, 1H), 5.04 (d, J=13.6 Hz, 2H), 4.05 (s, 2H), 3.67 (d, J=7.1 Hz, 2H), 2.33 (s, 3H), 2.21 (s, 3H).

### Example 25

The following compound was prepared according to procedures AAH, AAI, L, N, O, F, G, H, and I using appropriate starting materials.

### (Z)-4-((1-(4-amino-2-fluorobut-2-en-1-yl)-3,5-dimethyl-1H-pyrazol-4-yl)methyl)-N-methyl-2-(trifluoromethoxy)benzenesulfonamide hydrochloride (Compound 45)

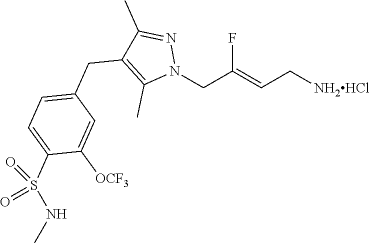

### Procedure AAH: Preparation of 1-(4-methoxyphenyl)-N-methylmethanamine

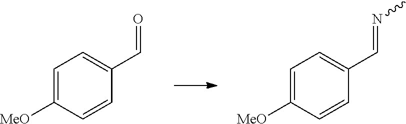

To a stirred solution of 4-methoxybenzaldehyde (10.0 g, 73.4 mmol) in ethanol (30 mL) at 40° C. was added methylamine (40 w % in water; 7.63 mL, 88.1 mmol). Stirring was continued for 2 h. The reaction mixture was concentrated under reduced pressure. The residue was partitioned between CH₂Cl₂ (20 mL) and water (20 mL), and the organic layer was washed with further water. The organics were then dried over Na₂SO₄, and concentrated in vacuo to afford 1-(4-methoxyphenyl)-N-methylmethanamine (11.0 g, 100%) as a brown oil. ¹H-NMR (300 MHz, CDCl₃) δ ppm: 8.19 (q, J=1.7 Hz, 1H), 7.69-7.59 (m, 2H), 6.94-6.87 (m, 2H), 3.81 (d, J=0.7 Hz, 3H), 3.46 (d, J=1.5 Hz, 3H).

### Procedure AAI: Preparation of 1-(4-methoxyphenyl)-N-methylmethanamine

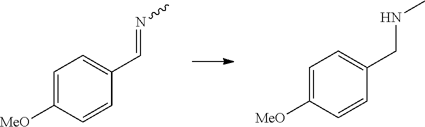

To a stirring solution of 1-(4-methoxyphenyl)-N-methylmethanamine (11.0 g, 74.0 mmol) in ethanol (160 mL) at 0° C. was added sodium borohydride (13.9 g, 0.37 mol) portion-wise over 5 min. The resulting mixture was warmed to rt and stirring was continued overnight. The reaction mixture was diluted with water (80 mL) and then concentrated under reduced pressure. The aqueous mixture was extracted with CH₂Cl₂ (3×50 mL). The combined organics were then washed with aqueous HCl (2×100 mL). The combined aqueous was washed with ethyl acetate (30 mL), and the pH adjusted to 9 using aqueous 4 M NaOH. The product was extracted with ethyl acetate (2×50 mL), and the combined organics were washed with water (20 mL), dried over Na₂SO₄ and concentrated in vacuo to afford 1-(4-methoxyphenyl)-N-methylmethanamine (8.40 g, 75%) as a yellow oil. ¹H-NMR (300 MHz, CDCl₃) δ 7.26-7.19 (m, 2H), 6.90-6.80 (m, 2H), 3.78 (dt, J=3.1, 1.8 Hz, 3H), 3.68 (d, J=2.2 Hz, 2H), 2.43 (dd, J=2.1, 1.0 Hz, 3H).

### (Z)-4-((1-(4-amino-2-fluorobut-2-en-1-yl)-3,5-dimethyl-1H-pyrazol-4-yl)methyl)-N-methyl-2-(trifluoromethoxy)benzenesulfonamide hydrochloride (Compound 45)

¹H-NMR (300 MHz, CD₃OD) δ ppm: 7.92 (d, J=8.1 Hz, 1H), 7.33 (dd, J=2.2, 1.2 Hz, 1H), 7.28 (ddt, J=8.4, 1.6, 0.7 Hz, 1H), 5.42-5.24 (m, 1H), 5.17-5.08 (m, 2H), 4.02 (s, 2H), 3.73-3.64 (m, 2H), 2.59 (s, 3H), 2.37 (s, 3H), 2.27 (s, 3H).

### Example 26

The following compound was prepared according to the procedures E, AAJ, AAK, AAL, V, W and I using appropriate starting materials.

### (Z)-4-((4-(4-amino-2-fluorobut-2-en-1-yl)-3,5-dimethyl-1H-pyrazol-1-yl)methyl)-3-chloro-N,N-dimethylbenzenesulfonamide hydrochloride (Compound 47)

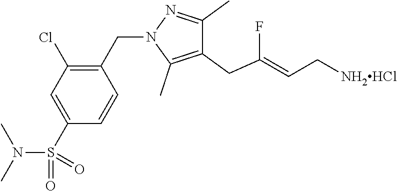

### Procedure AAJ: Preparation of 3-chloro-4-formyl-N,N-dimethylbenzeneulfonamide

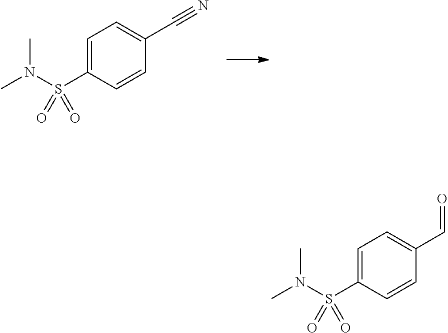

To a stirring solution of 3-chloro-4-cyano-N,N-dimethylbenzenesulfonamide (1.03 g, 4.21 mmol) in formic acid (98% v/v, 5.00 mL) was added Raney nickel (700 mg, 4.21 mmol) as a suspension in water (2.0 mL). This resulting suspension was heated at 100° C. for 2 h. After cooling to rt, the reaction mixture was diluted with ethyl acetate (40 mL) and filtered through Celite®. The filtrate was washed with sat. aq. NaHCO₃, sat. aq. NaCl, dried over Na₂SO₄, and then concentrated in vacuo. Purification over silica gel, eluting with 30% ethyl acetate in hexanes gave 3-chloro-4-formyl-N,N-dimethylbenzenesulfonamide (665 mg, 66%) as white solid. ¹H-NMR (300 MHz, CDCl₃) δ ppm: 10.55 (d, J=0.8 Hz, 1H), 8.10 (dd, J=8.1, 0.4 Hz, 1H), 7.91 (dd, J=1.7, 0.4 Hz, 1H), 7.78 (ddd, J=8.1, 1.7, 0.8 Hz, 1H), 2.81 (s, 6H).

### Procedure AAK: Preparation of tert-butyl 2-(2-chloro-4-(N,N-dimethylsulfamoyl)benzylidene)hydrazine-1-carboxylate

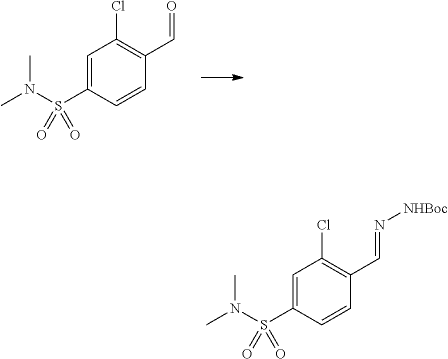 [SMILES: CN(C)S(=O)(=O)c1ccc(C=O)c(Cl)c1 ; CN(C)S(=O)(=O)c1ccc(/C=N/NC(=O)OC(C)(C)C)c(Cl)c1]

To a stirring solution of 3-chloro-4-formyl-N,N-dimethylbenzenesulfonamide (665 mg, 2.68 mmol) in ethanol (10 mL), was added tert-butyl carbazate (373 mg, 2.82 mmol). The resulting mixture was stirred at rt for 5 mins, then acetic acid (0.2 mL) was added. Stirring was continued at rt for a further 30 min. The reaction mixture was concentrated in vacuo, and the resulting residue was partitioned between sat. aq. NaHCO₃ (20 mL) and ethyl acetate (30 mL). The organic layer was washed with brine, dried over Na₂SO₄, and then concentrated in vacuo to afford tert-butyl 2-(2-chloro-4-(N,N-dimethylsulfamoyl)benzylidene)hydrazine-1-carboxylate (903 mg, 93%) as a white foam. ¹H-NMR (300 MHz, CDCl₃) δ ppm: 8.30 (s, 1H), 8.26 (d, J=8.3 Hz, 1H), 7.78 (dd, J=1.8, 0.4 Hz, 1H), 7.64 (ddd, J=8.3, 1.8, 0.7 Hz, 1H), 2.75 (s, 6H), 1.56 (s, 10H).

### Procedure AAL: Preparation of tert-butyl 2-(2-chloro-4-(N,N-dimethylsulfamoyl)benzyl)hydrazine-1-carboxylate

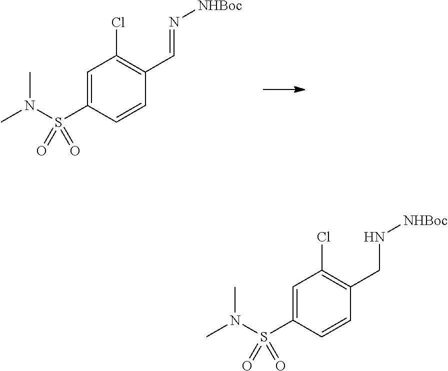 [SMILES: CN(C)S(=O)(=O)c1ccc(/C=N/NC(=O)OC(C)(C)C)c(Cl)c1 ; CN(C)S(=O)(=O)c1ccc(CNNC(=O)OC(C)(C)C)c(Cl)c1]

To a stirring solution of tert-butyl 2-(2-chloro-4-(N,N-dimethylsulfamoyl)benzylidene)hydrazine-1-carboxylate (900 mg, 2.49 mmol) in THF (10 mL) at rt was added acetic acid (6.0 mL) followed by sodium cyanoborohydride (469 mg, 7.46 mmol). The resulting mixture was left to stir at rt overnight. The solvent was removed in vacuo, and the residue was partitioned between sat. aq. NaHCO₃ and ethyl acetate. The organic layer was washed with brine, dried over Na₂SO₄ and then concentrated in vacuo. The crude material was taken up in methanol (10 mL) and to this was added aqueous NaOH (2.0 M, 5.0 mL). The resulting mixture was then stirred at rt for 90 mins. The reaction mixture was then partitioned between diethyl ether (40 mL), ethyl acetate (10 mL) and brine (40 mL). The organic layer was dried over Na₂SO₄, and concentrated in vacuo to afford tert-butyl 2-(2-chloro-4-(N,N-dimethylsulfamoyl)benzyl)hydrazine-1-carboxylate (550 mg, 61%) as a white foam. ¹H-NMR (300 MHz, CDCl₃) δ ppm: 7.80 (t, J=1.1 Hz, 1H), 7.66 (d, J=1.2 Hz, 2H), 6.07 (s, 1H), 4.42 (s, 1H), 4.23-4.16 (m, 2H), 2.75 (s, 6H), 1.47 (s, 9H).

### (Z)-4-((4-(4-amino-2-fluorobut-2-en-1-yl)-3,5-dimethyl-1H-pyrazol-1-yl)methyl)-3-chloro-N,N-dimethylbenzenesulfonamide hydrochloride (Compound 47)

¹H-NMR (300 MHz, DMSO-d₆) δ ppm: 7.95 (s, 3H), 7.81 (d, J=1.8 Hz, 1H), 7.71 (dd, J=8.1, 1.9 Hz, 1H), 6.80 (d, J=8.1 Hz, 1H), 5.38 (s, 2H), 4.76 (dt, J=36.1, 7.3 Hz, 1H), 3.45 (t, J=6.4 Hz, 2H), 3.42-3.33 (m, 2H), 2.65 (s, 6H), 2.16 (s, 3H), 2.10 (s, 3H).

### Example 27

The following compound was prepared according to the procedures E, AAJ, AAK, AAL, V, W and I using appropriate starting materials.

### (Z)-4-((4-(4-amino-2-fluorobut-2-en-1-yl)-3,5-dimethyl-1H-pyrazol-1-yl)methyl)-2-chloro-N,N-dimethylbenzenesulfonamide hydrochloride (Compound 46)

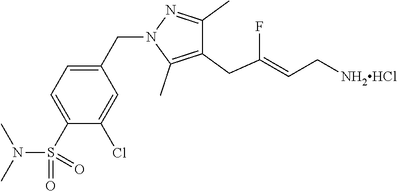

¹H-NMR (300 MHz, CD₃OD) δ ppm: 8.05 (d, J=8.2 Hz, 1H), 7.48 (dd, J=1.5, 0.8 Hz, 1H), 7.26 (ddd, J=8.2, 1.8, 0.9 Hz, 1H), 5.56 (s, 2H), 5.06-4.87 (m, 1H), 3.62 (d, J=8.0 Hz, 2H), 3.60-3.53 (m, 2H), 2.88 (s, 6H), 2.38 (s, 3H), 2.37 (s, 3H).

### Example 28

The following compound was prepared according to the procedures F, G, H, AAO, AAP and I using appropriate starting materials.

### (Z)—N-(4-((1-(4-amino-2-fluorobut-2-en-1-yl)-3,5-dimethyl-1H-pyrazol-4-yl)methyl)phenyl)methane-sulfonamide hydrochloride (Compound 48)

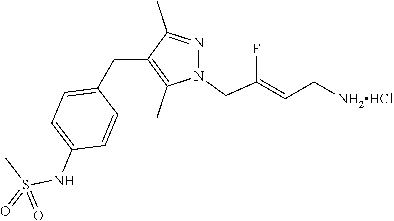

### Procedure AAO: Preparation of tert-butyl (Z)(4-(4-(4-aminobenzyl)-3,5-dimethyl-1H-pyrazol-1-yl)-3-fluorobut-2-en-1-yl)carbamate

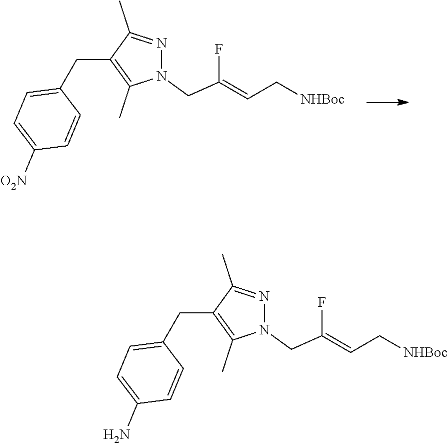 [SMILES: Cc1nn(C/C(F)=C/CNC(=O)OC(C)(C)C)c(C)c1Cc1ccc([N+](=O)[O-])cc1 ; Cc1nn(C/C(F)=C/CNC(=O)OC(C)(C)C)c(C)c1Cc1ccc(N)cc1]

To a stirring solution of tert-butyl (Z)-(4-(3,5-dimethyl-4-(4-nitrobenzyl)-1H-pyrazol-1-yl)-3-fluorobut-2-en-1-yl)carbamate (100 mg, 0.24 mmol) in THF (2.0 mL) and sat. aq. NH₄Cl (1.0 mL) at rt was added zinc powder (326 mg, 4.99 mmol) in one lot. The resulting suspension was stirred at rt for 6 h. The reaction mixture was filtered through a plug of Celite® and the filtrate was partitioned between water (10 mL) and ethyl acetate (10 mL). The aqueous layer was extracted with ethyl acetate (10 mL) and the combined organics were dried over Na₂SO₄ and concentrated in vacuo to afford tert-butyl (Z)-(4-(4-(4-aminobenzyl)-3,5-dimethyl-1H-pyrazol-1-yl)-3-fluorobut-2-en-1-yl)carbamate (69.0 mg, 74%). ¹H-NMR (300 MHz, CDCl₃) δ ppm: 6.91-6.85 (m, 2H), 6.64-6.57 (m, 2H), 4.87-4.57 (m, 3H), 3.81 (s, 2H), 3.62 (s, 2H), 2.14 (s, 3H), 2.12 (s, 3H), 1.45 (s, 9H).

### Procedure AAP: Preparation of tert-butyl (Z)-(4-(3,5-dimethyl-4-(4-(methylsulfonamido)benzyl)-1H-pyrazol-1-yl)-3-fluorobut-2-en-1-yl)carbamate

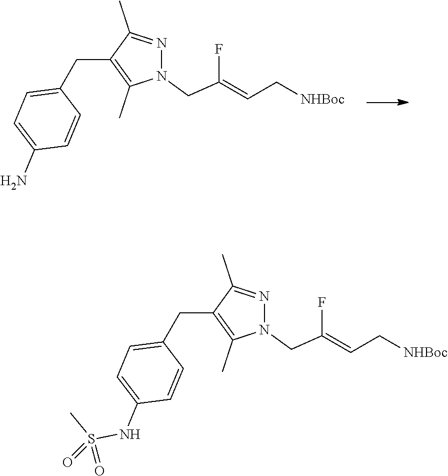 [SMILES: Cc1nn(C/C(F)=C/CNC(=O)OC(C)(C)C)c(C)c1Cc1ccc(N)cc1 ; Cc1nn(C/C(F)=C/CNC(=O)OC(C)(C)C)c(C)c1Cc1ccc(NS(C)(=O)=O)cc1]

To a stirring solution of tert-butyl (Z)-(4-(4-(4-aminobenzyl)-3,5-dimethyl-1H-pyrazol-1-yl)-3-fluorobut-2-en-1-yl)carbamate (340 mg, 0.88 mmol), triethylamine (0.31 mL, 2.19 mmol) and 4-dimethylaminopyridine (10.7 mg, 0.09 mmol) in CH₂Cl₂ (8.0 mL) at 0° C. was added methanesulfonyl chloride (0.07 mL, 0.92 mmol). The reaction mixture was partitioned between aqueous HCl (2.0 M; 10 mL) and CH₂Cl₂ (10 mL) and the aqueous layer was extracted with further CH₂Cl₂ (10 mL). The combined organics were dried over Na₂SO₄ and concentrated in vacuo. The crude material was purified over silica gel (40 g) eluting with 60% ethyl acetate in hexanes to afford tert-butyl (Z)-(4-(3,5-dimethyl-4-(4-(methylsulfonamido)benzyl)-1H-pyrazol-1-yl)-3-fluorobut-2-en-1-yl)carbamate (155 mg, 38%) as an off-white solid. ¹H-NMR (300 MHz, CDCl₃) δ ppm: 7.29-7.24 (m, 2H), 7.22-7.16 (m, 2H), 4.69 (dd, J=11.5, 1.1 Hz, 2H), 3.83 (t, J=6.7 Hz, 2H), 3.77 (s, 2H), 3.40 (s, 3H), 2.16 (s, 3H), 2.13 (s, 3H), 1.45 (s, 9H).

### (Z)—N-(4-((1-(4-amino-2-fluorobut-2-en-1-yl)-3,5-dimethyl-1H-pyrazol-4-yl)methyl)phenyl-)methane-sulfonamide hydrochloride (Compound 48)

¹H-NMR (300 MHz, CD₃OD) δ ppm: 7.25-7.19 (m, 2H), 7.16 (d, J=8.7 Hz, 2H), 5.49 (dt, J=33.9, 7.3 Hz, 1H), 5.25 (d, J=15.4 Hz, 2H), 3.89 (s, 2H), 3.76-3.66 (m, 2H), 2.94 (s, 3H), 2.42 (s, 3H), 2.35 (s, 3H).

### Example 29

The following compound was prepared according to procedures AAQ, AAR, O, F, G, H and I using appropriate starting materials.

### (Z)-4-(4-(3-chloro-4-(isopropylsulfonyl)benzyl)-3,5-dimethyl-1H-pyrazol-1-yl)-3-fluorobut-2-en-1-amine hydrochloride (Compound 49)

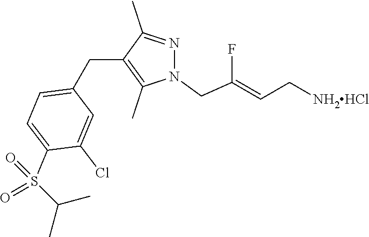

### Procedure AAO: Preparation of 3-chloro-4-(isopropylsulfonyl)benzaldehyde

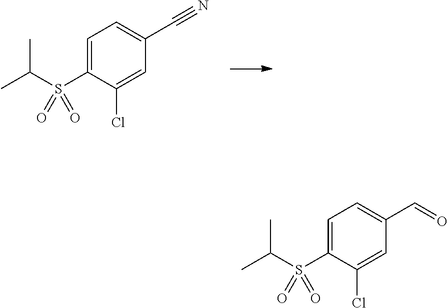

To a stirring solution of 3-chloro-4-(isopropylsulfonyl)benzonitrile (880 mg, 3.61 mmol) in formic acid (4.6 mL) at rt was added Raney nickel (651 mg) as a slurry in water (1.75 mL). The resulting mixture was heated at reflux for 2 h. The reaction mixture was cooled to rt, diluted with ethyl acetate (160 mL) and then filtered through a Celite® pad. The filtrate was washed with water (2×100 mL) sat. aq. NaHCO₃ (3×80 mL), brine (2×50 mL), dried over Na₂SO₄ and then concentrated in vacuo to afford the 3-chloro-4-(isopropylsulfonyl)benzaldehyde (780 mg, 88%) as a yellow solid. ¹H-NMR (300 MHz, CDCl₃) δ ppm: 10.10 (d, J=0.4 Hz, 1H), 8.33 (dt, J=8.1, 0.4 Hz, 1H), 8.06 (dd, J=1.6, 0.5 Hz, 1H), 7.97 (dd, J=8.1, 1.6 Hz, 1H), 3.93-3.80 (m, 1H), 1.36 (d, J=6.8 Hz, 7H).

### Procedure AAR: Preparation of (3-chloro-4-(isopropylsulfonyl)phenyl)methanol

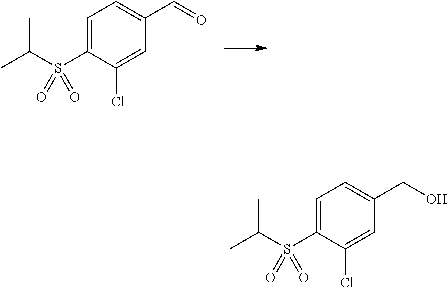

To a stirring solution of 3-chloro-4-(isopropylsulfonyl)benzaldehyde (730 mg, 2.96 mmol) in methanol (15 mL) at 0° C. was added sodium borohydride (134 mg, 3.55 mmol). The resulting mixture was left to stir at 0° C. for 10 min. The reaction mixture was diluted with ethyl acetate (50 mL). The organics were washed with water (40 mL), brine (2×20 mL), dried over Na₂SO₄ and concentrated in vacuo to afford (3-chloro-4-(isopropylsulfonyl)phenyl)methanol (720 mg, 98%) as a clear oil. ¹H-NMR (300 MHz, CDCl₃) δ ppm: 8.02 (d, J=8.2 Hz, 1H), 7.57 (dt, J=1.7, 0.8 Hz, 1H), 7.42 (ddt, J=8.1, 1.6, 0.8 Hz, 1H), 4.80 (s, 2H), 3.78 (p, J=6.9 Hz, 1H), 1.32 (d, J=6.8 Hz, 6H).

### (Z)-4-(4-(3-chloro-4-(isopropylsulfonyl)benzyl)-3,5-dimethyl-1H-pyrazol-1-yl)-3-fluorobut-2-en-1-amine hydrochloride (Compound 49)

¹H-NMR (300 MHz, CD₃OD) δ ppm: 8.01 (d, J=8.1 Hz, 1H), 7.49 (d, J=1.7 Hz, H), 7.36 (ddt, J=8.2, 1.6, 0.7 Hz, 1H), 5.45-5.26 (m, 1H), 5.20-5.11 (m, 2H), 4.02 (s, 2H), 3.80 (p, J=6.8 Hz, 1H), 3.72-3.66 (m, 2H), 2.38 (s, 3H), 2.29 (s, 3H), 1.27 (d, J=6.8 Hz, 6H).

### Example 30

The following compound was prepared according to procedures AAS, G, H and I using appropriate starting materials.

### (Z)-4-((1-(4-amino-2-fluorobut-2-en-1-yl)-3-(tert-butyl)-5-methyl-1H-pyrazol-4-yl)methyl)-N,N-dimethylbenzenesulfonamide hydrochloride (Compound 50)

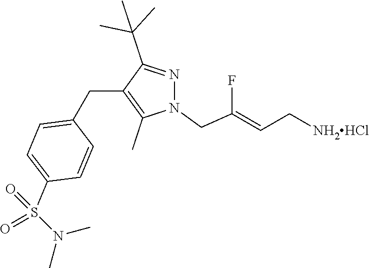

### Procedure AAS: Preparation of 4-(2-acetyl-4,4-dimethyl-3-oxopentyl)-N,N-dimethylbenzenesulfonamide

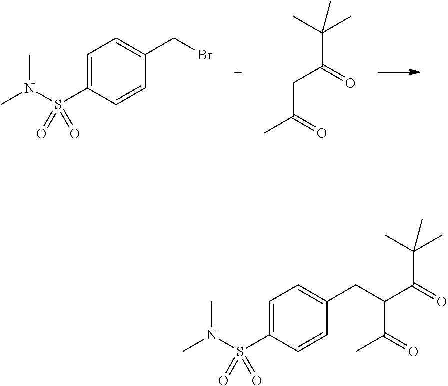

To a stirring solution of 5,5-dimethylhexane-2,4-dione (0.88 mL, 6.00 mmol) in THF (12.0 mL) at 0° C. under nitrogen was added sodium bis(trimethylsilyl)amide (1.0 M in THF; 4.00 mL, 4.00 mmol). After stirring for 10 min at 0° C., a solution of 4-(bromomethyl)-N,N-dimethylbenzenesulfonamide (556 mg, 2.00 mmol) in THF (4.0 mL) was added dropwise. The resulting mixture was then heated at reflux for 4 h. After cooling to rt, the reaction mixture was partitioned between ethyl acetate and water. The organic layer was washed with further water, brine, and then dried over Na₂SO₄, and concentrated in vacuo. The crude material was purified over silica gel (12 g), eluting with 10%-20% ethyl acetate in hexanes to afford 4-(2-acetyl-4,4-dimethyl-3-oxopentyl)-N,N-dimethylbenzenesulfonamide (457 mg, 67%) as a clear oil that gave way to a white solid upon standing. ¹H-NMR (300 MHz, CDCl₃) δ ppm: 7.73-7.67 (m, 2H), 7.39-7.33 (m, 2H), 4.39-4.28 (m, 1H), 3.31 (dd, J=13.4, 9.0 Hz, 1H), 3.12 (dd, J=13.4, 5.7 Hz, 1H), 2.68 (s, 6H), 2.20 (d, J=0.3 Hz, 3H), 0.97 (s, 9H).

### (Z)-4-((1-(4-amino-2-fluorobut-2-en-1-yl)-3-(tert-butyl)-5-methyl-1H-pyrazol-4-yl)methyl)-N,N-dimethylbenzenesulfonamide hydrochloride (Compound 50)

¹H-NMR (300 MHz, CD₃OD) δ ppm: 7.76-7.70 (m, 2H), 7.38-7.32 (m, 2H), 5.37-5.16 (m, 3H), 4.21 (s, 2H), 3.70 (d, J=7.4 Hz, 2H), 2.68 (s, 6H), 2.27 (s, 3H), 1.34 (s, 9H).

### Example 31

The following compound was prepared according to procedures AAT, AAU, M, N, O, F, G, H and I using appropriate starting materials.

### (Z)-4-((1-(4-amino-2-fluorobut-2-en-1-yl)-3,5-dimethyl-1H-pyrazol-4-yl)methyl)-2-chlorobenzenesulfonamide hydrochloride (Compound 51)

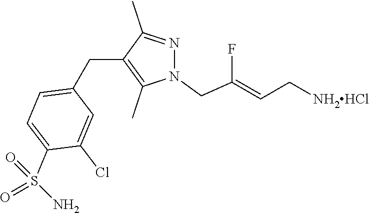

### Procedure AAT: Preparation of 4-bromo-2-chloro-N-(4-methoxybenzyl)benzenesulfonamide

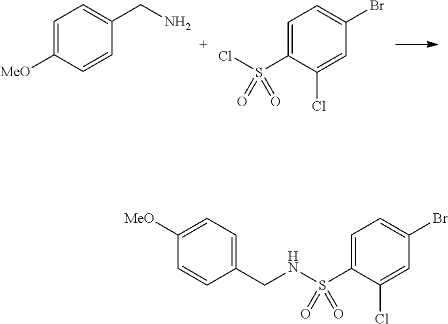 [SMILES: COc1ccc(CN)cc1 ; O=S(=O)(Cl)c1ccc(Br)cc1Cl ; COc1ccc(CNS(=O)(=O)c2ccc(Br)cc2Cl)cc1]

To a stirring solution of (4-methoxyphenyl)methanamine (0.57 mL, 4.40 mmol) and pyridine (2.0 mL) in CH₂Cl₂ (8.0 mL) at 0° C. was added a solution of 4-bromo-2-chlorobenzenesulfonyl chloride (1.16 g, 4.0 mmol) in CH₂Cl₂ (8.0 mL). The resulting mixture was stirred at 0° C. for 20 min and the at rt for a further 20 mins. Tlc analysis after this time indicated the presence of unreacted 4-bromo-2-chlorobenzenesulfonyl chloride. A further amount of pyridine (1.0 mL) and (4-methoxyphenyl)methanamine (0.39 mL, 2.20 mmol) was added and the reaction was stirred at rt for 10 min. The solvent was removed under vacuum, and the residue was partitioned between ethyl acetate (40 mL) and aqueous HCl (2.0 M; 30 mL). The organic layer was washed with further aqueous HCl (2.0 M; 30 mL), water, brine, dried over MgSO₄ and then concentrated in vacuo to afford 4-bromo-2-chloro-N-(4-methoxybenzyl)benzenesulfonamide (1.24 g, 79%). ¹H-NMR (300 MHz, CDCl₃) δ ppm: 7.91 (d, J=8.4 Hz, 1H), 7.64 (d, J=1.9 Hz, 1H), 7.53 (dd, J=8.5, 1.9 Hz, 1H), 7.12-7.06 (m, 2H), 6.81-6.75 (m, 2H), 5.20 (t, J=6.1 Hz, 1H), 4.12-4.07 (m, 2H), 3.79 (s, 3H).

### Procedure AAU: Preparation of 4-bromo-2-chloro-N,N-bis(4-methoxybenzyl)benzenesulfonamide

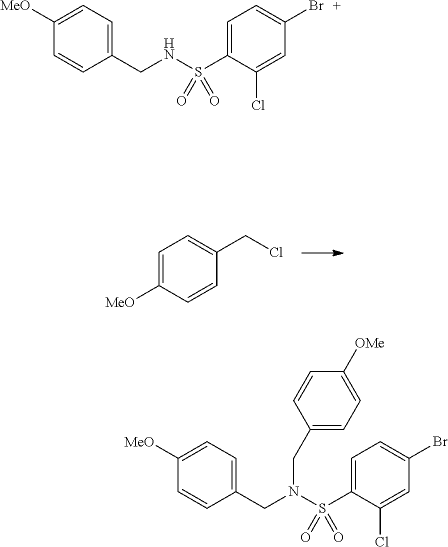 [SMILES: COc1ccc(CNS(=O)(=O)c2ccc(Br)cc2Cl)cc1 ; COc1ccc(CCl)cc1 ; COc1ccc(CN(Cc2ccc(OC)cc2)S(=O)(=O)c2ccc(Br)cc2Cl)cc1]

To a stirring solution of 4-bromo-2-chloro-N-(4-methoxybenzyl)benzenesulfonamide (1.24 g, 3.17 mmol) in DMF (7.0 mL) at rt was added potassium carbonate (526 mg, 3.81 mmol). After stirring for 10 min, 4-methoxybenzyl chloride (0.47 mL, 3.33 mmol) was added dropwise. The resulting mixture was stirred at rt for 2 h and then heated at 80° C. for 4 h. After cooling to rt, the reaction mixture was partitioned between ethyl acetate (50 mL) and water (20 mL). The organic layer was washed with further water, brine, dried over MgSO₄ and then concentrated in vacuo to afford 4-bromo-2-chloro-N,N-bis(4-methoxybenzyl)benzenesulfonamide (1.57 g, 97%) as a yellow oil that solidified upon standing. ¹H-NMR (300 MHz, CDCl₃) δ ppm: 7.93 (d, J=8.5 Hz, 1H), 7.71 (d, J=1.9 Hz, 1H), 7.52-7.48 (m, 1H), 7.04-6.97 (m, 4H), 6.85-6.78 (m, 4H), 4.34 (s, 4H), 3.81 (s, 6H).

### (Z)-4-((1-(4-amino-2-fluorobut-2-en-1-yl)-3,5-dimethyl-1H-pyrazol-4-yl)methyl)-2-chlorobenzenesulfonamide hydrochloride (Compound 51)

¹H-NMR (300 MHz, CD₃OD) δ ppm: 8.00 (d, J=8.1 Hz, 1H), 7.38 (d, J=1.7 Hz, 1H), 7.24 (dd, J=8.2, 1.7 Hz, 1H), 5.30 (dt, J=33.8, 7.3 Hz, 1H), 5.11 (d, J=14.3 Hz, 2H), 3.95 (s, 2H), 3.68 (d, J=7.3 Hz, 2H), 2.35 (s, 3H), 2.26 (s, 3H).

### Example 32

The following compound was prepared according to procedures E, M, N, O, AC, AD, AE, AAV and AF using appropriate starting materials.

### (Z)-4-((1-(4-amino-2-fluorobut-2-en-1-yl)-5-(2-hydroxypropan-2-yl)-3-methyl-1H-pyrazol-4-yl)methyl)-2-chloro-N,N-dimethylbenzenesulfonamide hydrochloride (Compound 54)

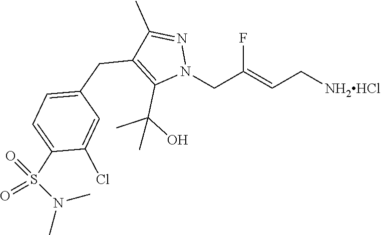

### Procedure AAV: Preparation of tert-butyl (Z)-(4-(4-(3-chloro-4-(N,N-dimethylsulfamoyl)benzyl)-5-(2-hydroxypropan-2-yl)-3-methyl-1H-pyrazol-1-yl)-3-fluorobut-2-en-1-yl)carbamate

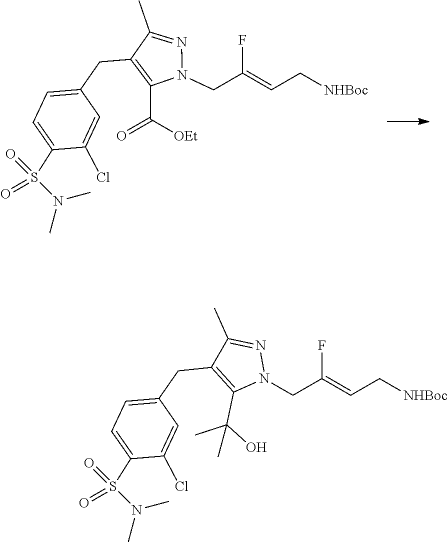 [SMILES: CCOC(=O)c1c(Cc2ccc(S(=O)(=O)N(C)C)c(Cl)c2)c(C)nn1C/C(F)=C/CNC(=O)OC(C)(C)C ; Cc1nn(C/C(F)=C/CNC(=O)OC(C)(C)C)c(C(C)(C)O)c1Cc1ccc(S(=O)(=O)N(C)C)c(Cl)c1]

To a stirring solution of ethyl (Z)-1-(4-((tert-butoxycarbonyl)amino)-2-fluorobut-2-en-1-yl)-4-(3-chloro-4-(N,N-dimethylsulfamoyl)benzyl)-3-methyl-1H-pyrazole-5-carboxylate (150 mg, 0.26 mmol) in THF (4.0 mL) at 0° C. was added methylmagnesium bromide (3.0 M in diethyl ether; 0.44 mL, 1.32 mmol) dropwise. The resulting solution was stirred at 0° C. for 20 min. The reaction mixture was partitioned between sat. aq. NH₄Cl (50 mL) and ethyl acetate (20 mL). The organic layer was washed with further ethyl acetate (2×20 mL), brine, dried over Na₂SO₄, and the concentrated in vacuo. The crude material was purified over silica gel to afford tert-butyl (Z)-(4-(4-(3-chloro-4-(N,N-dimethylsulfamoyl)benzyl)-5-(2-hydroxypropan-2-yl)-3-methyl-1H-pyrazol-1-yl)-3-fluorobut-2-en-1-yl)carbamate (47.0 mg, 32%) as a yellow oil. ¹H-NMR (300 MHz, CDCl₃) δ ppm: 7.95 (d, J=8.2 Hz, H), 7.20 (d, J=1.7 Hz, 1H), 7.05 (ddt, J=8.2, 1.9, 0.9 Hz, 1H), 5.20-5.12 (m, 2H), 4.77 (s, 1H), 4.72-4.52 (m, 1H), 3.94 (s, 2H), 3.84-3.76 (m, 2H), 2.89 (s, 6H), 2.10 (s, 3H), 1.50 (s, 6H), 1.43 (s, 9H).

### (Z)-4-((1-(4-amino-2-fluorobut-2-en-1-yl)-5-(2-hydroxypropan-2-yl)-3-methyl-1H-pyrazol-4-yl)methyl)-2-chloro-N,N-dimethylbenzenesulfonamide hydrochloride (Compound 54)

¹H-NMR (300 MHz, CD₃OD) δ ppm: 7.94 (d, J=8.0 Hz, 1H), 7.31 (s, 1H), 7.19 (d, J=8.0 Hz, 1H), 5.39 (d, J=10.9 Hz, 2H), 4.92 (dt, J=34.5, 7.4 Hz, 1H), 4.07 (s, 2H), 3.76-3.59 (m, 2H), 2.85 (s, 6H), 2.12 (s, 3H), 1.52 (s, 6H).

### Example 33

The following compound was prepared according to procedures E, AAW, AAX, AC, AD, AE, AJ and AF using appropriate starting materials.

### (Z)-4-((1-(4-amino-2-fluorobut-2-en-1-yl)-5-(hydroxymethyl)-3-methyl-1H-pyrazol-4-yl)methyl)-3-chloro-N,N-dimethylbenzenesulfonamide hydrochloride (Compound 55)

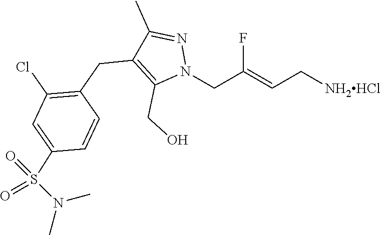

### Procedure AAW: Preparation of 3-chloro-4-(N,N-dimethylsulfamoyl)benzoic Acid

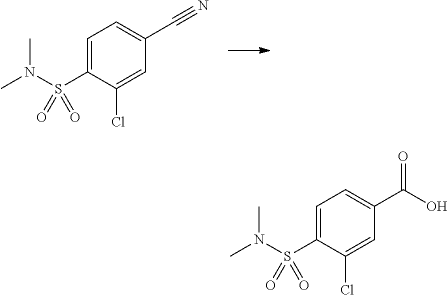 [SMILES: CN(C)S(=O)(=O)c1ccc(C#N)cc1Cl ; CN(C)S(=O)(=O)c1ccc(C(=O)O)cc1Cl]

To a stirring solution of 3-chloro-4-cyano-N,N-dimethyl-benzenesulfonamide (1.92 g, 7.85 mmol) in ethanol and water (2:1; 20 mL) was added sodium hydroxide (3.14 g, 78.5 mmol). The resulting mixture was heated at 100° C. for 2 h. The reaction mixture concentrated in vacuo and the residue was diluted with water, cooled to 0° C. and then acidified to pH 1-2 by the dropwise addition of 5N HCl. Upon acidification, solid precipitated out. The product was extracted into ethyl acetate, and the organic layer was washed water and then brine, dried over Na₂SO₄, and concentrated in vacuo to 3-chloro-4-(N,N-dimethylsulfamoyl)benzoic acid (1.75 g, 85%). ¹H-NMR (300 MHz, CDCl₃) δ ppm: 8.15 (d, J=8.2 Hz, 1H), 7.93 (d, J=1.7 Hz, 1H), 7.77 (dd, J=8.2, 1.8 Hz, 1H), 2.81 (s, 6H).

### Procedure AAX: Preparation of 2-chloro-4-(hydroxymethyl)-N,N-dimethylbenzenesulfonamide

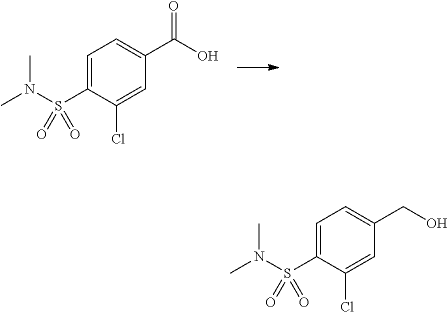 [SMILES: CN(C)S(=O)(=O)c1ccc(C(=O)O)cc1Cl ; CN(C)S(=O)(=O)c1ccc(CO)cc1Cl]

To a stirring solution of 2-chloro-4-(dimethylsulfamoyl)benzoic acid (875 mg, 3.32 mmol) in THF (12.0 mL) at 0° C. was added borane (dimethyl sulfide complex) solution (2.0 M; 2.00 mL, 4.00 mmol) dropwise. The resulting solution was heated at 75° C. for 2 h. The reaction mixture was cooled to 0° C. and water slowly added. The product was extracted with ethyl acetate, and the organics were washed with water and then brine, dried over Na₂SO₄, and concentrated in vacuo to afford 2-chloro-4-(hydroxymethyl)-N,N-dimethylbenzenesulfonamide (850 mg, 100%) as a pale orange oil. ¹H-NMR (300 MHz, CDCl₃) δ ppm: 7.83-7.69 (m, 3H), 2.76 (s, 6H).

### (Z)-4-((1-(4-amino-2-fluorobut-2-en-1-yl)-5-(hydroxymethyl)-3-methyl-1H-pyrazol-4-yl)methyl)-3-chloro-N,N-dimethylbenzenesulfonamide hydrochloride (Compound 55)

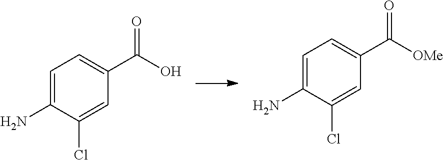 [SMILES: Nc1ccc(C(=O)O)cc1Cl ; COC(=O)c1ccc(N)c(Cl)c1]

¹H-NMR (300 MHz, CD₃OD) δ ppm: 7.83 (d, J=1.8 Hz, 1H), 7.65 (dd, J=8.1, 1.9 Hz, 1H), 7.33 (d, J=8.1 Hz, 1H), 5.33-5.14 (m, 3H), 4.67 (s, 2H), 4.10 (s, 2H), 3.69 (d, J=7.4 Hz, 2H), 2.72 (s, 6H), 2.18 (s, 3H).

### Example 34

The following compound was prepared according to procedures E, AAW, AAX, AAY, AD, AE, AJ, AL, AM and AF using appropriate starting materials.

### (Z)-4-((1-(4-amino-2-fluorobut-2-en-1-yl)-5-(1-hydroxyethyl)-3-isopropyl-1H-pyrazol-4-yl)methyl)-3-chloro-N,N-dimethylbenzenesulfonamide hydrochloride (Compound 58)

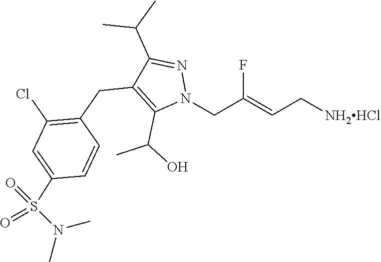

### Procedure AAY: Preparation of ethyl 3-(4-(N,N-dimethylsulfamoyl)benzyl)-5-methyl-2,4-dioxohexanoate

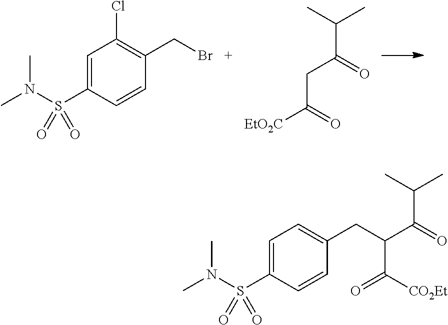 [SMILES: CN(C)S(=O)(=O)c1ccc(CBr)c(Cl)c1 ; CCOC(=O)C(=O)CC(=O)C(C)C ; CCOC(=O)C(=O)C(Cc1ccc(S(=O)(=O)N(C)C)cc1)C(=O)C(C)C]

To a stirring solution of ethyl 5-methyl-2,4-dioxohexanoate (0.26 g, 1.42 mmol) and 4-(bromomethyl)-3-chloro-N,N-dimethylbenzenesulfonamide (0.37 g, 1.18 mmol) in DMF (3.0 mL) at rt was added potassium carbonate (0.20 g, 1.42 mmol) in one lot. The resulting mixture was left to stir at rt for 1 h. The reaction mixture was diluted with sat. aq. NH₄Cl (10 mL), and the product was extracted with ethyl acetate (3×15 mL). The combined organics were washed with water (2×10 mL), dried over Na₂SO₄, and then concentrated in vacuo to afford ethyl 3-(4-(N,N-dimethylsulfamoyl)benzyl)-5-methyl-2,4-dioxohexanoate (540 mg) as a viscous oil. This material was used immediately in the next step without purification.

### (Z)-4-((1-(4-amino-2-fluorobut-2-en-1-yl)-5-(1-hydroxyethyl)-3-isopropyl-1H-pyrazol-4-yl)methyl)-3-chloro-N,N-dimethylbenzenesulfonamide hydrochloride (Compound 58)

¹H-NMR (300 MHz, CD₃OD) δ ppm: 7.84 (d, J=1.9 Hz, 1H), 7.62 (dd, J=8.1, 1.9 Hz, 1H), 7.19 (d, J=8.1 Hz, 1H), 5.32-4.93 (m, 4H), 4.14 (d, J=18.3 Hz, 1H), 4.07 (d, J=18.0 Hz, 2H), 3.68 (d, J=7.5 Hz, 2H), 2.82 (hept, J=6.9 Hz, 1H), 2.70 (s, 6H), 1.39 (d, J=6.7 Hz, 3H), 1.16 (dd, J=6.9, 2.2 Hz, 7H).

### Example 35

The following compound was prepared according to procedures E, M, N, O, AAY, AD, AE, AJ, AL, AM and AF using appropriate starting materials.

### (Z)-4-((1-(4-amino-2-fluorobut-2-en-1-yl)-5-(1-hydroxyethyl)-3-isopropyl-1H-pyrazol-4-yl)methyl)-2-chloro-N,N-dimethylbenzenesulfonamide hydrochloride (Compound 59)

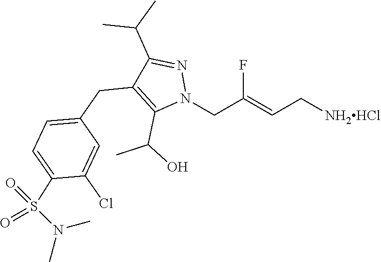

¹H-NMR (300 MHz, CD₃OD) δ ppm: 7.95 (d, J=8.2 Hz, 1H), 7.43 (d, J=1.7 Hz, 1H), 7.30 (dd, J=8.2, 1.7 Hz, 1H), 5.34 (dd, J=16.8, 11.1 Hz, 1H), 5.25-5.04 (m, 3H), 4.08 (s, 2H), 3.69 (d, J=7.5 Hz, 2H), 2.96 (p, J=7.0 Hz, 1H), 2.85 (s, 6H), 1.43 (d, J=6.7 Hz, 3H), 1.23-1.17 (m, 6H).

### Example 36

### Preparation of (Z)-4-((1-(4-amino-2-fluorobut-2-en-1-yl)-3-methyl-5-(trifluoromethyl)-1H-pyrazol-4-yl)methyl)-2-chloro-N,N-dimethylbenzenesulfonamide hydrochloride (Compound 56) and (Z)-4-((1-(4-amino-2-fluorobut-2-en-1-yl)-5-methyl-3-(trifluoromethyl)-1H-pyrazol-4-yl)methyl)-2-chloro-N,N-dimethylbenzenesulfonamide hydrochloride (Compound 57)

### Procedure AAZ: Preparation of methyl 4-amino-3-chlorobenzoate

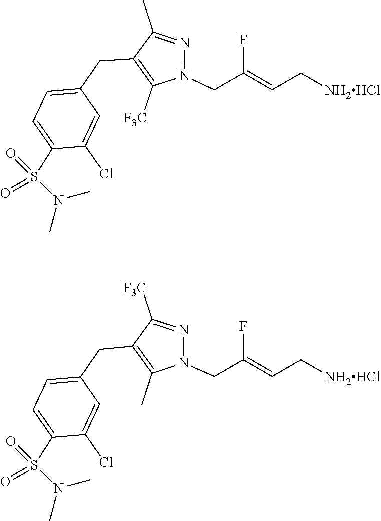

To a stirring solution of 4-amino-3-chlorobenzoic acid (2.00 g, 11.7 mmol) in methanol (20.0 mL) at 0° C. was added concentrated H₂SO₄ (2.00 mL) slowly. The resulting mixture was heated at 80° C. for 6 h. The reaction mixture was cooled to rt and then concentrated under reduced pressure. The residue was diluted with sat. aq. NaHCO₃ and extracted with ethyl acetate (2×50 mL). The combined organics were washed with brine, dried over anhydrous Na₂SO₄ and then concentrated in vacuo to afford methyl 4-amino-3-chlorobenzoate (2.10 g, 97%). ¹H-NMR (400 MHz, DMSO-d₆): δ ppm: 7.70 (d, J=1.6 Hz, 1H), 7.58 (dd, J=8.4, 2.0 Hz, 1H), 6.77 (d, J=8.8 Hz, 1H), 6.24 (s, 2H), 3.73 (s, 3H)

### Procedure AAAA: Preparation of methyl 3-chloro-4-(chlorosulfonyl)benzoate

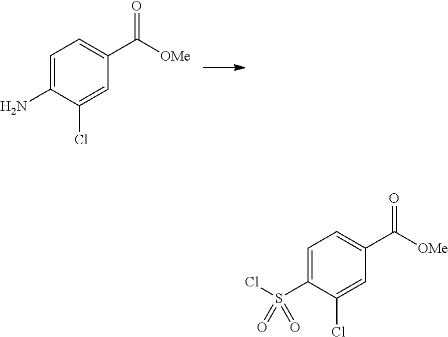 [SMILES: COC(=O)c1ccc(N)c(Cl)c1 ; COC(=O)c1ccc(S(=O)(=O)Cl)c(Cl)c1]

To a stirring solution of methyl 4-amino-3-chlorobenzoate (1.00 g, 54.1 mmol) at 0° C. was added sequentially concentrated HCl (5.0 mL), water (3.0 mL) and NaNO₂ (440 mg, 64.86 mmol). The resulting mixture was stirred at 0° C. for 1 h and then treated with acetic acid (7.0 mL), CuCl (27.0 mg, 0.27 mmol) and CuCl₂ (460 mg, 3.40 mmol). Sulfur dioxide gas was then purged through the reaction mixture for 20 min. The resulting reaction mixture was stirred at 0° C. for a further 1 h. The reaction mixture was diluted with water (30 mL) and extracted with ethyl acetate (2×50 mL). The combined organics were washed with brine and dried over anhydrous Na₂SO₄. The solvent was concentrated in vacuo. The crude material was purified over silica gel, eluting with 3%-10% ethyl acetate in hexane to afford methyl 3-chloro-4-(chlorosulfonyl)benzoate (0.92 g, 64%). ¹H-NMR (400 MHz, CDCl₃): δ ppm: 8.27 (d, J=2.0 Hz, 1H), 8.22 (d, J=8.4 Hz, 1H), 8.11 (dd, J=8.4, 2.0 Hz, 1H), 3.99 (s, 3H).

### Procedure AAAB: Preparation of methyl 3-chloro-4-(N,N-dimethylsulfamoyl)benzoate

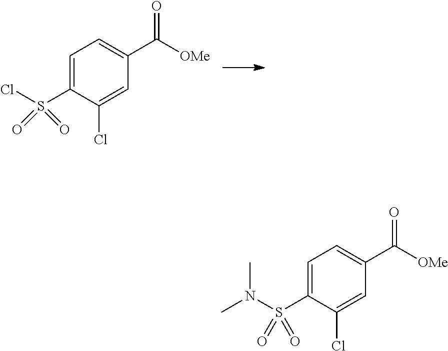 [SMILES: COC(=O)c1ccc(S(=O)(=O)Cl)c(Cl)c1 ; COC(=O)c1ccc(S(=O)(=O)N(C)C)c(Cl)c1]

To a stirring solution of methyl 3-chloro-4-(chlorosulfonyl)benzoate (0.90 g, 33.6 mmol) in THF (10.0 mL) at 0° C. was added N,N-dimethyl amine hydrochloride (0.55 g, 67.2 mmol) and DIPEA (2.33 mL, 134 mmol). The resulting mixture was stirred at 0° C. for 1 h. The reaction mixture was diluted with water (40 mL) and extracted with ethyl acetate (2×30 mL). The combined organics were washed with brine and dried over anhydrous Na₂SO₄. The solvent was removed in vacuo to afford methyl 3-chloro-4-(N,N-dimethylsulfamoyl)benzoate (0.80 g, 98%) as a white solid. ¹H-NMR (400 MHz, CDCl₃): δ ppm: 8.18 (d, J=1.6 Hz, 1H), 8.14 (d, J=8.4 Hz, 1H), 8.03 (dd, J=8.4, 2.0 Hz, 1H), 3.98 (s, 3H), 2.92 (s, 6H).

### Procedure AAAC: Preparation of 2-chloro-4-(hydroxymethyl)-N,N-dimethylbenzenesulfonamide

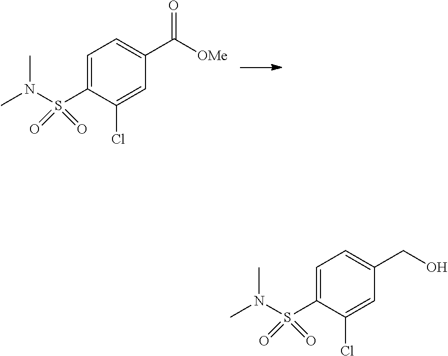 [SMILES: COC(=O)c1ccc(S(=O)(=O)N(C)C)c(Cl)c1 ; CN(C)S(=O)(=O)c1ccc(CO)cc1Cl]

To a stirring solution of methyl 3-chloro-4-(N,N-dimethylsulfamoyl)benzoate (5.00 g, 18.0 mmol) in methanol (20 mL) and THF (30 mL) at 0° C. under N₂ was added sodium borohydride (3.40 g, 89.5 mmol). The resulting mixture was stirred at 0° C. for 1 h. The reaction mixture was concentrated under reduced pressure and the resulting residue was diluted with water, and extracted with ethyl acetate (2×80 mL). The combined organics were washed with brine, dried over anhydrous Na₂SO₄ and then concentrated in vacuo to afford 2-chloro-4-(hydroxymethyl)-N,N-dimethylbenzenesulfonamide (4.30 g, 96%). ¹H-NMR (400 MHz, CDCl₃): δ ppm: 7.98 (d, J=10.8 Hz, 1H), 7.53 (s, 1H), 7.36-7.33 (m, 1H), 4.76 (s, 2H), 2.87 (s, 6H).

### Procedure AAAD: Preparation of 2-chloro-4-formyl-N,N-dimethylbenzenesulfonamide

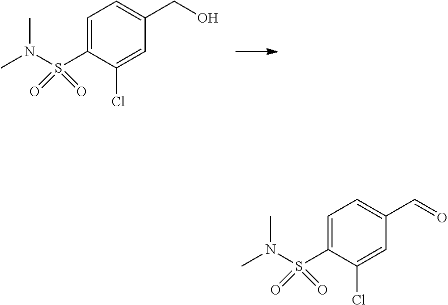

To a stirring solution of 2-chloro-4-(hydroxymethyl)-N,N-dimethylbenzenesulfonamide (3.00 g, 12.0 mmol) in CH₂Cl₂ (50.0 mL) at rt was added MnO₂ (20.8 g, 239 mmol). The resulting mixture was stirred at rt for 12 h. The reaction mixture was filtered over a Celite® pad and the filtrate was concentrated under reduced pressure. The residue was purified over silica gel, eluting with 30%-35% ethyl acetate in hexanes to afford 2-chloro-4-formyl-N,N-dimethylbenzenesulfonamide (2.20 g, 74%). ¹H-NMR (300 MHz, CDCl₃): δ ppm: 10.05 (s, 1H), 8.23 (d, J=8.1 Hz, 1H), 8.01 (s, 1H), 7.88 (dd, J=8.1, 1.8 Hz, 1H), 2.92 (s, 6H).

### Procedure AAAE: Preparation of 3-methyl-1-(tetrahydro-2H-pyran-2-yl)-5-(trifluoromethyl)-1H-pyrazole

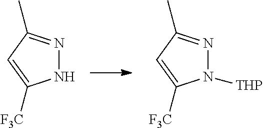

To a stirring solution of 3-methyl-5-(trifluoromethyl)-1H-pyrazole (2.00 g, 13.3 mmol) and p-toluenesulphonic acid (0.23 g, 1.33 mmol) in CHCl₃ (20 mL) at 0° C. was added 3,4-dihydropyran (1.23 g, 14.6 mmol). The reaction mixture was stirred at 0° C. for 12 h. The reaction mixture was concentrated under reduced pressure and the residue was diluted with water (50 mL), and the product was extracted with ethyl acetate (2×50 mL). The combined organics were dried over Na₂SO₄ and then concentrated in vacuo. The crude material was purified over silica gel, eluting with 15%-20% ethyl acetate in hexanes to afford 3-methyl-1-(tetrahydro-2H-pyran-2-yl)-5-(trifluoromethyl)-1H-pyrazole (1.80 g, 58%). ¹H-NMR (400 MHz, CDCl₃): δ ppm: 6.29 (s, 1H), 5.32 (dd, J=7.2 Hz, 2.4 Hz 1H), 4.01 (d, J=11.2 Hz, 1H), 3.65 (t, J=10.8 Hz, 1H), 2.37 (m, 1H), 2.18-1.98 (m, 2H), 1.70-1.57 (m, 5H).

### Procedure AAAF: Preparation of 4-bromo-3-methyl-1-(tetrahydro-2H-pyran-2-yl)-5-trifluoromethyl) 1H-pyrazole

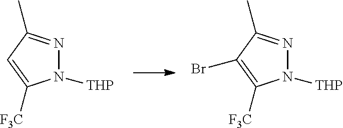

To a stirring solution of 3-methyl-1-(tetrahydro-2H-pyran-2-yl)-5-(trifluoromethyl)-1H-pyrazole (6.50 g, 27.8 mmol) in DMF (50.0 mL) at 0° C. under N₂ was added N-bromosuccinimide (7.41 g, 41.7 mmol) portion-wise. The resulting mixture was stirred at rt for 12 h. The reaction mixture was concentrated under reduced pressure and the residue obtained was diluted with water (50 mL), and extracted with ethyl acetate (2×50 mL). The combined organics were dried over Na₂SO₄ and then concentrated in vacuo. The crude material was purified over silica gel, eluting with 20%-30% ethyl acetate in hexanes to afford 4-bromo-3-methyl-1-(tetrahydro-2H-pyran-2-yl)-5-(trifluoromethyl)-1H-pyrazole (6.02 g, 69%). ¹H-NMR (300 MHz, CDCl₃): δ ppm: 5.36-5.32 (m, 1H), 4.01-3.97 (m, 1H), 3.68-3.64 (m, 1H), 2.32 (s, 3H), 2.13-1.93 (m, 2H), 1.72-1.58 (m, 4H).

### Procedure AAAG: Preparation of 2-chloro-4-(hydroxy(3-methyl-1-(tetrahydro-2H-pyran-2-yl)-5-(trifluoromethyl)-1H-pyrazol-4-yl)methyl)-N,N-dimethylbenzenesulfonamide

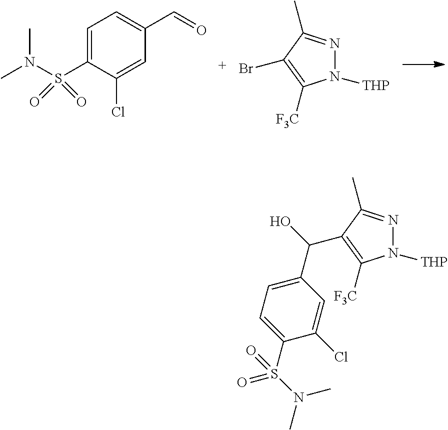

To a stirring solution of 4-bromo-3-methyl-1-(tetrahydro-2H-pyran-2-yl)-5-(trifluoromethyl)-1H-pyrazole (0.45 g, 1.44 mmol) in THF (5 mL) at −78° C. under N₂ was added n-BuLi (1.6 M in hexanes, 1.35 mL, 2.18 mmol) dropwise. The resulting reaction mixture was stirred at −78° C. for 10 min and then treated with a solution of 2-chloro-4-formyl-N,N-dimethylbenzenesulfonamide (synthesized according to procedures AAZ-AAAD) (0.53 g, 2.16 mmol) in THF (3.0 mL) dropwise over a period of 10 min. The reaction mixture was gradually warmed to rt and stirring was continued for 10 h. The reaction mixture was quenched with saturated NH₄Cl solution and extracted with ethyl acetate (2×30 mL). The combined organics were washed with brine, dried over anhydrous Na₂SO₄, and then concentrated in vacuo. The crude material was purified over silica gel, eluting with 25%-30% ethyl acetate in hexanes to afford 2-chloro-4-(hydroxy(3-methyl-1-(tetrahydro-2H-pyran-2-yl)-5-(trifluoromethyl)-1H-pyrazol-4-yl)methyl)-N,N-dimethylbenzenesulfonamide (0.52 g, 75%). ¹H-NMR (300 MHz, CDCl₃): δ ppm: 7.96 (dd, J=8.4, 2.7 Hz, 1H), 7.56 (d, J=5.6 Hz, 1H), 7.33 (d, J=8.1 Hz, 1H), 6.04 (d, J=2.7 Hz, 1H), 5.30-5.27 (m, 1H), 4.03-3.99 (m, 1H), 3.67-3.6 (m, 1H), 2.87 (s, 6H), 2.43-2.36 (m, 2H), 2.17 (s, 3H), 1.98-1.94 (m, 2H), 1.69-1.6 (m, 3H).

### Procedure AAAH: Preparation of 2-chloro-N,N-dimethyl-4-((3-methyl-5-(trifluoromethyl)-1H-pyrazol-4-yl)methyl)benzenesulfonamide

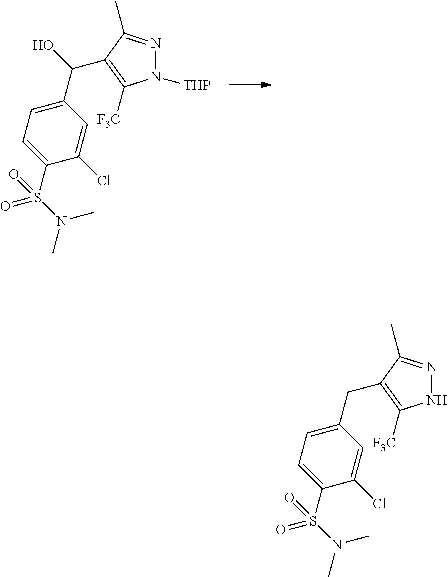

To a stirring solution of 2-chloro-4-(hydroxy(3-methyl-1-(tetrahydro-2H-pyran-2-yl)-5-(trifluoromethyl)-1H-pyrazol-4-yl)methyl)-N,N-dimethylbenzenesulfonamide (0.45 g, 0.94 mmol) in trifluoroacetic acid (5.0 mL) at 0° C. under N₂ was added triethylsilane (0.75 mL, 4.67 mmol). The reaction mixture was heated at 60° C. and stirring was continued for 6 h. The reaction mixture was cooled to rt and quenched with saturated NaHCO₃ solution before extracting with CH₂Cl₂ (2×25 mL). The combined organics were washed with brine, dried over anhydrous Na₂SO₄, and concentrated in vacuo. The crude material was purified over silica gel, eluting with 40%-50% ethyl acetate in hexanes to afford 2-chloro-N,N-dimethyl-4-((3-methyl-5-(trifluoromethyl)-1H-pyrazol-4-yl)methyl)benzenesulfonamide (0.25 g, 70%). ¹H-NMR (300 MHz, CDCl₃): δ ppm: 7.94 (d, J=8.4 Hz, 1H), 7.23 (s, 1H), 7.11 (dd, J=8.1, 1.8 Hz, 1H), 3.92 (s, 2H), 2.87 (s, 6H), 2.22 (s, 3H).

### Procedure AAAI: Preparation of tert-butyl (Z)-(4-(4-(3-chloro-4-(N,N-dimethylsulfamoyl)benzyl)-5-methyl-3-(trifluoromethyl)-1H-pyrazol-1-yl)-3-fluorobut-2-en-1-yl)carbamate and tert-butyl (Z)-(4-(4-(3-chloro-4-(N,N-dimethylsulfamoyl)benzyl)-3-methyl-5-(trifluoromethyl)-1H-pyrazol-1-yl)-3-fluorobut-2-en-1-ylcarbamate

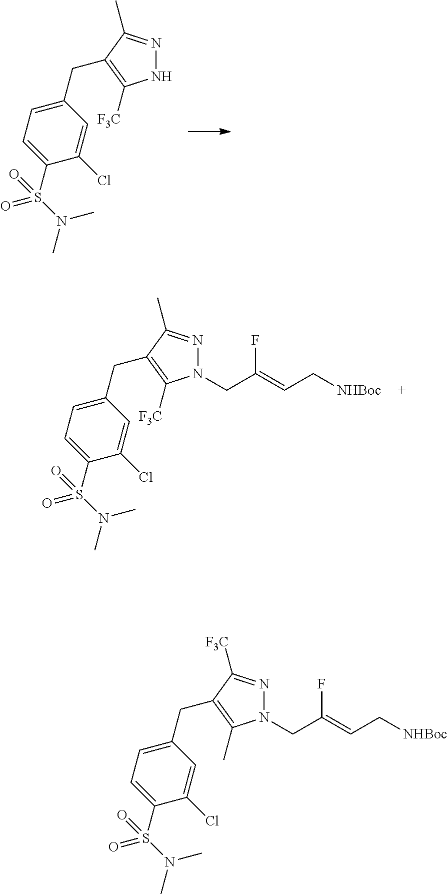 [SMILES: Cc1n[nH]c(C(F)(F)F)c1Cc1ccc(S(=O)(=O)N(C)C)c(Cl)c1 ; Cc1nn(C/C(F)=C/CNC(=O)OC(C)(C)C)c(C(F)(F)F)c1Cc1ccc(S(=O)(=O)N(C)C)c(Cl)c1 ; Cc1c(Cc2ccc(S(=O)(=O)N(C)C)c(Cl)c2)c(C(F)(F)F)nn1C/C(F)=C/CNC(=O)OC(C)(C)C]

To a stirred solution of 2-chloro-N,N-dimethyl-4-((3-methyl-5-(trifluoromethyl)-1H-pyrazol-4-yl)methyl)benzenesulfonamide (1.40 g, 3.66 mmol) in DMF (10 mL) at rt was treated sequentially with tert-butyl (Z)-(4-bromo-3-fluorobut-2-en-1-yl)carbamate (1.96 g, 1.05 mmol) and K₂CO₃ (1.01 g, 7.33 mmol). The reaction mixture was heated to 80° C. and stirring was continued for 6 h. The reaction mixture was cooled to rt, diluted with water (30 mL) and extracted with ethyl acetate (2×30 mL). The combined organics were washed with brine, dried over anhydrous Na₂SO₄ and then concentrated in vacuo. The crude material was purified over silica gel, eluting with 40% ethyl acetate in hexane to afford a mixture of isomers which was further purified by preparative HPLC [Zorbax Eclipze XDB C18 (150 mm×21.2 mm), 5.0p, A=H₂O; B=MeCN, 40% to 90% MeCN, Flow-15.0 mL/min]. The HPLC fractions were lyophilized to afford tert-butyl (Z)-(4-(4-(3-chloro-4-(N,N-dimethylsulfamoyl)benzyl)-3-methyl-5-(trifluoromethyl)-1H-pyrazol-1-yl)-3-fluorobut-2-en-1-yl)carbamate (50.0 mg, 2%) and tert-butyl (Z)-(4-(4-(3-chloro-4-(N,N-dimethylsulfamoyl)benzyl)-5-methyl-3-(trifluoromethyl)-1H-pyrazol-1-yl)-3-fluorobut-2-en-1-yl)carbamate (300 mg, 14%).

### Tert-butyl (Z)-(4-(4-(3-chloro-4-(N,N-dimethylsulfamoyl)benzyl)-5-methyl-3-(trifluoromethyl)-1H-pyrazol-1-yl)-3-fluorobut-2-en-1-yl)carbamate

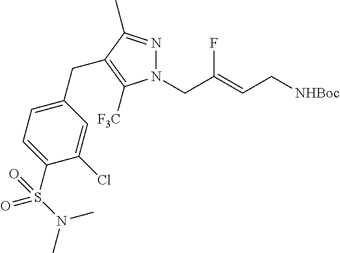 [SMILES: Cc1nn(C/C(F)=C/CNC(=O)OC(C)(C)C)c(C(F)(F)F)c1Cc1ccc(S(=O)(=O)N(C)C)c(Cl)c1]

¹H-NMR (300 MHz, CDCl₃): δ ppm: 7.94 (d, J=8.4 Hz, 1H), 7.2 (s, 1H), 7.06 (d, J=8.1 Hz, 1H), 5.0 (br, 0.5H), 4.85 (d, J=13.2 Hz, 2H), 4.61 (br, 0.5H), 3.9 (s, 2H), 3.83 (br, 2H), 2.87 (s, 6H), 2.14 (s, 3H), 1.43 (s, 9H).

### Tert-butyl (Z)-(4-(4-(3-chloro-4-(N,N-dimethylsulfamoyl)benzyl)-3-methyl-5-(trifluoromethyl)-1H-pyrazol-1-yl)-3-fluorobut-2-en-1-yl)carbamate

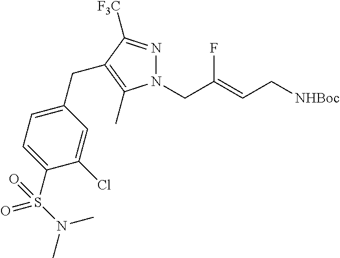 [SMILES: Cc1c(Cc2ccc(S(=O)(=O)N(C)C)c(Cl)c2)c(C(F)(F)F)nn1C/C(F)=C/CNC(=O)OC(C)(C)C]

¹H-NMR (300 MHz, CDCl₃): δ ppm: 7.93 (d, J=8.4 Hz, 1H), 7.21 (d, J=1.5 Hz, 1H), 7.08 (dd, J=8.1, 1.8 Hz, 1H), 4.99-4.66 (m, 4H), 3.9 (s, 2H), 3.82 (br, 2H), 2.86 (s, 6H), 2.18 (s, 3H), (br, 0.5H), 3.9 (s, 2H), 3.83 (br, 2H), 2.87 (s, 6H), 2.14 (s, 3H), 1.42 (s, 9H).

### Procedure AAAJ: Preparation of (Z)-4-((1-(4-amino-2-fluorobut-2-en-1-yl)-3-methyl-5-(trifluoromethyl)-1H-pyrazol-4-yl)methyl)-2-chloro-N,N-dimethylbenzenesulfonamide hydrochloride (Compound 56)

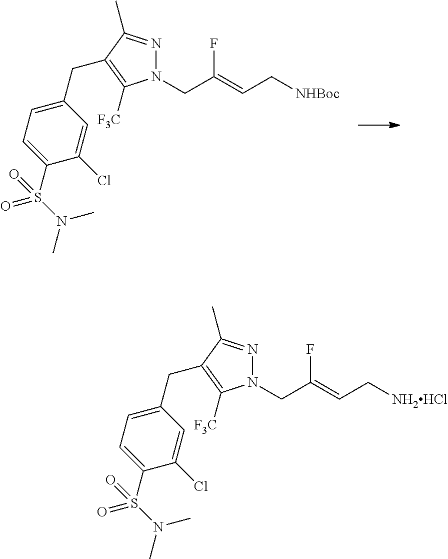

To a stirring solution of tert-butyl (Z)-(4-(4-(3-chloro-4-(N,N-dimethylsulfamoyl)benzyl)-5-methyl-3-(trifluoromethyl)-1H-pyrazol-1-yl)-3-fluorobut-2-en-1-yl)carbamate (50.0 mg, 0.09 mmol) in 1,4-dioxane at 0° C. was added HCl (4.0 M in 1,4-dioxane; 4.00 mmol, 1.00 mL). The resulting mixture was warmed to rt and stirring was continued for 12 h. The reaction mixture was concentrated under reduced pressure and the residue obtained was washed with diethyl ether and n-pentane to afford (Z)-4-((1-(4-amino-2-fluorobut-2-en-1-yl)-3-methyl-5-(trifluoromethyl)-1H-pyrazol-4-yl)methyl)-2-chloro-N,N-dimethylbenzenesulfonamide hydrochloride (23.0 mg, 51%). ¹H-NMR (400 MHz, CD₃OD): δ ppm: 7.92 (d, J=8.4 Hz, 1H), 7.31 (s, 1H), 7.19 (dd, J=8.4, 0.8 Hz, 1H), 5.19-5.10 (m, 1H), 5.04 (d, J=13.6 Hz, 2H), 4.03 (s, 2H), 3.64 (d, J=6.8 Hz, 2H), 2.83 (s, 6H), 2.15 (s, 3H).

### Procedure AAAK: Preparation of (Z)-4-((1-(4-amino-2-fluorobut-2-en-1-yl)-3-methyl-5-(trifluoromethyl)-1H-pyrazol-4-yl)methyl)-2-chloro-N,N-dimethylbenzenesulfonamide hydrochloride (Compound 57)

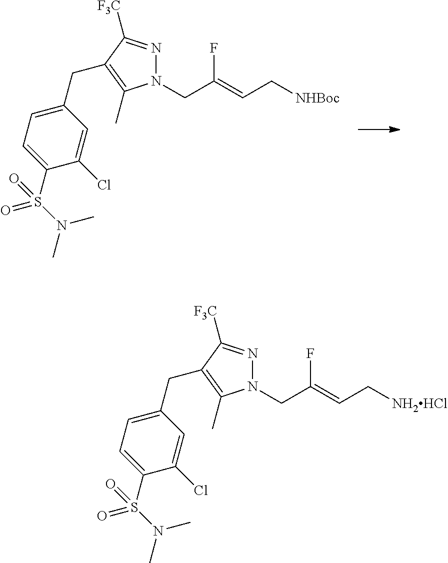

To a stirring solution of tert-butyl (Z)-(4-(4-(3-chloro-4-(N,N-dimethylsulfamoyl)benzyl)-5-methyl-3-(trifluoromethyl)-1H-pyrazol-1-yl)-3-fluorobut-2-en-1-yl)carbamate (300 mg, 0.09 mmol) in 1,4-dioxane at 0° C. was added HCl (4.0 M in 1,4-dioxane; 4.00 mmol, 1.00 mL). The resulting mixture was warmed to rt and stirring was continued for 12 h. The reaction mixture was concentrated under reduced pressure and the residue obtained was washed with diethyl ether and n-pentane to afford (Z)-4-((1-(4-amino-2-fluorobut-2-en-1-yl)-3-methyl-5-(trifluoromethyl)-1H-pyrazol-4-yl)methyl)-2-chloro-N,N-dimethylbenzenesulfonamide hydrochloride (120 mg, 45%). ¹H-NMR (400 MHz, CD₃OD): δ ppm: 7.91 (d, J=8.0 Hz, 1H), 7.31 (s, 1H), 7.20 (dd, J=6.8, 1.6 Hz, 1H), 5.19-5.1 (m, 1H), 5.04 (d, J=14 Hz, 2H), 3.99 (s, 2H), 3.65 (d, J=7.2 Hz, 2H), 2.82 (s, 6H), 2.29 (s, 3H).

### Example 37

### Preparation of (Z)-4-((1-(4-amino-2-fluorobut-2-en-1-yl)-3,5-diisopropyl-1H-pyrazol-4-yl)methyl)-2-chloro-N,N-dimethylbenzenesulfonamide hydrochloride (Compound 60)

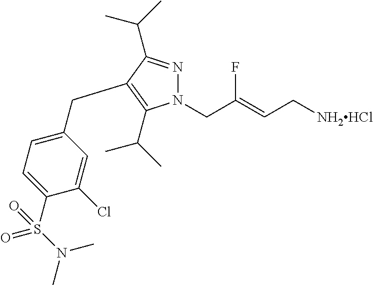

### Procedure AAAL: Preparation of 2-chloro-4-(2-isobutyryl-4-methyl-3-oxopentyl)-N,N-dimethylbenzenesulfonamide

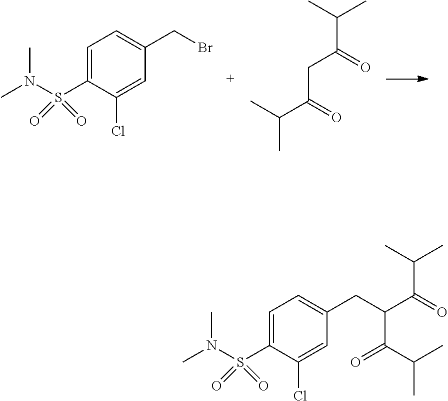 [SMILES: CN(C)S(=O)(=O)c1ccc(CBr)cc1Cl ; CC(C)C(=O)CC(=O)C(C)C ; CC(C)C(=O)C(Cc1ccc(S(=O)(=O)N(C)C)c(Cl)c1)C(=O)C(C)C]

To a stirring solution of 2,6-dimethylheptane-3,5-dione (2.00 g, 6.41 mmol) in THF (5.0 mL) and DMF (5.0 mL) at 0° C. under N₂ was added NaHMDS (1.0 M in THF; 9.61 mL, 9.61 mmol). After stirring for 10 min, 4-(bromomethyl)-2-chloro-N,N-dimethylbenzenesulfonamide (3.00 g, 19.2 mmol) was added. The resulting mixture was gradually warmed to rt and stirring was continued for 3 h. The reaction mixture was poured into ice cold water (50 mL) and the product was extracted with ethyl acetate (2×50 mL). The combined organics were washed with brine, dried over anhydrous Na₂SO₄ and concentrated in vacuo. The residue obtained (1.81 g) was progressed to the next step without any purification.

### Procedure AAAM: Preparation of 2-chloro-4-((3,5-diisopropyl-1H-pyrazol-4-yl)methyl)-N,N-dimethylbenzenesulfonamide

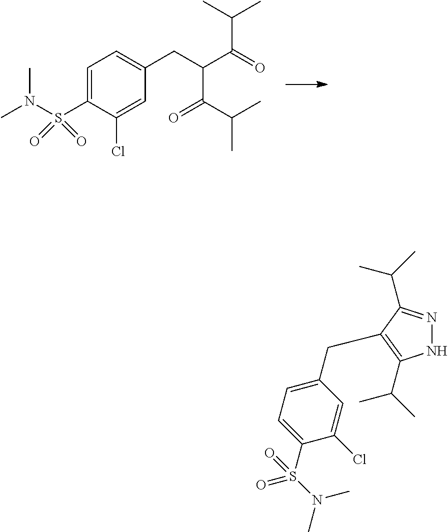 [SMILES: CC(C)C(=O)C(Cc1ccc(S(=O)(=O)N(C)C)c(Cl)c1)C(=O)C(C)C ; CC(C)c1n[nH]c(C(C)C)c1Cc1ccc(S(=O)(=O)N(C)C)c(Cl)c1]

To a stirring solution of 2-chloro-4-(2-isobutyryl-4-methyl-3-oxopentyl)-N,N-dimethylbenzenesulfonamide (1.50 g, 3.86 mmol) in ethanol (50.0 mL) at rt was added hydrazine hydrate (5.0 mL). The resulting mixture was heated to reflux and stirring was continued for 12 h. The reaction mixture was cooled to rt, diluted with water (50 mL) and extracted with ethyl acetate (2×50 mL). The combined organics were washed with brine, dried over anhydrous Na₂SO₄ and concentrated in vacuo to afford 2-chloro-4-((3,5-diisopropyl-1H-pyrazol-4-yl)methyl)-N,N-dimethylbenzenesulfonamide (1.30 g, 88%). ¹H-NMR (300 MHz, CDCl₃) δ ppm: 7.92 (d, J=8.1 Hz, 1H), 7.24 (d, J=1.5 Hz, 1H), 7.12-7.09 (m, 1H), 3.84 (s, 2H), 2.86 (s, 6H), 1.20 (d, J=6.9 Hz, 12H).

### Procedure AAAN: Preparation of tert-butyl (Z)-(4-(4-(3-chloro-4-(N,N-dimethylsulfamoyl)benzyl)-3,5-diisopropyl-1H-pyrazol-1-yl)-3-fluorobut-2-en-1-yl)carbamate

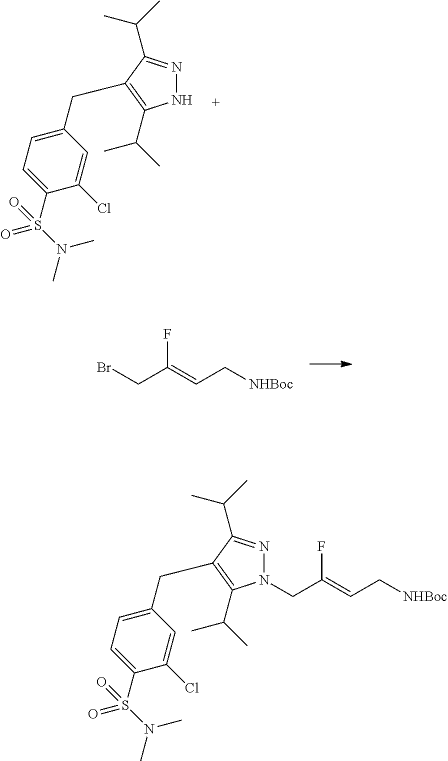 [SMILES: CC(C)c1n[nH]c(C(C)C)c1Cc1ccc(S(=O)(=O)N(C)C)c(Cl)c1 ; CC(C)(C)OC(=O)NC/C=C(\F)CBr ; CC(C)c1nn(C/C(F)=C/CNC(=O)OC(C)(C)C)c(C(C)C)c1Cc1ccc(S(=O)(=O)N(C)C)c(Cl)c1]

To a stirring solution of 2-chloro-4-((3,5-diisopropyl-1H-pyrazol-4-yl)methyl)-N,N-dimethylbenzenesulfonamide (1.00 g, 2.61 mmol) in DMSO (20.0 mL) at rt was added KOH (0.31 g, 5.48 mmol) followed by portion wise addition of tert-butyl (Z)-(4-bromo-3-fluorobut-2-en-1-yl)carbamate (1.04 g, 3.91 mmol). The resulting mixture was stirred at rt for 2 h. The reaction mixture was diluted with water (50 mL) and extracted with ethyl acetate (2×50 mL). The combined organics were washed with brine, dried over anhydrous Na₂SO₄ and concentrated in vacuo. The crude material was purified over silica gel, eluting with 10%-30% ethyl acetate in hexane to afford tert-butyl (Z)-(4-(4-(3-chloro-4-(N,N-dimethylsulfamoyl)benzyl)-3,5-diisopropyl-1H-pyrazol-1-yl)-3-fluorobut-2-en-1-yl)carbamate (0.45 g, 30%).

### Procedure AAAO: Preparation of (Z)-4-((1-(4-amino-2-fluorobut-2-en-1-yl)-3,5-diisopropyl-1H-pyrazol-4-yl)methyl-2-chloro-N,N-dimethylbenzenesulfonamide hydrochloride (Compound 60)

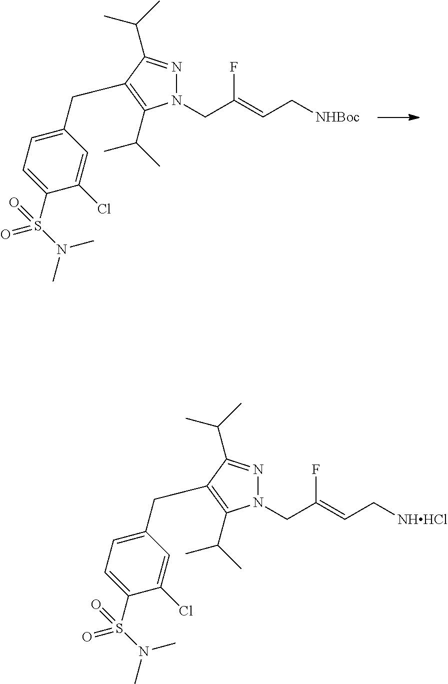

To a stirring solution of tert-butyl (Z)-(4-(4-(3-chloro-4-(N,N-dimethylsulfamoyl)benzyl)-3,5-diisopropyl-1H-pyrazol-1-yl)-3-fluorobut-2-en-1-yl)carbamate (0.25 g, 0.44 mmol) in 1,4-dioxane (10 mL) at 0° C. was added HCl (4.0 M in 1,4-dioxane; 12.0 mmol, 3.0 mL). The reaction temperature was gradually raised to rt and stirring was continued for 1 h. The reaction mixture was concentrated under reduced pressure. The crude material was purified using preparative HPLC [Waters Xbridge C18 (150 mm×21.20 mm), A=0.05% HCl in water; B=MeCN; Gradient, 10-50% B, Flow: 15.0 mL/min]. The fractions were lyophilized to afford (Z)-4-((1-(4-amino-2-fluorobut-2-en-1-yl)-3,5-diisopropyl-1H-pyrazol-4-yl)methyl)-2-chloro-N,N-dimethylbenzenesulfonamide hydrochloride (Compound 60) (123 mg, 60%). ¹H-NMR (300 MHz, CD₃OD) δ ppm: 7.92 (d, J=8.1 Hz, 1H), 7.32 (s, 1H), 7.23-7.20 (m, 1H), 5.20-5.0 (m, 2H), 4.06 (s, 2H), 3.66 (d, J=6.9 Hz, 2H), 3.25-3.22 (m, 1H), 2.90-2.85 (m, 1H), 2.83 (s, 6H), 1.24 (d, J=7.2 Hz, 6H), 1.16 (d, J=7.2 Hz, 6H).

### Example 38

### Preparation of (Z)-1-(4-amino-2-fluorobut-2-en-1-yl)-4-(3-chloro-4-(N,N-dimethylsulfamoyl)benzyl)-N,N,3-trimethyl-1H-pyrazole-5-carboxamide hydrochloride (Compound 61)

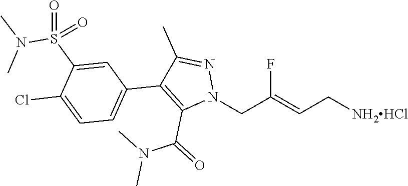

### Procedure AAAP: Preparation of ethyl (Z)-2-(methoxyimino)-4-oxopentanoate

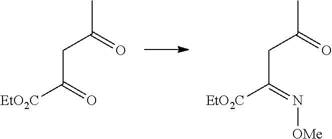

To a stirring solution of ethyl 2,4-dioxopentanoate (10.0 g, 63.29 mmol) in DMF (50.0 mL) at rt was added O-methylhydroxylamine hydrochloride (5.81 mg, 69.6 mmol) followed by powdered 4 Å molecular sieves (20.0 g). The resulting mixture was stirred at rt for 14 h. The reaction mixture was partitioned between water (150 mL) and ethyl acetate (200 mL). The aqueous layer was extracted with ethyl acetate (100 mL). The combined organics were washed with water (2×100 mL), brine (100 mL), dried over anhydrous Na₂SO₄ and concentrated in vacuo. The crude material was purified over silica gel, eluting with 15%-20% ethyl acetate in hexane to afford ethyl (Z)-2-(methoxyimino)-4-oxopentanoate (5.60 g, 47%). ¹H-NMR (300 MHz, CDCl₃): δ ppm: 4.33 (q, J=7.2 Hz, 2H), 4.05 (s, 3H), 3.70 (s, 2H), 2.19 (s, 3H), 1.34 (t, J=6.9 Hz, 3H).

### Procedure AAAO: Preparation of ethyl (Z)-3-(3-chloro-4-(N,N-dimethylsulfamoyl)benzyl)-2-(methoxyimino)-4-oxopentanoate

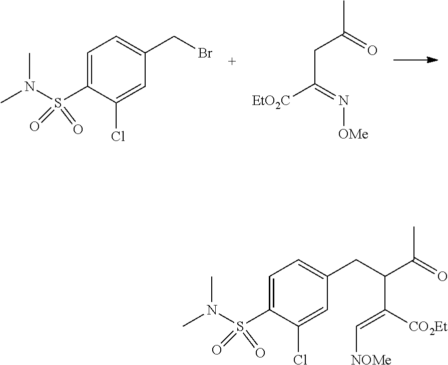

To a stirring solution of ethyl (Z)-2-(methoxyimino)-4-oxopentanoate (2.30 g, 12.3 mmol) in DMF (30.0 mL) at rt under N₂ was added K₂CO₃ (4.24 g, 30.72 mmol) followed by 4-(bromomethyl)-2-chloro-N,N-dimethylbenzenesulfonamide (synthesized according to procedures M, N and O) (3.84 g, 12.3 mmol). The resulting mixture was stirred at rt for 3 h. The reaction mixture was diluted with water and extracted with ethyl acetate (2×50 mL). The combined organics were washed with water, brine, dried over anhydrous Na₂SO₄ and concentrated in vacuo. The crude material was purified over silica gel, eluting with 20%-25% ethyl acetate in hexane to afford ethyl (Z)-3-(3-chloro-4-(N,N-dimethylsulfamoyl)benzyl)-2-(methoxyimino)-4-oxopentanoate (2.80 g, 54%). ¹H-NMR (300 MHz, CDCl₃): δ ppm: 7.92 (d, J=8.1 Hz, 1H), 7.30 (d, J=1.5 Hz, 1H), 7.16 (dd, J=8.1, 1.8 Hz, 1H), 4.27 (q, J=6.9 Hz, 2H), 4.03 (s, 3H), 3.44-3.37 (m, 1H), 2.97-2.87 (m, 2H), 2.85 (s, 6H), 2.08 (s, 3H), 1.28 (t, J=7.2 Hz, 3H).

### Procedure AAAR: Preparation of ethyl 4-(3-chloro-4-(N,N-dimethylsulfamoyl)benzyl)-3-methyl-1H-pyrazole-5-carboxylate

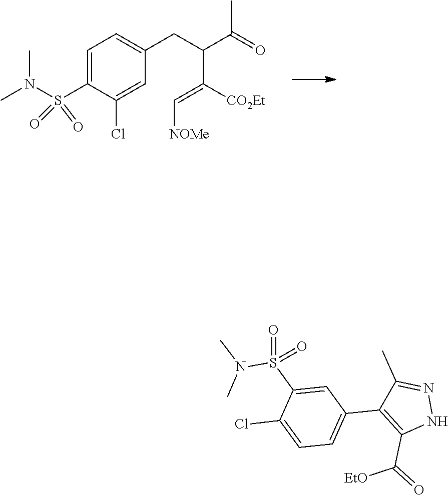

To a stirring solution of ethyl (Z)-3-(3-chloro-4-(N,N-dimethylsulfamoyl)benzyl)-2-(methoxyimino)-4-oxopentanoate (2.80 g, 6.68 mmol) in ethanol (60.0 mL) and water (22.0 mL) was added hydrazine sulphate (1.30 g, 10.0 mmol) followed by powdered 4 Å molecular sieves (3.0 g). The resulting mixture was heated at reflux for 8 h, cooled to rt and stirred for a further 6 h. The reaction mixture was diluted with aq. HCl (2 M; 150 mL) and extracted with ethyl acetate (2×100 mL). The combined organics were washed with sat. aq. NaHCO₃, water, and brine, dried over anhydrous Na₂SO₄ and then concentrated in vacuo. The crude material was purified over silica gel, eluting with 60%-70% ethyl acetate in hexanes to afford ethyl 4-(3-chloro-4-(N,N-dimethylsulfamoyl)benzyl)-3-methyl-1H-pyrazole-5-carboxylate (1.60 g, 62%). ¹H-NMR (300 MHz, CDCl₃): δ ppm: 7.92 (d, J=8.4 Hz, 1H), 7.27 (d, J=1.5 Hz, 1H), 7.17-7.14 (m, 1H), 4.35 (q, J=7.2 Hz, 2H), 4.12 (s, 2H), 2.85 (s, 6H), 2.24 (s, 3H), 1.31 (t, J=7.2 Hz, 3H).

### Procedure AAAS: Preparation of ethyl (Z)-1-(4-((tert-butoxycarbonyl)amino)-2-fluorobut-2-en-1-yl)-4-(3-chloro-4-(N,N-dimethylsulfamoyl)benzyl)-3-methyl-1H-pyrazole-5-carboxylate and ethyl (Z)-1-(4-((tert-butoxycarbonyl)amino)-2-fluorobut-2-en-1-yl)-4-(3-chloro-4-(N,N-dimethylsulfamoyl)benzyl)-5-methyl-1H-pyrazole-3-carboxylate

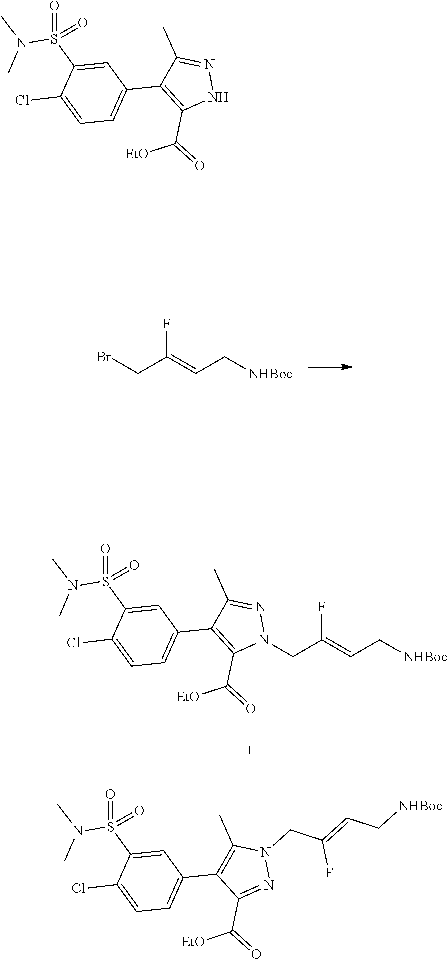 [SMILES: CCOC(=O)c1[nH]nc(C)c1-c1ccc(Cl)c(S(=O)(=O)N(C)C)c1 ; CC(C)(C)OC(=O)NC/C=C(\F)CBr ; CCOC(=O)c1c(-c2ccc(Cl)c(S(=O)(=O)N(C)C)c2)c(C)nn1C/C(F)=C/CNC(=O)OC(C)(C)C ; CCOC(=O)c1nn(C/C(F)=C/CNC(=O)OC(C)(C)C)c(C)c1-c1ccc(Cl)c(S(=O)(=O)N(C)C)c1]

To a stirring solution of ethyl 4-(3-chloro-4-(N,N-dimethylsulfamoyl)benzyl)-3-methyl-1H-pyrazole-5-carboxylate (1.40 g, 3.62 mmol) in DMF (30.0 mL) at rt under N₂ was added Cs₂CO₃ (2.95 g, 9.06 mmol) followed by tert-butyl (Z)-(4-bromo-3-fluorobut-2-en-1-yl)carbamate (1.06 g, 3.98 mmol). The resulting mixture was heated at 60° C. for 2 h. The reaction mixture was diluted water (100 mL) and extracted with ethyl acetate (2×50 mL). The organic layers were combined, washed with water, brine, dried over anhydrous Na₂SO₄ and concentrated in vacuo. The crude material was purified over silica gel, eluting with 20%-25% ethyl acetate in hexanes to afford the desired regio-isomer ethyl (Z)-1-(4-((tert-butoxycarbonyl)amino)-2-fluorobut-2-en-1-yl)-4-(3-chloro-4-(N,N-dimethylsulfamoyl)benzyl)-3-methyl-1H-pyrazole-5-carboxylate (1.00 g, 48%) followed by undesired regio-isomer ethyl (Z)-1-(4-((tert-butoxycarbonyl)amino)-2-fluorobut-2-en-1-yl)-4-(3-chloro-4-(N,N-dimethylsulfamoyl)benzyl)-5-methyl-1H-pyrazole-3-carboxylate (0.40 g, 19%).

### Ethyl (Z)-1-(4-((tert-butoxycarbonyl)amino)-2-fluorobut-2-en-1-yl)-4-(3-chloro-4-(N,N-dimethylsulfamoyl)benzyl)-3-methyl-1H-pyrazole-5-carboxylate

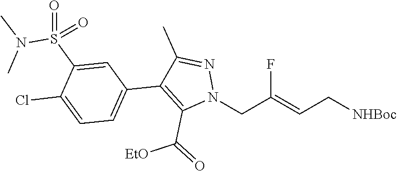 [SMILES: CCOC(=O)c1c(-c2ccc(Cl)c(S(=O)(=O)N(C)C)c2)c(C)nn1C/C(F)=C/CNC(=O)OC(C)(C)C]

¹H-NMR (300 MHz, CDCl₃): δ ppm: 7.94 (d, J=8.1 Hz, 1H), 7.25 (d, J=6.9 Hz, 1H), 7.11 (d, J=8.4 Hz, 1H), 5.24 (d, J=12.6 Hz, 2H), 4.94-4.77 (m, 1H), 4.58 (bs, 1H), 4.30 (q, J=7.2 Hz, 2H), 4.08 (s, 2H), 3.80 (bs, 2H), 2.86 (s, 6H), 2.18 (s, 3H), 1.42 (s, 9H), 1.27 (t, J=7.2 Hz, 3H).

### Ethyl (Z)-1-(4-((tert-butoxycarbonyl)amino)-2-fluorobut-2-en-1-yl)-4-(3-chloro-4-(N,N-dimethylsulfamoyl)benzyl)-5-methyl-1H-pyrazole-3-carboxylate

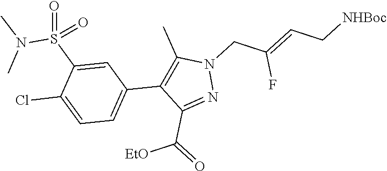 [SMILES: CCOC(=O)c1nn(C/C(F)=C/CNC(=O)OC(C)(C)C)c(C)c1-c1ccc(Cl)c(S(=O)(=O)N(C)C)c1]

¹H-NMR (300 MHz, CDCl₃): δ ppm: 7.92 (d, J=6.3 Hz, 1H), 7.25 (s, 1H), 7.14 (dd, J=3.0, 1H), 4.94 (m, 1H), 4.86 (m, 2.5H), 4.62 (m, 1H), 4.36 (q, J=7.2, 2H), 4.12 (s, 2H), 3.81 (bs, 2H), 2.85 (s, 6H), 2.22 (s, 3H), 1.42 (s, 9H), 1.32 (t, J=7.2 Hz, 3H).

### Procedure AAAT: Preparation of (Z)-1-(4-((tert-butoxycarbonyl)amino)-2-fluorobut-2-en-1-yl)-4-(3-chloro-4-(N,N-dimethylsulfamoyl)benzyl)-3-methyl-1H-pyrazole-5-carboxylic Acid

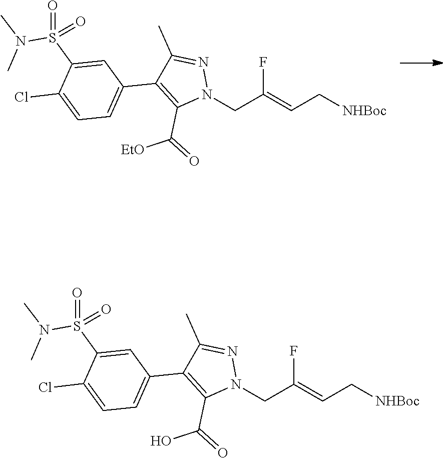 [SMILES: CCOC(=O)c1c(-c2ccc(Cl)c(S(=O)(=O)N(C)C)c2)c(C)nn1C/C(F)=C/CNC(=O)OC(C)(C)C ; Cc1nn(C/C(F)=C/CNC(=O)OC(C)(C)C)c(C(=O)O)c1-c1ccc(Cl)c(S(=O)(=O)N(C)C)c1]

To a stirring solution of ethyl (Z)-1-(4-((tert-butoxycarbonyl)amino)-2-fluorobut-2-en-1-yl)-4-(3-chloro-4-(N,N-dimethylsulfamoyl)benzyl)-3-methyl-1H-pyrazole-5-carboxylate (0.80 g, 1.39 mmol) in methanol (15.0 mL) at rt was added 10 w % aq. KOH (5.0 mL). The resulting mixture was stirred at rt for 1 h. The reaction mixture was concentrated under reduced pressure. The residue was diluted with water, acidified to pH 5 with aqueous HCl (2.0 M) and extracted with CH₂Cl₂ (2×50 mL). The combined organics were washed with water, brine, dried over anhydrous Na₂SO₄ and concentrated in vacuo to afford (Z)-1-(4-((tert-butoxycarbonyl)amino)-2-fluorobut-2-en-1-yl)-4-(3-chloro-4-(N,N-dimethylsulfamoyl)benzyl)-3-methyl-1H-pyrazole-5-carboxylic acid (0.65 g, 85%) as a white solid. ¹H-NMR (300 MHz, CDCl₃): δ ppm: 7.92 (d, J=8.4 Hz, 1H), 7.25 (s, 1H), 7.14 (d, J=9.0 Hz, 1H), 5.25 (bs, 2H), 4.96-4.84 (m, 1H), 4.13 (s, 2H), 3.79 (bs, 2H), 2.86 (s, 6H), 2.17 (s, 3H), 1.42 (s, 9H).

### Procedure AAAU: Preparation of tert-butyl (Z)-(4-(4-(3-chloro-4-(N,N-dimethylsulfamoyl)benzyl)-5-(dimethylcarbamoyl)-3-methyl-1H-pyrazol-1-yl)-3-fluorobut-2-en-1-yl)carbamate

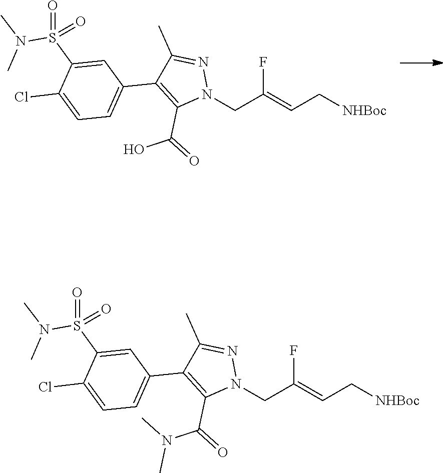 [SMILES: Cc1nn(C/C(F)=C/CNC(=O)OC(C)(C)C)c(C(=O)O)c1-c1ccc(Cl)c(S(=O)(=O)N(C)C)c1 ; Cc1nn(C/C(F)=C/CNC(=O)OC(C)(C)C)c(C(=O)N(C)C)c1-c1ccc(Cl)c(S(=O)(=O)N(C)C)c1]

To a stirring solution of (Z)-1-(4-((tert-butoxycarbonyl)amino)-2-fluorobut-2-en-1-yl)-4-(3-chloro-4-(N,N-dimethylsulfamoyl)benzyl)-3-methyl-1H-pyrazole-5-carboxylic acid (0.30 g, 0.55 mmol) in DMF (10.0 mL) at rt under N₂ was added sequentially triethylamine (0.16 g, 1.65 mmol), HATU (0.31 g, 0.96 mmol) and dimethylamine hydrochloride (0.08 g, 0.83 mmol). The resulting mixture was stirred at rt for 2 h. The reaction mixture was diluted water (30 mL) and extracted with ethyl acetate (2×50 mL). The combined organics were washed with aq. HCl (1.0 M; 20 mL), water, sat. aq. NaHCO₃, brine, dried over anhydrous Na₂SO₄ and then concentrated in vacuo. The residue obtained was purified over silica gel, eluting with 80%-90% ethyl acetate in hexanes to afford the tert-butyl (Z)-(4-(4-(3-chloro-4-(N,N-dimethylsulfamoyl)benzyl)-5-(dimethylcarbamoyl)-3-methyl-1H-pyrazol-1-yl)-3-fluorobut-2-en-1-yl)carbamate (0.25 g, 80%). ¹H-NMR (300 MHz, CDCl₃): δ ppm: 7.93 (d, J=8.1 Hz, 1H), 7.26 (s, 1H), 7.12 (dd, J=8.1, 0.9 Hz, 1H), 5.07-4.79 (m, 1H), 4.70-4.64 (m, 3H), 3.76-3.75 (m, 4H), 3.00 (s, 3H), 2.85 (s, 6H), 2.82 (s, 3H), 2.10 (s, 3H), 1.42 (s, 9H).

### Procedure AAAV: Preparation of (Z)-1-(4-amino-2-fluorobut-2-en-1-yl)-4-(3-chloro-4-(N,N-dimethylsulfamoyl)benzyl)-N,N,3-trimethyl-1H-pyrazole-5-carboxamide hydrochloride Compound 61)

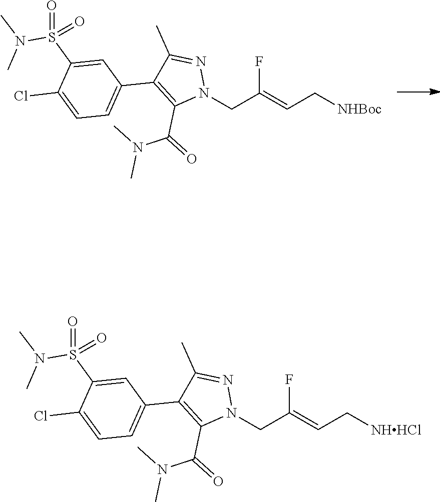

To a stirring solution of tert-butyl (Z)-(4-(4-(3-chloro-4-(N,N-dimethylsulfamoyl)benzyl)-5-(dimethylcarbamoyl)-3-methyl-1H-pyrazol-1-yl)-3-fluorobut-2-en-1-yl)carbamate (250 mg, 0.43 mmol) in 1,5-dioxane (10 mL) at rt was added HCl (4.0 M in 1,4-dioxane; 3.0 mL, 12.0 mmol). The resulting mixture was stirred at rt for 1 h. The reaction mixture was concentrated under reduced pressure. The residue was triturated with ethyl acetate and diethyl ether gave gummy solid. Drying under high vacuum afforded the (Z)-1-(4-amino-2-fluorobut-2-en-1-yl)-4-(3-chloro-4-(N,N-dimethylsulfamoyl)benzyl)-N,N,3-trimethyl-1H-pyrazole-5-carboxamide hydrochloride (Compound 61) (151 mg, 73%) as an off white solid. ¹H-NMR (300 MHz, CD₃OD): δ ppm: 7.91 (d, J=8.1 Hz, 1H), 7.39 (d, J=1.5 Hz, 1H), 7.26 (dd, J=8.1, 1.5 Hz, 1H), 5.18-5.05 (m, 1H), 4.86-4.79 (m, 2H), 3.87 (d, J=7.2 Hz, 2H), 3.62 (d, J=7.5 Hz, 2H), 2.97 (s, 3H), 2.86 (s, 3H), 2.83 (s, 6H), 2.15 (s, 3H).

### Example 39

The following compound was prepared according to procedures AAAP, AAAQ, AAAR, AAAS, AAAT, AAAW and AAAV.

### (Z)-1-(4-amino-2-fluorobut-2-en-1-yl)-N-(tert-butyl)-4-(3-chloro-4-(N,N-dimethylsulfamoyl)benzyl)-3-methyl-1H-pyrazole-5-carboxamide hydrochloride (Compound 62)

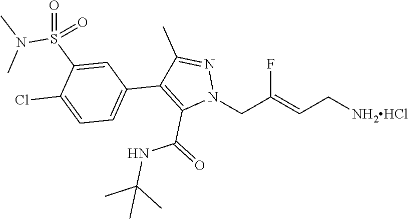

### Procedure AAAW: Preparation of tert-butyl (Z)-(4-(5-(tert-butylcarbamoyl)-4-(3-chloro-4-(N,N-dimethylsulfamoyl)benzyl)-3-methyl-1H-pyrazol-1-yl)-3-fluorobut-2-en-1-yl)carbamate

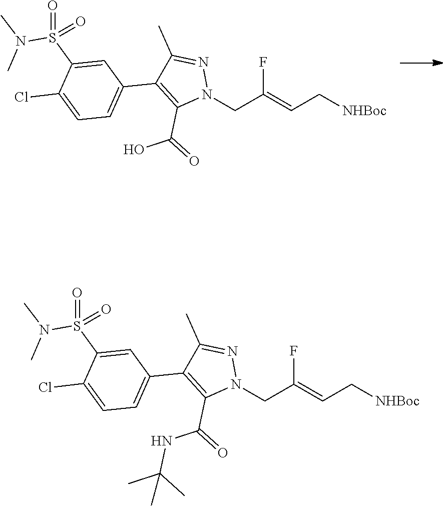 [SMILES: Cc1nn(C/C(F)=C/CNC(=O)OC(C)(C)C)c(C(=O)O)c1-c1ccc(Cl)c(S(=O)(=O)N(C)C)c1 ; Cc1nn(C/C(F)=C/CNC(=O)OC(C)(C)C)c(C(=O)NC(C)(C)C)c1-c1ccc(Cl)c(S(=O)(=O)N(C)C)c1]

To a stirring solution of (Z)-1-(4-((tert-butoxycarbonyl)amino)-2-fluorobut-2-en-1-yl)-4-(3-chloro-4-(N,N-dimethylsulfamoyl)benzyl)-3-methyl-1H-pyrazole-5-carboxylic acid (0.35 g, 0.64 mmol) in DMF (10 mL) at rt under N₂ was added sequentially triethylamine (0.19 g, 1.92 mmol), HATU (0.36 g, 0.96 mmol) and tert-butyl amine (0.070 g, 0.96 mmol). The resulting mixture was stirred at rt for 2 h. The reaction mixture was diluted with water (30 mL) and extracted with ethyl acetate (2×50 mL). The combined organics were washed with aq. HCl (1.0 M; 20 mL), water, sat. aq. NaHCO₃, brine, dried over anhydrous Na₂SO₄ and then concentrated in vacuo. The crude material was purified over silica gel, eluting with 80%-90% ethyl acetate in hexanes to afford the tert-butyl (Z)-(4-(5-(tert-butylcarbamoyl)-4-(3-chloro-4-(N,N-dimethylsulfamoyl)benzyl)-3-methyl-1H-pyrazol-1-yl)-3-fluorobut-2-en-1-yl)carbamate (0.26 g, 68%). ¹H-NMR (400 MHz, CDCl₃): δ ppm: 7.99 (d, J=7.6 Hz, 1H), 7.29 (d, J=1.6 Hz, 1H), 7.13 (dd, J=8.4, 1.6 Hz, 1H), 5.03-4.93 (m, 3H), 3.92 (s, 2H), 3.82 (bs, 2H), 2.86 (s, 6H), 2.16 (s, 3H), 1.42 (s, 9H), 1.27 (s, 9H).

### Example 40

The following compound was prepared according to procedures AAAP, AAAX, AAAY, AAAZ, AAAAA, AAAAB, AAAAC and AAAAD.

### (Z)-4-((4-(4-amino-2-fluorobut-2-en-1-yl)-5-(l-hydroxyethyl)-3-methyl-1H-pyrazol-1-yl)methyl)-2-chloro-N,N-dimethylbenzenesulfonamide hydrochloride (Compound 63)

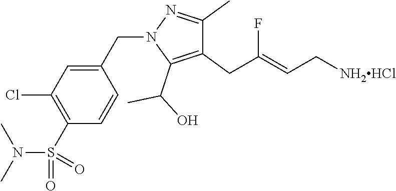

### Procedure AAAX: Preparation of ethyl (2Z,5Z)-3-acetyl-7-((tert-butoxycarbonyl)amino)-5-fluoro-2-(methoxyimino)hept-5-enoate

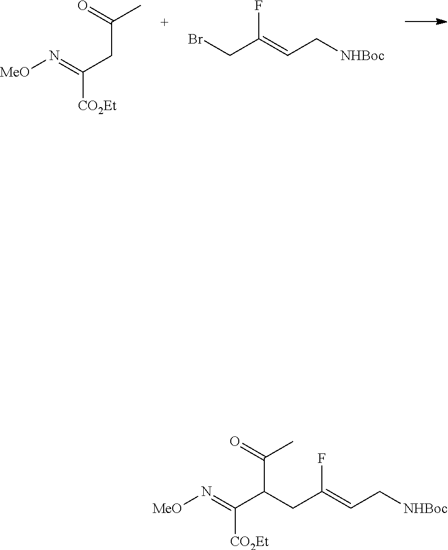 [SMILES: CCOC(=O)/C(CC(C)=O)=N\OC ; CC(C)(C)OC(=O)NC/C=C(\F)CBr ; CCOC(=O)/C(=N\OC)C(C/C(F)=C/CNC(=O)OC(C)(C)C)C(C)=O]

To a stirring solution of ethyl 2-(methoxyimino)-4-oxopentanoate (0.50 g, 2.67 mmol) in DMF (10 mL) at rt was added K₂CO₃ (0.92 g, 6.68 mmol) followed by (tert-butyl (Z)-(4-bromo-3-fluorobut-2-en-1-yl) carbamate (0.79 g, 2.94 mmol). The resulting mixture was stirred at rt for 3 h. The reaction mixture was diluted with water (25 mL) and extracted with ethyl acetate (3×25 mL). The combined organics were washed with brine, dried over anhydrous Na₂SO₄ and then concentrated in vacuo. The crude material was purified over silica gel, eluting with 0%-20% ethyl acetate in hexane to afford ethyl (2Z,5Z)-3-acetyl-7-((tert-butoxycarbonyl)amino)-5-fluoro-2-(methoxyimino)hept-5-enoate (350 mg, 35%).

### Procedure AAAY: Preparation of ethyl (Z)-4-(4-((tert-butoxycarbonyl)amino)-2-fluorobut-2-en-1-yl)-3-meth 1H-Pyrazole-5-carboxylate

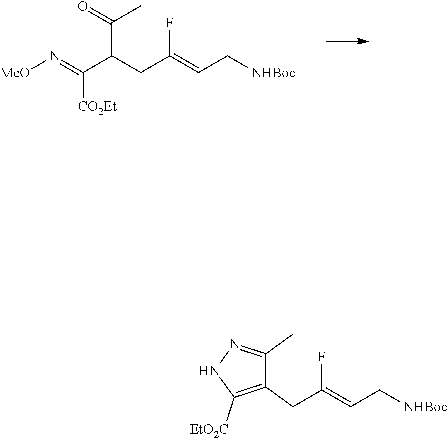 [SMILES: CCOC(=O)/C(=N\OC)C(C/C(F)=C/CNC(=O)OC(C)(C)C)C(C)=O ; CCOC(=O)c1[nH]nc(C)c1C/C(F)=C/CNC(=O)OC(C)(C)C]

To a stirring solution of ethyl ethyl (2Z,5Z)-3-acetyl-7-((tert-butoxycarbonyl)amino)-5-fluoro-2-(methoxyimino)hept-5-enoate (5.00 g, 13.4 mmol) in ethanol (100 mL) at rt was added hydrazine sulfate (2.08 g, 16.0 mmol) and water (40 mL). The resulting mixture was heated at reflux and stirring was continued for 48 h. The reaction mixture was diluted with water (60 mL) and extracted with ethyl acetate (2×60 mL). The combined organics were washed with brine, dried over anhydrous Na₂SO₄ and concentrated in vacuo. The crude material was purified over silica gel, eluting with 20%-30% ethyl acetate in hexanes to afford ethyl (Z)-4-(4-((tert-butoxycarbonyl)amino)-2-fluorobut-2-en-1-yl)-3-methyl-1H-pyrazole-5-carboxylate (1.38 g, 30%).

### Procedure AAAZ: Preparation of ethyl (Z)-4-(4-((tert-butoxycarbonyl)amino)-2-fluorobut-2-en-1-yl)-1-(3-chloro-4-(N,N-dimethylsulfamoyl)benzyl)-3-methyl-1H-pyrazole-5-carboxylate and ethyl (Z)-4-(4-((tert-butoxycarbonyl)amino)-2-fluorobut-2-en-1-yl)-1-(3-chloro-4-(N,N-dimethylsulfamoyl)benzyl)-5-methyl-1H-pyrazole-3-carboxylate

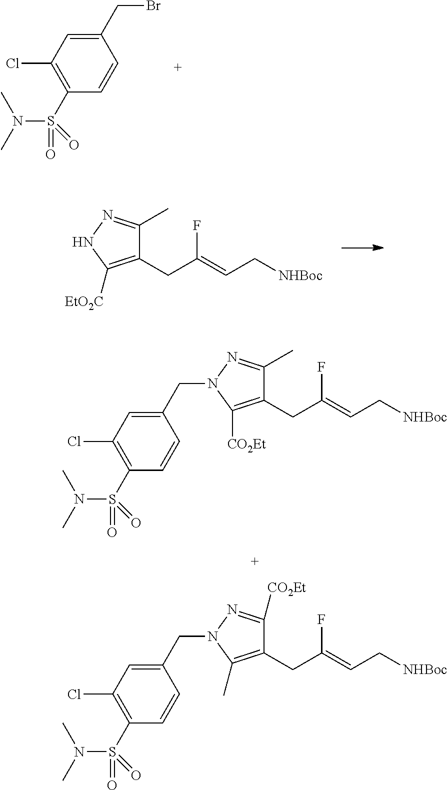 [SMILES: CN(C)S(=O)(=O)c1ccc(CBr)cc1Cl ; CCOC(=O)c1[nH]nc(C)c1C/C(F)=C/CNC(=O)OC(C)(C)C ; CCOC(=O)c1c(C/C(F)=C/CNC(=O)OC(C)(C)C)c(C)nn1Cc1ccc(S(=O)(=O)N(C)C)c(Cl)c1 ; CCOC(=O)c1nn(Cc2ccc(S(=O)(=O)N(C)C)c(Cl)c2)c(C)c1C/C(F)=C/CNC(=O)OC(C)(C)C]

To a stirring solution of ethyl (Z)-4-(4-((tert-butoxycarbonyl)amino)-2-fluorobut-2-en-1-yl)-3-methyl-1H-pyrazole-5-carboxylate (1.30 g, 38.1 mmol) in DMF (20 mL) at rt was added Cs₂CO₃ (3.10 g, 9.55 mmol) and 4-(bromomethyl)-2-chloro-N,N-dimethylbenzenesulfonamide (1.18 g, 38.1 mmol). The resulting suspension was heated at 60° C. and stirring was continued for 12 h. The reaction mixture was diluted with water (50 mL) and extracted with ethyl acetate (2×60 mL). The combined organics were washed with brine, dried over anhydrous Na₂SO₄ and then concentrated in vacuo. The crude material was purified over silica gel, eluting with 25% ethyl acetate in hexanes to afford ethyl (Z)-4-(4-((tert-butoxycarbonyl)amino)-2-fluorobut-2-en-1-yl)-1-(3-chloro-4-(N,N-dimethylsulfamoyl)benzyl)-3-methyl-1H-pyrazole-5-carboxylate (0.45 g, 21%) followed by ethyl (Z)-4-(4-((tert-butoxycarbonyl)amino)-2-fluorobut-2-en-1-yl)-1-(3-chloro-4-(N,N-dimethylsulfamoyl)benzyl)-5-methyl-1H-pyrazole-3-carboxylated (0.23 g, 11%).

### Ethyl (Z)-4-(4-((tert-butoxycarbonyl)amino)-2-fluorobut-2-en-1-yl)-1-(3-chloro-4-(N,N-dimethylsulfamoyl)benzyl)-3-methyl-1H-pyrazole-5-carboxylate

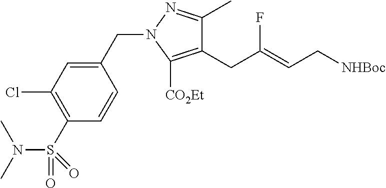 [SMILES: CCOC(=O)c1c(C/C(F)=C/CNC(=O)OC(C)(C)C)c(C)nn1Cc1ccc(S(=O)(=O)N(C)C)c(Cl)c1]

¹H-NMR (400 MHz, CDCl₃) δ 7.96 (d, J=8.0 Hz, 1H), 7.29 (d, J=1.6 Hz, 1H), 7.15 (dd, J=8.4, 2.0 Hz, 1H), 4.65-4.5 (m, 2H), 4.3 (q, J=7.6 Hz, 2H), 3.78-3.71 (br, 2H), 3.57 (d, J=12.4 Hz, 2H), 2.86 (s, 6H), 2.25 (s, 3H), 1.41 (s, 9H), 1.32 (t, J=6.8 Hz, 3H).

### Ethyl (Z)-4-(4-((tert-butoxycarbonyl)amino)-2-fluorobut-2-en-1-yl)-1-(3-chloro-4-(N,N-dimethylsulfamoyl)benzyl)-5-methyl-1H-pyrazole-3-carboxylate

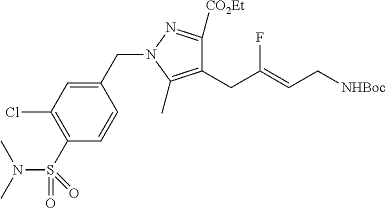 [SMILES: CCOC(=O)c1nn(Cc2ccc(S(=O)(=O)N(C)C)c(Cl)c2)c(C)c1C/C(F)=C/CNC(=O)OC(C)(C)C]

¹H-NMR (300 MHz, CDCl₃) δ ppm: 7.98 (d, J=8.1 Hz, 1H), 7.25 (s, 1H), 7.03 (d, J=8.1 Hz, 1H), 5.39 (s, 2H), 4.7-4.36 (m, 4H), 3.73-3.61 (m, 4H), 2.85 (s, 6H), 2.13 (s, 3H), 1.40 (s, 9H).

### Procedure AAAAA: Preparation of tert-butyl (Z)-(4-(1-(3-chloro-4-(N,N-dimethylsulfamoyl)benzyl)-5-(hydroxymethyl)-3-methyl-1H-pyrazol-4-yl)-3-fluorobut-2-en-1-ylcarbamate

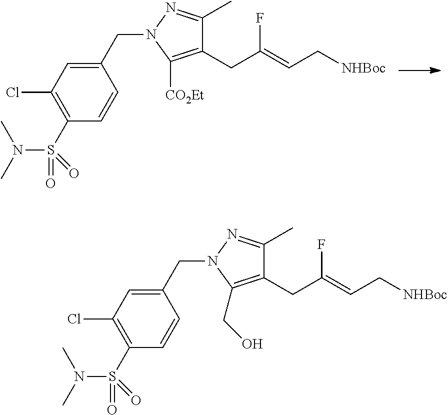 [SMILES: CCOC(=O)c1c(C/C(F)=C/CNC(=O)OC(C)(C)C)c(C)nn1Cc1ccc(S(=O)(=O)N(C)C)c(Cl)c1 ; Cc1nn(Cc2ccc(S(=O)(=O)N(C)C)c(Cl)c2)c(CO)c1C/C(F)=C/CNC(=O)OC(C)(C)C]

To a stirring solution of ethyl (Z)-4-(4-((tert-butoxycarbonyl)amino)-2-fluorobut-2-en-1-yl)-1-(3-chloro-4-(N,N-dimethylsulfamoyl)benzyl)-3-methyl-1H-pyrazole-5-carboxylate (0.35 g, 0.61 mmol) in THF (10 mL) at −78° C. under N₂ was added DIBAL-H (1.0 M in THF, 5.00 mL, 5.00 mmol). The reaction mixture was gradually raised to rt and stirring was continued for 48 h. The reaction mixture was quenched with sat. aq. NH₄Cl (30 mL) and filtered through a bed of Celite®. The filtrate was extracted with ethyl acetate (2×50 mL). The combined organics were washed with brine, dried over anhydrous Na₂SO₄ and concentrated under in vacuo to afford the crude material (0.3 g) which was used for the next step without any purification. ¹H-NMR (300 MHz, CDCl₃) δ 7.97 (d, J=8.0 Hz, 1H), 7.14-7.12 (m, 1H), 5.38 (s, 2H), 4.72-4.61 (m, 2H), 4.51 (s, 2H), 3.74-3.71 (m, 2H), 3.28 (d, J=11.6 Hz, 2H), 2.86 (s, 6H), 2.04 (s, 3H), 1.41 (s, 9H).

### Procedure AAAAB: Preparation of tert-butyl (Z)-(4-(1-(3-chloro-4-(N,N-dimethylsulfamoylbenzyl)-5-formyl-3-methyl-1H-pyrazol-4-yl)-3-fluorobut-2-en-1-yl)carbamate

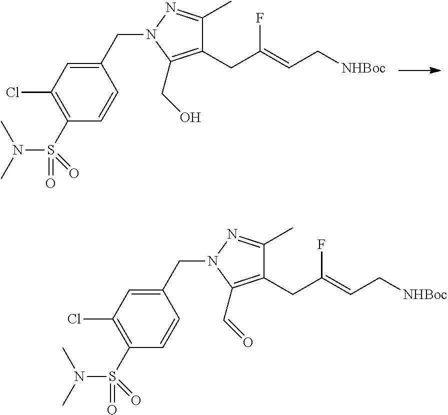 [SMILES: Cc1nn(Cc2ccc(S(=O)(=O)N(C)C)c(Cl)c2)c(CO)c1C/C(F)=C/CNC(=O)OC(C)(C)C ; Cc1nn(Cc2ccc(S(=O)(=O)N(C)C)c(Cl)c2)c(C=O)c1C/C(F)=C/CNC(=O)OC(C)(C)C]

To a stirring solution of tert-butyl (Z)-(4-(1-(3-chloro-4-(N,N-dimethylsulfamoyl)benzyl)-5-(hydroxymethyl)-3-methyl-1H-pyrazol-4-yl)-3-fluorobut-2-en-1-yl)carbamate (0.40 g, 0.75 mmol) in CH₂Cl₂ (20 mL) at rt was added MnO₂ (4.00 g, 46.0 mmol). The resulting mixture was stirred at rt for 12 h. The reaction mixture was filtered through a plug of Celite®. The filtrate was concentrated under reduced pressure to get the tert-butyl (Z)-(4-(1-(3-chloro-4-(N,N-dimethylsulfamoyl)benzyl)-5-formyl-3-methyl-1H-pyrazol-4-yl)-3-fluorobut-2-en-1-yl)carbamate (0.35 g, 88%). ¹H-NMR (300 MHz, CDCl₃) δ ppm: 9.85 (s, 1H), 7.97 (d, J=8.1 Hz, 1H), 7.35 (d, J=1.5 Hz, 1H), 7.23-7.2 (m, 1H), 5.65 (s, 2H), 4.79-4.51 (m, 2H), 3.8-3.7 (br, 2H), 3.55 (d, J=14.1 Hz, 2H), 2.86 (s, 6H), 2.27 (s, 3H), 1.42 (s, 9H).

### Procedure AAAAC: Preparation of tert-butyl (Z)-(4-(1-(3-chloro-4-(N,N-dimethylsulfamoyl)benzyl)-5-(1-hydroxyethyl)-3-methyl-1H-pyrazol-4-yl)-3-fluorobut-2-en-1-yl)carbamate

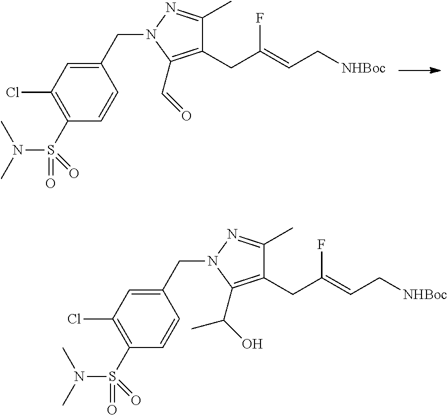 [SMILES: Cc1nn(Cc2ccc(S(=O)(=O)N(C)C)c(Cl)c2)c(C=O)c1C/C(F)=C/CNC(=O)OC(C)(C)C ; Cc1nn(Cc2ccc(S(=O)(=O)N(C)C)c(Cl)c2)c(C(C)O)c1C/C(F)=C/CNC(=O)OC(C)(C)C]

To a stirring solution of tert-butyl (Z)-(4-(1-(3-chloro-4-(N,N-dimethylsulfamoyl)benzyl)-5-formyl-3-methyl-1H-pyrazol-4-yl)-3-fluorobut-2-en-1-yl)carbamate (0.25 g, 0.47 mmol) at −78° C. was added methyl magnesium chloride (3.0 M in diethyl ether, 0.44 mL, 1.32 mmol). The resulting mixture was gradually warmed to rt and stirring was continued for 2 h. The reaction mixture was quenched with sat. aq. NH₄Cl (20 mL) and extracted with ethyl acetate (2×30 mL). The combined organics were washed with brine, dried over anhydrous Na₂SO₄ and then concentrated in vacuo. The crude material was purified over silica gel, eluting with 20%-70% ethyl acetate in hexanes to afford tert-butyl (Z)-(4-(l-(3-chloro-4-(N,N-dimethylsulfamoyl)benzyl)-5-(1-hydroxyethyl)-3-methyl-1H-pyrazol-4-yl)-3-fluorobut-2-en-1-yl)carbamate (0.11 g, 42%).

### Procedure AAAAD: Preparation of (Z)-4-((4-(4-amino-2-fluorobut-2-en-1-yl)-5-(1-hydroxyethyl)-3-methyl-1H-pyrazol-1-ylmethyl)-2-chloro-N,N-dimethylbenzenesulfonamide hydrochloride (Compound 63)

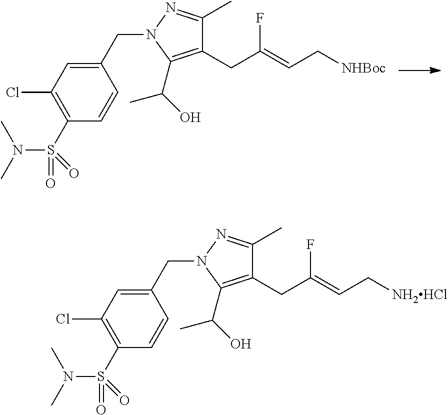

To a stirring solution of tert-butyl (Z)-(4-(1-(3-chloro-4-(N,N-dimethylsulfamoyl)benzyl)-5-(1-hydroxyethyl)-3-methyl-1H-pyrazol-4-yl)-3-fluorobut-2-en-1-yl)carbamate (0.28 g, 0.51 mmol) in 1,4-dioxane (10 mL) at 0° C. was added HCl (4.0 M 1,4-dioxane; 5.00 mL, 20.0 mmol). The resulting mixture was gradually raised to rt and stirring was continued for 1 h. The reaction mixture was concentrated under reduced pressure. The crude material was purified using preparative HPLC [Waters Xbridge C18 (150 mm×21.20 mm), A=0.05% HCl in water; B=MeCN; Gradient, 10-50% B, Flow: 15.0 mL/min]. The fractions were lyophilized to afford (Z)-4-((4-(4-amino-2-fluorobut-2-en-1-yl)-5-(1-hydroxyethyl)-3-methyl-1H-pyrazol-1-yl)methyl)-2-chloro-N,N-dimethylbenzenesulfonamide hydrochloride (130 mg, 53%). ¹H-NMR (300 MHz, CD₃OD) δ ppm: 7.98 (d, J=8.1 Hz, 1H), 7.43 (d, J=1.5 Hz, 1H), 7.22 (dd, J=8.1, 1.5 Hz, 1H), 5.66 (d, J=16.8 Hz, 1H), 5.57 (d, J=16.8 Hz, 1H), 5.15 (q, J=6.9 Hz, 1H), 4.82-4.77 (m, 1H), 3.70-3.53 (m, 4H), 2.84 (s, 6H), 2.26 (s, 3H), 1.41 (d, J=6.9 Hz, 3H).

### Example 41

### Preparation of (Z)-4-((4-(4-amino-2-fluorobut-2-en-1-yl)-3,5-diisopropyl-1H-pyrazol-1-yl)methyl)-2-chloro-N,N-dimethylbenzenesulfonamide hydrochloride (Compound 64)

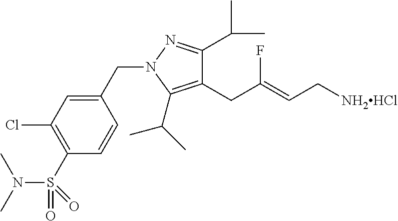

### Procedure AAAAE: Preparation of tert-butyl (Z)-(3-fluoro-5-isobutyryl-7-methyl-6-oxooct-2-en-1-yl)carbamate

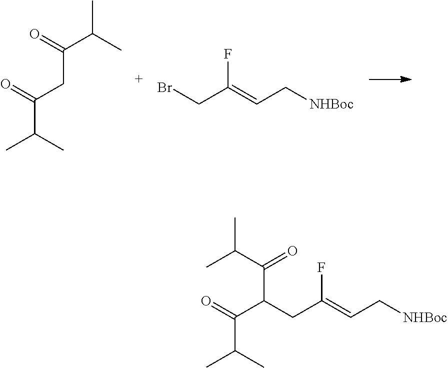 [SMILES: CC(C)C(=O)CC(=O)C(C)C ; CC(C)(C)OC(=O)NC/C=C(\F)CBr ; CC(C)C(=O)C(C/C(F)=C/CNC(=O)OC(C)(C)C)C(=O)C(C)C]

To a stirring solution of 2,6-dimethylheptane-3,5-dione (3.10 g, 20.1 mmol) in THF (10 mL) and DMF (10 mL) at 0° C. under N₂ was added NaHMDS (1.0 M in THF; 20.1 mL, 20.1 mmol). The resulting solution was stirred at 0° C. for 10 min and then tert-butyl (Z)-(4-bromo-3-fluorobut-2-en-1-yl) carbamate (1.80 g, 6.71 mmol) in THF (10 mL) was added. The resulting mixture was gradually warmed to rt and stirring was continued for 3 h. The reaction mixture was poured into ice cold water (100 mL) and extracted with ethyl acetate (2×100 mL). The combined organics were washed with brine, dried over anhydrous Na₂SO₄ and then concentrated in vacuo. The residue obtained (2.3 g) was progressed to the next step without purification.

### Procedure AAAAF: Preparation of tert-butyl (Z)-(4-(3,5-diisopropyl-1H-pyrazol-4-yl)-3-fluorobut-2-en-1-yl)carbamate

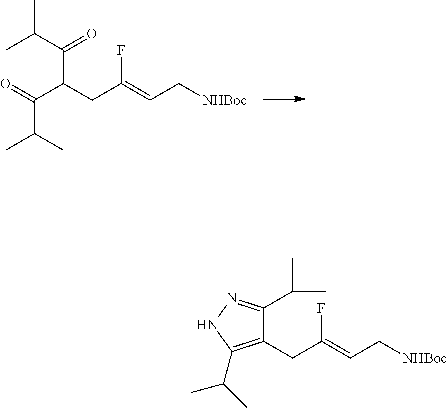 [SMILES: CC(C)C(=O)C(C/C(F)=C/CNC(=O)OC(C)(C)C)C(=O)C(C)C ; CC(C)c1n[nH]c(C(C)C)c1C/C(F)=C/CNC(=O)OC(C)(C)C]

To a solution of tert-butyl (Z)-(3-fluoro-5-isobutyryl-7-methyl-6-oxooct-2-en-1-yl) carbamate (2.5 g, 3.86 mmol) in ethanol (50 mL) at rt was added hydrazine hydrate (5.00 mL). The resulting mixture was heated to reflux for 12 h. The reaction mixture was cooled to rt, diluted with water (100 mL) and extracted with ethyl acetate (2×100 mL). The combined organics were washed with brine, dried over anhydrous Na₂SO₄ and concentrated in vacuo to afford tert-butyl (Z)-(4-(3,5-diisopropyl-1H-pyrazol-4-yl)-3-fluorobut-2-en-1-yl)carbamate (0.92 g, 37%). ¹H-NMR (300 MHz, CDCl₃) δ ppm: 4.62-4.50 (m, 1H), 3.74 (bs, 2H), 3.28 (d, J=9.9 Hz, 2H), 2.99-2.90 (m, 2H), 1.41 (s, 9H), 1.26 (d, J=6.9 Hz, 12H).

### Procedure AAAAG: Preparation of tert-butyl (Z)-(4-(1-(3-chloro-4-(N,N-dimethylsulfamoylbenzyl-3,5-diisopropyl-1H-pyrazol-4-yl)-3-fluorobut-2-en-1-yl)carbamate

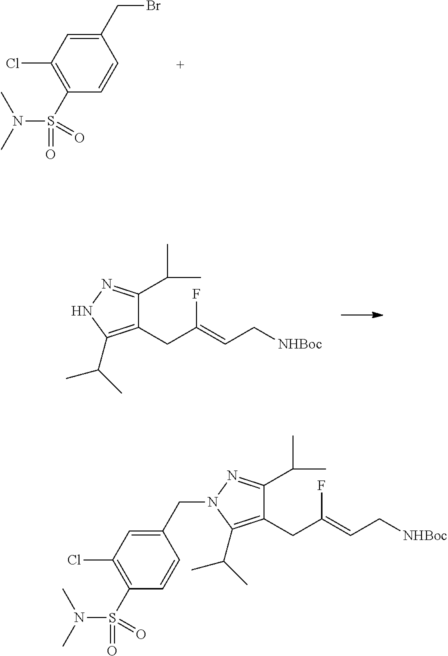 [SMILES: CN(C)S(=O)(=O)c1ccc(CBr)cc1Cl ; CC(C)c1n[nH]c(C(C)C)c1C/C(F)=C/CNC(=O)OC(C)(C)C ; CC(C)c1nn(Cc2ccc(S(=O)(=O)N(C)C)c(Cl)c2)c(C(C)C)c1C/C(F)=C/CNC(=O)OC(C)(C)C]

To a solution of tert-butyl (Z)-(4-(3,5-diisopropyl-1H-pyrazol-4-yl)-3-fluorobut-2-en-1-yl)carbamate (0.7 g, 2.06 mmol) in DMSO (10 mL) at rt was added KOH (0.23 g, 4.12 mmol). The resulting mixture was stirred for 10 min at rt and then 4-(bromomethyl)-2-chloro-N,N-dimethylbenzenesulfonamide (0.66 g, 2.47 mmol) was added portion-wise. The reaction mixture was stirred at rt for 2 h. The reaction mixture was diluted with water (50 mL) and extracted with ethyl acetate (2×50 mL). The combined organics were washed with brine, dried over anhydrous Na₂SO₄ and concentrated in vacuo. The crude material was purified over silica gel, eluting with 25%-30% ethyl acetate in hexane to afford tert-butyl (Z)-(4-(1-(3-chloro-4-(N,N-dimethylsulfamoyl)benzyl)-3,5-diisopropyl-1H-pyrazol-4-yl)-3-fluorobut-2-en-1-yl)carbamate (0.62 g, 53%).

### Procedure AAAAH: Preparation of (Z)-4-((4-(4-amino-2-fluorobut-2-en-1-yl)-3,5-diisopropyl-1H-pyrazol-1-yl)methyl)-2-chloro-N,N-dimethylbenzenesulfonamide hydrochloride (Compound 64)

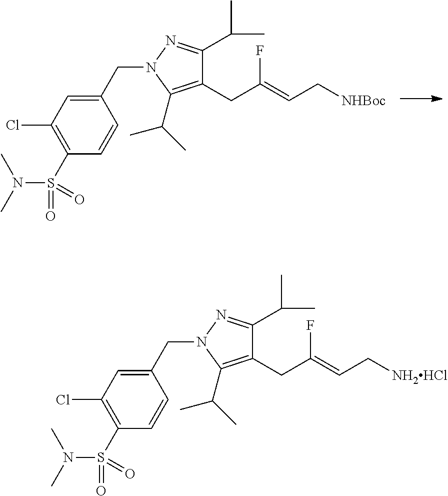

To a stirring solution of tert-butyl (Z)-(4-(1-(3-chloro-4-(N,N-dimethylsulfamoyl)benzyl)-3,5-diisopropyl-1H-pyrazol-4-yl)-3-fluorobut-2-en-1-yl)-carbamate (0.30 g, 0.53 mmol) in 1,4-dioxane (10 mL) at 0° C. was added HCl (4.0 M in 1,4-dioxane; 2.00 mL, 8.00 mmol). The resulting mixture was gradually raised to rt and stirring was continued for 1 h. The reaction mixture was concentrated under reduced pressure. The crude material was purified using preparative HPLC [Waters Xbridge C18 (150 mm×21.20 mm), A=0.05% HCl in water; B=MeCN; Gradient, 10-50% B, Flow: 15.0 mL/min] and fractions were lyophilized to afford (Z)-4-((4-(4-amino-2-fluorobut-2-en-1-yl)-3,5-diisopropyl-1H-pyrazol-1-yl)methyl)-2-chloro-N,N-dimethylbenzenesulfonamide hydrochloride (134 mg, 54%). ¹H-NMR (300 MHz, DMSO-d₆) δ ppm: 7.97 (brs, 3H), 7.92 (d, J=8.4 Hz, 1H), 7.33 (s, 1H), 7.07 (d, J=8.1 Hz, 1H), 5.44 (s, 2H), 4.76-4.59 (m, 1H), 3.45 (d, J=8.7 Hz, 4H), 3.09-3.02 (m, 1H), 2.93-2.86 (m, 1H), 2.78 (s, 6H), 1.19 (d, J=6.9 Hz, 6H), 1.11 (d, J=7.2 Hz, 6H).

### Example 42

### Preparation of (Z)-4-((1-(4-amino-2-fluorobut-2-en-1-yl)-5-isopropyl-3-methyl-1H-pyrazol-4-yl)methyl)-2-chloro-N,N-dimethylbenzenesulfonamide hydrochloride (Compound 65) and (Z)-4-((1-(4-amino-2-fluorobut-2-en-1-yl)-3-isopropyl-5-methyl-1H-pyrazol-4-yl)methyl)-2-chloro-N,N-dimethylbenzenesulfonamide hydrochloride (Compound 66)

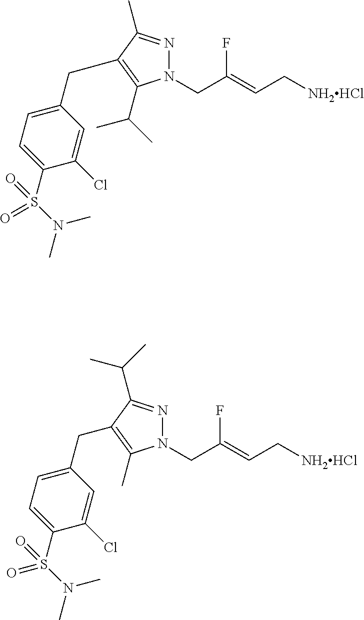

### Procedure AAAAI: Preparation of 4-(2-acetyl-4-methyl-3-oxopentyl)-2-chloro-N,N-dimethylbenzenesulfonamide

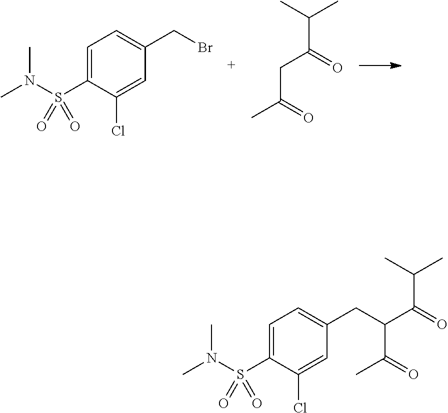

To a stirring solution of 5-methylhexane-2, 4-dione (1.99 mL, 14.39 mmol) in ethanol (15.0 mL) at rt was added sodium ethoxide (21 w % in ethanol; 1.71 mL, 5.27 mmol). After stirring for 10 min, potassium iodide (797 mg, 4.79 mmol) was added followed by 4-(bromomethyl)-2-chloro-N,N-dimethylbenzenesulfonamide (1.5 g, 4.79 mmol). The resulting mixture was stirred at rt for 10 h. The reaction mixture was concentrated under reduced pressure. The residue was diluted with ethyl acetate (200 mL) and washed with water (50 mL), brine (50 mL) dried over Na₂SO₄ and then concentrated in vacuo. The crude material was purified over silica gel, eluting with 15% ethyl acetate in hexane to afford 4-(2-acetyl-4-methyl-3-oxopentyl)-2-chloro-N,N-dimethylbenzenesulfonamide (1.34 g, 78%) as a colorless gum. ¹H-NMR (400 MHz, DMSO-d₆) δ ppm: 7.82 (d, J=8.0 Hz, 1H), 7.58 (d, J=1.6 Hz, 1H), 7.36-7.38 (m, 1H), 4.58 (t, J=8.0 Hz, 1H), 3.12-3.00 (m, 2H), 2.76 (s, 6H), 2.76-2.65 (m, 1H), 2.16 (s, 3H), 0.97 (d, J=6.4 Hz, 3H), 0.79 (d, J=6.4 Hz, 3H).

### Procedure AAAAJ: Preparation of 2-chloro-4-((5-isopropyl-3-methyl-1H-pyrazol-4-yl)methyl)-N,N-dimethylbenzenesulfonamide

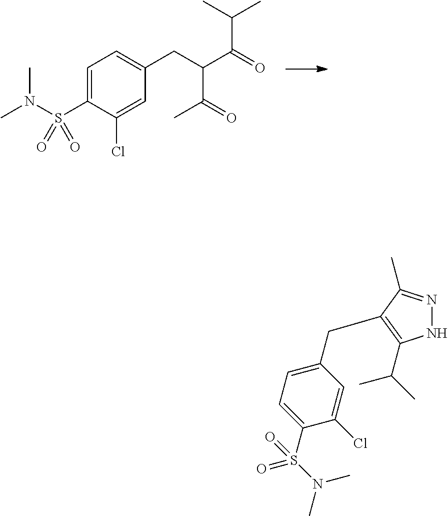 [SMILES: CC(=O)C(Cc1ccc(S(=O)(=O)N(C)C)c(Cl)c1)C(=O)C(C)C ; Cc1n[nH]c(C(C)C)c1Cc1ccc(S(=O)(=O)N(C)C)c(Cl)c1]

To a stirring solution of 4-(2-acetyl-4-methyl-3-oxopentyl)-2-chloro-N,N-dimethyl benzene sulfonamide (1.34 g, 3.73 mmol) in ethanol (15.0 mL) at rt was added hydrazine hydrate (0.36 mL, 7.46 mmol). The resulting mixture was heated at reflux and for 2 h. The reaction mixture was concentrated under reduced pressure. The residue was diluted with ethyl acetate (200 mL) and washed with water (50 mL), brine (50 mL) dried over Na₂SO₄ and then concentrated in vacuo. The crude material was purified over silica gel, eluting with 50% ethyl acetate in hexanes to afford 2-chloro-4-((5-isopropyl-3-methyl-1H-pyrazol-4-yl)methyl)-N,N-dimethylbenzenesulfonamide (1.10 g, 83%) as an off white gum. ¹H-NMR (400 MHz, DMSO-d₆) δ ppm: 12.10 (bs, 1H), 7.83 (d, J=8.0 Hz, 1H), 7.37 (d, J=1.2 Hz, 1H), 7.23 (dd, J=7.6, 1.2 Hz, 1H), 4.04 (s, 2H), 2.85 (bs, 1H), 2.76 (s, 6H), 2.03 (s, 3H), 1.08 (d, J=6.8, 6H).

### Procedure AAAAK: Preparation of tert-butyl (Z)-(4-(4-(3-chloro-4-(N,N-dimethylsulfamoyl)benzyl)-5-isopropyl-3-methyl-1H-pyrazol-1-yl)-3-fluorobut-2-en-1-yl)carbamate

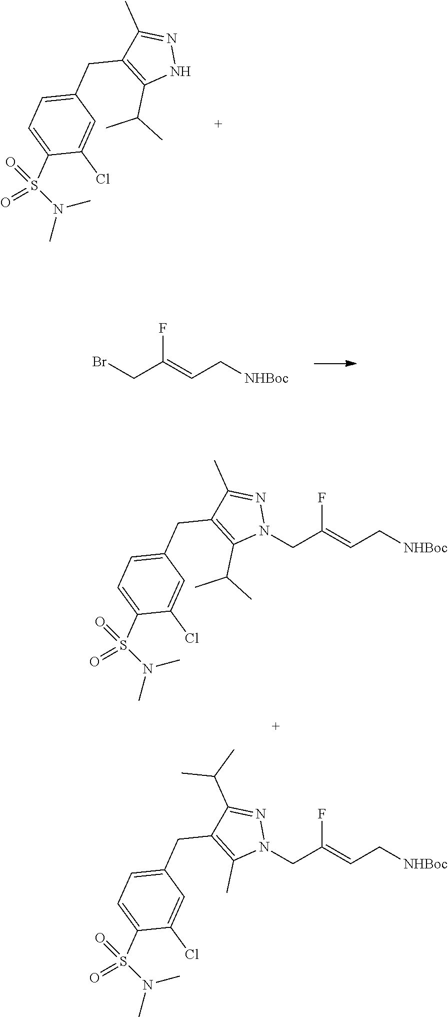 [SMILES: Cc1n[nH]c(C(C)C)c1Cc1ccc(S(=O)(=O)N(C)C)c(Cl)c1 ; CC(C)(C)OC(=O)NC/C=C(\F)CBr ; Cc1nn(C/C(F)=C/CNC(=O)OC(C)(C)C)c(C(C)C)c1Cc1ccc(S(=O)(=O)N(C)C)c(Cl)c1 ; Cc1c(Cc2ccc(S(=O)(=O)N(C)C)c(Cl)c2)c(C(C)C)nn1C/C(F)=C/CNC(=O)OC(C)(C)C]

To a stirring suspension of sodium hydride (60 w % in paraffin oil; 248 mg, 6.18 mmol) in THF (40 mL) at 0° C., was added a solution of 2-chloro-4-((5-isopropyl-3-methyl-1H-pyrazol-4-yl) methyl)-N,N-dimethylbenzenesulfonamide (1.10 g, 3.09 mmol) in THF (10 mL). After stirring for 10 min, tert-butyl (Z)-(4-bromo-3-fluorobut-2-en-1-yl) carbamate (1.65 g, 6.18 mmol) was added. The resulting mixture was stirred at rt for 3 h. The reaction mixture was quenched with ice cold water (20 mL) and the product was extracted with ethyl acetate (2×100 mL). The combined organics were washed with water (2×50 mL), brine (100 mL), dried over Na₂SO₄ and then concentrated in vacuo. The crude material was purified over silica gel, eluting with 15% ethyl acetate in hexanes to afford a mixture of isomers. The mixture was further purified by preparative HPLC [Phenomenex Luna C-18 (250 mm×19 mm), A=10 mM ammonium acetate in water, B=MeCN; Gradient, 0/25, 10/78, flow: 15.0 mL/min]. The fractions were lyophilized to afford tert-butyl (Z)-(4-(4-(3-chloro-4-(N,N-dimethylsulfamoyl)benzyl)-5-isopropyl-3-methyl-1H-pyrazol-1-yl)-3-fluorobut-2-en-1-yl)carbamate (220 mg, 13%) followed by tert-butyl (Z)-(4-(4-(3-chloro-4-(N,N-dimethylsulfamoyl)benzyl)-3-isopropyl-5-methyl-1H-pyrazol-1-yl)-3-fluorobut-2-en-1-yl)carbamate (380 mg, 24%).

Tert-butyl (Z)-(4-(4-(3-chloro-4-(N,N-dimethylsulfamoyl)benzyl)-5-isopropyl-3-methyl-1H-pyrazol-1-yl)-3-fluorobut-2-en-1-yl)carbamate.

¹H-NMR (400 MHz, DMSO-d₆): δ ppm: 7.48 (d, J=8.4 Hz, 1H), 7.37 (s, 1H), 7.17 (d, J=8.0 Hz, 1H), 7.02 (br s, 1H), 4.82 (d, J=12.0 Hz, 2H), 4.81 (dt, J=34.6, 7.2 Hz, 1H), 3.91 (s, 2H), 3.57 (br, 2H), 3.14-3.12 (m, 1H), 2.77 (s, 6H), 1.91 (s, 3H), 1.33 (s, 9H), 1.11 (d, J=6.8, 3H).

### Procedure AAAAL: Preparation of (Z)-4-((1-(4-amino-2-fluorobut-2-en-1-yl)-5-isopropyl-3-methyl-1H-pyrazol-4-yl)methyl)-2-chloro-N,N-dimethylbenzenesulfonamide hydrochloride (Compound 65)

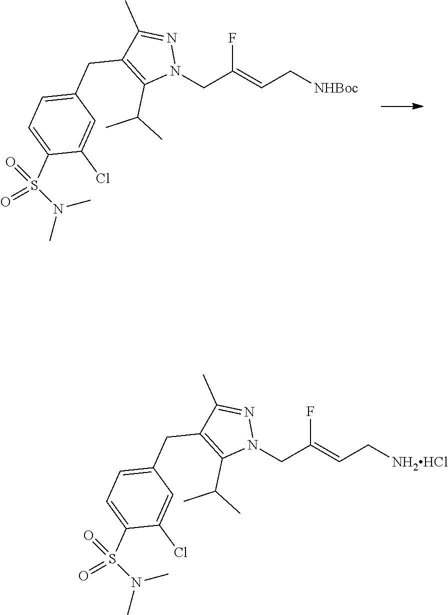

To a stirring solution of tert-butyl (Z)-(4-(4-(3-chloro-4-(N,N-dimethylsulfamoyl)benzyl)-5-isopropyl-3-methyl-1H-pyrazol-1-yl)-3-fluorobut-2-en-1-yl)carbamate (200 mg, 0.36 mmol) in methanol (0.2 mL) at rt was added HCl (2.0 M in diethyl ether; 3.00 mL, 6.00 mmol). The resulting mixture was stirred at rt for 1 h. The reaction mixture was concentrated in vacuo. The residue was triturated with diethyl ether (20 mL) and the resulting solid was lyophilized to afford (Z)-4-((1-(4-amino-2-fluorobut-2-en-1-yl)-5-isopropyl-3-methyl-1H-pyrazol-4-yl)methyl)-2-chloro-N,N-dimethylbenzenesulfonamide hydrochloride (118 mg, 67%) as an off white solid. ¹H-NMR (400 MHz, DMSO-d₆): δ ppm: 8.18 (br s, 3H), 7.85 (d, J=8.4 Hz, 1H), 7.38 (d, J=1.2 Hz, 1H), 7.19 (dd, J=8.0, 1.2 Hz, 1H), 4.98 (dt, J=35.1, 7.4 Hz, 1H), 4.92 (d, J=12.8 Hz, 2H), 3.93 (s, 2H), 3.49 (t, J=5.2 Hz, 2H), 3.17-3.14 (m, 1H), 2.77 (s, 6H), 1.93 (s, 3H), 1.13 (d, J=7.6 Hz, 6H).

### Procedure AAAAM: Preparation of (Z)-4-((1-(4-amino-2-fluorobut-2-en-1-yl)-3-isopropyl-5-methyl-1H-pyrazol-4-yl)methyl)-2-chloro-N,N-dimethylbenzenesulfonamide hydrochloride (Compound 66)

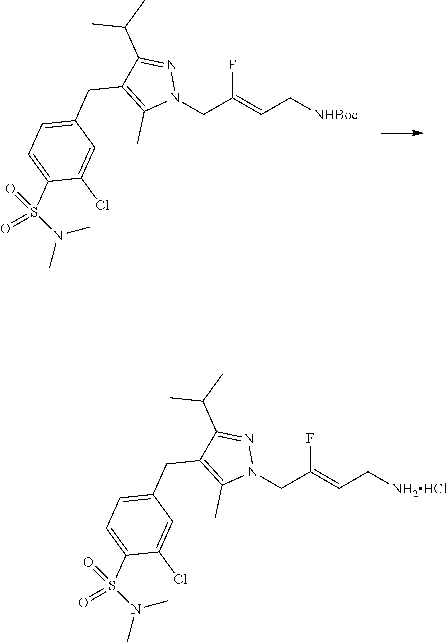

To a stirring solution of tert-butyl (Z)-(4-(4-(3-chloro-4-(N,N-dimethylsulfamoyl)benzyl)-3-isopropyl-5-methyl-1H-pyrazol-1-yl)-3-fluorobut-2-en-1-yl)carbamate (360 mg, 0.66 mmol) in methanol (0.4 mL) at rt was added HCl (2.0 M in diethyl ether, 6.00 mL, 12.0 mmol). The resulting mixture was stirred at rt for 1 h. The reaction mixture was concentrated under reduced pressure. The residue was triturated with diethyl ether (20 mL) and the resulting solid was lyophilized to afford (Z)-4-((1-(4-amino-2-fluorobut-2-en-1-yl)-3-isopropyl-5-methyl-1H-pyrazol-4-yl)methyl)-2-chloro-N,N-dimethylbenzenesulfonamide hydrochloride (220 mg, 70%) as an off white solid. ¹H-NMR (300 MHz, CD₃OD) δ ppm: 7.97 (d, J=8.1 Hz, 1H), 7.44 (s, 1H), 7.33-7.24 (m, 1H), 5.42 (d, J=12.2 Hz, 2H), 5.26 (dt, J=34.6, 7.2 Hz, 1H), 4.05 (s, 2H), 3.71 (d, J=7.3 Hz, 2H), 3.16 (t, J=7.1 Hz, 1H), 2.86 (s, 6H), 2.41 (d, J=2.3 Hz, 3H), 1.27 (dd, J=7.0, 1.2 Hz, 6H).

### Example 43

The following compounds were prepared according to procedures M, N, O, AA, AC, AD, AE, AJ, AL, AM, AAAAN and AF. The absolute stereochemistry of the separated enantiomers was not, unambiguously, ascertained. Stereochemistry has been assigned arbitrarily.

### (Z)-4-((1-(4-amino-2-fluorobut-2-en-1-yl)-5-(l-hydroxyethyl)-3-methyl-1H-pyrazol-4-yl)methyl)-2-chloro-N,N-dimethylbenzenesulfonamide hydrochloride; Enantiomer 1 (Compound 52)

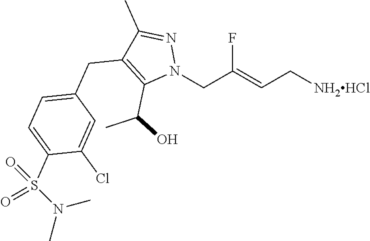

### (Z)-4-((1-(4-amino-2-fluorobut-2-en-1-yl)-5-(1-hydroxyethyl)-3-methyl-1H-pyrazol-4-yl)methyl)-2-chloro-N,N-dimethylbenzenesulfonamide hydrochloride; Enantiomer 2 (Compound 53)

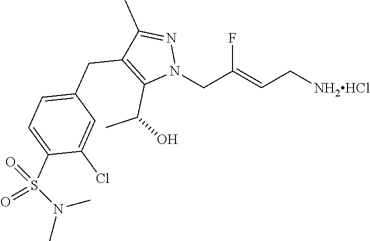

### Procedure AAAAN: Chiral separation of rac-tert-butyl (Z)-(4-(4-(3-chloro-4-(N,N-dimethylsulfamoyl)benzyl)-5-(1-hydroxyethyl)-3-methyl-1H-pyrazol-1-yl)-3-fluorobut-2-en-1-yl)carbamate

Tert-butyl (Z)-(4-(4-(3-chloro-4-(N,N-dimethylsulfamoyl)benzyl)-5-(I-hydroxyethyl)-3-methyl-1H-pyrazol-1-yl)-3-fluorobut-2-en-1-yl)carbamate; Enantiomer 1 (62.0 mg) and tert-butyl (Z)-(4-(4-(3-chloro-4-(N,N-dimethylsulfamoyl)benzyl)-5-(1-hydroxyethyl)-3-methyl-1H-pyrazol-1-yl)-3-fluorobut-2-en-1-yl)carbamate; Enantiomer 2 (70.0 mg) were both obtained from rac tert-butyl (Z)-(4-(4-(3-chloro-4-(N,N-dimethylsulfamoyl)benzyl)-5-(1-hydroxyethyl)-3-methyl-1H-pyrazol-1-yl)-3-fluorobut-2-en-1-yl)carbamate (250 mg) via chiral HPLC separation (Chiral Pak IA-3, 0.46 cm I.D.×25 cm length, eluting isocratically with 70% ethyl acetate in CH₂Cl₂ (containing 0.1% diethylamine), flow rate 0.5 mL/min), wherein tert-butyl (Z)-(4-(4-(3-chloro-4-(N,N-dimethylsulfamoyl)benzyl)-5-(1-hydroxyethyl)-3-methyl-1H-pyrazol-1-yl)-3-fluorobut-2-en-1-yl)carbamate; Enantiomer 1 was the first to elute and tert-butyl (Z)-(4-(4-(3-chloro-4-(N,N-dimethylsulfamoyl)benzyl)-5-(1-hydroxyethyl)-3-methyl-1H-pyrazol-1-yl)-3-fluorobut-2-en-1-yl)carbamate; Enantiomer 2 was the second to elute.

### Tert-butyl (Z)-(4-(4-(3-chloro-4-(N,N-dimethylsulfamoyl)benzyl)-5-(1-hydroxyethyl)-3-methyl-1H-pyrazol-1-yl)-3-fluorobut-2-en-1-yl)carbamate; Enantiomer 1

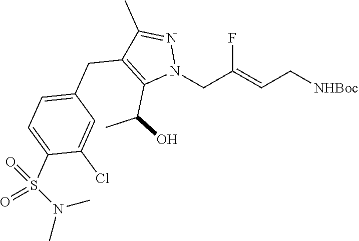 [SMILES: Cc1nn(C/C(F)=C/CNC(=O)OC(C)(C)C)c([C@H](C)O)c1Cc1ccc(S(=O)(=O)N(C)C)c(Cl)c1]

¹H-NMR (300 MHz, CDCl₃) δ ppm: 7.93 (d, J=8.1 Hz, 1H), 7.27-7.24 (m, 1H), 7.11 (ddt, J=8.2, 1.6, 0.8 Hz, 1H), 5.04 (q, J=6.7 Hz, 1H), 4.89 (d, J=9.9 Hz, 2H), 4.73 (dt, J=36.5, 7.2 Hz, 1H), 3.91 (d, J=2.4 Hz, 2H), 3.80 (t, J=6.4 Hz, 2H), 2.88 (s, 6H), 2.07 (s, 3H), 1.43 (s, 9H), 1.41 (d, J=6.3 Hz, 3H). Chiral HPLC analysis: R, =6.74 min (Chiral Pak IA-3, 0.46 cm I.D.×25 cm length, eluting isocratically with 70% ethyl acetate in CH₂Cl₂ (containing 0.1% diethylamine) over 8 mins, flow rate 0.5 mL/min).

### Tert-butyl (Z)-(4-(4-(3-chloro-4-(N,N-dimethylsulfamoyl)benzyl)-5-(1-hydroxyethyl)-3-methyl-1H-pyrazol-1-yl)-3-fluorobut-2-en-1-yl)carbamate; Enantiomer 2

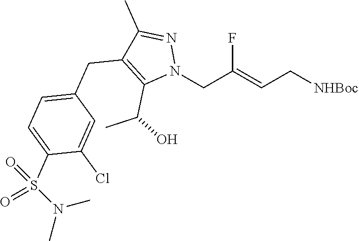 [SMILES: Cc1nn(C/C(F)=C/CNC(=O)OC(C)(C)C)c([C@@H](C)O)c1Cc1ccc(S(=O)(=O)N(C)C)c(Cl)c1]

¹H-NMR (300 MHz, CDCl₃) δ ppm: 7.93 (d, J=8.1 Hz, 1H), 7.27-7.24 (m, 1H), 7.11 (ddt, J=8.2, 1.6, 0.8 Hz, 1H), 5.04 (q, J=6.7 Hz, 1H), 4.89 (d, J=9.9 Hz, 2H), 4.73 (dd, J=36.5, 7.2 Hz, 1H), 3.91 (d, J=2.4 Hz, 2H), 3.80 (t, J=6.4 Hz, 2H), 2.88 (s, 6H), 2.07 (s, 3H), 1.43 (s, 9H), 1.41 (d, J=6.3 Hz, 3H). Chiral HPLC analysis: R, =7.41 min (Chiral Pak IA-3, 0.46 cm I.D.×25 cm length, eluting isocratically with 70% ethyl acetate in CH₂Cl₂ (containing 0.1% diethylamine) over 8 mins, flow rate 0.5 mL/min).

### (Z)-4-((1-(4-amino-2-fluorobut-2-en-1-yl)-5-(1-hydroxyethyl)-3-methyl-1H-pyrazol-4-yl)methyl)-2-chloro-N,N-dimethylbenzenesulfonamide hydrochloride; Enantiomer 1 (Compound 52)

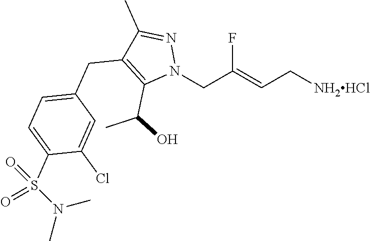

¹H-NMR (300 MHz, CD₃OD) δ ppm: 7.94 (d, J=8.2 Hz, 1H), 7.41 (d, J=1.6 Hz, 1H), 7.29 (d, J=8.2 Hz, 1H), 5.32-5.00 (m, 4H), 4.01 (s, 2H), 3.67 (d, J=7.5 Hz, 2H), 2.86 (s, 6H), 2.14 (s, 3H), 1.41 (d, J=6.7 Hz, 3H).

### (Z)-4-((1-(4-amino-2-fluorobut-2-en-1-yl)-5-(1-hydroxyethyl)-3-methyl-1H-pyrazol-4-yl)methyl)-2-chloro-N,N-dimethylbenzenesulfonamide hydrochloride; Enantiomer 2 (Compound 53)

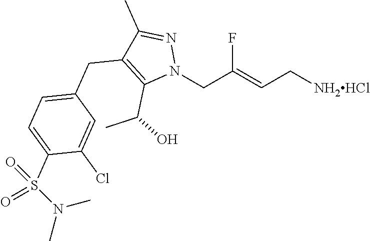

¹H-NMR (300 MHz, DMSO-d₆) δ ppm: 8.17 (s, 3H), 7.84 (d, J=8.1 Hz, 1H), 7.45 (d, J=1.4 Hz, 1H), 7.28 (d, J=8.3 Hz, 1H), 6.08 (s, 1H), 5.13-4.89 (m, 4H), 3.91 (s, 2H), 3.49 (s, 2H), 2.77 (s, 6H), 1.95 (s, 3H), 1.26 (d, J=6.5 Hz, 3H).

### Example 44

### Preparation of (Z)-3-fluoro-4-(4-(3-(methylsulfonyl)phenyl)-1H-pyrazol-1-yl)but-2-en-1-amine hydrochloride (Compound 67)

### Procedure AAAAO: Preparation of 4-(3-methylsulfonyl)phenyl)-1H-pyrazole

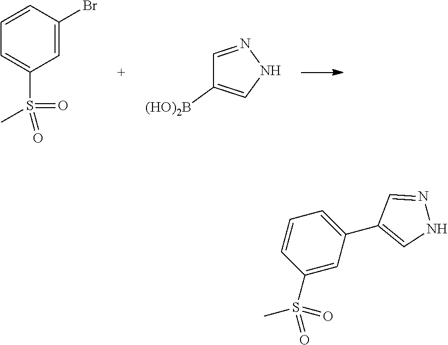

To a microwave vial equipped with a stirrer bar and charged with 1-bromo-3-(methylsulfonyl)benzene (0.60 g, 2.55 mmol) in DMF (9.0 mL) was added (1H-pyrazol-4-yl)boronic acid (0.34 g, 3.06 mmol). The reaction mixture was then purged with N₂ for 10 min. To this was added K₂CO₃ (1.05 g, 7.65 mmol) in water (3.0 mL) followed by PdCl₂(dppf).CH₂Cl₂ (0.21 g, 0.25 mmol). The resulting mixture was heated at 120° C. in the microwave, and stirring was continued for 1.5 h. The reaction mixture was cooled to rt and poured into ice water. The aqueous mixture was extracted with ethyl acetate (2×100 mL). The combined organic layers were washed with brine (30 mL), dried over Na₂SO₄, and then concentrated in vacuo. The crude material was purified over silica gel, eluting with 50% ethyl acetate in hexanes to afford 4-(3-(methylsulfonyl)phenyl)-1H-pyrazole 3 (0.24 g, 42%) as a brown solid. LCMS (M+1): m/z=223.4.

### Procedure AAAAP: Preparation of tert-butyl (Z)-(3-fluoro-4-(4-(3-(methylsulfonyl)phenyl)-1H-pyrazol-1-yl)but-2-en-1-yl)carbamate

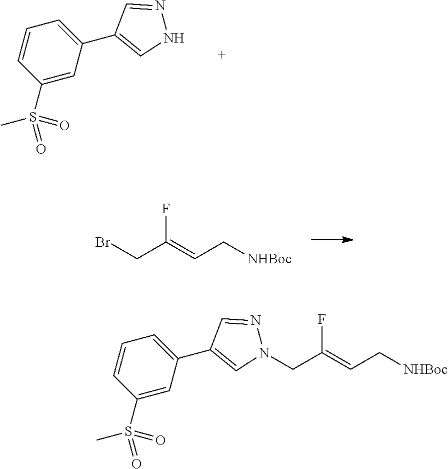 [SMILES: CS(=O)(=O)c1cccc(-c2cn[nH]c2)c1 ; CC(C)(C)OC(=O)NC/C=C(\F)CBr ; CC(C)(C)OC(=O)NC/C=C(\F)Cn1cc(-c2cccc(S(C)(=O)=O)c2)cn1]

To a stirring solution of 4-(3-(methylsulfonyl)phenyl)-1H-pyrazole 3 (0.310 g, 1.39 mmol) in DMF (10 mL) at 0° C. was added, sequentially, cesium carbonate (1.36 g, 4.18 mmol) and tert-butyl (Z)-(4-bromo-3-fluorobut-2-en-1-yl)carbamate 4 (0.450 g, 1.67 mmol). Then resulting mixture was warmed to rt and stirring was continued for 3 h. The reaction mixture was dilute with cold water, and the product was extracted with ethyl acetate (2×150 mL). The combined organics were washed with brine solution (2×20 mL), dried over Na₂SO₄, and then concentrated in vacuo. The crude material was purified over silica gel, eluting with 40% ethyl acetate in hexanes to afford tert-butyl (Z)-(3-fluoro-4-(4-(3-(methylsulfonyl)phenyl)-1H-pyrazol-1-yl)but-2-en-1-yl)carbamate 5 (0.32 g, 56%) as a brown gum. LCMS (M+1-C₄H1): m/z=354.1

### Procedure AAAAO: Preparation of (Z)-3-ffluoro-4-(4-(3-(methylsulfonyl)phenyl)-1H-pyrazol-1-yl)but-2-en-1-amine hydrochloride (Compound 67)

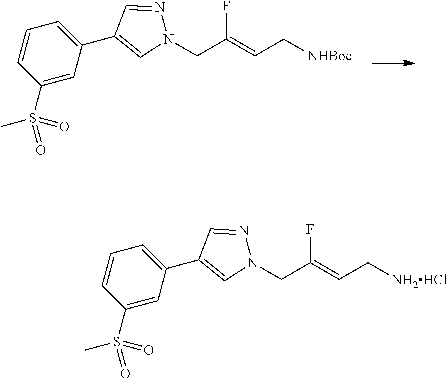

To a stirring solution of tert-butyl (Z)-(3-fluoro-4-(4-(3-(methylsulfonyl)phenyl)-1H-pyrazol-1-yl)but-2-en-1-yl)carbamate (0.25 g, 0.61 mmol) in diethyl ether (10 mL) and methanol (1.0 mL) at 0° C. was added HCl (2.0 M in diethyl ether, 9.10 mL, 18.2 mmol). The resulting mixture was warm to rt, and stirring was continued for 3 h. The reaction mixture was concentrated under reduced pressured. The crude residue was triturated with diethyl ether and the resulting solid was dried under high vacuum to afford (Z)-3-fluoro-4-(4-(3-(methylsulfonyl)phenyl)-1H-pyrazol-1-yl)but-2-en-1-amine hydrochloride (0.17 g, 77% yield) as a white solid. ¹H-NMR (300 MHz, CD₃OD) ppm: δ 8.28 (d, J=0.8 Hz, 1H), 8.14 (td, J=1.8, 0.5 Hz, 1H), 8.07 (d, J=0.8 Hz, 1H), 7.95 (ddd, J=7.8, 1.8, 1.1 Hz, 1H), 7.84 (ddd, J=7.8, 1.9, 1.1 Hz, 1H), 7.66 (td, J=7.8, 0.5 Hz, 1H), 5.23 (dt, J=33.4, 7.4 Hz, 1H), 5.08 (dd, J=14.6, 0.9 Hz, 2H), 3.69 (d, J=7.5 Hz, 2H), 3.18 (s, 3H).

### Example 45

Method to Determine the Ability of Compounds of the Invention to Inhibit LOX and LOXL1-4 from Different Sources

Lysyl oxidase (LOX) is an extracellular copper dependent enzyme which oxidizes peptidyl lysine and hydroxylysine residues in collagen and lysine residues in elastin to produce peptidyl alpha-aminoadipic-delta-semialdehydes. This catalytic reaction can be irreversibly inhibited by β-aminopropionitrile (BAPN) that binds to the active site of LOX (Tang S. S., Trackman P. C. and Kagan H. M., Reaction of aortic lysyl oxidase with beta-aminoproprionitrile. J Biol Chem 1983; 258: 4331-4338). There are five LOX family members; these are LOX, LOXL1, LOXL2, LOXL3 and LOXL4. LOX and LOXL family members can be acquired as recombinant active proteins from commercial sources, or extracted from animal tissues like bovine aorta, tendons, pig skin; or prepared from cell cultures. The inhibitory effects of the compounds of the present invention were tested against the given LOX-LOXL preparation using a high-throughput coupled colorimetric method (Holt A. and Palcic M., A peroxidase-coupled continuous absorbance plate-reader assay for flavin monoamine oxidases, copper-containing amine oxidases and related enzymes. Nat. Protoc. 2006; 1: 2498-2505). The assay was developed using either 384 or 96 well format. Briefly, in a standard 384 well plate assay 25 μL of a dilution of any of the isoenzymes and orthologues in 1.2 M urea, 50 mM sodium borate buffer (pH 8.2) were added into each well in the presence of 1 μM mofegiline and 0.5 mM pargyline (to inhibit SSAO and MAO-B and MAO-A, respectively). Test compounds were dissolved in DMSO and tested in a Concentration Response Curve (CRC) with 11 data points, typically in the micromolar or nanomolar range after incubation with the enzyme for 30 min at 37° C. Twenty five μL of a reaction mixture containing twice the K_{M} concentration of putrescine (Sigma Aldrich, e.g. 20 mM for LOX, or 10 mM for LOXL2 and LOXL3), 120 μM Amplex Red (Sigma Aldrich) and 1.5 U/mL horseradish peroxidase (Sigma Aldrich) prepared in 1.2 M urea, 50 mM sodium borate buffer (pH 8.2) were then added to the corresponding wells. The above volumes were doubled in the case of 96 wells plate. The fluorescence (RFU) was read every 2.5 min for 30 min at a range of temperatures from 37° to 45° C., excitation 565 nm and emission 590 (Optima; BMG labtech). The slope of the kinetics for each well was calculated using MARS data analysis software (BMG labtech) and this value was used to deduce the IC₅₀ value (Dotmatics). The ability of the inventive compounds to inhibit the amine oxidase activity LOX and other family members is shown in Table 2.

TABLE 2
LOX and LOXL2 inhibitory activities of examples of compounds
of the invention

| | Bovine LOX | Human LOXL2 |
|---|---|---|
| | Activity IC₅₀ | Activity IC₅₀ |
| Compound | (micromolar) | (micromolar) |
| BAPN | <1 | <1 |
| 1 | <10 | <1 |
| 2 | <10 | <1 |
| 3 | <10 | <1 |
| 4 | <10 | <1 |
| 5 | >10 | <1 |
| 6 | <10 | <1 |
| 7 | <10 | <1 |
| 8 | <10 | <1 |
| 9 | <10 | <1 |
| 10 | <10 | <1 |
| 11 | <10 | <1 |
| 12 | <10 | <1 |
| 13 | <10 | <1 |
| 14 | <10 | <10 |
| 15 | <10 | <1 |
| 16 | <10 | <1 |
| 17 | >10 | <1 |
| 18 | <10 | <1 |
| 19 | <10 | <1 |
| 20 | <10 | <1 |
| 21 | <10 | <1 |
| 22 | <10 | <1 |
| 23 | <10 | <1 |
| 24 | <10 | <1 |
| 25 | <10 | <1 |
| 26 | <10 | <1 |
| 27 | <10 | <1 |
| 28 | <10 | <1 |
| 29 | <10 | <1 |
| 30 | >10 | <1 |
| 31 | >10 | <1 |
| 32 | <10 | <1 |
| 33 | <10 | <1 |
| 34 | <10 | <1 |
| 35 | <10 | <1 |
| 36 | >10 | <1 |
| 37 | <10 | <1 |
| 38 | <10 | <1 |
| 39 | <10 | <1 |
| 40 | <10 | <1 |
| 41 | <10 | <1 |
| 42 | <10 | <1 |
| 43 | <10 | <1 |
| 44 | <10 | <1 |
| 45 | <10 | <1 |
| 45 | <10 | <1 |
| 46 | <10 | <1 |
| 47 | <10 | <1 |
| 48 | <10 | <1 |
| 49 | <10 | <1 |
| 50 | <10 | <1 |
| 51 | <10 | <1 |
| 52 | >10 | <1 |
| 53 | >10 | <1 |
| 54 | >10 | >1 |
| 55 | <10 | <1 |
| 56 | <10 | <1 |
| 57 | <10 | <1 |
| 58 | >10 | <1 |
| 59 | >10 | <1 |
| 60 | <10 | <1 |
| 61 | >10 | <1 |
| 62 | <10 | <1 |
| 63 | <10 | <1 |
| 64 | <10 | <1 |
| 65 | <10 | <1 |
| 66 | <10 | <1 |
| 67 | <10 | <1 |

### Example 46

### Method to Determine the Ability of Compounds of Formula I to Inhibit Human Recombinant SSAO/VAP-1

Human recombinant SSAO/VAP-1 amine oxidase activity was determined using the coupled colorimetric method as described for monoamine oxidase, copper-containing amine oxidases and related enzymes (Holt A. and Palcic M., A peroxidase-coupled continuous absorbance plate-reader assay for flavin monoamine oxidases, copper-containing amine oxidases and related enzymes. Nat Protoc 2006; 1: 2498-2505). Briefly, a cloned cDNA template corresponding to residues 34-763 of human SSAO/VAP-1, and incorporating a mouse Ig kappa (κ) signal sequence, N-terminal flag epitope tag and tobacco etch virus (TEV) cleavage site, was assembled in a mammalian expression vector (pLO-CMV) by Geneart AG. This vector containing human SSAO/VAP-1 residues was transfected into CHO-K1 glycosylation mutant cell line, Lec 8. A clone stably expressing human SSAO/VAP-1 was isolated and cultured in large scale. Active human SSAO/VAP-I was purified and recovered using immunoaffinity chromatography. This was used as the source for SSAO/VAP-1 activity. A high-throughput colorimetric assay was developed using either 96 or 384 well format. Briefly, in a standard 96 well plate assay 50 μL of purified human SSAO/VAP-1 (0.25 μg/mL) in 0.1 M sodium phosphate buffer (pH 7.4) was added into each well. Test compounds were dissolved in DMSO and tested in a Concentration Response Curve (CRC) with 4-11 data points, typically in the micromolar or nanomolar range after incubation with human SSAO/VAP-1 for 30 min at 37° C. After 30 min incubation, 50 μL of the reaction mixture containing 600 μM benzylamine (Sigma Aldrich), 120 μM Amplex Red (Sigma Aldrich) and 1.5 U/mL horseradish peroxidase (Sigma Aldrich) prepared in 0.1 M sodium phosphate buffer (pH 7.4) were added to the corresponding well. The fluorescence unit (RFU) was read every 2.5 min for 30 min at 37° C. excitation 565 nm and emission 590 (Optima; BMG labtech). The slope of the kinetics for each well was calculated using MARS data analysis software (BMG labtech) and this value was used to deduce the IC₅₀ value (Dotmatics). The ability of the compounds of Formula I to inhibit SSAO/VAP-I is shown in Table 3.

### Example 47

### Method to Determine the Ability of Compounds of Formula I to Inhibit Human Recombinant MAO-B

The specificity of the compounds of this invention was tested by determining their ability to inhibit MAO-B activities in vitro. Recombinant human MAO-B (0.06 mg/mL; Sigma Aldrich) was used as source of MAO-B enzyme activities. The assay was performed in a similar way as for human SSAO/VAP-1 (Example 46) except, the substrate benzylamine was used at 100 μM. The ability of compounds of Formula I to inhibit MAO-B is shown in Table 3

TABLE 3
Selectivity of Compounds of Formula I for LOX and LOXL2 compared
to SSAO/VAP-1 and MAO-B

| | SSAO/VAP-1 | MAO-B Activity |
|---|---|---|
| | Activity IC₅₀ | IC₅₀ |
| Compound | (micromolar) | (micromolar) |
| BAPN | >100 | >100 |
| 1 | <10 | >10 |
| 2 | >30 | >30 |
| 3 | >30 | >30 |
| 4 | >30 | >30 |
| 5 | >30 | >30 |
| 6 | >30 | >30 |
| 7 | >10 | >30 |
| 8 | >10 | >30 |
| 9 | >10 | >10 |
| 10 | <10 | >10 |
| 11 | <10 | >30 |
| 12 | <10 | >30 |
| 13 | >30 | >30 |
| 14 | >30 | >30 |
| 15 | <10 | >30 |
| 16 | >10 | >30 |
| 17 | >10 | >30 |
| 18 | >30 | >30 |
| 19 | >30 | >30 |
| 20 | <10 | >30 |
| 21 | <10 | >30 |
| 22 | >10 | >30 |
| 23 | >30 | >30 |
| 24 | >30 | >30 |
| 25 | >30 | >30 |
| 26 | <10 | >30 |
| 27 | >30 | >30 |
| 28 | >30 | >30 |
| 29 | >30 | >30 |
| 30 | >30 | >30 |
| 31 | >30 | >30 |
| 32 | >30 | >30 |
| 33 | — | — |
| 34 | — | — |
| 35 | — | — |
| 36 | — | >30 |
| 37 | — | — |
| 38 | — | — |
| 39 | — | — |
| 40 | — | — |
| 41 | — | — |
| 42 | — | — |
| 43 | — | — |
| 44 | — | — |
| 45 | — | — |
| 46 | — | — |
| 47 | — | — |
| 48 | — | — |
| 49 | — | — |
| 50 | — | — |
| 51 | — | — |
| 52 | >30 | >30 |
| 53 | >30 | >30 |
| 54 | — | — |
| 55 | — | — |
| 56 | — | — |
| 57 | — | — |
| 58 | — | — |
| 59 | >30 | — |
| 60 | >30 | — |
| 61 | >30 | — |
| 62 | >30 | — |
| 63 | — | — |
| 64 | — | — |
| 65 | — | — |
| 66 | — | — |
| 67 | — | — |

LOX and LOXL1-4 enzymes are members of a large family of flavin-dependent and copper-dependent amine oxidases, which includes SSAO/VAP-1 and monoamine oxidase-B (MAO-B). Compounds of the present invention selectively inhibit members of the LOX family of enzymes with respect to SSAO/VAP-1, MAO-B and other family member amine oxidases. Examples of the magnitude of selectivity can be seen in Table 3.

### Example 48

### Inhibition of CCl₄ Induced Liver Fibrosis

An analysis of the use of LOXL2 inhibitors to treat inflammatory/fibrotic diseases is performed through the use of a CCl₄ induced liver fibrosis model. Liver injury is frequently followed by complete parenchymal regeneration due to regenerative potency of hepatocytes. Continuous liver injury due to the administration of CCl₄ leads to extracellular matrix accumulation, accompanied by recurrent hepatocyte necrosis, inflammation, and regenerative processes, causing liver fibrosis and consequently liver cirrhosis (see Natsume, M., et al., Attenuated liver fibrosis and depressed serum albumin levels in carbon tetrachloride-treated IL-6-deficient mice. J. Leukoc. Biol., 1999, 66, 601-608 also See Yao, Q, Y., et al. Inhibition by curcumin of multiple sites of the transforming growth factor-beta1 signalling pathway ameliorates the progression of liver fibrosis induced by carbon tetrachloride in rats. BMC Complement Altern Med. 2012 Sep. 16; 12(1):156.).

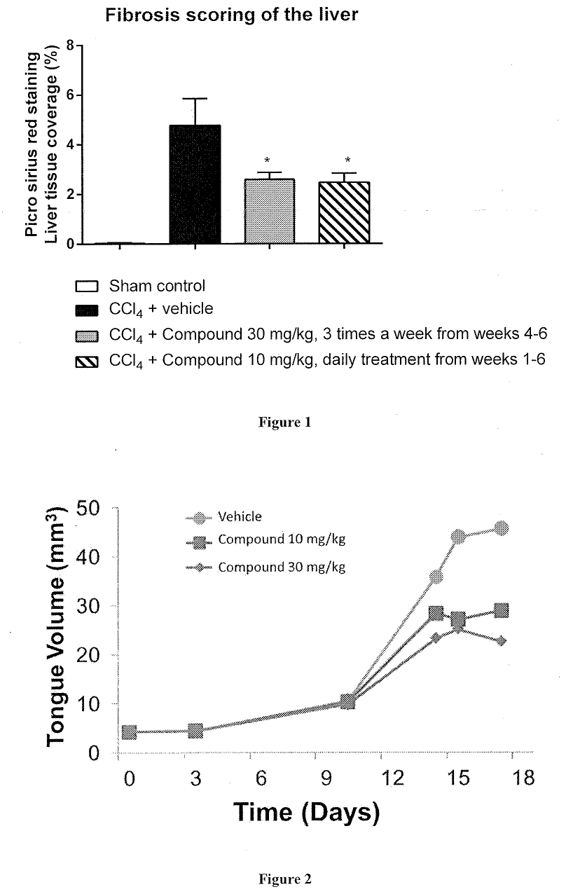

Mice are injected intraperitoneally with CCl₄ (i.p.) 1 ml/kg 25% CCl₄ in olive oil, twice per week for a total period of 6 weeks. Compound 15 is given 0.1-100 mg/Kg throughout the period of the experimental procedure or only 3 weeks after CCl₄ administration and then throughout the entire study. Compared with the vehicle-treated group that show increases in fibrosis in the liver, Compound 15 administration shows up to 50% reduction as demonstrated by liver sirius red staining with quantification (see FIG. 1).

### Example 49

### Inhibition of Metastatic Cancer

An analysis of the use of LOXL2 inhibitors to treat cancer diseases is performed through the use of an oral metastatic cancer mouse model.

Mice are injected with a metastatic oral cancer cell line expressing DsRed for tracking. Mice are monitored weekly by in vivo imaging (IVIS) based on DsRed expression and by caliper measurements of mouse tongues over a period of up to five weeks. Compound 15 is given 0.1-100 mg/Kg throughout the period of the experimental procedure or only two times a week throughout the entire study. Compared with the vehicle-treated group that show increases in tongue cancer volume as well as metastasis throughout the body, Compound 15 administration shows a significant reduction as demonstrated by a decrease in tongue volume and spread of metastasis (See FIG. 2).

1. A compound of Formula I: 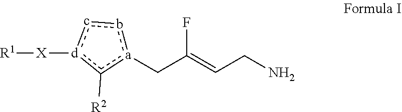
or a stereoisomer, pharmaceutically acceptable salt, polymorphic form, solvate, tautomeric form, thereof; wherein:
each [symbol] is independently a single or double bond arranged so as to provide a pyrazole ring;
a is C or N;
b is C(R³) or N;
c is C(R⁴) or N;
d is C or N;
and 2 of a, b, c and d are N, wherein the 2 N atoms are adjacent to each other;
R², R³ and R⁴ are independently selected from the group consisting of hydrogen, halogen, C₁₋₄alkyl, —C₃₋₅cycloalkyl, —O—C₁₋₄alkyl, —O—C₃₋₅cycloalkyl, —C(O) OR⁵, —C(O)NR⁶R⁷ and —NR⁶C(O)R⁸; wherein each C₁₋₄alkyl is a straight or branched chain alkyl;
and wherein each C₁₋₄alkyl and C₃₋₅cycloalkyl is optionally substituted by one or more substituents selected from the group consisting of halogen, —OH, —SH, —C₁₋₃alkyl, —O—C₁₋₃alkyl, —CF₃, —CH₂CF₃ and —O—CF₃;
X is O or —(CH₂)ₘ—;
R¹ is selected from the group consisting of aryl and heteroaryl; wherein each R¹ is optionally substituted by one or more R⁹;
R⁵ is selected from the group consisting of hydrogen, —C₁₋₆alkyl, and —C₃₋₇cycloalkyl; wherein each C₁₋₆alkyl is a straight or branched chain alkyl, and wherein each C₁₋₆alkyl, and C₃₋₇cycloalkyl is optionally substituted by one or more substituents selected from the group consisting of halogen, —OH, —SH, —C₁₋₃alkyl, —O—C₁₋₃alkyl, —CF₃, —CH₂CF₃ and —O—CF₃;
R⁶ and R⁷ are independently selected from the group consisting of hydrogen, C₁₋₆alkyl and C₃₋₇cycloalkyl; wherein each C₁₋₆alkyl is a straight or branched chain alkyl; and wherein each C₁₋₆alkyl and C₃₋₇cycloalkyl is optionally substituted by one or more substituents selected from the group consisting of halogen, —OH, —SH, —C₁₋₃alkyl, —O—C₁₋₃alkyl, —CF₃, —CH₂CF₃, and —O—CF₃; or
R⁶ and R¹ when attached to the same nitrogen atom are combined to form a 3- to 7-membered ring having from 0 to 2 additional heteroatoms as ring members;
R⁸ is selected from the group consisting of C₁₋₆alkyl and C₃₋₇cycloalkyl; wherein each C₁₋₆alkyl is a straight or branched chain alkyl; and wherein each C₁₋₆alkyl and C₃₋₇cycloalkyl is optionally substituted by one or more substituents selected from the group consisting of halogen, —OH, —SH, —C₁₋₃alkyl, —O—C₁₋₃alkyl, —CF₃, —CH₂CF₃, and —O—CF₃; and
each R⁹ is independently selected from the group consisting of halogen, C₁₋₆alkyl, —O—C₁₋₆alkyl, —S—C₁₋₆alkyl, C₃₋₇cycloalkyl, —O—C₃₋₇cycloalkyl, —C(O)OR⁵, —C(O)NR⁶R⁷, —NR⁶C(O)R⁸, —S(O₂)NR⁶R⁷, —NR⁶S(O₂)R⁸, —S(O)R⁸ and —S(O₂)R⁸; wherein each C₁₋₆alkyl is a straight or branched chain alkyl; and wherein each C₁₋₆alkyl and C₃₋₇cycloalkyl is optionally substituted by one or more substituents selected from the group consisting of halogen, —OH, —C₁₋₃alkyl, —O—C₁₋₃alkyl, —CF₃, —CH₂CF₃, and —O—CF₃; and
m is 0 or 1.

2. A The compound according to claim 1, of Formula Ia 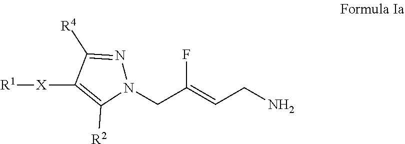
or a pharmaceutically acceptable salt or solvate thereof, wherein:
R² and R⁴ are independently selected from the group consisting of hydrogen, halogen, C₁₋₄alkyl, —C₃₋₅cycloalkyl, —O—C₁₋₄alkyl, —O—C₃₋₅cycloalkyl, —C(O)OR⁵, —C(O)NR⁶R⁷ and —NR⁶C(O)R⁸; wherein each C₁₋₄alkyl is a straight or branched chain alkyl; and wherein each C₁₋₄alkyl and C₃₋₅cycloalkyl is optionally substituted by one or more substituents selected from the group consisting of halogen, —OH, —SH, —C₁₋₃alkyl, —O—C₁₋₃alkyl, —CF₃, —CH₂CF₃ and —O—CF₃;
X is O or —(CH₂)ₘ—;
R¹ is selected from the group consisting of aryl and heteroaryl; wherein each R¹ is optionally substituted by one or more R⁹;
R⁵ is selected from the group consisting of hydrogen, —C₁₋₆alkyl, and —C₃₋₇cycloalkyl; wherein each C₁₋₆alkyl is a straight or branched chain alkyl, and wherein each C₁₋₆alkyl, and C₃₋₇cycloalkyl is optionally substituted by one or more substituents selected from the group consisting of halogen, —OH, —SH, —C₁₋₃alkyl, —O—C₁₋₃alkyl, —CF₃, —CH₂CF₃ and —O—CF₃;
R⁶ and R⁷ are independently selected from the group consisting of hydrogen, C₁₋₆alkyl and C₃₋₇cycloalkyl; wherein each C₁₋₆alkyl is a straight or branched chain alkyl; and wherein each C₁₋₆alkyl and C₃₋₇cycloalkyl is optionally substituted by one or more substituents selected from the group consisting of halogen, —OH, —SH, —C₁₋₃alkyl, —O—C₁₋₃alkyl, —CF₃, —CH₂CF₃, and —O—CF₃; or
R⁶ and R⁷ when attached to the same nitrogen atom are combined to form a 3- to 7-membered ring having from 0 to 2 additional heteroatoms as ring members;
R⁸ is selected from the group consisting of C₁₋₆alkyl and C₃₋₇cycloalkyl; wherein each C₁₋₆alkyl is a straight or branched chain alkyl; and wherein each C₁₋₆alkyl and C₃₋₇cycloalkyl is optionally substituted by one or more substituents selected from the group consisting of halogen, —OH, —SH, —C₁₋₃alkyl, —O—C₁₋₃alkyl, —CF₃, —CH₂CF₃, and —O—CF₃; and
each R⁹ is independently selected from the group consisting of halogen, C₁₋₆alkyl, —O—C₁₋₆alkyl, —S—C₁₋₆alkyl, C₃₋₇cycloalkyl, —O—C₃₋₇cycloalkyl, —C(O)OR⁵, —C(O)NR⁶R⁷, —NR⁶C(O)R⁸, —S(O₂)NR⁶R⁷, —NR⁶S(O₂)R⁸, —S(O)R⁸ and —S(O₂)R⁸; wherein each C₁₋₆alkyl is a straight or branched chain alkyl; and wherein each C₁₋₆alkyl and C₃₋₇cycloalkyl is optionally substituted by one or more substituents selected from the group consisting of halogen, —OH, —C₁₋₃alkyl, —O—C₁₋₃alkyl, —CF₃, —CH₂CF₃, and —O—CF₃; and
m is 0 or 1.

3. A The compound according to claim 1 of Formula Ib 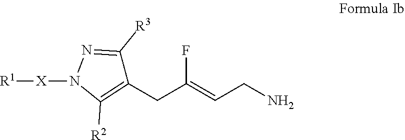
or a pharmaceutically acceptable salt or solvate thereof, wherein:
R² and R³ are independently selected from the group consisting of hydrogen, halogen, C₁₋₄alkyl, —C₃₋₅cycloalkyl, —O—C₁₋₄alkyl, —O—C₃₋₅cycloalkyl, —C(O)OR⁵, —C(O)NR⁶R⁷ and —NR⁶C(O)R⁸; wherein each C₁₋₄alkyl is a straight or branched chain alkyl; and wherein each C₁₋₄alkyl and C₃₋₅cycloalkyl is optionally substituted by one or more substituents selected from the group consisting of halogen, —OH, —SH, —C₁₋₃alkyl, —O—C₁₋₃alkyl, —CF₃, —CH₂CF₃ and —O—CF₃;
X is O or —(CH₂)ₘ—;
R¹ is selected from the group consisting of aryl and heteroaryl; wherein each R¹ is optionally substituted by one or more R⁹;
R⁵ is selected from the group consisting of hydrogen, —C₁₋₆alkyl, and —C₃₋₇cycloalkyl; wherein each C₁₋₆alkyl is a straight or branched chain alkyl, and wherein each C₁₋₆alkyl, and C₃₋₇cycloalkyl is optionally substituted by one or more substituents selected from the group consisting of halogen, —OH, —SH, —C₁₋₃alkyl, —O—C₁₋₃alkyl, —CF₃, —CH₂CF₃ and —O—CF₃;
R⁶ and R⁷ are independently selected from the group consisting of hydrogen, C₁₋₆alkyl and C₃₋₇cycloalkyl; wherein each C₁₋₆alkyl is a straight or branched chain alkyl; and wherein each C₁₋₆alkyl and C₃₋₇cycloalkyl is optionally substituted by one or more substituents selected from the group consisting of halogen, —OH, —SH, —C₁₋₃alkyl, —O—C₁₋₃alkyl, —CF₃, —CH₂CF₃, and —O—CF₃; or
R⁶ and R⁷ when attached to the same nitrogen atom are combined to form a 3- to 7-membered ring having from 0 to 2 additional heteroatoms as ring members;
R⁸ is selected from the group consisting of C₁₋₆alkyl and C₃₋₇cycloalkyl; wherein each C₁₋₆alkyl is a straight or branched chain alkyl; and wherein each C₁₋₆alkyl and C₃₋₇cycloalkyl is optionally substituted by one or more substituents selected from the group consisting of halogen, —OH, —SH, —C₁₋₃alkyl, —O—C₁₋₃alkyl, —CF₃, —CH₂CF₃, and —O—CF₃; and
each R⁹ is independently selected from the group consisting of halogen, C₁₋₆alkyl, —O—C₁₋₆alkyl, —S—C₁₋₆alkyl, C₃-7cycloalkyl, —O—C₃₋₇cycloalkyl, —C(O) OR⁵, —C(O)NR⁶R⁷, —NR⁶C(O)R⁸, —S(O₂)NR⁶R⁷, —NR⁶S(O₂)R⁸, —S(O)R⁸ and —S(O₂)R⁸; wherein each C₁₋₆alkyl is a straight or branched chain alkyl; and wherein each C₁₋₆alkyl and C₃₋₇cycloalkyl is optionally substituted by one or more substituents selected from the group consisting of halogen, —OH, —C₁₋₃alkyl, —O—C₁₋₃alkyl, —CF₃, —CH₂CF₃, and —O—CF₃; and
m is 0 or 1.

4. The compound according to claim 1, wherein m is 0.

5. The compound according to claim 1, wherein m is 1.

6. The compound according to claim 1, wherein R¹ is selected from phenyl, naphthyl or pyridyl.

7. The compound according to claim 1, wherein R², R³ and R⁴ are independently selected from the group consisting of hydrogen, halogen, C₁₋₄alkyl, —C(O)OR⁵, and —C(O)NR⁶R⁷; wherein each C₁₋₄alkyl is a straight or branched chain alkyl; and wherein each C₁₋₄alkyl is optionally substituted by one or more substituents selected from the group consisting of halogen, —OH and —O—C₁₋₃alkyl.

8. The compound according to claim 1, wherein R², R³ and R⁴ are independently selected from the group consisting of hydrogen, chlorine, methyl, ethyl, isopropyl, tert-butyl, —CF₃, —CH₂OH, CHOHCH₃, —C(CH₃)₂OH, —C(O)OEt, —C(O)OH, —C(O)N(CH₃)₂, C(O)NHC(CH₃)₃, —CHCH₃OH and —CH₂OCH₃.

9. The compound according to claim 1, wherein R⁵ is selected from the group consisting of hydrogen and C₁₋₆alkyl.

10. The compound according to claim 1, wherein R⁶ and R⁷ are independently selected from the group consisting of hydrogen and C₁₋₆alkyl; wherein each C₁₋₆alkyl is a straight or branched chain alkyl; or
R⁶ and R⁷ when attached to the same nitrogen atom are combined to form a 3- to 7-membered ring having from 0 to 2 additional heteroatoms as ring members.

11. (canceled)

12. The compound according to claim 1, wherein each R⁹ is independently selected from the group consisting of halogen, C₁₋₆alkyl, —O—C₁₋₆alkyl, —C(O)NR⁶R⁷, —S(O₂)NR⁶R⁷, and —S(O₂)R⁸; wherein each C₁₋₆alkyl is a straight or branched chain alkyl; and wherein each C₁₋₆alkyl is optionally substituted by one or more halogen.

13. The compound according to claim 1, wherein each R⁹ is independently selected from the group consisting of fluorine, chlorine, —CF₃, —OCF₃, —C(O) N(CH₃)₂, —S(O₂)NR⁶R⁷, —S(O₂) CF₃, —S(O₂)CH(CH₃)₂ and —S(O₂)CH₃.

14. The compound according to claim 1, selected from the group consisting of:

| Compound | Structure | Name |
|---|---|---|
| 1 | 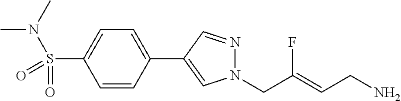 [SMILES: CN(C)S(=O)(=O)c1ccc(-c2cnn(C/C(F)=C/CN)c2)cc1] | (Z)-4-(1-(4-amino-2- fluorobut-2-en-1-yl)-1H- pyrazol-4-yl)-N,N- dimethylbenzenesulfonamide |
| 2 | 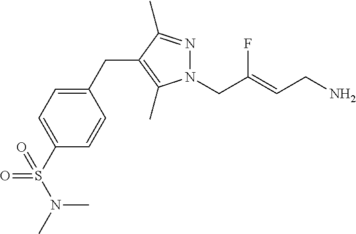 [SMILES: Cc1nn(C/C(F)=C/CN)c(C)c1Cc1ccc(S(=O)(=O)N(C)C)cc1] | (Z)-4-((1-(4-amino-2- fluorobut-2-en-1-yl)-3,5- dimethyl-1H-pyrazol-4-yl) methyl)-N,N- dimethylbenzenesulfonamide |
| 3 | 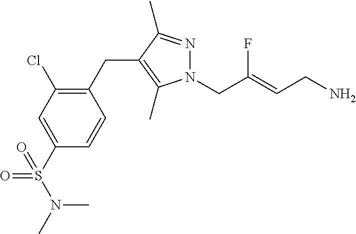 [SMILES: Cc1nn(C/C(F)=C/CN)c(C)c1Cc1ccc(S(=O)(=O)N(C)C)cc1Cl] | (Z)-4-((1-(4-amino-2- fluorobut-2-en-1-yl)-3,5- dimethyl-1H-pyrazol-4- yl)methyl)-3-chloro-N,N- dimethylbenzenesulfonamide |
| 4 | 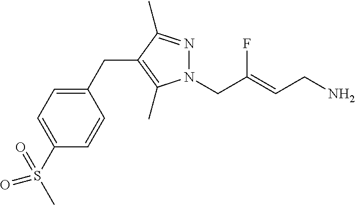 [SMILES: Cc1nn(C/C(F)=C/CN)c(C)c1Cc1ccc(S(C)(=O)=O)cc1] | (Z)-4-(3,5-dimethyl-4-(4- (methylsulfonyl)benzyl)- 1H-pyrazol-1-yl)-3- fluorobut-2-en-1-amine |
| 5 | 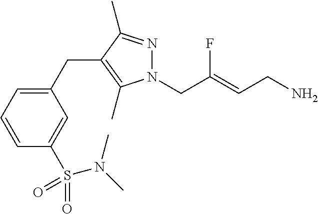 [SMILES: Cc1nn(C/C(F)=C/CN)c(C)c1Cc1cccc(S(=O)(=O)N(C)C)c1] | (Z)-3-((1-(4-amino-2- fluorobut-2-en-1-yl)-3,5- dimethyl-1H-pyrazol-4- yl)methyl)-N,N- dimethylbenzenesulfonamide |
| 6 | 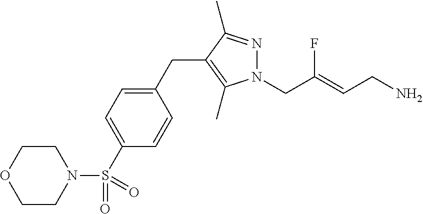 [SMILES: Cc1nn(C/C(F)=C/CN)c(C)c1Cc1ccc(S(=O)(=O)N2CCOCC2)cc1] | (Z)-4-(3,5-dimethyl-4-(4- (morpholinosulfonyl)benzyl)- 1H-pyrazol-1-yl)-3-fluorobut- 2-en-1-amine |
| 7 | 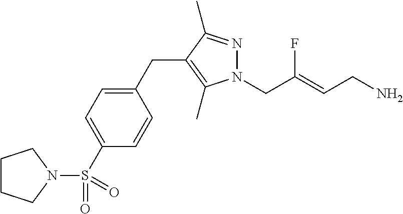 [SMILES: Cc1nn(C/C(F)=C/CN)c(C)c1Cc1ccc(S(=O)(=O)N2CCCC2)cc1] | (Z)-4-(3,5-dimethyl-4-(4- (pyrrolidin-1-ylsulfonyl) benzyl)-1H-pyrazol-1-yl)-3- fluorobut-2-en-1-amine |
| 8 | 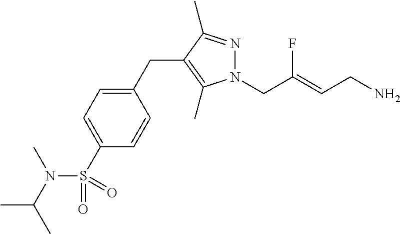 [SMILES: Cc1nn(C/C(F)=C/CN)c(C)c1Cc1ccc(S(=O)(=O)N(C)C(C)C)cc1] | (Z)-4-((1-(4-amino-2- fluorobut-2-en-1-yl)-3,5- dimethyl-1H-pyrazol-4- yl)methyl)-N-isopropyl-N- methylbenzenesulfonamide |
| 9 | 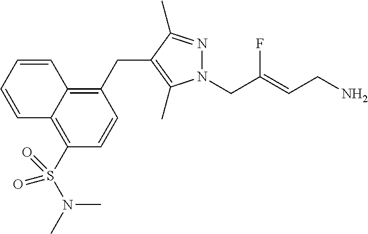 [SMILES: Cc1nn(C/C(F)=C/CN)c(C)c1Cc1ccc(S(=O)(=O)N(C)C)c2ccccc12] | (Z)-4-((1-(4-amino-2- fluorobut-2-en-1-yl)-3,5- dimethyl-1H-pyrazol-4- yl)methyl)-N,N- dimethylnaphthalene-1- sulfonamide |
| 10 | 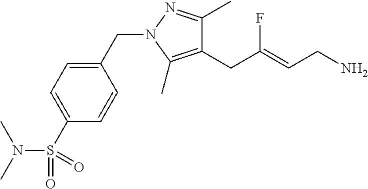 [SMILES: Cc1nn(Cc2ccc(S(=O)(=O)N(C)C)cc2)c(C)c1C/C(F)=C/CN] | (Z)-4-((4-(4-amino-2- fluorobut-2-en-1-yl)-3,5- dimethyl-1H-pyrazol-1- yl)methyl)-N,N- dimethylbenzenesulfonamide |
| 11 | 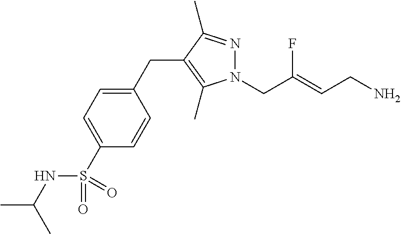 [SMILES: Cc1nn(C/C(F)=C/CN)c(C)c1Cc1ccc(S(=O)(=O)NC(C)C)cc1] | (Z)-4-((1-(4-amino-2- fluorobut-2-en-1-yl)-3,5- dimethyl-1H-pyrazol-4- yl)methyl)-N- isopropylbenzenesulfonamide |
| 12 | 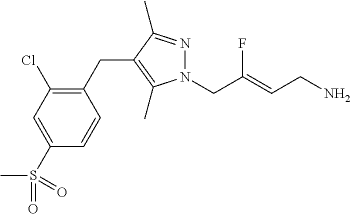 [SMILES: Cc1nn(C/C(F)=C/CN)c(C)c1Cc1ccc(S(C)(=O)=O)cc1Cl] | (Z)-4-(4-(2-chloro-4- (methylsulfonyl)benzyl)- 3,5-dimethyl-1H-pyrazol- 1-yl)-3-fluorobut-2-en-1- amine |
| 13 | 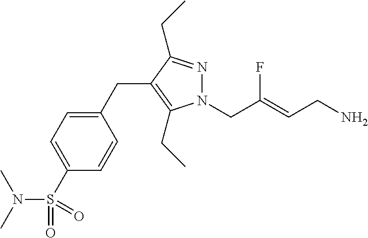 [SMILES: CCc1nn(C/C(F)=C/CN)c(CC)c1Cc1ccc(S(=O)(=O)N(C)C)cc1] | (Z)-4-((1-(4-amino-2- fluorobut-2-en-1-yl)-3,5- diethyl-1H-pyrazol-4- yl)methyl)-N,N- dimethylbenzenesulfonamide |
| 14 | 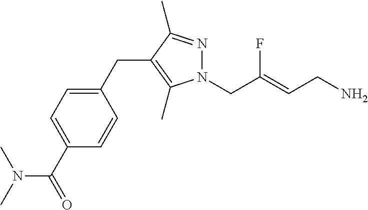 [SMILES: Cc1nn(C/C(F)=C/CN)c(C)c1Cc1ccc(C(=O)N(C)C)cc1] | (Z)-4-((1-(4-amino-2- fluorobut-2-en-1-yl)-3,5- dimethyl-1H-pyrazol-4- yl)methyl)-N,N- dimethylbenzamide |
| 15 | 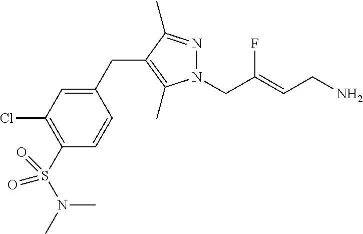 [SMILES: Cc1nn(C/C(F)=C/CN)c(C)c1Cc1ccc(S(=O)(=O)N(C)C)c(Cl)c1] | (Z)-4-((1-(4-amino-2- fluorobut-2-en-1-yl)-3,5- dimethyl-1H-pyrazol-4- yl)methyl)-2-chloro-N,N- dimethylbenzenesulfonamide |
| 16 | 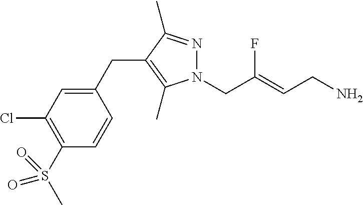 [SMILES: Cc1nn(C/C(F)=C/CN)c(C)c1Cc1ccc(S(C)(=O)=O)c(Cl)c1] | (Z)-4-(4-(3-chloro-4- (methylsulfonyl)benzyl)- 3,5-dimethyl-1H-pyrazol- 1-yl)-3-fluorobut-2-en-1- amine |
| 17 | 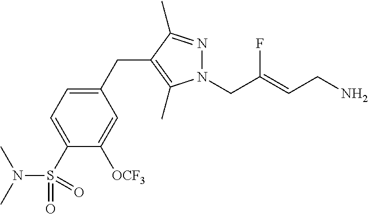 [SMILES: Cc1nn(C/C(F)=C/CN)c(C)c1Cc1ccc(S(=O)(=O)N(C)C)c(OC(F)(F)F)c1] | (Z)-4-((1-(4-amino-2- fluorobut-2-en-1-yl)-3,5- dimethyl-1H-pyrazol-4- yl)methyl)-N,N-dimethyl-2- (trifluoromethoxy)benzene- sulfonamide |
| 18 | 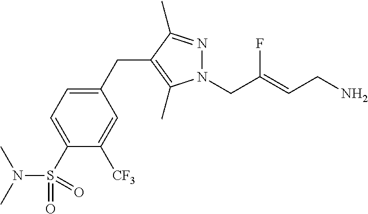 [SMILES: Cc1nn(C/C(F)=C/CN)c(C)c1Cc1ccc(S(=O)(=O)N(C)C)c(C(F)(F)F)c1] | (Z)-4-((1-(4-amino-2- fluorobut-2-en-1-yl)-3,5- dimethyl-1H-pyrazol-4- yl)methyl)-N,N-dimethyl-2- (trifluoromethyl)benzene- sulfonamide |
| 19 | 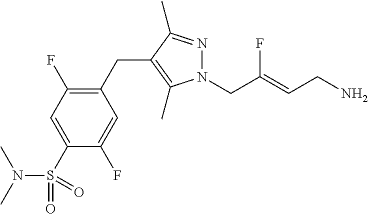 [SMILES: Cc1nn(C/C(F)=C/CN)c(C)c1Cc1cc(F)c(S(=O)(=O)N(C)C)cc1F] | (Z)-4-((1-(4-amino-2- fluorobut-2-en-1-yl)-3,5- dimethyl-1H-pyrazol-4- yl)methyl)-2,5-difluoro-N,N- dimethylbenzenesulfonamide |
| 20 | 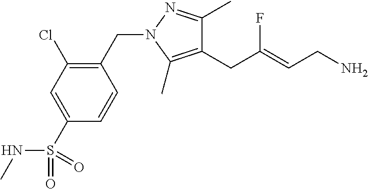 [SMILES: CNS(=O)(=O)c1ccc(Cn2nc(C)c(C/C(F)=C/CN)c2C)c(Cl)c1] | (Z)-4-((4-(4-amino-2- fluorobut-2-en-1-yl)-3,5- dimethyl-1H-pyrazol-1- yl)methyl)-3-chloro-N- methylbenzenesulfonamide |
| 21 | 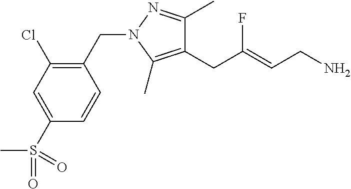 [SMILES: Cc1nn(Cc2ccc(S(C)(=O)=O)cc2Cl)c(C)c1C/C(F)=C/CN] | (Z)-4-(1-(2-chloro-4- (methylsulfonyl)benzyl)-3,5- dimethyl-1H-pyrazol-4-yl)-3- fluorobut-2-en-1-amine |
| 22 | 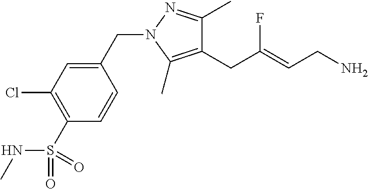 [SMILES: CNS(=O)(=O)c1ccc(Cn2nc(C)c(C/C(F)=C/CN)c2C)cc1Cl] | (Z)-4-((4-(4-amino-2- fluorobut-2-en-1-yl)-3,5- dimethyl-1H-pyrazol-1- yl)methyl)-2-chloro-N- methylbenzenesulfonamide |
| 23 | 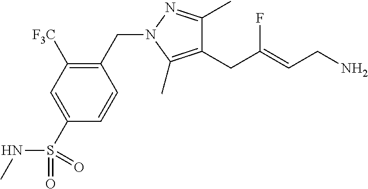 [SMILES: CNS(=O)(=O)c1ccc(Cn2nc(C)c(C/C(F)=C/CN)c2C)c(C(F)(F)F)c1] | (Z)-4-((4-(4-amino-2- fluorobut-2-en-1-yl)-3,5- dimethyl-1H-pyrazol-1- yl)methyl)-N-methyl-3- (trifluoromethyl)benzene sulfonamide |
| 24 | 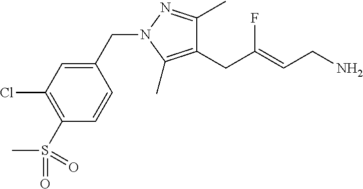 [SMILES: Cc1nn(Cc2ccc(S(C)(=O)=O)c(Cl)c2)c(C)c1C/C(F)=C/CN] | (Z)-4-(1-(3-chloro-4- (methylsulfonyl)benzyl)-3,5- dimethyl-1H-pyrazol-4-yl)-3- fluorobut-2-en-1-amine |
| 25 | 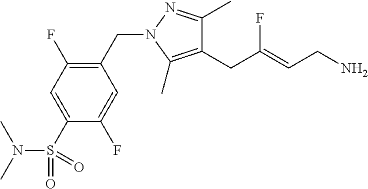 [SMILES: Cc1nn(Cc2cc(F)c(S(=O)(=O)N(C)C)cc2F)c(C)c1C/C(F)=C/CN] | (Z)-4-((4-(4-amino-2- fluorobut-2-en-1-yl)-3,5- dimethyl-1H-pyrazol-1- yl)methyl)-2,5-difluoro-N,N- dimethylbenzenesulfonamide |
| 26 | 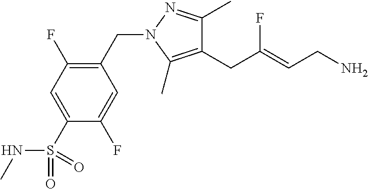 [SMILES: CNS(=O)(=O)c1cc(F)c(Cn2nc(C)c(C/C(F)=C/CN)c2C)cc1F] | (Z)-4-((4-(4-amino-2- fluorobut-2-en-1-yl)-3,5- dimethyl-1H-pyrazol-1- yl)methyl)-2,5-difluoro-N- methylbenzenesulfonamide |
| 27 | 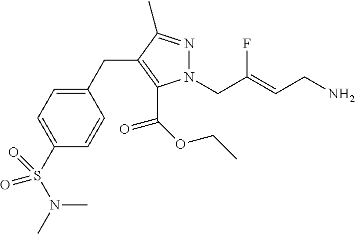 [SMILES: CCOC(=O)c1c(Cc2ccc(S(=O)(=O)N(C)C)cc2)c(C)nn1C/C(F)=C/CN] | ethyl (Z)-1-(4-amino-2- fluorobut-2-en-1-yl)-4-(4- (N,N-dimethylsulfamoyl) benzyl)-3-methyl-1H- pyrazole-5-carboxylate |
| 28 | 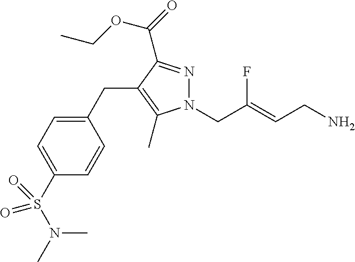 [SMILES: CCOC(=O)c1nn(C/C(F)=C/CN)c(C)c1Cc1ccc(S(=O)(=O)N(C)C)cc1] | ethyl (Z)-1-(4-amino-2- fluorobut-2-en-1-yl)-4-(4- (N,N-dimethylsulfamoyl) benzyl)-5-methyl-1H- pyrazole-3-carboxylate |
| 29 | 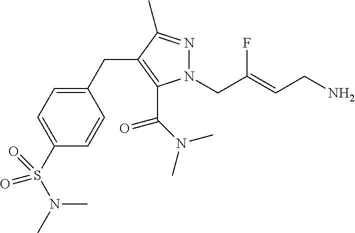 [SMILES: Cc1nn(C/C(F)=C/CN)c(C(=O)N(C)C)c1Cc1ccc(S(=O)(=O)N(C)C)cc1] | (Z)-1-(4-amino-2-fluorobut- 2-en-1-yl)-4-(4-(N,N- dimethylsulfamoyl)benzyl)- N,N,3-trimethyl-1H-pyrazole- 5-carboxamide |
| 30 | 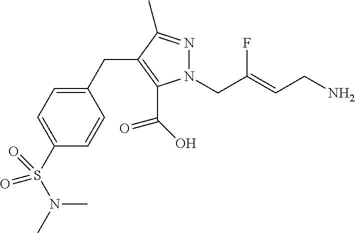 [SMILES: Cc1nn(C/C(F)=C/CN)c(C(=O)O)c1Cc1ccc(S(=O)(=O)N(C)C)cc1] | (Z)-1-(4-amino-2-fluorobut- 2-en-1-yl)-4-(4-(N,N- dimethylsulfamoyl)benzyl)- 3-methyl-1H-pyrazole-5- carboxylic acid |
| 31 | 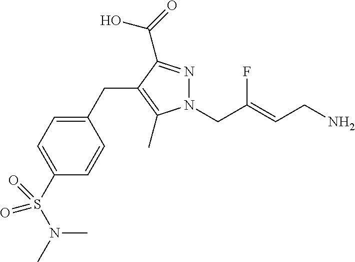 [SMILES: Cc1c(Cc2ccc(S(=O)(=O)N(C)C)cc2)c(C(=O)O)nn1C/C(F)=C/CN] | (Z)-1-(4-amino-2-fluorobut- 2-en-1-yl)-4-(4-(N,N- dimethylsulfamoyl)benzyl)- 5-methyl-1H-pyrazole-3- carboxylic acid |
| 32 | 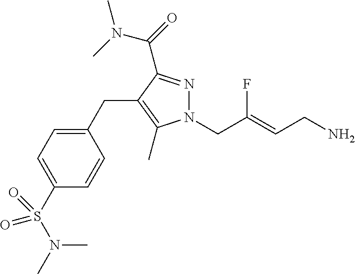 [SMILES: Cc1c(Cc2ccc(S(=O)(=O)N(C)C)cc2)c(C(=O)N(C)C)nn1C/C(F)=C/CN] | (Z)-1-(4-amino-2-fluorobut- 2-en-1-yl)-4-(4-(N,N- dimethylsulfamoyl)benzyl)- N,N,5-trimethyl-1H- pyrazole-3-carboxamide |
| 33 | 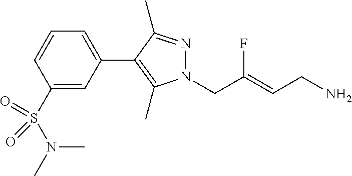 [SMILES: Cc1nn(C/C(F)=C/CN)c(C)c1-c1cccc(S(=O)(=O)N(C)C)c1] | (Z)-3-(1-(4-amino-2- fluorobut-2-en-1-yl)-3,5- dimethyl-1H-pyrazol-4-yl)- N,N- dimethylbenzenesulfonamide |
| 34 | 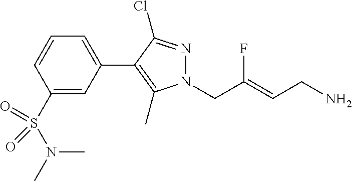 [SMILES: Cc1c(-c2cccc(S(=O)(=O)N(C)C)c2)c(Cl)nn1C/C(F)=C/CN] | (Z)-3-(1-(4-amino-2- fluorobut-2-en-1-yl)-3- chloro-5-methyl-1H-pyrazol- 4-yl)-N,N- dimethylbenzenesulfonamide |
| 35 | 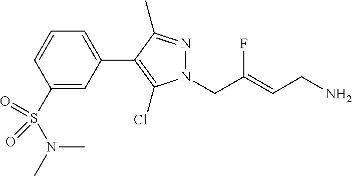 [SMILES: Cc1nn(C/C(F)=C/CN)c(Cl)c1-c1cccc(S(=O)(=O)N(C)C)c1] | (Z)-3-(1-(4-amino-2- fluorobut-2-en-1-yl)-5- chloro-3-methyl-1H-pyrazol- 4-yl)-N,N- dimethylbenzenesulfonamide |
| 36 | 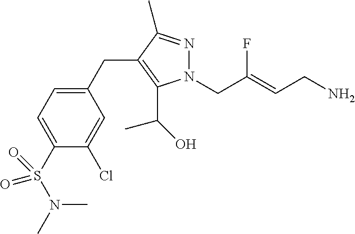 [SMILES: Cc1nn(C/C(F)=C/CN)c(C(C)O)c1Cc1ccc(S(=O)(=O)N(C)C)c(Cl)c1] | (Z)-4-((1-(4-amino-2- fluorobut-2-en-1-yl)-5-(1- hydroxyethyl)-3-methyl-1H- pyrazol-4-yl)methyl)-2- chloro-N,N- dimethylbenzenesulfonamide |
| 37 | 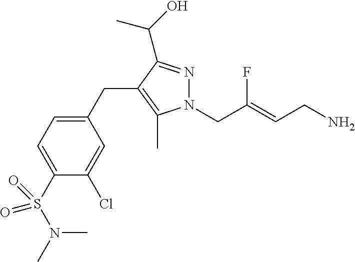 [SMILES: Cc1c(Cc2ccc(S(=O)(=O)N(C)C)c(Cl)c2)c(C(C)O)nn1C/C(F)=C/CN] | (Z)-4-((1-(4-amino-2- fluorobut-2-en-1-yl)-3-(1- hydroxyethyl)-5-methyl-1H- pyrazol-4-yl)methyl)-2- chloro-N,N- dimethylbenzenesulfonamide |
| 38 | 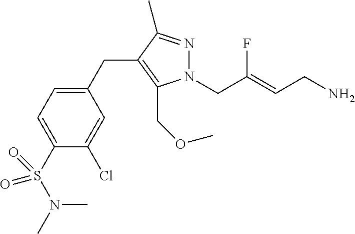 [SMILES: COCc1c(Cc2ccc(S(=O)(=O)N(C)C)c(Cl)c2)c(C)nn1C/C(F)=C/CN] | (Z)-4-((1-(4-amino-2- fluorobut-2-en-1-yl)-5- (methoxymethyl)-3-methyl- 1H-pyrazol-4-yl)methyl)-2- chloro-N,N- dimethylbenzenesulfonamide |
| 39 | 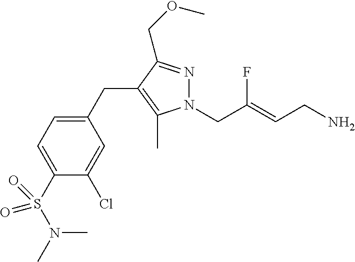 [SMILES: COCc1nn(C/C(F)=C/CN)c(C)c1Cc1ccc(S(=O)(=O)N(C)C)c(Cl)c1] | (Z)-4-((1-(4-amino-2- fluorobut-2-en-1-yl)-3- (methoxymethyl)-5-methyl- 1H-pyrazol-4-yl)methyl)-2- chloro-N,N- dimethylbenzenesulfonamide |
| 40 | 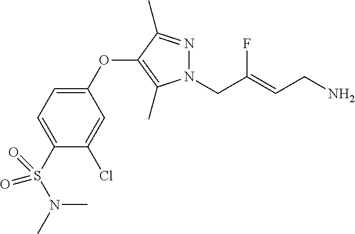 [SMILES: Cc1nn(C/C(F)=C/CN)c(C)c1Oc1ccc(S(=O)(=O)N(C)C)c(Cl)c1] | (Z)-4-((1-(4-amino-2- fluorobut-2-en-1-yl)-3,5- dimethyl-1H-pyrazol-4- yl)oxy)-2-chloro-N,N- dimethylbenzenesulfonamide |
| 41 | 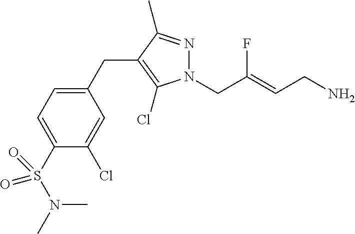 [SMILES: Cc1nn(C/C(F)=C/CN)c(Cl)c1Cc1ccc(S(=O)(=O)N(C)C)c(Cl)c1] | (Z)-4-((1-(4-amino-2- fluorobut-2-en-1-yl)-5- chloro-3-methyl-1H-pyrazol- 4-yl)methyl)-2-chloro-N,N- dimethylbenzenesulfonamide |
| 42 | 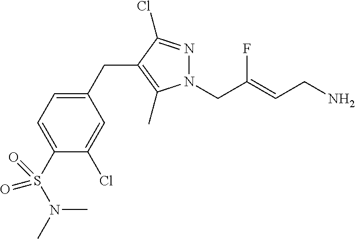 [SMILES: Cc1c(Cc2ccc(S(=O)(=O)N(C)C)c(Cl)c2)c(Cl)nn1C/C(F)=C/CN] | (Z)-4-((1-(4-amino-2- fluorobut-2-en-1-yl)-3- chloro-5-methyl-1H-pyrazol- 4-yl)methyl)-2-chloro-N,N- dimethylbenzenesulfonamide |
| 43 | 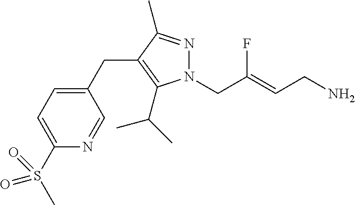 [SMILES: Cc1nn(C/C(F)=C/CN)c(C(C)C)c1Cc1ccc(S(C)(=O)=O)nc1] | (Z)-3-fluoro-4-(5-isopropyl- 3-methyl-4-((6- (methylsulfonyl)pyridin-3- yl)methyl)-1H-pyrazol-1- yl)but-2-en-1-amine |
| 44 | 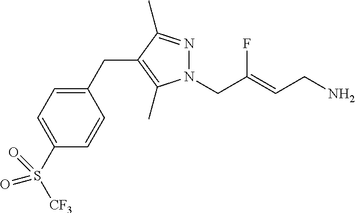 [SMILES: Cc1nn(C/C(F)=C/CN)c(C)c1Cc1ccc(S(=O)(=O)C(F)(F)F)cc1] | (Z)-4-(3,5-dimethyl-4-(4- ((trifluoromethyl)sulfonyl)- benzyl)-1H-pyrazol-1-yl)-3- fluorobut-2-en-1-amine |
| 45 | 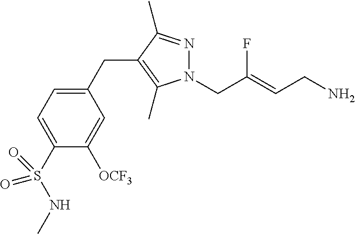 [SMILES: CNS(=O)(=O)c1ccc(Cc2c(C)nn(C/C(F)=C/CN)c2C)cc1OC(F)(F)F] | (Z)-4-((1-(4-amino-2- fluorobut-2-en-1-yl)-3,5- dimethyl-1H-pyrazol-4- yl)methyl)-N-methyl-2- (trifluoromethoxy)- benzenesulfonamide |
| 46 | 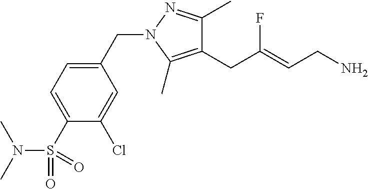 [SMILES: Cc1nn(Cc2ccc(S(=O)(=O)N(C)C)c(Cl)c2)c(C)c1C/C(F)=C/CN] | (Z)-4-((4-(4-amino-2- fluorobut-2-en-1-yl)-3,5- dimethyl-1H-pyrazol-1- yl)methyl)-2-chloro-N,N- dimethylbenzenesulfonamide |
| 47 | 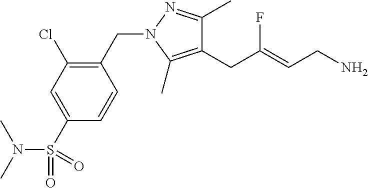 [SMILES: Cc1nn(Cc2ccc(S(=O)(=O)N(C)C)cc2Cl)c(C)c1C/C(F)=C/CN] | (Z)-4-((4-(4-amino-2- fluorobut-2-en-1-yl)-3,5- dimethyl-1H-pyrazol-1- yl)methyl)-3-chloro-N,N- dimethylbenzenesulfonamide |
| 48 | 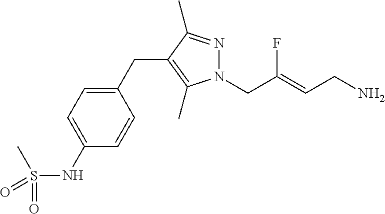 [SMILES: Cc1nn(C/C(F)=C/CN)c(C)c1Cc1ccc(NS(C)(=O)=O)cc1] | (Z)-N-(4-((1-(4-amino-2- fluorobut-2-en-1-yl)-3,5- dimethyl-1H-pyrazol-4- yl)methyl)phenyl)methane- sulfonamide |
| 49 | 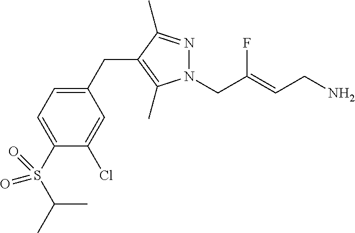 [SMILES: Cc1nn(C/C(F)=C/CN)c(C)c1Cc1ccc(S(=O)(=O)C(C)C)c(Cl)c1] | (Z)-4-(4-(3-chloro-4- (isopropylsulfonyl)benzyl)- 3,5-dimethyl-1H-pyrazol-1- yl)-3-fluorobut-2-en-1-amine |
| 50 | 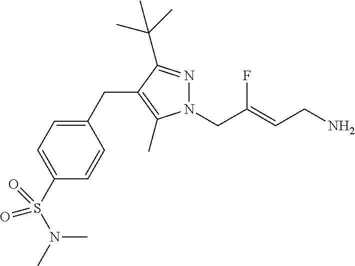 [SMILES: Cc1c(Cc2ccc(S(=O)(=O)N(C)C)cc2)c(C(C)(C)C)nn1C/C(F)=C/CN] | (Z)-4-(1-(4-amino-2- fluorobut-2-en-1-yl)-3-(tert- butyl)-5-methyl-1H-pyrazol- 4-yl)methyl)-N,N- dimethylbenzenesulfonamide |
| 51 | 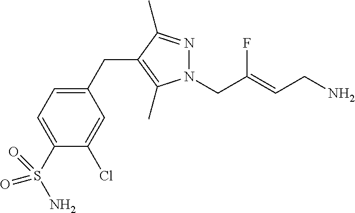 [SMILES: Cc1nn(C/C(F)=C/CN)c(C)c1Cc1ccc(S(N)(=O)=O)c(Cl)c1] | (Z)-4-((1-(4-amino-2- fluorobut-2-en-1-yl)-3,5- dimethyl-1H-pyrazol-4- yl)methyl)-2- chlorobenzenesulfonamide |
| 52 | 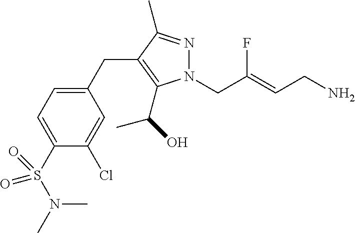 [SMILES: Cc1nn(C/C(F)=C/CN)c([C@H](C)O)c1Cc1ccc(S(=O)(=O)N(C)C)c(Cl)c1] | (Z)-4-((1-(4-amino-2- fluorobut-2-en-1-yl)-5-(1- hydroxyethyl)-3-methyl-1H- pyrazol-4-yl)methyl)-2- chloro-N,N- dimethylbenzenesulfonamide (Enantiomer 1) |
| 53 | 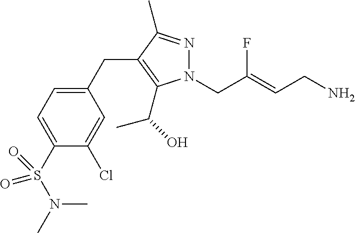 [SMILES: Cc1nn(C/C(F)=C/CN)c([C@@H](C)O)c1Cc1ccc(S(=O)(=O)N(C)C)c(Cl)c1] | (Z)-4-((1-(4-amino-2- fluorobut-2-en-1-yl)-5-(1- hydroxyethyl)-3-methyl-1H- pyrazol-4-yl)methyl)-2- chloro-N,N- dimethylbenzenesulfonamide (Enantiomer 2) |
| 54 | 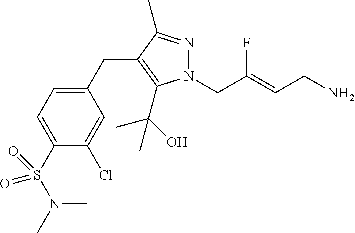 [SMILES: Cc1nn(C/C(F)=C/CN)c(C(C)(C)O)c1Cc1ccc(S(=O)(=O)N(C)C)c(Cl)c1] | (Z)-4-((1-(4-amino-2- fluorobut-2-en-1-yl)-5-(2- hydroxypropan-2-yl)-3- methyl-1H-pyrazol-4- yl)methyl)-2-chloro-N,N- dimethylbenzenesulfonamide |
| 55 | 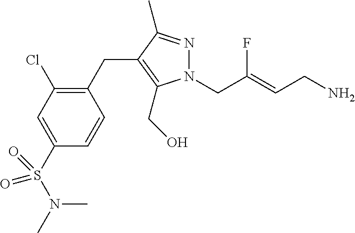 [SMILES: Cc1nn(C/C(F)=C/CN)c(CO)c1Cc1ccc(S(=O)(=O)N(C)C)cc1Cl] | (Z)-4-((1-(4-amino-2- fluorobut-2-en-1-yl)-5- (hydroxymethyl)-3-methyl- 1H-pyrazol-4-yl)methyl)-3- chloro-N,N- dimethylbenzenesulfonamide |
| 56 | 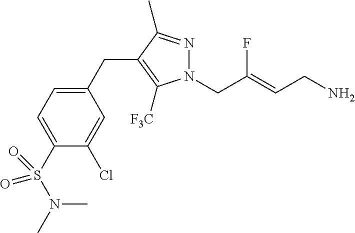 [SMILES: Cc1nn(C/C(F)=C/CN)c(C(F)(F)F)c1Cc1ccc(S(=O)(=O)N(C)C)c(Cl)c1] | (Z)-4-((1-(4-amino-2- fluorobut-2-en-1-yl)-3- methyl-5-(trifluoromethyl)- 1H-pyrazol-4-yl)methyl)-2- chloro-N,N- dimethylbenzenesulfonamide |
| 57 | 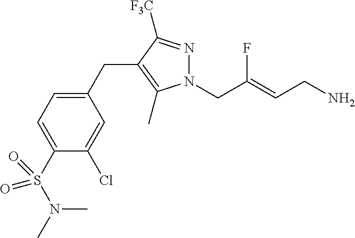 [SMILES: Cc1c(Cc2ccc(S(=O)(=O)N(C)C)c(Cl)c2)c(C(F)(F)F)nn1C/C(F)=C/CN] | (Z)-4-((1-(4-amino-2- fluorobut-2-en-1-yl)-5- methyl-3-(trifluoromethyl)- 1H-pyrazol-4-yl)methyl)-2- chloro-N,N- dimethylbenzenesulfonamide |
| 58 | 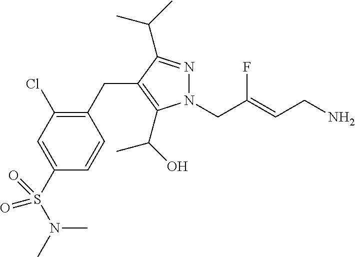 [SMILES: CC(C)c1nn(C/C(F)=C/CN)c(C(C)O)c1Cc1ccc(S(=O)(=O)N(C)C)cc1Cl] | (Z)-4-((1-(4-amino-2- fluorobut-2-en-1-yl)-5-(1- hydroxyethyl)-3-isopropyl- 1H-pyrazol-4-yl)methyl)-3- chloro-N,N- dimethylbenzenesulfonamide |
| 59 | 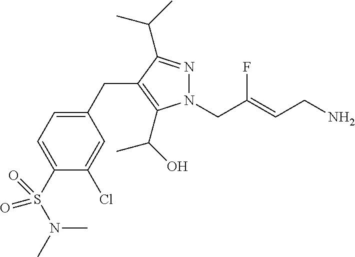 [SMILES: CC(C)c1nn(C/C(F)=C/CN)c(C(C)O)c1Cc1ccc(S(=O)(=O)N(C)C)c(Cl)c1] | (Z)-4-((1-(4-amino-2- fluorobut-2-en-1-yl)-5-(1- hydroxyethyl)-3-isopropyl- 1H-pyrazol-4-yl)methyl)-2- chloro-N,N- dimethylbenzenesulfonamide |
| 60 | 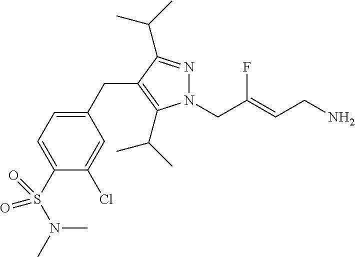 [SMILES: CC(C)c1nn(C/C(F)=C/CN)c(C(C)C)c1Cc1ccc(S(=O)(=O)N(C)C)c(Cl)c1] | (Z)-4-((1-(4-amino-2- fluorobut-2-en-1-yl)-3,5- diisopropyl-1H-pyrazol-4- yl)methyl)-2-chloro-N,N- dimethylbenzenesulfonamide |
| 61 | 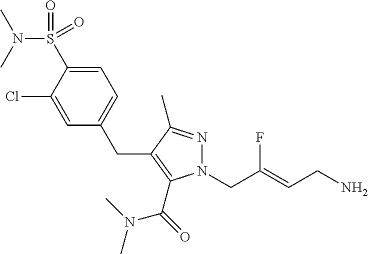 [SMILES: Cc1nn(C/C(F)=C/CN)c(C(=O)N(C)C)c1Cc1ccc(S(=O)(=O)N(C)C)c(Cl)c1] | (Z)-1-(4-amino-2-fluorobut- 2-en-1-yl)-4-(3-chloro-4- (N,N-dimethylsulfamoyl) benzyl)-N,N,3-trimethyl-1H- pyrazole-5-carboxamide |
| 62 | 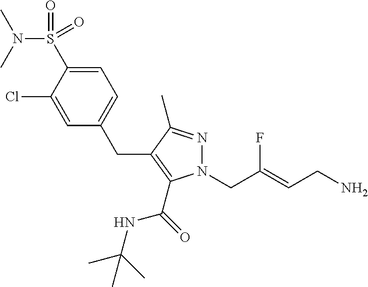 [SMILES: Cc1nn(C/C(F)=C/CN)c(C(=O)NC(C)(C)C)c1Cc1ccc(S(=O)(=O)N(C)C)c(Cl)c1] | (Z)-1-(4-amino-2-fluorobut- 2-en-1-yl)-N-(tert-butyl)- 4-(3-chloro-4-(N,N- dimethylsulfamoyl)benzyl)- 3-methyl-1H-pyrazole-5- carboxamide |
| 63 | 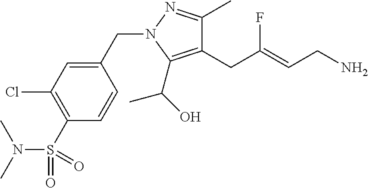 [SMILES: Cc1nn(Cc2ccc(S(=O)(=O)N(C)C)c(Cl)c2)c(C(C)O)c1C/C(F)=C/CN] | (Z)-4-((4-(4-amino-2- fluorobut-2-en-1-yl)-5-(1- hydroxyethyl)-3-methyl-1H- pyrazol-1-yl)methyl)-2- chloro-N,N- dimethylbenzenesulfonamide |
| 64 | 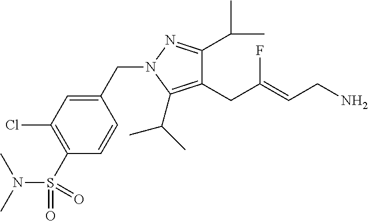 [SMILES: CC(C)c1nn(Cc2ccc(S(=O)(=O)N(C)C)c(Cl)c2)c(C(C)C)c1C/C(F)=C/CN] | (Z)-4-((4-(4-amino-2- fluorobut-2-en-1-yl)-3,5- diisopropyl-1H-pyrazol-1- yl)methyl)-2-chloro-N,N- dimethylbenzenesulfonamide |
| 65 | 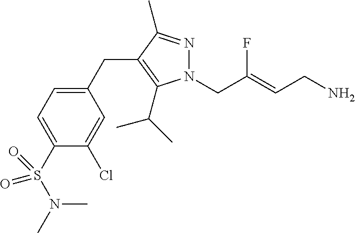 [SMILES: Cc1nn(C/C(F)=C/CN)c(C(C)C)c1Cc1ccc(S(=O)(=O)N(C)C)c(Cl)c1] | (Z)-4-((1-(4-amino-2- fluorobut-2-en-1-yl)-5- isopropyl-3-methyl-1H- pyrazol-4-yl)methyl)-2- chloro-N,N- dimethylbenzenesulfonamide |
| 66 | 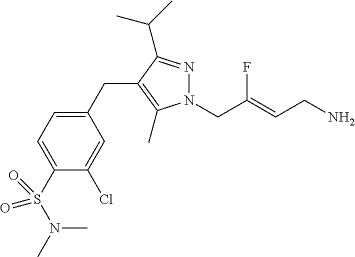 [SMILES: Cc1c(Cc2ccc(S(=O)(=O)N(C)C)c(Cl)c2)c(C(C)C)nn1C/C(F)=C/CN] | (Z)-4-((1-(4-amino-2- fluorobut-2-en-1-yl)-3- isopropyl-5-methyl-1H- pyrazol-4-yl)methyl)-2- chloro-N,N- dimethylbenzenesulfonamide |
| 67 | 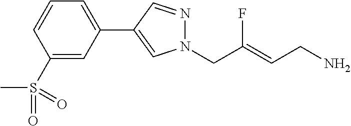 [SMILES: CS(=O)(=O)c1cccc(-c2cnn(C/C(F)=C/CN)c2)c1] | (Z)-3-fluoro-4-(4-(3- (methylsulfonyl)phenyl)- 1H-pyrazol-1-yl)but-2-en-1- amine |

or a pharmaceutically acceptable salt or solvate p thereof.

15. The pharmaceutical composition comprising a compound according to claim 1, or a pharmaceutically acceptable salt or solvate thereof, and at least one pharmaceutically acceptable excipient, carrier or diluent.

16. A method of inhibiting the amine oxidase activity of any one of LOX, LOXL1, LOXL2, LOXL3 or LOXL4 in a subject in need thereof, comprising administering to the subject an effective amount of a compound according to claim 1, or a pharmaceutically acceptable salt or solvate.

17. A method of treating a condition associated with any one of LOX, LOXL1, LOXL2, LOXL3 or LOXL4 protein, comprising administering to a subject in need thereof a therapeutically effective amount of compound according to claim 1, or a pharmaceutically acceptable salt or solvate thereof.

18. The method of claim 17, wherein the condition is a liver disorder, a kidney disorder, a cardiovascular disease, fibrosis, cancer or angiogenesis.

19. The method of claim 18, wherein in a case that the condition is a liver disorder, the liver disorder is selected from the group consisting of biliary atresia, cholestatic liver disease, chronic liver disease, nonalcoholic steatohepatitis (NASH), non-alcoholic fatty liver disease (NAFLD), fatty liver disease associated with disorders such as hepatitis or metabolic syndrome; hepatitis C infection, alcoholic liver disease, primary biliary cirrhosis (PBC), primary schlerosing cholangitis (PSC), liver damage due to progressive fibrosis, liver fibrosis and liver cirrhosis,
wherein in a case that the condition is a kidney disorder, the kidney disorder is selected from the group consisting of kidney fibrosis, renal fibrosis, acute kidney injury, chronic kidney disease, diabetic nephropathy, glomerulosclerosis, vesicoureteral reflux, tubulointerstitial renal fibrosis and glomerulonephritis,
wherein in a case that the condition is a cardiovascular disease, the cardiovascular disease is selected from the group consisting of atherosclerosis, arteriosclerosis, hypercholesteremia, and hyperlipidemia,
wherein in a case that the condition is fibrosis, the fibrosis is selected from the group consisting of liver fibrosis, lung fibrosis, kidney fibrosis, cardiac fibrosis, cystic fibrosis, idiopathic pulmonary fibrosis, radiation-induced fibrosis, ocular fibrosis, Peyronie's disease and scleroderma or is associated with respiratory disease, abnormal wound healing and repair, post-surgical operations, cardiac arrest and all conditions where excess or aberrant deposition of fibrous material is associated with disease, including Crohn's disease and inflammatory bowel disease, and
wherein in a case that the condition is cancer, the cancer is selected from the group consisting of lung cancer; breast cancer; colorectal cancer; anal cancer; pancreatic cancer; prostate cancer; ovarian carcinoma; liver and bile duct carcinoma; esophageal carcinoma; non-Hodgkin's lymphoma; bladder carcinoma; carcinoma of the uterus; glioma, glioblastoma, medullablastoma, and other tumors of the brain; myelofibrosis, kidney cancer; cancer of the head and neck; cancer of the stomach; multiple myeloma; testicular cancer; germ cell tumor; neuroendocrine tumor; cervical cancer; oral cancer, carcinoids of the gastrointestinal tract, breast, and other organs; signet ring cell carcinoma; mesenchymal tumors including sarcomas, fibrosarcomas, haemangioma, angiomatosis, haemangiopericytoma, pseudoangiomatous stromal hyperplasia, myofibroblastoma, fibromatosis, inflammatory myofibroblastic tumour, lipoma, angiolipoma, granular cell tumour, neurofibroma, schwannoma, angiosarcoma, liposarcoma, rhabdomyosarcoma, osteosarcoma, leiomyoma or a leiomysarcoma.

20-28. (canceled)

29. The method according to claim 17 further comprising administering a second therapeutic agent.

30. The method according to claim 29, wherein the second therapeutic agent is selected from the group consisting of an anti-cancer agent, anti-inflammatory agent, anti-hypertensive agent, an anti-fibrotic agent, an anti-angiogenic agent and an immunosuppressive agent.

31. (canceled)
